(19) United States
(12) Patent Application Publication
(10) Pub. No.: US 2022/0249674 A1
(43) Pub. Date: Aug. 11, 2022

(54) BIOACTIVE SAPONIN LINKED TO A FUNCTIONAL MOIETY

(71) Applicants: Sapreme Technologies B.V., Bilthoven (NL); Charité - Universitätsmedizin Berlin, Berlin (DE)

(72) Inventors: Hendrik Fuchs, Berlin (DE); Ruben Postel, Utrecht (NL)

(73) Assignees: Sapreme Technologies B.V., Bilthoven (NL); Charité - Universitätsmedizin Berlin, Berlin (DE)

(21) Appl. No.: 17/629,598

(22) PCT Filed: Jul. 27, 2020
(86) PCT No.: PCT/EP2020/071145

§ 371 Date: Jan. 24, 2022

(30) Foreign Application Priority Data
Jul. 25, 2019 (NL) 2023568

(51) Int. Cl. A61K 47/54; A61K 47/55; A61K 47/60; A61K 47/68; A61K 47/64

(52) U.S. Cl. CPC A61K 47/549; A61K 47/54; A61K 47/642; A61K 47/60; A61K 47/6807; A61K 47/55

(57) ABSTRACT

The invention relates to an endosomal and/or lysosomal escape enhancing conjugate comprising a saponin optionally linked to a targeting molecule such as an antibody and optionally linked to an effector molecule such as a toxin or an oligonucleotide. The invention also relates to a therapeutic combination of such an endosomal and/or lysosomal escape enhancing conjugate of the invention and a functionalized binding molecule comprising an effector molecule, wherein the endosomal and/or lysosomal escape enhancing conjugate comprises an enhancer of said effector molecule, i.e. a saponin. In particular the invention relates to such a therapeutic combination for use as a medicament, in particular for use in the treatment of a tumour. The invention further relates to a method of treating cancer or an autoimmune disease by administering an effective dose of the therapeutic combination to a patient in need thereof or by administering an effective dose of the endosomal and/or lysosomal escape enhancing conjugate comprising a saponin complexed with a targeting molecule such as an immunoglobulin specific for a tumor-cell surface molecule and complexed with an effector molecule, to a patient in need thereof. The invention also relates to a functionalized saponin with endosomal/lysosomal escape enhancing activity.

### TECHNICAL FIELD

The invention relates to an endosomal escape enhancing conjugate and/or lysosomal escape enhancing conjugate comprising a saponin optionally linked to a targeting molecule such as an antibody and optionally linked to an effector molecule such as a toxin. The invention also relates to a therapeutic combination of such an endosomal and/or lysosomal escape enhancing conjugate of the invention and a functionalized binding molecule comprising an effector molecule, wherein the endosomal and/or lysosomal escape enhancing conjugate comprises an enhancer of said effector molecule, i.e. a saponin. In particular the invention relates to such a therapeutic combination for use as a medicament, for example for use in the treatment of a tumour. The invention further relates to a method of treating cancer or an autoimmune disease by administering the therapeutic combination to a patient or by administering an effective dose of the endosomal and/or lysosomal escape enhancing conjugate comprising a saponin linked to a targeting molecule and complexed with an effector molecule, to a patient in need thereof. The invention also relates to enhancement of an effect of an effector molecule, such as a drug, a toxin, a polypeptide or a polynucleotide. In particular, the invention relates to a scaffold or a functionalized scaffold capable of enhancing the effect of an effector molecule, the scaffold comprising at least one glycoside molecule. The invention further relates to use of such scaffold or functionalized scaffold in medicine, in particular for use in the treatment of cancer or acquired or hereditary disorders, in particular monogenic deficiency disorders. The invention further relates to methods for preparing such scaffold or functionalized scaffold and to a pharmaceutical composition comprising such scaffold or functionalized scaffold. The invention also relates to a functionalized saponin endowed with endosomal/lysosomal escape enhancing activity, for application in the manufacture of an endosomal and/or lysosomal escape enhancing conjugate of the invention, the conjugate stimulating intracellular trafficking of payloads delivered across the outer cell membrane of target cells and with a desired intracellular activity outside endosomes and outside lysosomes.

### BACKGROUND

Molecules with a therapeutic biological activity are in many occasions in theory suitable for application as an effective therapeutic drug for the treatment of a disease such as a cancer in human patients in need thereof. A typical example are small-molecule biologically active moieties. However, many if not all potential drug-like molecules and therapeutics currently used in the clinic suffer from at least one of a plethora of shortcomings and drawbacks. When administered to a human body, therapeutically active molecules may exert off-target effects, in addition to the biologically activity directed to an aspect underlying a to-be-treated disease or health problem. Such off-target effects are undesired and bear a risk for induction of health- or even life-threatening side effects of the administered molecule. It is the occurrence of such adverse events that cause many drug-like compounds and therapeutic moieties to fail phase III clinical trials or even phase IV clinical trials (post-market entry follow-up). Therefore, there is a strong desire to provide drug molecules such as small-molecule therapeutics, wherein the therapeutic effect of the drug molecule should, e.g., (1) be highly specific for a biological factor or biological process driving the disease, (2) be sufficiently safe, (3) be sufficiently efficacious, (4) be sufficiently directed to the diseased cell with little to no off-target activity on non-diseased cells, (5) have a sufficiently timely mode of action (e.g. the administered drug molecule should reach the targeted site in the human patient within a certain time frame and should remain at the targeted site for a certain time frame), and/or (6) have sufficiently long lasting therapeutic activity in the patient's body, amongst others. Unfortunately, to date, ‘ideal’ therapeutics with many or even all of the beneficial characteristics here above outlined, are not available to the patients, despite already long-lasting and intensive research and despite the impressive progress made in several areas of the individually addressed encountered difficulties and drawbacks.

Chemotherapy is one of the most important therapeutic options for cancer treatment. However, it is often associated with a low therapeutic window because it has no specificity towards cancer cells over dividing cells in healthy tissue. The invention of monoclonal antibodies offered the possibility of exploiting their specific binding properties as a mechanism for the targeted delivery of cytotoxic agents to cancer cells, while sparing normal cells. This can be achieved by chemical conjugation of cytotoxic effectors (also known as payloads or warheads) to antibodies, to create antibody-drug conjugates (ADCs). Typically, very potent payloads such as emtansine (DM1) are used which have a limited therapeutic index (a ratio that compares toxic dose to efficacious dose) in their unconjugated forms. The conjugation of DM1 to trastuzumab (ado-trastuzumab emtansine), also known as Kadcycla, enhances the tolerable dose of DM1 at least two-fold in monkeys. In the past few decades tremendous efforts and investments have been made to develop therapeutic ADCs. However, it remains challenging to bring ADCs into the clinic, despite promising preclinical data. The first ADC approved for clinical use was gemtuzumab ozogamicin (Mylotarg, CD33 targeted, Pfizer/Wyeth) for relapsed acute myelogenous leukemia (AML) in 2000. Mylotarg was however, withdrawn from the market at the request of the Federal Drug Administration (FDA) due to a number of concerns including its safety profile. Patients treated with Mylotarg were more often found to die than patients treated with conventional chemotherapy. Mylotarg was admitted to the market again in 2017 with a lower recommended dose, a different schedule in combination with chemotherapy or on its own, and a new patient population. To date, only five ADCs have been approved for clinical use, and meanwhile clinical development of approximately fifty-five ADCs has been halted. However, interest remains high and approximately eighty ADCs are still in clinical development in nearly six-hundred clinical trials at present.

Despite the potential to use toxic payloads that are normally not tolerated by patients, a low therapeutic index (a ratio that compares toxic dose to efficacious dose) is a major problem accounting for the discontinuance of many ADCs in clinical development, which can be caused by several mechanisms such as off-target toxicity on normal cells, development of resistance against the cytotoxic agents and premature release of drugs in the circulation. A systematic review by the FDA of ADCs found that the toxicity profiles of most ADCs could be categorized according to the payload used, but not the antibody used, suggesting that toxicity is mostly determined by premature release of the payload. Of the approximately fifty-five ADCs that were discontinued, it is estimated that at least twenty-three were due to a poor therapeutic index. For example, development of a trastuzumab tesirine conjugate (ADCT-502, HER-2 targeted, ADC therapeutics) was recently discontinued due to a narrow therapeutic index, possibly due to an on-target, off-tissue effect in pulmonary tissue which expresses considerable levels of HER2. In addition, several ADCs in phase 3 trials have been discontinued due to missing primary endpoint. For example, phase 3 trials of a depatuxizumab mafodotin conjugate (ABT-414, EGFR targeted, AbbVie) tested in patients with newly diagnosed glioblastoma, and a mirvetuximab soravtansine conjugate (IMGN853, folate receptor alpha (FRα) targeted, ImmunoGen) tested in patients with platinum-resistant ovarian cancer, were recently stopped, showing no survival benefit. It is important to note that the clinically used dose of some ADCs may not be sufficient for its full anticancer activity. For example, ado-trastuzumab emtansine has an MTD of 3.6 mg/kg in humans. In preclinical models of breast cancer, ado-trastuzumab emtansine induced tumor regression at dose levels at or above 3 mg/kg, but more potent efficacy was observed at 15 mg/kg. This suggests that at the clinically administered dose, ado-trastuzumab emtansine may not exert its maximal potential anti-tumor effect.

ADCs are mainly composed of an antibody, a cytotoxic moiety such as a payload, and a linker. Several novel strategies have been proposed and carried out in the design and development of new ADCs to overcome the existing problems, targeting each of the components of ADCs. For example, by identification and validation of adequate antigenic targets for the antibody component, by selecting antigens which have high expression levels in tumor and little or no expression in normal tissues, antigens which are present on the cell surface to be accessible to the circulating ADCs, and antigens which allows internalizing of ADCs into the cell after binding; and alternative mechanisms of activity; design and optimize linkers which enhance the solubility and the drug-to-antibody ratio (DAR) of ADCs and overcome resistance induced by proteins that can transport the chemotherapeutic agent out of the cells; enhance the DAR ratio by inclusion of more payloads, select and optimize antibodies to improve antibody homogeneity and developability. In addition to the technological development of ADCs, new clinical and translational strategies are also being deployed to maximize the therapeutic index, such as, change dosing schedules through fractionated dosing; perform biodistribution studies; include biomarkers to optimize patient selection, to capture response signals early and monitor the duration and depth of response, and to inform combination studies.

An example of ADCs with clinical potential are those ADCs such as brentuximab vedotin, inotuzumab ozogamicin, moxetumomab pasudotox, and polatuzumab vedotin, which are evaluated as a treatment option for lymphoid malignancies and multiple myeloma. Polatuzumab vedotin, binding to CD79b on (malignant) B-cells, and pinatuzumab vedotin, binding to CD22, are tested in clinical trials wherein the ADCs each were combined with co-administered rituximab, a monoclonal antibody binding to CD20 and not provided with a payload [B. Yu and a Liu, Antibody-drug conjugates in clinical trials for lymphoid malignancies and multiple myeloma; Journal of Hematology & Oncology (2019) 1294]. Combinations of monoclonal antibodies such as these examples are yet a further approach and attempt to arrive at the ‘magic bullet’ which combines many or even all of the aforementioned desired characteristics of ADCs.

Meanwhile in the past few decades, nucleic acid-based therapeutics are under development. Therapeutic nucleic acids can be based on deoxyribonucleic acid (DNA) or ribonucleic acid (RNA), Anti-sense oligonucleotides (ASOs, AONs), and short interfering RNAs (siRNAs), MicroRNAs, and DNA and RNA aptamers, for approaches such as gene therapy, RNA interference (RNAi). Many of them share the same fundamental basis of action by inhibition of either DNA or RNA expression, thereby preventing expression of disease-related abnormal proteins. The largest number of clinical trials is being carried out in the field of gene therapy, with almost 2600 ongoing or completed clinical trials worldwide but with only about 4% entering phase 3. This is followed by clinical trials with ASOs. Similarly to ADCs, despite the large number of techniques being explored, therapeutic nucleic acids share two major issues during clinical development: delivery into cells and off-target effects. For instance, ASOs such as peptide nucleic acid (PNA), phosphoramidate morpholino oligomer (PMO), locked nucleic acid (LNA) and bridged nucleic acid (BNA), are being investigated as an attractive strategy to inhibit specifically target genes and especially those genes that are difficult to target with small molecules inhibitors or neutralizing antibodies. Currently, the efficacy of different ASOs is being studied in many neurodegenerative diseases such as Huntington's disease, Parkinson's disease, Alzheimer's disease, and amyotrophic lateral sclerosis and also in several cancer stages. The application of ASOs as potential therapeutic agents requires safe and effective methods for their delivery to the cytoplasm and/or nucleus of the target cells and tissues. Although the clinical relevance of ASOs has been demonstrated, inefficient cellular uptake, both in vitro and in vivo, limit the efficacy of ASOs and has been a barrier to therapeutic development. Cellular uptake can be <2% of the dose resulting in too low ASO concentration at the active site for an effective and sustained outcome. This consequently requires an increase of the administered dose which induces off-target effects. Most common side-effects are activation of the complement cascade, the inhibition of the clotting cascade and toll-like receptor mediated stimulation of the immune system.

Chemotherapeutics are most commonly small molecules, however, their efficacy is hampered by the severe off-target side toxicity, as well as their poor solubility, rapid clearance and limited tumor exposure. Scaffold-small-molecule drug conjugates such as polymer-drug conjugates (PDCs) are macromolecular constructs with pharmacologically activity, which comprises one or more molecules of a small-molecule drug bound to a carrier scaffold (e.g. polyethylene glycol (PEG)).

Such conjugate principle has attracted much attention and has been under investigation for several decades. The majority of conjugates of small-molecule drugs under pre-clinical or clinical development are for oncological indications. However, up-to-date only one drug not related to cancer has been approved (Movantik, a PEG oligomer conjugate of opioid antagonist naloxone, AstraZeneca) for opioid-induced constipation in patients with chronic pain in 2014, which is a non-oncology indication. Translating application of drug-scaffold conjugates into treatment of human subjects provides little clinical success so far. For example, PK1 (N-(2-hydroxypropyl)methacrylamide (HPMA) copolymer doxorubicin; development by Pharmacia, Pfizer) showed great anti-cancer activity in both solid tumors and leukemia in murine models, and was under clinical investigation for oncological indications. Despite that it demonstrated significant reduction of nonspecific toxicity and improved pharmacokinetics in man, improvements in anticancer efficacy turned out to be marginal in patients, and as a consequence further development of PK1 was discontinued.

The failure of scaffold-small-molecule drug conjugates is at least partially attributed to its poor accumulation at the tumor site. For example, while in murine models PK1 showed 45-250 times higher accumulation in the tumor than in healthy tissues (liver, kidney, lung, spleen, and heart), accumulation in tumor was only observed in a small subset of patients in the clinical trial.

A potential solution to the aforementioned problems is application of nanoparticle systems for drug delivery such as liposomes. Liposomes are sphere-shaped vesicles consisting of one or more phospholipid bilayers, which are spontaneously formed when phospholipids are dispersed in water. The amphiphilicity characteristics of the phospholipids provide it with the properties of self-assembly, emulsifying and wetting characteristics, and these properties can be employed in the design of new drugs and new drug delivery systems. Drug encapsulated in a liposomal delivery system may convey several advantages over a direct administration of the drug, such as an improvement and control over pharmacokinetics and pharmacodynamics, tissue targeting property, decreased toxicity and enhanced drug activity. An example of such success is liposome-encapsulated form of a small molecule chemotherapy agent doxorubicin (Doxil: a pegylated liposome-encapsulated form of doxorubicin; Myocet: a non-pegylated liposomal doxorubicin), which have been approved for clinical use.

Therefore, a solution still needs to be found that allows for drug therapies such as anti-tumor therapies, applicable for non-systemic use when desired, wherein the drug has for example an acceptable safety profile, little off-target activity, sufficient efficacy, sufficiently low clearance rate from the patient's body, etc.

In order for a bioactive molecule, i.e. a functional moiety, to exert its effect, the molecule must be able to engage with its target, e.g. in the blood serum, on the outside of the cell surface or inside a cell or inside an organelle of a cell. The active moiety of for example almost all protein-based targeted toxins and nucleotide-based drugs must enter the cytosol of the target cell to mediate their target modulatory effect. In many constellations for example the toxin remains ineffective since the targeted toxin (1) is poorly internalized and remains bound to the outside of the cells, (2) is recycled back to the cell surface after internalization, or (3) transported to the endo-lysosomes where it is degraded. Even though these fundamental issues have been known for decades and more than 500 targeted toxins have been investigated in the past years, the problems have not yet been solved and only a limited number of antibody-targeted protein toxins have been approved for tumor therapeutic applications by the regulatory authorities to date, including Ontak, Lumoxiti and Elzonris. A further important problem relates to toxicity (often vascular leakage syndrome) induced by such protein toxins, most likely by premature release of the toxin and immunogenecity towards the protein. The therapeutic window is therefore limited. To overcome these problems, many strategies have been described, including approaches to redirect the toxins to endogenous cellular membrane transport complexes of the biosynthetic pathway in the endoplasmic reticulum, and techniques to disrupt or weaken the membrane integrity of endosomes, i.e. the compartments of the endocytic pathway in a cell, and thus facilitating the endosomal and/or lysosomal escape. This comprises the use of lysosomotropic amines, carboxylic ionophores, calcium channel antagonists, various cell-penetrating peptides of viral, bacterial, plant, animal, human and synthetic origin, other organic molecules, and light-induced techniques. Although the efficacy of the targeted toxins was typically augmented hundred or thousand fold in the targeted cells, in exceptional cases more than million fold, the requirement to co-administer endosomal and/or lysosomal escape enhancers with other substances harbors new problems including additional side effects, loss of target specificity, difficulties to determine the therapeutic window and cell type-dependent variations. All strategies, including physicochemical techniques, require enhancer molecules that interact more or less directly with membranes and comprise essentially small chemical molecules, secondary metabolites, peptides and proteins. A common feature of all these substances is that they are not target cell-specific per se and are distributed through other kinetics than the targeted toxins. This is one major drawback of the approaches known up till now.

Examples of substances that show a very promising potential to enhance the endosomal and/or lysosomal escape of targeted toxins, although suffering from the previously mentioned drawbacks, are some secondary plant metabolites. In particular some glycosylated triterpenoids (saponins) of the oleanane type isolated from Gypsophila paniculata L. (baby's breath) and Saponaria officinalis L. (common soapwort) have the ability to augment the cytotoxicity of several ribosome-inactivating proteins (RIPs). This augmentation is neither due to a permeabilization of the plasma membrane—it occurs at non-permeabilizing concentrations of the glycosylated triterpenoid—nor due to an increase of the rate of endocytic events, but rather due to the mediation of an enhanced endosomal and/or lysosomal escape into the cytosol, after the proteins have been taken up into the endosomes. The acidic environment in the endosomes, such as the late endosomes, is a prerequisite for the synergistic action of the saponins in combination with a toxin, since inhibition of vesicle acidification by bafilomycin A or chloroquine restored the survival of the tested cells. It is postulated that the protonation of the glucuronic acid, which is present in this particular group of saponins, and interaction with endosomal cholesterol may play an important role. Targeted toxins can become enhanced in their cell killing efficacy by these saponins, dependent on cell line and amount of target receptor expression, by 3,000-fold up to 4,000,000-fold, which resulted in a broadening of the therapeutic window in mice between 10-fold to 500-fold. After injection of the targeted toxins, a substantial reduction in the tumor volume occurred and complete remissions were seen in many cases of different tumor models. The tumor regression across all these studies was in average more than 90% and the required toxin dose was only 2% of the dose used for a treatment without saponins.

SO1861 from Saponaria officinalis L. and SA1641 from Saponinum album (a saponin composite from Gypsophila spec.) are two of the few saponins that were found to display such tremendous synergism with several type I RIPs, such as saporin, dianthin or agrostin. In contrast, the cytotoxicity of the A chain from the type II RIP ricin was only enhanced 16-fold and the cytotoxicity of the bacterial Pseudomonas exotoxin A remained unaffected by the combinatory treatment. This can be most likely attributed to the divergent intracellular routing of these toxins omitting those acidic compartments and vesicles of the cell that are the site of action of the mentioned saponins, such as for example late endosomes and lysosomes.

In European patent EP1623715B1, a composition comprising a pharmacologically active agent coupled to a target-cell specific component, combined with a saponin, has been described. The pharmacologically active agent is for example a toxin.

### SUMMARY

Up to the present invention, approaches to enhance endosomal and/or lysosomal escape of targeted toxins rely on glycosides passively diffusing through the plasma membrane and reaching the endosomal membranes at the same time as the targeted toxin. Such approaches have, however, several drawbacks such as lack of timely synchronization of the targeted toxin and the glycoside and non-specific effects of the glycoside in non-target cells. The present invention provides novel approaches to redirect both the effector and the endosomal and/or lysosomal escape enhancer via targeting ligands to the acidic compartments of the endocytic pathway of the target cell.

An aspect of the invention relates to a functionalized glycoside moiety having endosomal and/or lysosomal escape enhancing activity and having a molecular structure comprising at least one S moiety and at least one connector moiety L*, with general structure (0):

S-(L*)ₘ structure (0),

wherein the S moiety is a glycoside,
wherein
the at least one L* moiety is at least one W* moiety,

wherein the at least one W* moiety is any one or more of:
a reactive group ‘*’ on the at least one S moiety, providing S*, the reactive group ‘*’ for linking the at least one S* moiety to at least a first moiety L* via the reactive group ‘*’ on the S* moiety,
a linker, the linker comprising a reactive group ‘*’ for linking of the at least one S moiety to a further moiety F;
a first proteinaceous molecule;
a scaffold, consisting of, or comprising
   an oligomeric structure, or
   a polymeric structure,
      wherein the oligomeric structure and the polymeric structure comprises, or is selected from, any of:
         a polymer;
         an oligomer;
         a dendrimer;
         a dendron;
         a dendronized polymer;
         a dendronized oligomer;
         an assembly of any of a polymer, an oligomer, a dendrimer, a dendron, a dendronized polymer, a dendronized oligomer,
         wherein the polymer, oligomer, dendrimer, dendron, dendronized polymer, dendronized oligomer, are any of
         linear;
         branched; or
         cyclic,
            wherein the scaffold comprises a single reactive group ‘*’ for coupling a single S moiety, or
            wherein the scaffold comprises more than one reactive group ‘*’, each group for coupling a single S moiety,
            wherein the scaffold comprises a single binding site for binding a further moiety F, or
            wherein the scaffold comprises multiple binding sites for binding multiple further moieties F,
            said binding sites for one or more further moieties F on the scaffold moiety W* being reactive groups ‘*’ on the scaffold moiety W* for provision of a bond with at least one further moiety F,
            wherein the at least one S moiety is linked, coupled or bound to the reactive group ‘*’ on the W* moiety through a bond,

wherein m is at least 1 and at most equal to the number of reactive groups ‘*’ on the at least one S moiety,
wherein the L* moieties are the same or different for m>1;
wherein the W* moieties are the same or different for m>1;
   or
   wherein the at least one L* moiety is an O* moiety,

wherein the O* moiety is a trifunctional linker comprising three reactive groups ‘*’ for linking one S moiety and two further moieties F, or for linking two S moieties and one further moiety F, or wherein the O* moiety is a linker with at least three functionalities comprising at least three reactive groups ‘*’ for linking at least one S moiety and at least two further moieties F, or for linking at least two S moieties and at least one further moiety F
wherein the three reactive groups ‘*’ or the at least three reactive groups ‘*’ are the same or different;
wherein the O* moieties are the same or different for m>1;
   or wherein the at least one L* moiety is one or more W* moieties and/or one or more O* moieties, wherein more than two W* moieties and O* moieties together are coupled in a linear fashion or are coupled in a branched order relative to the S moiety,
   wherein ‘*’ depicts a binding site or reactive group for binding an S moiety, a W moiety, an O moiety, to a further moiety S, W*, O*, or a further moiety F.

wherein F moieties are the same or different when the functionalized glycoside moiety encompasses more than one F moiety.

An aspect of the invention relates to a functionalized glycoside moiety having endosomal and/or lysosomal escape enhancing activity and having a molecular structure comprising at least one S moiety and at least one connector moiety L*, with general structure (0):

S-(L*)ₘ structure (0),

wherein the S moiety is a glycoside,

preferably the at least one S moiety is any of
a bisdesmosidic triterpene,
a bisdesmosidic triterpene saponin,
a bisdesmosidic triterpene saponin belonging to the type of a 12,13-dehydrooleanane with an aldehyde function, in position 23,
a saponin isolatable from species Gypsophila,
a saponin isolatable from species Saponaria,
a saponin selected from:
   SA1641 or a diastereomer thereof,
   SO1861 or a diastereomer thereof, and
   GE1741 or a diastereomer thereof;
   and preferably the at least one S moiety is SO1861;
      wherein
      the at least one L* moiety is at least one W* moiety,

wherein the at least one W* moiety is any one or more of:
a reactive group ‘*’ on the at least one S moiety, providing S*, the reactive group ‘*’ preferably selected from
   an aldehyde group,
   a carboxylic acid group,
   an alkenyl group,
   an hydroxyl group,
for linking the at least one S* moiety to at least a first moiety L* via the reactive group ‘*’ on the S* moiety,
a linker, such as a chemical linker or a linear or non-linear stretch of amino-acid residues complexed through peptide bonds and/or disulphide bonds, the linker comprising a reactive group ‘*’ for linking of the at least one S moiety to a further moiety F, preferably the linker is N-ε-maleimidocaproic acid hydrazide for conjugating a sulfhydryl, such as in a cysteine, to a carbonyl such as in an aldehyde or in a ketone, or preferably the linker is succinimidyl 3-(2-pyridyldithio)propionate, wherein the F moiety is any one or more of a payload, a further S moiety, a further linker, a scaffold, a ligand, an effector molecule, an antibody, EGF, a toxin, an oligonucleotide such as an RNA, a BNA, a DNA, an LNA;
a first proteinaceous molecule such as a first peptide, a first polypeptide, or a first protein, preferably the first protein is an antibody, an immunoglobulin, or a binding domain thereof or a binding fragment thereof, such as an immunoglobulin G, a Fab fragment, an scFv, at least one Vh domain, at least one VHH domain;
   wherein the first proteinaceous molecule comprises a single reactive group ‘*’ for coupling a single S moiety, or
   wherein the first proteinaceous molecule comprises more than one reactive group ‘*’, each group for coupling a single S moiety,
   wherein the first proteinaceous molecule comprises a single binding site for a single further moiety F, or
   wherein the first proteinaceous molecule comprises multiple binding sites for multiple further moieties F,
      said binding sites on the first proteinaceous molecule being reactive groups ‘*’ on the first proteinaceous molecule for provision of a bond with a further moiety F, such as a covalent bond, a non-covalent bond, an electrostatic interaction, a hydrogen bond, a salt bridge, a van der Waals interaction, a hydrophobic interaction, preferably a covalent bond,
a scaffold, consisting of, or comprising
   an oligomeric structure, or
   a polymeric structure,
      wherein the oligomeric structure and the polymeric structure comprises, or is selected from, any of:
         a polymer;
         an oligomer;
         a dendrimer;
         a dendron;
         a dendronized polymer;
         a dendronized oligomer;
         an assembly of any of a polymer, an oligomer, a dendrimer, a dendron, a dendronized polymer, a dendronized oligomer,
         wherein the polymer, oligomer, dendrimer, dendron, dendronized polymer, dendronized oligomer, are any of
         linear;
         branched; or
         cyclic,
            wherein the scaffold comprises a single reactive group ‘*’ for coupling a single S moiety, preferably a terminal S moiety, or
            wherein the scaffold comprises more than one reactive group ‘*’, each group for coupling a single S moiety, preferably a terminal S moiety,
            wherein the scaffold comprises a single binding site for binding a further moiety F, or
            wherein the scaffold comprises multiple binding sites for binding multiple further moieties F,
            said binding sites for one or more further moieties F on the scaffold moiety W* being reactive groups ‘*’ on the scaffold moiety W* for provision of a bond with at least one further moiety F, such as a covalent bond, a non-covalent bond, an electrostatic interaction, a hydrogen bond, a salt bridge, a van der Waals interaction, a hydrophobic interaction, preferably a covalent bond,
            wherein the at least one S moiety is linked, coupled or bound to the reactive group ‘*’ on the W* moiety through a bond, such as a covalent bond, a non-covalent bond, an electrostatic interaction, a hydrogen bond, a salt bridge, a van der Waals interaction, a hydrophobic interaction, preferably a covalent bond,

wherein said (covalent) bond is optionally a cleavable bond, wherein said cleavable bond is preferably subject to cleavage under any one or more of:
acidic conditions, preferably at a pH of lower than 6.5 such as pH 4.0-6.5, and preferably at a pH ≤5.5;
reductive conditions;
enzymatic conditions; and
light-induced conditions,
   wherein the cleavable bond is optionally selected from:

an imine bond;

a hydrazone bond;

a 1,3-dioxolane bond; and

an ester bond, and/or
wherein the cleavable bond is a disulfide bond or a peptide bond or an amide bond,

wherein m is at least 1 and at most equal to the number of reactive groups on the at least one S moiety, the reactive groups ‘*’ selected preferably from an aldehyde group, a carboxylic acid group, an alkenyl group, and an hydroxyl group, for linking the at least one S moiety to a further L* moiety, and preferably m=1,
wherein the L* moieties are the same or different for m>1;
wherein the W* moieties are the same or different for m>1;
   or
   wherein the at least one L* moiety is an O* moiety,

wherein the O* moiety is a trifunctional linker comprising three reactive groups ‘*’ for linking one S moiety and two further moieties F, or for linking two S moieties and one further moiety F, or wherein the O* moiety is a linker with at least three functionalities comprising at least three reactive groups ‘*’ for linking at least one S moiety and at least two further moieties F, or for linking at least two S moieties and at least one further moiety F
wherein the three reactive groups ‘*’ or the at least three reactive groups ‘*’ are the same or different;
wherein the O* moieties are the same or different for m>1;
   or wherein the at least one L* moiety is one or more W* moieties and/or one or more O* moieties, wherein more than two W* moieties and O* moieties together are coupled in a linear fashion or are coupled in a branched order relative to the S moiety, such as for example S-W*-O*-O*, S-O*-W*-O*, S-O*-O*-W*, S-W*(-O*)₂, S-O*(-W*)(-O*),
   wherein ‘*’ depicts a binding site or reactive group for binding an S moiety, a W moiety, an O moiety, to a further moiety S, W*, O*, or a further moiety F.

wherein F moieties are the same or different when the functionalized glycoside moiety encompasses more than one F moiety.

An aspect of the invention relates to an endosomal and/or lysosomal escape enhancing conjugate having a molecular structure comprising at least one S moiety, at least one connector moiety L and at least one E moiety, with general structure (I):

S(-L-E)ₙ structure (I),

wherein the at least one S moiety is a glycoside;
wherein
the at least one L moiety is at least one W moiety,

wherein the at least one W moiety is any one or more of:
a reactive group ‘*’ on the at least one S moiety, for linking the at least one S moiety to at least a first moiety L via the reactive group ‘*’,
a linker comprising a reactive group ‘*’ for direct linking of the at least one S moiety to a single E moiety;
   a first proteinaceous molecule wherein the first proteinaceous molecule comprises a single reactive group ‘*’ for coupling a single S moiety, or wherein the first proteinaceous molecule comprises more than one reactive group ‘*’, each group ‘*’ for coupling a single S moiety,
   wherein the first proteinaceous molecule comprises a single binding site for a single moiety E, or
   wherein the first proteinaceous molecule comprises multiple binding sites for multiple moieties E,
      said binding sites on the first proteinaceous molecule being reactive groups ‘*’ on the first proteinaceous molecule for provision of a bond with a moiety E,
a scaffold, consisting of, or comprising
   an oligomeric structure, or
   a polymeric structure,
      wherein the oligomeric structure and the polymeric structure comprises, or is selected from, any of:
         a polymer;
         an oligomer;
         a dendrimer;
         a dendron;
         a dendronized polymer;
         a dendronized oligomer;
         an assembly of any of a polymer, an oligomer, a dendrimer, a dendron, a dendronized polymer, a dendronized oligomer,
         wherein the polymer, oligomer, dendrimer, dendron, dendronized polymer, dendronized oligomer, are any of
         linear;
         branched; or
         cyclic,
            wherein the scaffold comprises a single reactive group ‘*’ for coupling a single S moiety, or
            wherein the scaffold comprises more than one reactive group ‘*’, each group ‘*’ for coupling a single S moiety,
            wherein the scaffold comprises a single binding site for binding a single E moiety, or
            wherein the scaffold comprises multiple binding sites for binding multiple E moieties,
            said binding sites for one or more E moieties on the scaffold moiety W being reactive groups ‘*’ on the scaffold moiety W for provision of a bond with at least one E moiety,
            wherein the at least one S moiety is linked, coupled or bound to the reactive group ‘*’ on the W moiety through a bond,

wherein n is at least 1 and at most equal to the number of reactive groups ‘*’ on the at least one S moiety,
wherein the L moieties are the same or different for n>1;
wherein the W moieties are the same or different for n>1;
   or
   wherein the at least one L moiety is an O moiety,

wherein the O moiety is a trifunctional linker comprising three reactive groups ‘*’ for linking one S moiety and two E moieties, or for linking two S moieties and one E moiety, or wherein the O moiety is a linker with at least three functionalities comprising at least three reactive groups ‘*’ for linking at least one S moiety and at least two E moieties, or for linking at least two S moieties and at least one E moiety
wherein the O moieties are the same or different for n>1;
   or wherein the at least one L moiety is one or more W moieties and/or one or more O moieties, wherein more than two W moieties and O moieties together are coupled in a linear fashion or are coupled in a branched order, relative to a first coupled E moiety,

wherein the at least one E moiety is any one or more of:
(i) one S moiety or more than one S moieties, wherein the more than one S moieties are the same or different;
(ii) at least one payload selected from one effector moiety or more effector moieties; and
(iii) one ligand or more ligands,
   wherein the at least one S moiety is a glycoside;
   wherein the effector moiety or the effector moieties is/are selected from any one or more of:
a molecule with pharmaceutical activity;
a toxin;
a nucleotide;
an enzyme;
a second protein; and
a second peptide,
   wherein the ligand(s) is/are selected from any one or more of:
a binding partner for a target cell surface molecule; and
an immunoglobulin or a binding domain or binding fragment thereof, for binding to such a cell surface molecule,
   wherein the effector moiety/moieties and the ligand(s) are directly coupled to any of the scaffold, the at least one S moiety, the trifunctional linker O, the linker, the first proteinaceous molecule,

and/or wherein a first effector moiety or a first ligand is directly coupled to any of the scaffold, the at least one S moiety, the trifunctional linker O, the linker, the first proteinaceous molecule,
wherein the E moieties are the same or are different for n>1;
wherein effector moieties are the same or different for n>1; and
wherein ligands are the same or different for n>1.

An aspect of the invention relates to an endosomal and/or lysosomal escape enhancing conjugate having a molecular structure comprising at least one S moiety, at least one connector moiety L and at least one E moiety, with general structure (I):

S(-L-E)ₙ structure (I),

wherein the at least one S moiety is a glycoside,

preferably the at least one S moiety is any of
a bisdesmosidic triterpene,
a bisdesmosidic triterpene saponin,
a bisdesmosidic triterpene saponin belonging to the type of a 12,13-dehydrooleanane with an aldehyde function, in position 23,
a saponin isolatable from species Gypsophila,
a saponin isolatable from species Saponaria,

a saponin selected from:
SA1641 or a diastereomer thereof,
SO1861 or a diastereomer thereof, and
GE1741 or a diastereomer thereof;
and preferably the at least one S moiety is SO1861;
   wherein
   the at least one L moiety is at least one W moiety,

wherein the at least one W moiety is any one or more of:
a reactive group ‘*’ on the at least one S moiety, preferably selected from
   an aldehyde group,
   a carboxylic acid group,
   an alkenyl group,
   an hydroxyl group,
   for linking the at least one S moiety to at least a first moiety L via the reactive group ‘*’,
a linker, such as a chemical linker or a linear or non-linear stretch of amino-acid residues complexed through peptide bonds and/or disulphide bonds and/or chemical bonds, the linker comprising a reactive group ‘*’ for direct linking of the at least one S moiety to a single E moiety through preferably a single bond, preferably the linker is N-ε-maleimidocaproic acid hydrazide for conjugating a sulfhydryl, such as in a cysteine, to a carbonyl such as in an aldehyde or in a ketone, or preferably the linker is succinimidyl 3-(2-pyridyldithio)propionate;
a first proteinaceous molecule such as a first peptide, a first polypeptide, or a first protein, preferably the first protein is an antibody, an immunoglobulin, or a binding domain thereof or a binding fragment thereof, such as an immunoglobulin G, a Fab fragment, an scFv, at least one Vh domain, at least one VHH domain;
   wherein the first proteinaceous molecule comprises a single reactive group ‘*’ for coupling a single S moiety, or
   wherein the first proteinaceous molecule comprises more than one reactive group ‘*’, each group ‘*’ for coupling a single S moiety,
   wherein the first proteinaceous molecule comprises a single binding site for a single moiety E, or
   wherein the first proteinaceous molecule comprises multiple binding sites for multiple moieties E,
      said binding sites on the first proteinaceous molecule being reactive groups ‘*’ on the first proteinaceous molecule for provision of a bond with a moiety E, such as a covalent bond, a non-covalent bond, an electrostatic interaction, a hydrogen bond, a salt bridge, a van der Waals interaction, a hydrophobic interaction, preferably a covalent bond,
a scaffold, consisting of, or comprising
   an oligomeric structure, or
   a polymeric structure,
      wherein the oligomeric structure and the polymeric structure comprises, or is selected from, any of:
         a polymer;
         an oligomer;
         a dendrimer;
         a dendron;
         a dendronized polymer;
         a dendronized oligomer;
         an assembly of any of a polymer, an oligomer, a dendrimer, a dendron, a dendronized polymer, a dendronized oligomer,
         wherein the polymer, oligomer, dendrimer, dendron, dendronized polymer, dendronized oligomer, are any of
         linear;
         branched; or
         cyclic,
            wherein the scaffold comprises a single reactive group ‘*’ for coupling a single S moiety, or
            wherein the scaffold comprises more than one reactive group ‘*’, each group ‘*’ for coupling a single S moiety,
            wherein the scaffold comprises a single binding site for binding a single E moiety, or
            wherein the scaffold comprises multiple binding sites for binding multiple E moieties,
            said binding sites for one or more E moieties on the scaffold moiety W being reactive groups ‘*’ on the scaffold moiety W for provision of a bond with at least one E moiety, such as a covalent bond, a non-covalent bond, an electrostatic interaction, a hydrogen bond, a salt bridge, a van der Waals interaction, a hydrophobic interaction, preferably a covalent bond,
            wherein the at least one S moiety is linked, coupled or bound to the reactive group ‘*’ on the W moiety through a bond, such as a covalent bond, a non-covalent bond, an electrostatic interaction, a hydrogen bond, a salt bridge, a van der Waals interaction, a hydrophobic interaction, preferably a covalent bond,

wherein said (covalent) bond is optionally a cleavable bond, wherein said cleavable bond is preferably subject to cleavage under any one or more of:
acidic conditions, preferably at a pH of lower than 6.5 such as pH 4.0-6.5, preferably at a pH ≤5.5;
reductive conditions;
enzymatic conditions; and
light-induced conditions,
wherein the cleavable bond is optionally selected from:

an imine bond;

a hydrazone bond;

a 1,3-dioxolane bond; and

an ester bond, and/or
wherein the cleavable bond is a disulfide bond or a peptide bond or an amide bond,

wherein n is at least 1 and at most equal to the number of reactive groups ‘*’ on the at least one S moiety, the reactive groups ‘*’ selected preferably from an aldehyde group, a carboxylic acid group, an alkenyl group, and an hydroxyl group, for linking the at least one S moiety to a further L moiety, and preferably n=1,
wherein the L moieties are the same or different for n>1;
wherein the W moieties are the same or different for n>1;
   or
   wherein the at least one L moiety is an O moiety,

wherein the O moiety is a trifunctional linker comprising three reactive groups ‘*’ for linking one S moiety and two E moieties, or for linking two S moieties and one E moiety, or wherein the O moiety is a linker with at least three functionalities comprising at least three reactive groups ‘*’ for linking at least one S moiety and at least two E moieties, or for linking at least two S moieties and at least one E moiety
wherein the O moieties are the same or different for n>1;
   or wherein the at least one L moiety is one or more W moieties and/or one or more O moieties, wherein more than two W moieties and O moieties together are coupled in a linear fashion or are coupled in a branched order, relative to a first coupled E moiety, such as for example in the branched order S-W*-O*-O*, S-O*-W*-O*, S-O*-O*-W*, S-W*(-O*)₂, S-O*(-W*)(-O*),

wherein the at least one E moiety is any one or more of:
(i) one S moiety or more than one S moieties, wherein the more than one S moieties are the same or different;
(ii) at least one payload selected from one effector moiety or more effector moieties; and
(iii) one ligand or more ligands,
   wherein the at least one S moiety is a glycoside,
   preferably the at least one S moiety is any of
a bisdesmosidic triterpene,
a bisdesmosidic triterpene saponin,
a bisdesmosidic triterpene saponin belonging to the type of a 12,13-dehydrooleanane with an aldehyde function, in position 23,
a saponin isolatable from species Gypsophila,
a saponin isolatable from species Saponaria,
a saponin selected from:
   SA1641 or a diastereomer thereof,
   SO1861 or a diastereomer thereof, and
   GE1741 or a diastereomer thereof;
      and preferably the at least one S moiety is SO1861;
      wherein the effector moiety or the effector moieties is/are selected from any one or more of:
a molecule with pharmaceutical activity, such as a drug molecule, including, but not being limited to a macromolecule or a small molecule;
a toxin, such as a macromolecular cell-killing agent, a protein toxin, an immunotoxin, saporin, dianthin, ribosomal inactivating protein, a small molecule cell-killing agent, a small molecule toxin;
a nucleotide, preferably an oligonucleotide, an RNA, a DNA, an LNA, a BNA, (bridged nucleic acid), an aptamer, a nucleic acid, a plasmid, a vector, a gene, an ASO (allele-specific oligonucleotide), an antisense oligonucleotide (ASO), an miRNA (microRNA), an siRNA (small interfering RNA);

an enzyme;

a second protein; and

a second peptide,

wherein the ligand(s) is/are selected from any one or more of:
a binding partner for a target cell surface molecule, preferable a target cell surface molecule specific for an aberrant cell such as a tumor cell, the target cell surface molecule preferably selected from any of HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CEACAM5, HER3, CD74, PTK7, Notch3, FGF2, C4.4A, FLT3, CD71, CD38, FGFR3, CD123, DLL3, such as the binding partner EGF for cell-surface receptor EGFR or transferrin for transferrin receptor; and
an immunoglobulin or a binding domain or binding fragment thereof, for binding to for example such a cell surface molecule such as cell-surface receptor HER2 and cell-surface receptor EGFR, such as immunoglobulin trastuzumab for binding to HER2 and immunoglobulin cetuximab for binding to EGFR and anti-CD71 monoclonal antibody for binding to cell-surface receptor CD71 (transferrin receptor),
   wherein the S moiety/moieties is/are preferably (a) terminal moiety/moieties,
   wherein the effector moiety/moieties and the ligand(s) are directly coupled to any of the scaffold, the at least one S moiety, the trifunctional linker O, the linker such as a chemical linker, the first proteinaceous molecule such as the first peptide, the first polypeptide, and the first protein,

and/or wherein a first effector moiety or a first ligand is directly coupled to any of the scaffold, the at least one S moiety, the trifunctional linker O, the linker, preferably a chemical linker, the first proteinaceous molecule such as the first peptide, the first polypeptide, the first protein, and wherein optionally a second, a third and further effector moiety/moieties and/or optionally a second, a third and further ligand(s) is/are coupled to said first, second or third effector moiety or is/are coupled to said first, second, or third ligand, either directly, or through a linker, in linear fashion in any order of two or more effector moieties and/or two or more ligands, and/or in branched fashion,
wherein optionally one or more S moiety/moieties is/are coupled to said first, second, third and further effector moiety/moieties and/or to said first, second, third and further ligand(s), preferably S moiety/moieties is/are coupled directly to an effector moiety or to a ligand, or is/are coupled to an effector moiety or to a ligand via an L moiety such as a linker, a trifunctional linker, and/or a scaffold, wherein the scaffold is preferably a dendron or a dendrimer and wherein the S moiety is preferably linked to the scaffold via a linker or a trifunctional linker, wherein the bond between an S moiety and an L moiety is a non-cleavable bond or a cleavable bond, preferably a cleavable bond, wherein said cleavable bond is preferably subject to cleavage under any one or more of:
acidic conditions, preferably at a pH of lower than 6.5 such as pH 4.0-6.5, preferably at a pH of ≤5.5;
reductive conditions;
enzymatic conditions; and
light-induced conditions,
wherein the cleavable bond is optionally selected from:
   an imine bond;
   a hydrazone bond;
   a 1,3-dioxolane bond; and
   an ester bond,
   and/or wherein the cleavable bond is a disulfide bond or a peptide bond or an amide bond,
wherein the E moieties are the same or are different for n>1;
wherein effector moieties are the same or different for n>1; and
wherein ligands are the same or different for n>1.

An aspect of the invention relates to a combination of an endosomal and/or lysosomal escape enhancing conjugate according to the invention and a binding moiety, wherein the binding moiety comprises at least one covalently or non-covalently bound effector molecule as outlined for the endosomal and/or lysosomal escape enhancing conjugate, wherein the endosomal and/or lysosomal escape enhancing conjugate and the binding moiety are, independently from one another, able to bind to a target cell surface molecule or target cell surface structure, such as specifically binding to a target cell surface molecule or target cell surface structure, wherein the target cell surface molecule and target cell surface structure are preferably a target cell surface molecule and a target cell surface structure specifically exposed on the target cell surface, thereby inducing receptor-mediated endocytosis of the endosomal and/or lysosomal escape enhancing conjugate, and of the binding moiety.

An aspect of the invention relates to a combination of a first binding molecule and a second binding molecule, wherein the first binding molecule comprises at least one glycoside molecule, wherein the second binding molecule comprises at least one effector molecule, wherein the first binding molecule and the second binding molecule are, independently from one another, able to specifically bind to a target cell-specific surface molecule or structure, thereby inducing receptor-mediated endocytosis of a complex of the first binding molecule and the target cell-specific surface molecule, and of the complex of the second binding molecule and the target cell-specific surface molecule.

In one aspect, the invention provides a combination of two binding molecules, wherein a first binding molecule comprises at least one glycoside molecule and wherein a second binding molecule comprises at least one effector molecule, wherein the glycoside molecule and the effector molecule are not bound to one and the same binding molecule and wherein the first and the second binding molecule are, independently from one another, able to specifically bind to a target cell-specific surface molecule or structure, thereby inducing receptor-mediated endocytosis of the complex of binding molecule and target cell-specific surface molecule. This approach increases the specific uptake of the saponin and, therefore, the effect of said saponin on the targeted cell, whereas at the same time, a non-specific effect on non-targeted cells is decreased.

In a further aspect, the invention provides the combination comprising the first binding molecule and the second binding molecule according to the invention for use as a medicament, in particular for use in the treatment of cancer. The increase in effect of the effector molecule in target cells and the decrease in effect in non-target cells increases the therapeutic window of the effector molecule, thereby making the combination according to the invention very useful as a medicament.

A further aspect of the invention relates to a composition comprising at least one endosomal and/or lysosomal escape enhancing conjugate according to the invention, such as one such conjugate or two such conjugates, and one or more of at least one saponin bearing endosomal/lysosomal escape enhancing activity, at least one effector moiety as outlined for the endosomal and/or lysosomal escape enhancing conjugate, and at least one ligand as outlined for the endosomal and/or lysosomal escape enhancing conjugate.

In a further aspect, the invention provides a pharmaceutical composition comprising the combination comprising the first binding molecule and the second binding molecule according to the invention or comprising the composition of the invention, and a pharmaceutically acceptable excipient. Such pharmaceutical composition is very useful for use in a combination therapy, for instance for the treatment of cancer.

In a further aspect, the invention provides a method for treating a patient in need thereof with the combination comprising the first binding molecule and the second binding molecule according to the invention or comprising the composition of the invention or a pharmaceutical composition according to the invention.

An aspect of the invention relates to a kit comprising a first container containing the first binding molecule according to the invention and a second container containing the second binding molecule according to the invention, the kit further comprising instructions for using the content of both containers.

An “endosomal and/or lysosomal escape enhancing conjugate” is herein defined as a conjugate facilitating the endosomal and/or lysosomal escape of a moiety, molecule, peptide, DNA, LNA, toxin, immunotoxin, oligonucleotide, vector, RNA, BNA, protein, enzyme, immunotoxin, etc., etc., wherein the term “endosomal and/or lysosomal” has its regular scientific meaning throughout the specification and the claims, and here includes all acidic compartments and vesicles of the endocytic pathway, degradation pathway and recycling pathway of a cell.

An embodiment is the endosomal and/or lysosomal escape enhancing conjugate wherein the E moiety is at least a ligand, such as an immunoglobulin.

An embodiment is the endosomal and/or lysosomal escape enhancing conjugate of the invention, wherein S is a bisdesmosidic triterpene saponin belonging to the type of a 12,13-dehydrooleanane with an aldehyde function, in position 23, and wherein S is preferably a saponin that can be isolated from Gypsophila or Saponaria species, more preferably S is the saponin SO1861 or any of its diastereomers, and n=1, wherein E is an immunoglobulin or at least a binding domain thereof for binding to a cell surface molecule, wherein preferably the cell surface molecule is selected from any of HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CD38, FGFR3, CD123, DLL3, CEACAM5, HER3, CD74, PTK7, Notch3, FGF2, C4.4A, FLT3, CD71, L is a linker coupled to the glycoside via a cleavable bond, and E is an immunoglobulin, wherein preferably said cleavable bond is subject to cleavage under acidic, reductive, enzymatic or light-induced conditions, and preferably the cleavable bond is a covalent bond, preferably an imine bond, a hydrazone bond, an oxime bond, a 1,3-dioxolane bond or an ester bond. Preferred is a cleavable bond, wherein the cleavable bond is a disulfide bond or a peptide bond. For example, such a peptide bond is cleavable by a proteolytic enzyme.

An embodiment is the endosomal and/or lysosomal escape enhancing conjugate of the invention, wherein n is 1.

An embodiment is the endosomal and/or lysosomal escape enhancing conjugate of the invention, wherein the S moiety is a terminal saponin, preferably the saponin SO1861, the L moiety is a chemical linker, and E is a terminal single ligand moiety or a terminal single immunoglobulin such as trastuzumab or cetuximab, the linker preferably providing a cleavable bond between the terminal S moiety and the terminal E moiety, and n is 1.

An aspect of the invention relates to a combination of an endosomal and/or lysosomal escape enhancing conjugate of the invention and a binding moiety, wherein the binding moiety comprises at least one effector molecule, wherein the endosomal and/or lysosomal escape enhancing conjugate and the binding moiety are, independently from one another, able to specifically bind to a target cell-specific surface molecule or structure, thereby inducing receptor-mediated endocytosis of a complex of the endosomal and/or lysosomal escape enhancing conjugate and the target cell-specific surface molecule, and of the complex of the binding moiety and the target cell-specific surface molecule.

A further aspect of the invention relates to a pharmaceutical composition comprising a combination according to the invention and a pharmaceutically acceptable excipient.

An aspect of the invention relates to the combination or to the pharmaceutical composition for use as a medicament, in particular for use in a method of treating cancer or in a method for treating an autoimmune disease.

An aspect of the invention relates to a method of treating cancer, the method comprising administering a combination according to the invention to a patient in need thereof. A further aspect of the invention relates to a method of treating cancer, the method comprising administering a pharmaceutical composition of the invention, to a patient in need thereof.

An aspect of the invention relates to a kit comprising a first container containing the endosomal and/or lysosomal escape enhancing conjugate according to the invention and a second container containing the binding moiety according to the invention.

The use of a targeted saponin of the invention for improving endolysosomal escape of a payload facilitates the opportunity to lower the concentration of an ADC-protein toxin to be administered to a patient in need thereof, and therewith the application of a targeted saponin of the invention improves the therapeutic window. By using the targeted saponin of the invention in a therapeutic regimen comprising the administration of an ADC-protein toxin, the effective dose of said ADC-protein toxin to be administered to a patient in need thereof is lower than when the targeted saponin would not be applied.

### BRIEF DESCRIPTION OF THE FIGURES

FIG. 1: The targeted 2-component approach. SO1861 and toxin (ribosomal inactivating protein) are each, separately, conjugated to an antibody (mAb) for delivery and internalization into target cells. 1) mAb-toxin and mAb-SO1861 bind to their corresponding cell surface receptor, 2) receptor-mediated endocytosis of both conjugates occurs, 3) at low endolysosomal pH and appropriate concentration, SO1861 becomes active to enable endolysosomal escape, 4) release of toxin into cytoplasm occurs and 5) toxin induces cell death. Top row: the monoclonal antibody conjugated with the saponin is the same type of monoclonal antibody conjugated with the toxin, targeting the same cell-surface molecule such as a receptor (mAb1). Bottom row: the monoclonal antibody (mAb1) conjugated with the saponin is different from the monoclonal antibody conjugated with the toxin (mAb2). Here, mAb1 and mAb2 bind to different cell-surface molecules such as two different receptors.

FIG. 2. 1-target 2-component (EGFR high expression). EGFR targeted cell killing in A431 (EGFR⁺⁺⁺) and CaSki (EGFR⁺⁺), by a therapeutic combination according to the invention. A, B) cetuximab-SO1861 titration in combination with a fixed concentration of 10 pM cetuximab-saporin shows that a 100-400 fold reduced concentration of conjugated SO1861 is required, versus unconjugated SO1861, to induce cell killing by cetuximab-saporin. C, D), Cetuximab-saporin titration in combination with 278 nM cetuximab-SO1861 can kill cells in contrast to 300 nM unconjugated SO1861+cetuximab-saporin. 1500 nM unconjugated SO1861+cetuximab-saporin is more efficient compared to the therapeutic combination, since both cetuximab conjugates compete for the same EGFR receptor. Only simultaneous targeted delivery of both cetuximab conjugates leads to efficient cell-killing, in contrast to monotherapy with either conjugate alone.

FIG. 3: 1-target 2-component (EGFR no/low expression). EGFR targeted cell killing in HeLa (EGFR⁺) and A2058 (EGFR⁻) cells, by a therapeutic combination according to the invention. A, B) cetuximab-SO1861 titration in combination with a fixed concentration of 10 pM cetuximab-saporin do not induce cell killing by cetuximab-saporin. C, D), Cetuximab-saporin titration in combination with 278 nM cetuximab-SO1861 cannot induce cell killing. Low EGFR receptor expression is prohibitive for sufficient SO1861 to be delivered via antibody-mediated delivery, while 1500 nM of unconjugated SO1861 induces efficient cell killing.

FIG. 4: 1-target 2-component (HER2 high expression). HER2 targeted cell killing in SK-BR3 (HER2⁺⁺⁺) cells by a therapeutic combination according to the invention. A) Trastuzumab-SO1861 titration in combination with a fixed concentration of 673 pM trastuzumab-saporin shows that a 1000 fold reduced concentration of conjugated SO1861 is required, versus unconjugated SO1861, to induce cell killing by trastuzumab-saporin. C, D), Trastuzumab-saporin titration in combination with 9.4 nM Trastuzumab-SO1861 can kill cells in contrast to 10 nM unconjugated SO1861+trastuzumab-saporin. 1075 nM unconjugated SO1861+trastuzumab-saporin is more efficient compared to the therapeutic combination, since both trastuzumab conjugates compete for the same HER2 receptor. Only simultaneous targeted delivery of both trastuzumab conjugates leads to efficient cell-killing, in contrast to monotherapy with either conjugate alone.

FIG. 5: 1-target 2-component (HER2 no/low expression). HER2 targeted cell killing in JIMT-1 (HER2⁺) and A431 (HER2^{+/-}) cells, by a therapeutic combination according to the invention. A, B) trastuzumab-SO1861 titration in combination with a fixed concentration of 50 pM trastuzumab-saporin does not induce cell killing by trastuzumab-saporin. C, D), Trastuzumab-saporin titration in combination with 10 nM trastuzumab-SO1861 cannot induce cell killing. Low HER2 receptor expression is prohibitive for sufficient SO1861 to be delivered via antibody-mediated delivery, while 1500 nM of unconjugated SO1861 induces efficient cell killing.

FIG. 6: 2-target 2-component (EGFR high expression and HER2 low expression). EGFR/HER2 targeted cell killing in A431 (EGFR⁺⁺⁺/HER2^{+/-}) and CaSki (EGFR⁺⁺/HER2^{+/-}) cells by a therapeutic combination according to the invention. A, B) Cetuximab-SO1861 titration in combination with a fixed concentration of 50 pM trastuzumab-saporin shows a 100 fold reduced concentration of conjugated SO1861 is required, versus unconjugated SO1861, to induce cell killing by trastuzumab-saporin. C, D), Trastuzumab-saporin titration in combination with 278 nM cetuximab-SO1861 can kill cells in contrast to 300 nM unconjugated SO1861+trastuzumab-saporin. 1500 nM unconjugated SO1861+trastuzumab-saporin has comparable cell killing efficiency compared to the therapeutic combination, 278 nM cetuximab-SO1861+trastuzumab-saporin, since both conjugates do not compete for the same receptor. Only simultaneous targeted delivery of both conjugates leads to efficient cell-killing, in contrast to monotherapy with either conjugate alone.

FIG. 7. 2-target 2-component (EGFR low expression and HER2 no/low expression). EGFR/HER2 targeted cell killing in HeLa (EGFR⁺/HER2^{+/-}) and A2058 (EGFR⁻/HER2^{+/-}) cells by a therapeutic combination according to the invention. A, B) Cetuximab-SO1861 titration in combination with a fixed concentration of 50 pM trastuzumab-saporin does not induce cell killing by trastuzumab-saporin. C, D), Trastuzumab-saporin titration in combination with 278 nM cetuximab-SO1861 does not potentiate cell killing, while 1500 nM of unconjugated SO1861 induces efficient cell killing. Low EGFR receptor expression is prohibitive for sufficient SO1861 to be delivered via antibody-mediated delivery.

FIG. 8. 2-target 2-component (HER2 high expression and EGFR low expression). HER2 targeted cell killing in SK-BR-3 (HER2⁺⁺⁺/EGFR^{+/-}) cells by a therapeutic combination according to the invention. A) Trastuzumab-SO1861 titration in combination with a fixed concentration of 1.5 pM EGFdianthin shows that a 400-fold reduced concentration of conjugated SO1861 is required, versus unconjugated SO1861, to induce cell killing by EGFdianthin. B), EGFdianthin titration in combination with 9.4 nM trastuzumab-SO1861 can kill cells in contrast to 10 nM unconjugated SO1861+EGFdianthin. 1075 nM unconjugated SO1861+EGFdianthin has comparable cell killing efficiency compared to the therapeutic combination, 9.4 nM trastuzumab-SO1861+EGFdianthin, since both conjugates do not compete for the same receptor. Only simultaneous targeted delivery of both conjugates leads to efficient cell-killing, in contrast to monotherapy with either conjugate alone.

FIG. 9. 2-target 2-component (HER2 low expression and EGFR low or high expression). HER2 targeted cell killing in JIMT-1 (HER2⁺) and A431 (HER2^{+/-}) cells, by a therapeutic combination according to the invention. A, B) trastuzumab-SO1861 titration in combination with a fixed concentration of 5 pM cetuximab-saporin does not induce cell killing by cetuximab-saporin. C, D), Cetuximab-saporin titration in combination with 10 nM trastuzumab-SO1861 cannot induce cell killing. Low HER2 receptor expression is prohibitive for sufficient SO1861 to be delivered via antibody-mediated delivery, while 1500 nM of unconjugated SO1861 induces efficient cell killing. Even a high EGFR receptor expression level (D) for delivery of cetuximab-saporin does not change its potency in the presence of trastuzumab-SO1861, indicating that the bottleneck for cell-killing activity is a too low HER2 expression level, leading to insufficient SO1861 inside target cells to switch on endosomal and/or lysosomal escape.

FIG. 10. 2-target 2-component versus T-DM1. Cells with high EGFR expression and low HER2 expression (A431) can efficiently be killed with the therapeutic combination according to the invention. However, T-DM1 is not effective at such low toxin concentrations. T-DM1 is Trastuzumab-emtansine (Kadcyla®), carrying ˜3.5 DM1 toxin molecules per antibody.

FIG. 11. A-E displays the relative cell viability when trastuzumab (FIG. 11A), cetuximab (FIG. 11B) or T-DM1 (FIG. 11C), free toxins saporin (FIG. 11D) and dianthin (FIG. 11D), saporin coupled to a non-cell binding IgG (FIG. 11D), and saporin coupled to a non-cell binding IgG combined with free saponin SO1861 (FIG. 11E) are contacted with the indicated cell lines SK-BR-3, JIMT-1, MDA-MB-468, A431, CaSki, HeLa, A2058, BT-474.

FIG. 12. FIG. 12A displays a cartoon of an IgG with four effector moieties, here toxin molecules, covalently linked to the light chains and the heavy chains of the IgG (via lysines and cysteines of the antibody). FIG. 12B shows a ligand fora cell-surface receptor, here EGF for the EGFR, covalently coupled to a toxin. FIG. 12C displays an immunoglobulin with four endosomal and/or lysosomal escape enhancing molecules attached to it, here four saponin molecules linked to the light chain and the heavy chain of the IgG through chemical linkers (via lysines and cysteines of the immunoglobulin).

FIG. 13. Shown is the coupling reaction of the linking of four moieties of a plant-derived saponin ‘SPT001’ (here, saponin SO1861) to four cysteines of an antibody. First, the disulphide bonds in the IgG are disrupted under influence of exposure to TCEP (Tris(2-carboxyethyl)phosphine) for a controlled time-span; second, the saponin SPT001 comprising a chemical linker bound to it, is added together with trifluoro acetic acid, and four saponin moieties are linked to the IgG. For producing cleavable ‘ready to conjugate’ saponins the aldehyde group of SO1861 was reacted with an EMCH (ε-maleimidocaproic acid hydrazide) linker. The hydrazide group of EMCH forms an acid cleavable hydrazone bond with the aldehyde of SO1861. At the same time the EMCH linker presents a maleimide group that is thiol (sulfhydryl group) reactive and thus can be conjugated to thiols of the IgG, i.e. the ligand moiety. Herewith, an endosomal and/or lysosomal escape enhancing conjugate of the invention is provided, and/or a first binding molecule of the invention is provided.

FIG. 14: Basic scaffold with click chemistry function to link any desired effector molecule. The user determines the position of the click chemistry position in the effector molecule and all further properties of the effector molecule, e.g. choice and position of an optional ligand.

FIG. 15: Functionalized scaffold with pre-bound effector molecule and click chemistry function to link any desired ligand. Optionally, a pH-sensitive linkage can be provided to release the effector molecule from the scaffold after reaching the endosomes.

FIG. 16: Scaffold precursor with four amino groups for saponin linkage and an azide group for click chemistry.

FIG. 17: Evidence for the coupling of saponins to the model scaffold. The inset shows the theoretically expected peaks and intensity distribution for coupled saponins. The experimental data obtained by LC-MS/ESI-MS show almost exactly the same peaks at m/z 758-760 Da proving successful saponin coupling.

FIG. 18: Cytotoxicity assays using the targeted toxin dianthin-Epidermal Growth Factor (dianthin-EGF). Untreated cells were normalized to 1. The polymeric structure (Pentrimer) has no influence on cell viability neither in the presence nor in the absence of Dianthin-EGF and saponin (SA1641) indicating no intrinsic cytotoxicity of the polymeric structure. The clickable targeted toxin (Dianthin-EGF-Alkyne) has a markedly reduced activity, which is a result of the toxin modification but does not have any relation to the scaffold. The functionalized polymeric structure has the same activity as the unclicked targeted toxin, indicating that the functionalization of the scaffold does not impair effector molecule activity. The effect of saponins is identical in the presence and absence of the polymeric structure showing that the polymeric structure does not impair the efficacy of the saponins in the two-component system.

FIG. 19: H-NMR spectrum of (A) SO1861 and (B) SO1861-EMCH (EMCH=N-ε-maleimidocaproic acid hydrazide). (A) The peak at 9.43 ppm (H^{a}) corresponds to the aldehyde proton of SO1861. (B) The peak at 6.79 ppm (H^{c}) corresponds to the maleimide protons of SO1861-EMCH, while the peak at 7.68 ppm (H^{b}) corresponds to the hydrazone proton. The absence of the signal at 9.43 ppm indicates a quantitative conversion of the aldehyde group.

FIG. 20: (A) MALDI-TOF-MS spectrum of SO1861-EMCH and (B) SO1861-EMCH-mercaptoethanol. (A) RP mode: m/z 2124 Da ([M+K]⁺, saponin-EMCH), m/z 2109 Da ([M+K]⁺, SO1861-EMCH), m/z 2094 Da ([M+Na]⁺, SO1861-EMCH). (B) RP mode: m/z 2193 Da ([M+K]⁺, saponin-EMCH-mercaptoethanol), m/z 2185 Da ([M+K]⁺, SO1861-EMCH-mercaptoethanol), m/z 2170 Da ([M+Na]⁺, SO1861-EMCH-mercaptoethanol).

FIG. 21: SO1861 structure with highlighted chemical groups for conjugation of endosomal escape enhancing saponins to a polymeric structure. Highlighted groups are aldehyde (black circle), carboxylic acid (dashed circle), alkene (dashed pentagon), and alcohol (dashed box). The aldehyde group (arrow) is most suitable group for chemoselective and reversible conjugation reactions.

FIG. 22: Strategy for producing (A) stable and (B) cleavable ‘ready-to conjugate’ endosomal escape enhancer saponins.

FIG. 23: Hydrolysis of the hydrazone bond of SO1861-EMCH under acidic conditions.

FIG. 24: SO1861-EMCH structure. (A) Standard molecular structure, and (B) 3D model. Maleimide group is marked with a circle.

FIG. 25: (A) SO1861-EMCH synthesis scheme. (B) MALDI-TOF-MS spectra of SO1861 (m/z 1861 Da) and (C) SO1861-EMCH (m/z 2068 Da) in negative reflector mode. TFA: trifluoroacetic acid, r.t: room temperature, h: hours, and MW: molecular weight.

FIG. 26: MALDI-TOF-MS spectra of SO1861-EMCH (A) before and (B) after hydrolysis in HCl solution at pH 3.

FIG. 27: Reaction scheme of SO1861-EMCH conjugation to any amine-bearing polymeric structure.

FIG. 28: MALDI-TOF-MS spectra of (A) BSA-SO1861 (m/z 70.0 kDa, 72.1 kDa, 74.2 kDa), and (B) BSA (m/z 66.6 kDa).

FIG. 29: Reaction scheme of (A) SO1861-EMCH and (B) SO1861-HATU (HATU=1-[Bis(dimethylamino)methylene]-1H-1,2,3-triazolo[4,5-b]pyridinium 3-oxid hexafluorophosphate) conjugation to a cyanine 3 dye labeled polyamidoamine (PAMAM) G5 dendrimer.

FIG. 30: MALDI-TOF-MS spectra of (A) Cy3-PAMAM, (B-D) Cy3-PAMAM-SO1861 with increasing SO1861-EMCH feed equivalents from (B) up to bottom (D). (B) corresponds to Cy3-PAMAM-SO1861 with 5 SO1861 attached per PAMAM, (C) corresponds to Cy3-PAMAM-SO1861 with 13 SO1861 attached per PAMAM, and (D) corresponds to Cy3-PAMAM-SO1861 with 51 SO1861 attached per PAMAM.

FIG. 31: MALDI-TOF-MS spectra of (A) Cy3-PAMAM-SO1861 with 5 equivalents feed SO1861-EMCH and (B) Cy3-PAMAM-SO1861 with 30 equivalents feed SO1861-EMCH.

FIG. 32: MALDI-TOF-MS spectra of Cy3-PAMAM-NC-SO1861 (NC=stable bond (“non-cleavable”).

FIG. 33: (A) Reaction scheme and MALDI-TOF-MS spectra of (B) Cy3-PAMAM-NC-SO1861-Dibenzocyclooctyne (DBCO), (C) Cy3-PAMAM-(SO1861)₅-DBCO, and (D) Cy3-PAMAM-(SO1861)27-DBCO.

FIG. 34: Reaction scheme of (A) dianthin-EGF-Alexa488 and (B) dianthin-EGF-Alexa488-SS-PEG-N₃. MALDI-TOF-MS spectra of (C) dianthin-EGF, (D) dianthin-EGF-Alexa488, and (E) dianthin-EGF-Alexa488-SS-PEG-N3; Alexa488: Alexa Fluor 488 dye.

FIG. 35: Reaction scheme of (A) dianthin-Alexa488 and (B) dianthin-Alexa488-SS-PEG-N3. MALDI-TOF-MS spectra of (C) dianthin, (D) dianthin-Alexa488, and (E) dianthin-Alexa488-SS-PEG-N3; Alexa488: Alexa Fluor 488 dye.

FIG. 36: Fluorescence images of SDS-PAGE gel performed on a VersaDoc imaging system. M=marker, P=Cy3-PAMAM-(SO1861)₂₇-DBCO, D=dianthin-EGF-Alexa488-SS-PEG-N3, C1=Cy3-PAMAM-(SO1861)₅-Dianthin-EGF-Alexa488, C2=Cy3-PAMAM-NC-SO1861-Dianthin-EGF-Alexa488, and C3=Cy3-PAMAM-(SO1861)₂₇-Dianthin-EGF-Alexa488.

FIG. 37: (A) Synthesis scheme of Cy3-PAMAM-NC-SO1861 via reductive amination. (B, and C) Respective MALDI-TOF-MS spectra.

FIG. 38: Reaction scheme for the generation of poly(SO1861) using SO1861-EMCH as monomer, the APS/TMEDA system as polymerization initiator, and aminopropanethiol as radical quencher.

FIG. 39: MALDI-TOF-MS spectra of poly(SO1861) reaction batches. (A) SO1861-EMCH at 60° C., (B) SO1861-EMCH+11⁻³ equivalents APS at 60° C., (C) SO1861-EMCH+11⁻³ equivalents APS/TMEDA at 60° C.

FIG. 40: DNA approach. Usage of the principle of DNA-origami to generate a DNA based scaffold that is able to conjugate and release glycoside molecules. In addition, one of the DNA strands obtains a click chemistry moiety that can be used for conjugation to a targeted toxin to form a functionalized scaffold. bp: base pair.

FIG. 41: Poly(peptide-SO1861) approach. Usage of a peptide sequence that can conjugate and release glycoside molecules and which can react with itself to form a poly(peptide-SO1861) construct. The poly(peptide) chain endings can be further modified with click chemistry moieties (e.g., BCN-NHS linker) that can be used for conjugation to a toxin.

FIG. 42. MALDI-TOF-MS spectra of (A) native peptide, (B) peptide-SO1861 conjugate.

FIG. 43. Molecular structure of G4-dendron with protected amino groups.

FIG. 44. Synthesis scheme for the generation of dendron based scaffolds and functional scaffolds.

FIG. 45. (A) Reaction scheme for partial dye labeling and deprotection of the G4-dendron. (B) MALDI-TOF-MS spectrum of deprotected and partially dye labeled G4-dendron.

FIG. 46. MALDI-TOF-MS spectra of G4-dendron-SO1861 scaffolds with (A) 22 feed equivalents of SO1861-EMCH, (B) 10 feed equivalents of SO1861-EMCH, and (C) 3 feed equivalents of SO1861-EMCH.

FIG. 47. Cell viability curves of HeLa cells treated with (A) EGFR cell surface expression as determined by FACS analyses of HeLa cells (B), cell viability of HeLa cells treated with SO1861+dianthin-EGF (Dia-EGF), SO1861+dianthin-EGF+500 nM chloroquine, SO1861+dianthin-EGF+500 nM PAMAM, SO1861+dianthin-EGF+667 nM dendron (C) cell viability of HeLa cells treated with SO1861+dianthin-EGF, SO1861+dianthin-EGF+500 nM chloroquine, SO1861+dianthin-EGF+500 nM PAMAM, SO1861+dianthin-EGF+500 nM PAMAM-(SH)₁₆, SO1861+dianthin-EGF+500 nM PAMAM-(SH)₆₅, SO1861+dianthin-EGF+500 nM PAMAM-(SH)₁₀₈ (D) cell viability of HeLa cells treated with SO1861+dianthin-EGF, SO1861+dianthin-EGF+500 nM chloroquine, SO1861+dianthin-EGF+500 nM PAMAM, SO1861+dianthin-EGF+500 nM PAMAM-(mPEG)₃, SO1861+dianthin-EGF+500 nM PAMAM-(mPEG)₈, SO1861+dianthin-EGF+500 nM PAMAM-(mPEG)₁₈.

FIG. 48. (A) Reaction scheme of the thiolation of PAMAM using the thiolation reagent 2-iminothiolane. MALDI-TOF-MS spectra of (B) native PAMAM, (C) thiolated PAMAM-(SH)₁₆, (D) thiolated PAMAM-(SH)₆₅, and (E) thiolated PAMAM-(SH)₁₀₈.

FIG. 49. (A) Reaction scheme of the PEGylation of PAMAM using the PEGylating reagent mPEG₂ₖ-NHS. MALDI-TOF-MS spectra of (B) native PAMAM, (C) PEGylated PAMAM-(mPEG₂ₖ)₃, (D) PEGylated PAMAM-(mPEG₂ₖ)₈, and (E) PEGylated PAMAM-(mPEG₂ₖ)₁₈.

FIG. 50. Scheme of the common glycoside structures present in QS-21: structure of 4 QS-21 isomers.

FIG. 51. Cartoons (A-E) displaying exemplifying molecules and conjugates of the present invention.

FIG. 52. Targeted 2-component activity requires endosomal acidification.

FIG. 53: Cell viability under influence of treatment of cells with Trastuzumab-SO1861 (DAR4, labile linker) vs trastuzumab-SO1861 (DAR2.1, labile linker) vs trastuzumab-SO1861 (DAR4, stable linker), when toxicity of trastuzumab-saporin is assessed under influence of a concentration series of the free saponin or the saponin conjugated with trastuzumab using the indicated linkers and applying the indicated DAR.

FIG. 54: Cetuximab-SO1861+HSP27 BNA oligo induces enhanced gene silencing of HSP27 target gene (HSP27 BNA is the ASO (BNA) hsp27BNA with sequence GGCACAGCCAGTGGCG).

FIG. 55 displays a Table with a Data summary of IC50 values for mAb, toxin, ligand toxin or mAb-toxin monotherapy with or without SO1861.

FIG. 56 displays a Table showing IC50 values and Saponin-Mediated Factors of enhancement for the toxin saporin-3 (Sap-3) and a chimeric saporin adapter-EGF SA2E on NIH-3T3 cells in the absence and presence of different Saponins (data adapted from CHRISTOPHER BACHRAN, MARK SUTHERLAND, IRING HEISLER, PHILIPP HEBESTREIT, MATTHIAS F. MELZIG, AND HENDRIK FUCHS, The Saponin-Mediated Enhanced Uptake of Targeted Saporin-Based Drugs Is Strongly Dependent on the Saponin Structure, Exp Biol Med 231:412-420, 2006).

FIG. 57 displays a Table with an overview of antibody-drug conjugates of which development was discontinued, inactive, withdrawn or the filing was rejected due to various reasons.

### DEFINITIONS

The term “proteinaceous” has its regular scientific meaning and here refers to a molecule that is protein-like, meaning that the molecule possesses, to some degree, the physicochemical properties characteristic of a protein, is of protein, relating to protein, containing protein, pertaining to protein, consisting of protein, resembling protein, or being a protein. The term “proteinaceous” as used in for example ‘proteinaceous molecule’ refers to the presence of at least a part of the molecule that resembles or is a protein, wherein ‘protein’ is to be understood to include a chain of amino-acid residues at least two residues long, thus including a peptide, a polypeptide and a protein and an assembly of proteins or protein domains. In the proteinaceous molecule, the at least two amino-acid residues are for example bound via (an) amide bond(s), such as (a) peptide bond(s). In the proteinaceous molecule, the amino-acid residues are natural amino-acid residues and/or artificial amino-acid residues such as modified natural amino-acid residues. In a preferred embodiment, a proteinaceous molecule is a molecule comprising at least two amino-acid residues, preferably between two and about 2.000 amino-acid residues. In one embodiment, a proteinaceous molecule is a molecule comprising from 2 to 20 (typical for a peptide) amino acids. In one embodiment, a proteinaceous molecule is a molecule comprising from 21 to 1.000 (typical for a polypeptide, a protein, a protein domain, such as an antibody, a Fab, an scFv, a ligand for a receptor such as EGF) amino acids. Preferably, the amino-acid residues are (typically) bound via (a) peptide bond(s). According to the invention, said amino-acid residues are or comprise (modified) (non-)natural amino acid residues.

The term “effector molecule”, or “effector moiety” when referring to the effector molecule as part of e.g. a covalent conjugate, has its regular scientific meaning and here refers to a molecule that can selectively bind to for example any one or more of the target molecules: a protein, a peptide, a carbohydrate, a saccharide such as a glycan, a (phospho)lipid, a nucleic acid such as DNA, RNA, an enzyme, and regulates the biological activity of such one or more target molecule(s). The effector molecule is for example a molecule selected from any one or more of a small molecule such as a drug molecule, a toxin such as a protein toxin, an oligonucleotide such as a BNA, a xeno nucleic acid or an siRNA, an enzyme, a peptide, a protein, or any combination thereof. Thus, for example, an effector molecule or an effector moiety is a molecule or moiety selected from any one or more of a small molecule such as a drug molecule, a toxin such as a protein toxin, an oligonucleotide such as a BNA, a xeno nucleic acid or an siRNA, an enzyme, a peptide, a protein, or any combination thereof, that can selectively bind to any one or more of the target molecules: a protein, a peptide, a carbohydrate, a saccharide such as a glycan, a (phospho)lipid, a nucleic acid such as DNA, RNA, an enzyme, and that upon binding to the target molecule regulates the biological activity of such one or more target molecule(s). Typically, an effector molecule can exert a biological effect inside a cell such as a mammalian cell such as a human cell, such as in the cytosol of said cell. Typical effector molecules are thus drug molecules, an enzyme, plasmid DNA, toxins such as toxins comprised by antibody-drug conjugates (ADCs), oligonucleotides such as siRNA, BNA, nucleic acids comprised by an antibody-oligonucleotide conjugate (AOC). For example, an effector molecule is a molecule which can act as a ligand that can increase or decrease (intracellular) enzyme activity, gene expression, or cell signalling.

The term “saponin” has its regular scientific meaning and here refers to a group of amphipatic glycosides which comprise one or more hydrophilic glycone moieties combined with a lipophilic aglycone core which is a sapogenin. The saponin may be naturally occurring or synthetic (i.e. non-naturally occurring). The term “saponin” includes naturally-occurring saponins, derivatives of naturally-occurring saponins as well as saponins synthesized de novo through chemical and/or biotechnological synthesis routes.

The term “aglycone core structure” has its regular scientific meaning and here refers to the aglycone core of a saponin without the one or two carbohydrate antenna or saccharide chains (glycans) bound thereto. For example, quillaic acid is the aglycone core structure for SO1861, QS-7, QS21. Typically, the glycans of a saponin are mono-saccharides or oligo-saccharides, such as linear or branched glycans.

The term “saccharide chain” has its regular scientific meaning and here refers to any of a glycan, a carbohydrate antenna, a single saccharide moiety (mono-saccharide) or a chain comprising multiple saccharide moieties (oligosaccharide, polysaccharide). The saccharide chain can consist of only saccharide moieties or may also comprise further moieties such as any one of 4E-Methoxycinnamic acid, 4Z-Methoxycinnamic acid, and 5-O-[5-O-Ara/Api-3,5-dihydroxy-6-methyl-octanoyl]-3,5-dihydroxy-6-methyl-octanoic acid), such as for example present in QS-21.

The term “Api/Xyl-” or “Api- or Xyl-” in the context of the name of a saccharide chain has its regular scientific meaning and here refers to the saccharide chain either comprising an apiose (Api) moiety, or comprising a xylose (Xyl) moiety.

The term “oligonucleotide” has its regular scientific meaning and here refers to amongst others any natural or synthetic string of nucleic acids encompassing DNA, modified DNA, RNA, mRNA, modified RNA, synthetic nucleic acids, presented as a single-stranded molecule or a double-stranded molecule, such as a BNA, an antisense oligonucleotide (ASO), a short or small interfering RNA (siRNA; silencing RNA), an anti-sense DNA, anti-sense RNA, etc.

The term “antibody-drug conjugate” or “ADC” has its regular scientific meaning and here refers to any conjugate of an antibody such as an IgG, a Fab, an scFv, an immunoglobulin, an immunoglobulin fragment, one or multiple VH domains, single-domain antibodies, a VHH, a camelid VH, etc., and any molecule that can exert a therapeutic effect when contacted with cells of a subject such as a human patient, such as an active pharmaceutical ingredient, a toxin, an oligonucleotide, an enzyme, a small molecule drug compound, etc.

The term “antibody-oligonucleotide conjugate” or “AOC” has its regular scientific meaning and here refers to any conjugate of an antibody such as an IgG, a Fab, an scFv, an immunoglobulin, an immunoglobulin fragment, one or multiple VH domains, single-domain antibodies, a VHH, a camelid VH, etc., and any oligonucleotide molecule that can exert a therapeutic effect when contacted with cells of a subject such as a human patient, such as an oligonucleotide selected from a natural or synthetic string of nucleic acids encompassing DNA, modified DNA, RNA, mRNA, modified RNA, synthetic nucleic acids, presented as a single-stranded molecule or a double-stranded molecule, such as a BNA, an antisense oligonucleotide (ASO), a short or small interfering RNA (siRNA; silencing RNA), an anti-sense DNA, anti-sense RNA, etc.

The term “bridged nucleic acid”, or “BNA” in short, or “locked nucleic acid” or “LNA” in short, has its regular scientific meaning and here refers to a modified RNA nucleotide. A BNA is also referred to as ‘constrained RNA molecule’ or ‘inaccessible RNA molecule’. A BNA monomer can contain a five-membered, six-membered or even a seven-membered bridged structure with a “fixed” C₃′-endo sugar puckering. The bridge is synthetically incorporated at the 2′,4′-position of the ribose to afford a 2′,4′-BNA monomer. A BNA monomer can be incorporated into an oligonucleotide polymeric structure using standard phosphoramidite chemistry known in the art. A BNA is a structurally rigid oligonucleotide with increased binding affinity and stability.

The term ‘S’ as used such as in an antibody-saponin conjugate comprising a linker, represents ‘stable linker’ which remains intact in the endosome and in the cytosol.

The term I′ as used such as in an antibody-saponin conjugate comprising a linker, represents ‘labile linker’ which is cleaved under slightly acid conditions in the endosome.

The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the embodiments of the invention can operate in other sequences than described or illustrated herein, unless specified otherwise.

The embodiments of the invention described herein can operate in combination and cooperation, unless specified otherwise.

Furthermore, the various embodiments, although referred to as “preferred” or “e.g.” or “for example” or “in particular” and the like are to be construed as exemplary manners in which the invention may be implemented rather than as limiting the scope of the invention.

The term “comprising”, used in the claims, should not be interpreted as being restricted to for example the elements or the method steps or the constituents of a compositions listed thereafter; it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present invention, the only enumerated steps of the method are A and B, and further the claim should be interpreted as including equivalents of those method steps. Thus, the scope of the expression “a composition comprising components A and B” should not be limited to a composition consisting only of components A and B, rather with respect to the present invention, the only enumerated components of the composition are A and B, and further the claim should be interpreted as including equivalents of those components.

In addition, reference to an element or a component by the indefinite article “a” or “an” does not exclude the possibility that more than one of the element or component are present, unless the context clearly requires that there is one and only one of the elements or components. The indefinite article “a” or “an” thus usually means “at least one”.

### DETAILED DESCRIPTION

The present invention relates to a combination of two binding molecules, wherein a first binding molecule comprises at least one glycoside molecule and wherein a second binding molecule comprises at least one effector molecule, wherein the glycoside molecule and the effector molecule are not bound to one and the same binding molecule and wherein the first and the second binding molecule are, independently from one another, able to specifically bind to a target cell-specific surface molecule or structure, thereby inducing receptor-mediated endocytosis of the complex of binding molecule and target cell-specific surface molecule.

The current invention also concerns an endosomal and/or lysosomal escape enhancing conjugate comprising a saponin linked to a targeting molecule such as an antibody, a therapeutic combination of said endosomal and/or lysosomal escape enhancing conjugate and a functionalized binding molecule comprising an effector molecule, wherein the endosomal and/or lysosomal escape enhancing conjugate comprises an enhancer of said effector molecule. In particular the current invention concerns a therapeutic combination for use as a medicament, in particular for use in the treatment of a tumour. The current invention concerns a method of treating cancer or an autoimmune disease by administering the therapeutic combination to a patient.

The present invention will be described with respect to particular embodiments but the invention is not limited thereto but only by the claims.

The embodiments of the invention described herein can operate in combination and cooperation, unless specified otherwise.

An aspect of the invention relates to a functionalized glycoside moiety having endosomal and/or lysosomal escape enhancing activity and having a molecular structure comprising at least one S moiety and at least one connector moiety L*, with general structure (0):

S-(L*)ₘ structure (0),

wherein the S moiety is a glycoside,
wherein
the at least one L* moiety is at least one W* moiety,

wherein the at least one W* moiety is any one or more of:
a reactive group ‘*’ on the at least one S moiety, providing S*, the reactive group ‘*’ for linking the at least one S* moiety to at least a first moiety L* via the reactive group ‘*’ on the S* moiety,
a linker, the linker comprising a reactive group ‘*’ for linking of the at least one S moiety to a further moiety F;
a first proteinaceous molecule;
a scaffold, consisting of, or comprising
   an oligomeric structure, or
   a polymeric structure,
      wherein the oligomeric structure and the polymeric structure comprises, or is selected from, any of:
         a polymer;
         an oligomer;
         a dendrimer;
         a dendron;
         a dendronized polymer;
         a dendronized oligomer;
         an assembly of any of a polymer, an oligomer, a dendrimer, a dendron, a dendronized polymer, a dendronized oligomer,
         wherein the polymer, oligomer, dendrimer, dendron, dendronized polymer, dendronized oligomer, are any of
         linear;
         branched; or
         cyclic,
            wherein the scaffold comprises a single reactive group ‘*’ for coupling a single S moiety, or
            wherein the scaffold comprises more than one reactive group ‘*’, each group for coupling a single S moiety,
            wherein the scaffold comprises a single binding site for binding a further moiety F, or
            wherein the scaffold comprises multiple binding sites for binding multiple further moieties F,
            said binding sites for one or more further moieties F on the scaffold moiety W* being reactive groups ‘*’ on the scaffold moiety W* for provision of a bond with at least one further moiety F,
            wherein the at least one S moiety is linked, coupled or bound to the reactive group ‘*’ on the W* moiety through a bond,

wherein m is at least 1 and at most equal to the number of reactive groups ‘*’ on the at least one S moiety,

wherein the L* moieties are the same or different for m>1;

wherein the W* moieties are the same or different for m>1;

or
wherein the at least one L* moiety is an O* moiety,

wherein the O* moiety is a trifunctional linker comprising three reactive groups ‘*’ for linking one S moiety and two further moieties F, or for linking two S moieties and one further moiety F, or wherein the O* moiety is a linker with at least three functionalities comprising at least three reactive groups ‘*’ for linking at least one S moiety and at least two further moieties F, or for linking at least two S moieties and at least one further moiety F
wherein the three reactive groups ‘*’ or the at least three reactive groups ‘*’ are the same or different;
wherein the O* moieties are the same or different for m>1;
   or wherein the at least one L* moiety is one or more W* moieties and/or one or more O* moieties, wherein more than two W* moieties and O* moieties together are coupled in a linear fashion or are coupled in a branched order relative to the S moiety,
   wherein ‘*’ depicts a binding site or reactive group for binding an S moiety, a W moiety, an O moiety, to a further moiety S, W*, O*, or a further moiety F.

wherein F moieties are the same or different when the functionalized glycoside moiety encompasses more than one F moiety.

An aspect of the invention relates to a functionalized glycoside moiety having endosomal and/or lysosomal escape enhancing activity and having a molecular structure comprising at least one S moiety and at least one connector moiety L*, with general structure (0):

S-(L*)ₘ structure (0),

wherein the S moiety is a glycoside,

preferably the at least one S moiety is any of
a bisdesmosidic triterpene,
a bisdesmosidic triterpene saponin,
a bisdesmosidic triterpene saponin belonging to the type of a 12,13-dehydrooleanane with an aldehyde function, in position 23,
a saponin isolatable from species Gypsophila,
a saponin isolatable from species Saponaria,
a saponin selected from:
   SA1641 or a diastereomer thereof,
   SO1861 or a diastereomer thereof, and
   GE1741 or a diastereomer thereof;
   and preferably the at least one S moiety is SO1861;
      wherein
      the at least one L* moiety is at least one W* moiety,

wherein the at least one W* moiety is any one or more of:
a reactive group ‘*’ on the at least one S moiety, providing S*, the reactive group ‘*’ preferably selected from
   an aldehyde group,
   a carboxylic acid group,
   an alkenyl group,
   an hydroxyl group,
   for linking the at least one S* moiety to at least a first moiety L* via the reactive group ‘*’ on the S* moiety,
a linker, such as a chemical linker or a linear or non-linear stretch of amino-acid residues complexed through peptide bonds and/or disulphide bonds, the linker comprising a reactive group ‘*’ for linking of the at least one S moiety to a further moiety F, preferably the linker is N-ε-maleimidocaproic acid hydrazide for conjugating a sulfhydryl, such as in a cysteine, to a carbonyl such as in an aldehyde or in a ketone, or preferably the linker is succinimidyl 3-(2-pyridyldithio)propionate, wherein the F moiety is any one or more of a payload, a further S moiety, a further linker, a scaffold, a ligand, an effector molecule, an antibody, EGF, a toxin, an oligonucleotide such as an RNA, a BNA, a DNA, an LNA;
a first proteinaceous molecule such as a first peptide, a first polypeptide, or a first protein, preferably the first protein is an antibody, an immunoglobulin, or a binding domain thereof or a binding fragment thereof, such as an immunoglobulin G, a Fab fragment, an scFv, at least one Vh domain, at least one VHH domain;
   wherein the first proteinaceous molecule comprises a single reactive group ‘*’ for coupling a single S moiety, or
   wherein the first proteinaceous molecule comprises more than one reactive group ‘*’, each group ‘*’ for coupling a single S moiety,
   wherein the first proteinaceous molecule comprises a single binding site for a single further moiety F, or
   wherein the first proteinaceous molecule comprises multiple binding sites for multiple further moieties F,
      said binding sites on the first proteinaceous molecule being reactive groups ‘*’ on the first proteinaceous molecule for provision of a bond with a further moiety F, such as a covalent bond, a non-covalent bond, an electrostatic interaction, a hydrogen bond, a salt bridge, a van der Waals interaction, a hydrophobic interaction, preferably a covalent bond,
scaffold, consisting of, or comprising
   an oligomeric structure, or
   a polymeric structure,
      wherein the oligomeric structure and the polymeric structure comprises, or is selected from, any of:
         a polymer;
         an oligomer;
         a dendrimer;
         a dendron;
         a dendronized polymer;
         a dendronized oligomer;
         an assembly of any of a polymer, an oligomer, a dendrimer, a dendron, a dendronized polymer, a dendronized oligomer,
         wherein the polymer, oligomer, dendrimer, dendron, dendronized polymer, dendronized oligomer, are any of
         linear;
         branched; or
         cyclic,
            wherein the scaffold comprises a single reactive group ‘*’ for coupling a single S moiety, preferably a terminal S moiety, or
            wherein the scaffold comprises more than one reactive group ‘*’, each group for coupling a single S moiety, preferably a terminal S moiety,
            wherein the scaffold comprises a single binding site for binding a further moiety F, or
            wherein the scaffold comprises multiple binding sites for binding multiple further moieties F,
            said binding sites for one or more further moieties F on the scaffold moiety W* being reactive groups ‘*’ on the scaffold moiety W* for provision of a bond with at least one further moiety F, such as a covalent bond, a non-covalent bond, an electrostatic interaction, a hydrogen bond, a salt bridge, a van der Waals interaction, a hydrophobic interaction, preferably a covalent bond,
            wherein the at least one S moiety is linked, coupled or bound to the reactive group ‘*’ on the W* moiety through a bond, such as a covalent bond, a non-covalent bond, an electrostatic interaction, a hydrogen bond, a salt bridge, a van der Waals interaction, a hydrophobic interaction, preferably a covalent bond,

wherein said (covalent) bond is optionally a cleavable bond, wherein said cleavable bond is preferably subject to cleavage under any one or more of:
acidic conditions, preferably at a pH of lower than 6.5 such as pH 4.0-6.5, and preferably at a pH ≤5.5;
reductive conditions;
enzymatic conditions; and
light-induced conditions,
   wherein the cleavable bond is optionally selected from:

an imine bond;

a hydrazone bond;

a 1,3-dioxolane bond; and

an ester bond, and/or
wherein the cleavable bond is a disulfide bond or a peptide bond or an amide bond,

wherein m is at least 1 and at most equal to the number of reactive groups on the at least one S moiety, the reactive groups ‘*’ selected preferably from an aldehyde group, a carboxylic acid group, an alkenyl group, and an hydroxyl group, for linking the at least one S moiety to a further L* moiety, and preferably m=1,
wherein the L* moieties are the same or different for m>1;
wherein the W* moieties are the same or different for m>1;
   or
   wherein the at least one L* moiety is an O* moiety,

wherein the O* moiety is a trifunctional linker comprising three reactive groups ‘*’ for linking one S moiety and two further moieties F, or for linking two S moieties and one further moiety F, or wherein the O* moiety is a linker with at least three functionalities comprising at least three reactive groups ‘*’ for linking at least one S moiety and at least two further moieties F, or for linking at least two S moieties and at least one further moiety F
wherein the three reactive groups ‘*’ or the at least three reactive groups ‘*’ are the same or different;
wherein the O* moieties are the same or different for m>1;
   or wherein the at least one L* moiety is one or more W* moieties and/or one or more O* moieties, wherein more than two W* moieties and O* moieties together are coupled in a linear fashion or are coupled in a branched order relative to the S moiety, such as for example S-W*-O*-O*, S-O*-W*-O*, S-O*-O*-W*, S-W*(-O*)₂, S-O*(-W*)(-O*),
   wherein ‘*’ depicts a binding site or reactive group for binding an S moiety, a W moiety, an O moiety, to a further moiety S, W*, O*, or a further moiety F.

wherein F moieties are the same or different when the functionalized glycoside moiety encompasses more than one F moiety.

An aspect of the invention relates to an endosomal and/or lysosomal escape enhancing conjugate having a molecular structure comprising at least one S moiety, at least one connector moiety L and at least one E moiety, with general structure (I):

S(-L-E)ₙ structure (I),

wherein the at least one S moiety is a glycoside;
wherein
the at least one L moiety is at least one W moiety,

wherein the at least one W moiety is any one or more of:
a reactive group ‘*’ on the at least one S moiety, for linking the at least one S moiety to at least a first moiety L via the reactive group ‘*’,
a linker comprising a reactive group ‘*’ for direct linking of the at least one S moiety to a single E moiety;
   a first proteinaceous molecule wherein the first proteinaceous molecule comprises a single reactive group ‘*’ for coupling a single S moiety, or wherein the first proteinaceous molecule comprises more than one reactive group ‘*’, each group ‘*’ for coupling a single S moiety,
   wherein the first proteinaceous molecule comprises a single binding site for a single moiety E, or
   wherein the first proteinaceous molecule comprises multiple binding sites for multiple moieties E,
      said binding sites on the first proteinaceous molecule being reactive groups ‘*’ on the first proteinaceous molecule for provision of a bond with a moiety E,
a scaffold, consisting of, or comprising
   an oligomeric structure, or
   a polymeric structure,
      wherein the oligomeric structure and the polymeric structure comprises, or is selected from, any of:
         a polymer;
         an oligomer;
         a dendrimer;
         a dendron;
         a dendronized polymer;
         a dendronized oligomer;
         an assembly of any of a polymer, an oligomer, a dendrimer, a dendron, a dendronized polymer, a dendronized oligomer,
         wherein the polymer, oligomer, dendrimer, dendron, dendronized polymer, dendronized oligomer, are any of
         linear;
         branched; or
         cyclic,
            wherein the scaffold comprises a single reactive group ‘*’ for coupling a single S moiety, or
            wherein the scaffold comprises more than one reactive group ‘*’, each group ‘*’ for coupling a single S moiety,
            wherein the scaffold comprises a single binding site for binding a single E moiety, or
            wherein the scaffold comprises multiple binding sites for binding multiple E moieties,
            said binding sites for one or more E moieties on the scaffold moiety W being reactive groups ‘*’ on the scaffold moiety W for provision of a bond with at least one E moiety,
            wherein the at least one S moiety is linked, coupled or bound to the reactive group ‘*’ on the W moiety through a bond,

wherein n is at least 1 and at most equal to the number of reactive groups ‘*’ on the at least one S moiety,
wherein the L moieties are the same or different for n>1;
wherein the W moieties are the same or different for n>1;
   or
   wherein the at least one L moiety is an O moiety,

wherein the O moiety is a trifunctional linker comprising three reactive groups ‘*’ for linking one S moiety and two E moieties, or for linking two S moieties and one E moiety, or wherein the O moiety is a linker with at least three functionalities comprising at least three reactive groups ‘*’ for linking at least one S moiety and at least two E moieties, or for linking at least two S moieties and at least one E moiety
wherein the O moieties are the same or different for n>1;
   or wherein the at least one L moiety is one or more W moieties and/or one or more O moieties, wherein more than two W moieties and O moieties together are coupled in a linear fashion or are coupled in a branched order, relative to a first coupled E moiety,

wherein the at least one E moiety is any one or more of:
(iv) one S moiety or more than one S moieties, wherein the more than one S moieties are the same or different;
(v) at least one payload selected from one effector moiety or more effector moieties; and
(vi) one ligand or more ligands, wherein the at least one S moiety is a glycoside;
   wherein the effector moiety or the effector moieties is/are selected from any one or more of:
a molecule with pharmaceutical activity;
a toxin;
a nucleotide;
an enzyme;
a second protein; and
a second peptide,
   wherein the ligand(s) is/are selected from any one or more of:
a binding partner for a target cell surface molecule; and
an immunoglobulin or a binding domain or binding fragment thereof, for binding to such a cell surface molecule,
   wherein the effector moiety/moieties and the ligand(s) are directly coupled to any of the scaffold, the at least one S moiety, the trifunctional linker O, the linker, the first proteinaceous molecule,

and/or wherein a first effector moiety or a first ligand is directly coupled to any of the scaffold, the at least one S moiety, the trifunctional linker O, the linker, the first proteinaceous molecule,
wherein the E moieties are the same or are different for n>1;
wherein effector moieties are the same or different for n>1; and
wherein ligands are the same or different for n>1.

An aspect of the invention relates to an endosomal and/or lysosomal escape enhancing conjugate having a molecular structure comprising at least one S moiety, at least one connector moiety L and at least one E moiety, with general structure (I):

S(-L-E)ₙ structure (I),

wherein the at least one S moiety is a glycoside,

preferably the at least one S moiety is any of
a bisdesmosidic triterpene,
a bisdesmosidic triterpene saponin,
a bisdesmosidic triterpene saponin belonging to the type of a 12,13-dehydrooleanane with an aldehyde function, in position 23,
a saponin isolatable from species Gypsophila,
a saponin isolatable from species Saponaria,
a saponin selected from:
   SA1641 or a diastereomer thereof,
   SO1861 or a diastereomer thereof, and
   GE1741 or a diastereomer thereof;
   and preferably the at least one S moiety is SO1861;
      wherein
      the at least one L moiety is at least one W moiety,
wherein the at least one W moiety is any one or more of:
   a reactive group ‘*’ on the at least one S moiety, preferably selected from
      an aldehyde group,
      a carboxylic acid group,
      an alkenyl group,
      an hydroxyl group,
      for linking the at least one S moiety to at least a first moiety L via the reactive group
   a linker, such as a chemical linker or a linear or non-linear stretch of amino-acid residues complexed through peptide bonds and/or disulphide bonds and/or chemical bonds, the linker comprising a reactive group ‘*’ for direct linking of the at least one S moiety to a single E moiety through preferably a single bond, preferably the linker is N-ε-maleimidocaproic acid hydrazide for conjugating a sulfhydryl, such as in a cysteine, to a carbonyl such as in an aldehyde or in a ketone, or preferably the linker is succinimidyl 3-(2-pyridyldithio)propionate;
   a first proteinaceous molecule such as a first peptide, a first polypeptide, or a first protein, preferably the first protein is an antibody, an immunoglobulin, or a binding domain thereof or a binding fragment thereof, such as an immunoglobulin G, a Fab fragment, an scFv, at least one Vh domain, at least one VHH domain;
      wherein the first proteinaceous molecule comprises a single reactive group ‘*’ for coupling a single S moiety, or
      wherein the first proteinaceous molecule comprises more than one reactive group ‘*’, each group ‘*’ for coupling a single S moiety,
      wherein the first proteinaceous molecule comprises a single binding site for a single moiety E, or
      wherein the first proteinaceous molecule comprises multiple binding sites for multiple moieties E,
         said binding sites on the first proteinaceous molecule being reactive groups ‘*’ on the first proteinaceous molecule for provision of a bond with a moiety E, such as a covalent bond, a non-covalent bond, an electrostatic interaction, a hydrogen bond, a salt bridge, a van der Waals interaction, a hydrophobic interaction, preferably a covalent bond,
   a scaffold, consisting of, or comprising
      an oligomeric structure, or
      a polymeric structure,
         wherein the oligomeric structure and the polymeric structure comprises, or is selected from, any of:
         a polymer;
         an oligomer;
         a dendrimer;
         a dendron;
         a dendronized polymer;
         a dendronized oligomer;
         an assembly of any of a polymer, an oligomer, a dendrimer, a dendron, a dendronized polymer, a dendronized oligomer,
         wherein the polymer, oligomer, dendrimer, dendron, dendronized polymer, dendronized oligomer, are any of
         linear;
         branched; or
         cyclic,
            wherein the scaffold comprises a single reactive group ‘*’ for coupling a single S moiety, or
            wherein the scaffold comprises more than one reactive group ‘*’, each group ‘*’ for coupling a single S moiety,
            wherein the scaffold comprises a single binding site for binding a single E moiety, or
            wherein the scaffold comprises multiple binding sites for binding multiple E moieties,
            said binding sites for one or more E moieties on the scaffold moiety W being reactive groups ‘*’ on the scaffold moiety W for provision of a bond with at least one E moiety, such as a covalent bond, a non-covalent bond, an electrostatic interaction, a hydrogen bond, a salt bridge, a van der Waals interaction, a hydrophobic interaction, preferably a covalent bond,
            wherein the at least one S moiety is linked, coupled or bound to the reactive group ‘*’ on the W moiety through a bond, such as a covalent bond, a non-covalent bond, an electrostatic interaction, a hydrogen bond, a salt bridge, a van der Waals interaction, a hydrophobic interaction, preferably a covalent bond,

wherein said (covalent) bond is optionally a cleavable bond, wherein said cleavable bond is preferably subject to cleavage under any one or more of:
acidic conditions, preferably at a pH of lower than 6.5 such as pH 4.0-6.5, preferably at a pH ≤5.5;
reductive conditions;
enzymatic conditions; and
light-induced conditions,
wherein the cleavable bond is optionally selected from:

an imine bond;

a hydrazone bond;

a 1,3-dioxolane bond; and

an ester bond, and/or wherein the cleavable bond is a disulfide bond or a peptide bond or an amide bond, wherein n is at least 1 and at most equal to the number of reactive groups ‘*’ on the at least one S moiety, the reactive groups ‘*’ selected preferably from an aldehyde group, a carboxylic acid group, an alkenyl group, and an hydroxyl group, for linking the at least one S moiety to a further L moiety, and preferably n=1,
wherein the L moieties are the same or different for n>1;
wherein the W moieties are the same or different for n>1;
   or
   wherein the at least one L moiety is an O moiety,

wherein the O moiety is a trifunctional linker comprising three reactive groups ‘*’ for linking one S moiety and two E moieties, or for linking two S moieties and one E moiety, or wherein the O moiety is a linker with at least three functionalities comprising at least three reactive groups ‘*’ for linking at least one S moiety and at least two E moieties, or for linking at least two S moieties and at least one E moiety
wherein the O moieties are the same or different for n>1;
   or wherein the at least one L moiety is one or more W moieties and/or one or more O moieties, wherein more than two W moieties and O moieties together are coupled in a linear fashion or are coupled in a branched order, relative to a first coupled E moiety, such as for example in the branched order S-W*-O*-O*, S-O*-W*-O*, S-O*-O*-W*, S-W*(-O*)₂, S-O*(-W*)(-O*),

wherein the at least one E moiety is any one or more of:
(vii) one S moiety or more than one S moieties, wherein the more than one S moieties are the same or different;
(viii) at least one payload selected from one effector moiety or more effector moieties; and
(ix) one ligand or more ligands,
   wherein the at least one S moiety is a glycoside,
   preferably the at least one S moiety is any of
a bisdesmosidic triterpene,
a bisdesmosidic triterpene saponin,
a bisdesmosidic triterpene saponin belonging to the type of a 12,13-dehydrooleanane with an aldehyde function, in position 23,
a saponin isolatable from species Gypsophila,
a saponin isolatable from species Saponaria,
a saponin selected from:
   SA1641 or a diastereomer thereof,
   SO1861 or a diastereomer thereof, and
   GE1741 or a diastereomer thereof;
      and preferably the at least one S moiety is SO1861;
      wherein the effector moiety or the effector moieties is/are selected from any one or more of:
a molecule with pharmaceutical activity, such as a drug molecule, including, but not being limited to a macromolecule or a small molecule;
a toxin, such as a macromolecular cell-killing agent, a protein toxin, an immunotoxin, saporin, dianthin, ribosomal inactivating protein, a small molecule cell-killing agent, a small molecule toxin;
a nucleotide, preferably an oligonucleotide, an RNA, a DNA, an LNA, a BNA, (bridged nucleic acid), an aptamer, a nucleic acid, a plasmid, a vector, a gene, an ASO (allele-specific oligonucleotide), an antisense oligonucleotide (ASO), an miRNA (microRNA), an siRNA (small interfering RNA);
an enzyme;
a second protein; and
a second peptide,
   wherein the ligand(s) is/are selected from any one or more of:
a binding partner for a target cell surface molecule, preferable a target cell surface molecule specific for an aberrant cell such as a tumor cell, the target cell surface molecule preferably selected from any of HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CEACAM5, HER3, CD74, PTK7, Notch3, FGF2, C4.4A, FLT3, CD71, CD38, FGFR3, CD123, DLL3, such as the binding partner EGF for cell-surface receptor EGFR or transferrin for transferrin receptor; and
an immunoglobulin or a binding domain or binding fragment thereof, for binding to for example such a cell surface molecule such as cell-surface receptor HER2 and cell-surface receptor EGFR, such as immunoglobulin trastuzumab for binding to HER2 and immunoglobulin cetuximab for binding to EGFR and anti-CD71 monoclonal antibody for binding to cell-surface receptor CD71 (transferrin receptor),
   wherein the S moiety/moieties is/are preferably (a) terminal moiety/moieties,
   wherein the effector moiety/moieties and the ligand(s) are directly coupled to any of the scaffold, the at least one S moiety, the trifunctional linker O, the linker such as a chemical linker, the first proteinaceous molecule such as the first peptide, the first polypeptide, and the first protein,

and/or wherein a first effector moiety or a first ligand is directly coupled to any of the scaffold, the at least one S moiety, the trifunctional linker O, the linker, preferably a chemical linker, the first proteinaceous molecule such as the first peptide, the first polypeptide, the first protein, and wherein optionally a second, a third and further effector moiety/moieties and/or optionally a second, a third and further ligand(s) is/are coupled to said first, second or third effector moiety or is/are coupled to said first, second, or third ligand, either directly, or through a linker, in linear fashion in any order of two or more effector moieties and/or two or more ligands, and/or in branched fashion,
wherein optionally one or more S moiety/moieties is/are coupled to said first, second, third and further effector moiety/moieties and/or to said first, second, third and further ligand(s), preferably S moiety/moieties is/are coupled directly to an effector moiety or to a ligand, or is/are coupled to an effector moiety or to a ligand via an L moiety such as a linker, a trifunctional linker, and/or a scaffold, wherein the scaffold is preferably a dendron or a dendrimer and wherein the S moiety is preferably linked to the scaffold via a linker or a trifunctional linker, wherein the bond between an S moiety and an L moiety is a non-cleavable bond or a cleavable bond, preferably a cleavable bond, wherein said cleavable bond is preferably subject to cleavage under any one or more of:
acidic conditions, preferably at a pH of lower than 6.5 such as pH 4.0-6.5, preferably at a pH of ≤5.5;
reductive conditions;
enzymatic conditions; and
light-induced conditions,
wherein the cleavable bond is optionally selected from:
   an imine bond;
   a hydrazone bond;
   a 1,3-dioxolane bond; and
   an ester bond,
   and/or wherein the cleavable bond is a disulfide bond or a peptide bond or an amide bond,
wherein the E moieties are the same or are different for n>1;
wherein effector moieties are the same or different for n>1; and
wherein ligands are the same or different for n>1.

An embodiment is the endosomal and/or lysosomal escape enhancing conjugate of the invention wherein the Structure (I) comprises the Structure (0) of the invention, wherein binding sites comprising a reactive group ‘*’ in Structure (0) are now occupied by L-, W-, O-, E- and/or S moieties, and wherein the F moiety/moieties is/are (an) E moiety/moieties.

An embodiment is the endosomal and/or lysosomal escape enhancing conjugate of the invention wherein the S moiety is a terminal moiety in the conjugate.

An embodiment is the endosomal and/or lysosomal escape enhancing conjugate of the invention wherein n is 1.

An embodiment is the endosomal and/or lysosomal escape enhancing conjugate according to the invention wherein the S moiety is a bisdesmosidic triterpene saponin belonging to the type of a 12,13-dehydrooleanane with an aldehyde function, in position 23, and wherein S is preferably a saponin that can be isolated from Gypsophila or Saponaria species, preferably the one or more saponins are selected from SA1641 or a diastereomer thereof, SO1861 or a diastereomer thereof, and GE1741 or a diastereomer thereof, or a combination thereof, more preferably S is the saponin SO1861 or any of its diastereomers, and n=1.

An embodiment is the endosomal and/or lysosomal escape enhancing conjugate of the invention, wherein the E moiety is at least a ligand, such as an immunoglobulin.

An embodiment is the endosomal and/or lysosomal escape enhancing conjugate of the invention, wherein E is an immunoglobulin or at least a binding domain thereof for binding to a cell surface molecule, wherein preferably the cell surface molecule is selected from any of HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CD38, FGFR3, CD123, DLL3, CEACAM5, HER3, CD74, PTK7, Notch3, FGF2, C4.4A, FLT3, CD71.

An embodiment is the endosomal and/or lysosomal escape enhancing conjugate according to the invention, wherein the L moiety is a linker coupled to the glycoside via a cleavable or non-cleavable bond encompassing an aldehyde group of the glycoside or encompassing a carbonyl group of the glycoside, and the E moiety is a ligand, wherein the ligand is an immunoglobulin, wherein preferably said cleavable bond is subject to cleavage under acidic, reductive, enzymatic or light-induced conditions, preferably acidic conditions at a pH of 4.0-5.5, and preferably the cleavable bond is a covalent bond, preferably an imine bond, a hydrazone bond, an oxime bond, a 1,3-dioxolane bond or an ester bond, wherein preferably the cleavable bond is a disulfide bond or a peptide bond, or an amide bond.

An embodiment is the endosomal and/or lysosomal escape enhancing conjugate according to the invention, wherein n is 1.

An embodiment is the endosomal and/or lysosomal escape enhancing conjugate according to the invention, wherein the S moiety is a terminal saponin, preferably the saponin SO1861, the L moiety is a chemical linker, and the E moiety is a terminal single ligand moiety or a terminal single immunoglobulin such as trastuzumab or cetuximab or anti-CD71 monoclonal antibody for binding to cell-surface receptor CD71 (transferrin receptor), the linker preferably providing a cleavable bond or a non-cleavable between the terminal S moiety and the terminal E moiety, and n is 1.

An embodiment is the endosomal and/or lysosomal escape enhancing conjugate according to the invention, wherein n is 1, the at least one S moiety is any one or more of SA1641, SO1861 and GE1741, or a combination thereof, more preferably the S moiety/moieties is/are SO1861, the L moiety is a linker and/or trifunctional linker and/or an immunoglobulin for linking one or more terminal S moieties, the E moiety is at least one ligand moiety, preferably an immunoglobulin or a binding domain thereof, the ligand moiety having specificity for a cell-surface molecule, preferably expressed at the surface of an aberrant cell such as a tumor cell and preferably not or to a lesser extent on the surface of a healthy cell.

An embodiment is the endosomal and/or lysosomal escape enhancing conjugate according to the invention, wherein:
i. the E moiety comprises or consists of a ligand moiety, wherein said ligand moiety is cetuximab or trastuzumab or anti-CD71 monoclonal antibody for binding to cell-surface receptor CD71 (transferrin receptor); and/or
ii. the S moiety/moieties is/are SO1861; and/or
iii. the L moiety is a dendron, a covalent bond, a linker such as a chemical linker; and/or
iv. said conjugate encompasses 1, 2, 4, 8, 12, 16, 32, 64 terminally linked S moieties, preferably linked to dendron moieties which are bound to the E moiety which is preferably an immunoglobulin with specificity for a cell-surface molecule exposed on an aberrant cell such as a tumor cell.

An embodiment is the endosomal and/or lysosomal escape enhancing conjugate according to the invention, wherein n is 1, the at least one S moiety is any one or more of SA1641, SO1861 and GE1741, or a combination thereof, more preferably the S moiety/moieties is/are SO1861, the L moiety is a linker and/or trifunctional linker and/or an immunoglobulin for linking one or more terminal S moieties, the E moiety is at least one ligand moiety, preferably an immunoglobulin or a binding domain thereof, the ligand moiety having specificity for a cell-surface molecule, preferably expressed at the surface of an aberrant cell such as a tumor cell and preferably not or to a lesser extent on the surface of a healthy cell, and wherein the E moiety is at least one payload such as one or more effector moieties selected from a toxin, an oligonucleotide, an enzyme, an active pharmaceutical ingredient, a therapeutically active substance, wherein the payload is linked to the ligand moiety, and wherein preferably the S moiety/moieties is/are terminally linked to the ligand moiety.

An embodiment is the endosomal and/or lysosomal escape enhancing conjugate according to the invention, wherein:
a) the E moiety comprises a ligand moiety, wherein said ligand moiety is cetuximab or trastuzumab or anti-CD71 monoclonal antibody for binding to cell-surface receptor CD71 (transferrin receptor); and/or
b) the S moiety/moieties is/are SO1861; and/or
c) the L moiety is a dendron, a covalent bond, a linker such as a chemical linker;
d) the E moiety comprises a payload, wherein said payload is at least one effector moiety selected from a toxin such as dianthin, saporin, and/or an enzyme such as Cre-recombinase, and/or an oligonucleotide such as an RNA, an LNA or a BNA such as HSP27 silencing ASO (BNA); and/or
e) said conjugate encompasses 1, 2, 4, 8, 12, 16, 32, 64 terminally linked S moieties, preferably linked to an E moiety, which is preferably an immunoglobulin, directly or via a (cleavable) bond or via dendron moieties which are bound to the E moiety which is preferably an immunoglobulin, said immunoglobulin endowed with specificity for a cell-surface molecule exposed on an aberrant cell such as a tumor cell.

An aspect of the invention relates to a combination of a first binding molecule of the invention and a second binding molecule of the invention, wherein the first binding molecule comprises at least one glycoside molecule and wherein the second binding molecule comprises at least one effector molecule, wherein the glycoside molecule and the effector molecule are not bound to one and the same binding molecule and wherein the first binding molecule and the second binding molecule are, independently from one another, able to specifically bind to a target cell-specific surface molecule or structure, thereby inducing receptor-mediated endocytosis of the complex of the second binding molecule and target cell-specific surface molecule and of the complex of the first binding molecule and target cell-specific surface molecule.

Preferably, the combination of the first binding molecule and the second binding molecule enables augmentation of endosomal and/or lysosomal escape of said effector molecule by said glycoside. By doing so, the combination preferably improves the effect of the effector molecule.

An aspect of the invention relates to a combination of an endosomal and/or lysosomal escape enhancing conjugate according to the invention and a binding moiety, wherein the binding moiety comprises at least one covalently or non-covalently bound effector molecule as outlined for the endosomal and/or lysosomal escape enhancing conjugate, wherein the endosomal and/or lysosomal escape enhancing conjugate and the binding moiety are, independently from one another, able to bind to a target cell surface molecule or target cell surface structure, such as specifically binding to a target cell surface molecule or target cell surface structure, wherein the target cell surface molecule and target cell surface structure are preferably a target cell surface molecule and a target cell surface structure specifically exposed on the target cell surface, thereby inducing receptor-mediated endocytosis of the endosomal and/or lysosomal escape enhancing conjugate, and of the binding moiety. Preferably, the combination of the endosomal and/or lysosomal escape enhancing conjugate and the binding moiety enables augmentation of endosomal and/or lysosomal escape of said effector molecule by said glycoside. By doing so, the combination preferably improves the effect of the effector molecule.

To explain the invention in more detail, the process of cellular uptake of substances and the used terminology in this invention is described first. The uptake of extracellular substances into a cell by vesicle budding is called endocytosis. Said vesicle budding can be characterized by (1) receptor-dependent ligand uptake mediated by the cytosolic protein clathrin, (2) lipid-raft uptake mediated by the cholesterol-binding protein caveolin, (3) unspecific fluid uptake (pinocytosis), or (4) unspecific particle uptake (phagocytosis). All types of endocytosis run into the following cellular processes of vesicle transport and substance sorting called the endocytic pathways. The endocytic pathways are complex and not fully understood. Earlier it was thought that organelles are formed de novo and mature into the next organelle along the endocytic pathway. Nowadays, it is hypothesized that the endocytic pathways involve stable compartments that are connected by vesicular traffic. A compartment is a complex, multifunctional membrane organelle that is specialized for a particular set of essential functions for the cell. Vesicles are considered to be transient organelles, simpler in composition, and are defined as membrane-enclosed containers that form de novo by budding from a preexisting compartment. In contrast to compartments, vesicles can undergo maturation, which is a physiologically irreversible series of biochemical changes. Early endosomes and late endosomes represent stable compartments in the endocytic pathway while primary endocytic vesicles, phagosomes, multivesicular bodies (also called endosome carrier vesicles), secretory granules, and even lysosomes represent vesicles. The endocytic vesicle, which arises at the plasma membrane, most prominently from clathrin-coated pits, first fuses with the early endosome, which is a major sorting compartment of approximately pH 6.5. A large part of the cargo and membranes internalized are recycled back to the plasma membrane through recycling vesicles (recycling pathway). Components that should be degraded are transported to the acidic late endosome (pH lower than 6) via multivesicular bodies. Lysosomes are vesicles that can store mature lysosomal enzymes and deliver them to a late endosomal compartment when needed. The resulting organelle is called the hybrid organelle or endolysosome. Lysosomes bud off the hybrid organelle in a process referred to as lysosome reformation. Late endosomes, lysosomes, and hybrid organelles are extremely dynamic organelles, and distinction between them is often difficult. Degradation of the endocytosed molecules occurs inside the endolysosomes. Endosomal and/or lysosomal escape is the active or passive release of a substance from the inner lumen of any kind of compartment or vesicle from the endocytic pathway, preferably from clathrin-mediated endocytosis, or recycling pathway into the cytosol. Endosomal and/or lysosomal escape thus includes but is not limited to release from endosomes, endolysosomes or lysosomes, including their intermediate and hybrid organelles. After entering the cytosol, said substance might move to other cell units such as the nucleus. Glycoside molecules in the context of the invention are compounds that are able to enhance the effect of an effector molecule, in particular by facilitating the endosomal and/or lysosomal escape. The glycoside molecules interact with the membranes of compartments and vesicles of the endocytic and recycling pathway and make them leaky for said effector molecules resulting in augmented endosomal and/or lysosomal escape.

An effector molecule in the context of this invention is any substance that affects the metabolism of a cell by interaction with an intracellular effector molecule target, wherein this effector molecule target is any molecule or structure inside cells excluding the lumen of compartments and vesicles of the endocytic and recycling pathway but including the membranes of these compartments and vesicles. Said structures inside cells thus include the nucleus, mitochondria, chloroplasts, endoplasmic reticulum, Golgi apparatus, other transport vesicles, the inner part of the plasma membrane and the cytosol. With the term “improving an effect of an effector molecule” is meant that the enhancer increases the functional efficacy of that effector molecule (e.g. the therapeutic index of a toxin or a drug; the metabolic efficacy of a modifier in biotechnological processes; the transfection efficacy of genes in cell culture research experiments), preferably by enabling or improving its target engagement. Acceleration, prolongation, or enhancement of antigen-specific immune responses are preferably not included. Therapeutic efficacy includes but is not limited to a stronger therapeutic effect with lower dosing and/or less side effects. “Improving an effect of an effector molecule” can also mean that an effector molecule, which could not be used because of lack of effect (and was e.g. not known as being an effector molecule), becomes effective when used in combination with the present invention. Any other effect, which is beneficial or desired and can be attributed to the combination of effector and enhancer in one molecule, as provided by the invention is considered to be “an improved effect”.

One major drawback of targeted toxin enhancement by glycosides, such as for instance saponins, up to the present invention is that the targeted toxins are internalized by receptor-mediated endocytosis while glycosides passively diffuse through the plasma membrane and reach the endosomal membranes presumably via interaction with cholesterol. In principal glycosides can enter any cell, also non-target cells, resulting in inefficient enhancer availability in the target cells for effective release of the targeted toxin and possible side effects in non-target cells. One major problem is that entry of the targeted toxin and the glycosides proceed with different kinetics and that these kinetics are different from cell line to cell line so that the correct time difference for the application of the two substances can widely vary from tumor cell line to tumor cell line. Moreover, in living organisms, liberation, absorption, distribution, metabolism and excretion of these substances is also different. Furthermore, the a-specific uptake of glycosides by non-targeted cells may induce unwanted effects in these cells. This can, e.g., be cytosolic delivery of compounds that should have been delivered to the lysosomes, disturbed antigen presentation, etc. Non-targeted administration of the glycoside and the targeted drug may also be problematic in drug development and may hinder or at least postpone marketing authorization by the relevant authorities (e.g. FDA or EMA). With targeted toxin or targeted drug in the context of the present invention is meant that a toxin or drug is specifically targeted to a membrane bound molecule on a target cell, e.g. a toxin or drug bound to a ligand of a membrane receptor or bound to an antibody that specifically recognizes a structure on the cell membrane of a target cell.

It is thus very useful to direct the glycoside via the same route as the effector molecule, e.g., via a targeting ligand to the target cell in order to be available at effective concentration inside the acidic compartments of the endocytic pathway of the target cell and to exhibit a synergistic effect with the toxin. The present invention, therefore, provides novel approaches to redirect both the effector and endosomal and/or lysosomal escape enhancer via targeting ligands to the acidic compartments of the endocytic pathway of the target cell.

In one preferred embodiment, a combination according to the invention is provided, wherein the first and the second binding molecule are able to specifically bind to the same target cell-specific surface molecule or structure. An embodiment is the combination of the invention wherein the endosomal and/or lysosomal escape enhancing conjugate and the binding moiety are able to specifically bind to the same target cell-specific surface molecule or structure. Preferably, the first binding molecule is able to compete with the second binding molecule for binding to the target cell-specific surface molecule or structure. An embodiment is the combination of the invention, wherein the endosomal and/or lysosomal escape enhancing conjugate is able to compete with the binding moiety for binding to the target cell-specific surface molecule or structure. Even more preferred, the first and the second binding molecule are, independently from one another, able to specifically bind to the same epitope. An embodiment is the combination of the invention wherein wherein the endosomal and/or lysosomal escape enhancing conjugate and the binding moiety are, independently from one another, able to specifically bind to the same epitope.

The invention surprisingly shows that a combination of two binding molecules that are able to compete with each other in binding to the same target cell specific surface molecule are very useful in targeting cells that highly express such surface molecules. If for instance a saponin and a toxin are coupled to antibodies that are specific for HER2, only HER2 high expressers are killed. The combination does not have any effect on cells that are low or intermediate expressers of HER2 or cells that do not express HER2. It is believed that the effect is abolished on cells that express HER2 intermediately by competition between the antibodies that are bound to saponin and the antibodies that are bound to the toxin, such that sufficient concentrations of saponin are not reached within the endosomes to enable endosomal and/or lysosomal escape.

Preferably, the target cell-specific surface molecule or structure is selected from the group consisting of HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CD38, FGFR3, CD123, DLL3, CEACAM5, HER3, CD74, PTK7, Notch3, FGF2, C4.4A, FLT3, CD71. An embodiment is the combination of the invention comprising an endosomal and/or lysosomal escape enhancing conjugate of the invention, wherein the target cell-specific surface molecule or structure is selected from HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CD38, FGFR3, CD123, DLL3, CEACAM5, HER3, CD74, PTK7, Notch3, FGF2, C4.4A, FLT3, CD71.

In another preferred embodiment, a combination according to the invention is provided, wherein the first binding molecule is able to specifically bind to a first epitope, which is different from a second epitope to which the second binding molecule is able to specifically bind. An embodiment is the combination of the invention comprising an endosomal and/or lysosomal escape enhancing conjugate of the invention, wherein the endosomal and/or lysosomal escape enhancing conjugate is able to specifically bind to a first epitope, which is different from a second epitope to which the binding moiety is able to specifically bind. Preferably, the first and the second epitope are present on two different target cell-specific surface molecules or structures, wherein the two different target cell-specific surface molecules or structures are co-expressed on at least one target cell. An embodiment is the combination, wherein the first and the second epitope are present on two different target cell-specific surface molecules or structures, wherein the two different target cell-specific surface molecules or structures are co-expressed on at least one target cell. The present invention surprisingly shows that such a combination of binding molecules or combinations of an endosomal and/or lysosomal escape enhancing conjugate and a binding moiety are particularly useful for target cells that are intermediate to high expressers of one of the surface molecule and low to intermediate expressers of the other surface molecule. It is useful in particular if at least the low to intermediate expressed surface molecule is very specific for the target cell. Without the inventive concept of the present invention, it was not possible to specifically target a cell that showed low to intermediate expression of a target-specific surface molecule with a glycoside as, in order for the glycoside to reach a sufficiently high concentration in the endosomes, it should be targeted to a highly expressed surface molecule. According to the present inventive concept, the glycoside is targeted to an intermediate to highly expressed surface receptor, preferably but not necessarily specific for the target cell, whereas the toxin is targeted to a low to intermediate expressed surface receptor, preferably specific for the target cell. It is preferred, however, that the combination of intermediate to highly expressed surface receptor and the low to intermediate expressed surface receptor is not present in a similar constellation in non-target cells in order to prevent off-target toxicity.

A number of preferred features can be formulated for endosomal and/or lysosomal escape enhancers, i.e. a glycoside according to the invention: (1) they are preferably not toxic and do not invoke an immune response, (2) they preferably do not mediate the cytosolic uptake of the effector molecule into off-target cells, (3) their presence at the site of action is preferably synchronized with the presence of the effector molecule, (4) they are preferably biodegradable or excretable, and (5) they preferably do not substantially interfere with biological processes of the organism unrelated to the biological activity of the effector molecule with which the endosomal and/or lysosomal escape enhancer is combined with, e.g. interact with hormones. Examples of glycoside molecules that fulfill the before mentioned criteria, at least to some extent, are bisdesmosidic triterpenes, preferably bisdesmosidic triterpene saponins.

In one preferred embodiment, a combination according to the invention is provided, wherein the glycoside molecule is a bisdesmosidic triterpene, preferably a bisdesmosidic triterpene saponin, more preferably a bisdesmosidic triterpene saponin belonging to the type of a 12,13-dehydrooleanane with an aldehyde function in position 23. Preferably, the saponin is a saponin that can be isolated from Gypsophila or Saponaria species. However, these saponins may be isolated from said or other species, but, may of course also be expressed in other organisms, preferably genetically modified plants or plant cells, more preferably in a large scale plant cell fermentation production process. Such large-scale processes have been developed and made available by contract research organizations. Alternatively, such glycosides may be synthesized chemically according to very complex multistep syntheses as.

In a preferred embodiment, the saponin is a SA1641, SO1861, GE1741 or any of their diastereomers, and SO1861 is preferred.

In a preferred embodiment, a combination according to the invention is provided, wherein the at least one glycoside molecule is bound to said first binding molecule via a cleavable bond, wherein preferably said cleavable bond is subject to cleavage under acidic, reductive, enzymatic or light-induced conditions. Preferably, said cleavable bond is a covalent bond, preferably an imine, hydrazone, oxime, 1,3-dioxolane or ester. Preferably, the cleavable bond is a disulfide bond or a peptide bond. For example, such a peptide bond is cleavable by a proteolytic enzyme.

In a particularly preferred embodiment, the linkage between glycoside molecule and the binding molecule occurs via an acid-labile bond that is stable at pH 7.4 and releases the saponin between pH 6.5 and 5.0. This is, e.g., realized via an imine formed by an amino group of the binding molecule and the aldehyde group of a saponin. Other chemical bonds that fulfill the pH-condition can also be used for aldehyde coupling, e.g. particular hydrazones or acetals, requiring hydrazides and hydroxyl groups as the functional group on the binding molecule, respectively. Such functional groups, if not present in sufficient amounts, may be added to the binding molecule prior to coupling to the glycoside. If the bond is a cleavable bond, and the glycoside is a saponin, the saponin is preferably attached to the binding molecule via the aldehyde function in position 23 or via one of the carboxyl groups in saponin, more preferably through the aldehyde function.

In a preferred embodiment, a combination according to the invention is provided, wherein the expression “the at least one glycoside molecule” is meant as a defined number of glycoside molecules or a defined range, rather than a random number. Preferably, a binding molecule present in a combination according to the invention comprises a defined number or defined range of glycoside molecules. An embodiment is the combination of the invention comprising the endosomal and/or lysosomal escape enhancing conjugate of the invention, wherein the endosomal and/or lysosomal escape enhancing conjugate comprises a defined number of glycosides or a defined range. This is especially advantageous for drug development in relation to market authorization. A defined number in this respect means that each binding molecule or each endosomal and/or lysosomal escape enhancing conjugate comprises a previously defined number of glycoside molecules. Depending on the expression level of the target surface molecule, the number or range maybe higher or lower. It is envisaged to offer a standard set of binding molecules or a standard set of endosomal and/or lysosomal escape enhancing conjugates, comprising, e.g., one, two, four, eight, fifteen, thirty, etc., glycoside molecules per binding molecule or per endosomal and/or lysosomal escape enhancing conjugate so that the optimal number can be easily picked, depending on the expression level of the targeted surface molecule. Preferably, such a defined range is between 1-30, more preferably between 1-20, even more preferably between 1-10, even more preferably between 1-6, even more preferably between 2-6, even more preferably between 2-5, even more preferably between 3-5, most preferably between 3-4 glycoside molecules.

In one preferred embodiment, a combination according to the invention is provided wherein said effector molecule is a pharmaceutically active substance, such as a toxin such as a proteinaceous toxin, a drug, a polypeptide or a polynucleotide. A pharmaceutically active substance in this invention is an effector molecule that is used to achieve a beneficial outcome in an organism, preferably a vertebrate, more preferably a human being. Benefits include diagnosis, prognosis, treatment, cure and prevention of diseases and/or symptoms. The pharmaceutically active substance may also lead to undesired harmful side effects. In this case, pros and cons must be weighed to decide whether the pharmaceutically active substance is suitable in the particular case. If the effect of the pharmaceutically active substance inside a cell is predominantly beneficial for the organism as a whole, the cell is called a target cell. If the effect inside a cell is predominantly harmful for the organism as a whole, the cell is called an off-target cell. In artificial systems such as cell cultures and bioreactors, target cells and off-target cells depend on the purpose and are defined by the user. Examples of effector molecules are a drug, a toxin, a polypeptide (such as an enzyme), and a polynucleotide, including polypeptides and polynucleotides that comprise non-natural amino acids or nucleic acids. Effector molecules include, amongst others: DNA: single stranded DNA (e.g. DNA for adenine phosphoribosyltransferase); linear doubled stranded DNA; circular double stranded DNA (e.g. plasmids); RNA: -mRNA (e.g. TAL effector molecule nucleases), tRNA, rRNA, siRNA, miRNA, asRNA, LNA and BNA; Protein and peptides: Cas9; toxins (e.g. saporin, dianthin, gelonin, (de)bouganin, agrostin, ricin (toxin A chain); pokeweed antiviral protein, apoptin, diphtheria toxin, pseudomonas exotoxin) metabolic enzymes (argininosuccinate lyase, argininosuccinate synthetase), enzymes of the coagulation cascade, repairing enzymes; enzymes for cell signalling; cell cycle regulation factors; gene regulating factors (transcription factors such as NF-κB or gene repressors such as methionine repressor). A toxin, as used in this invention, is defined as a pharmaceutically active substance that is able to kill or inactivate a cell. Preferably, a targeted toxin is a toxin that is only, or at least predominantly, toxic for target cells but not for off-target cells. The net effect of the targeted toxin is preferably beneficial for the organism as a whole.

In a preferred embodiment, a combination according to the invention is provided, wherein said target cell is a diseased or disease-related cell, preferably a tumour cell or a tumour-associated cell (e.g. tumour vascular cell), or an immune cell (e.g. a T regulatory cell). An embodiment is the combination of the invention comprising the endosomal and/or lysosomal escape enhancing conjugate of the invention, wherein said target cell is a diseased cell or a disease-related cell, preferably a tumor cell or a tumor-associated cell (e.g. tumor vascular cell), or an immune cell (e.g. a T regulatory cell), or an autoimmune cell.

Preferably, the effector molecule, whose effect is enhanced by the glycoside molecules (e.g. saponins), detaches from the binding molecule after being endocytosed. This can be achieved by a cleavable bond that breaks, e.g. under acidic, reductive, enzymatic or light-induced conditions. Therefore, in a preferred embodiment, a combination according to the invention is provided, wherein said at least one effector molecule is bound to said second binding molecule via a cleavable bond, wherein preferably said cleavable bond is subject to cleavage under acidic, reductive, enzymatic or light-induced conditions. Preferably the cleavable bond is an imine, hydrazone, oxime, 1,3-dioxolane, disulfide or ester, more preferably a disulfide or hydrazone bond. Typically, the cleavable bond is a disulfide bond or a peptide bond.

In another preferred embodiment, a combination according to the invention is provided, wherein said at least one effector molecule is bound to said binding molecule or to said binding moiety via a non-cleavable bond, e.g. through an amide coupling or amine formation. This is, e.g., realized via carbodiimide mediated amide bond formation by an amino group of the polymeric or assembled polymeric structure and an activated carboxylic acid group on the effector molecule.

An embodiment is the combination according to the invention, wherein the endosomal and/or lysosomal escape enhancing conjugate and the binding moiety are able to bind to the same target cell-specific surface molecule or -structure, preferably to specifically bind to the same target cell-specific surface molecule or -structure.

An embodiment is the combination comprising or consisting of the endosomal and/or lysosomal escape enhancing conjugate and the binding moiety according to the invention, wherein the endosomal and/or lysosomal escape enhancing conjugate is able to compete with the binding moiety for binding to the target cell surface molecule or -structure, preferably the target cell surface molecule or -structure is a target cell-specific surface molecule or -structure.

An embodiment is the combination comprising or consisting of the endosomal and/or lysosomal escape enhancing conjugate and the binding moiety according to the invention, wherein the endosomal and/or lysosomal escape enhancing conjugate is not able to compete with the binding moiety for binding to the target cell surface molecule or -structure, preferably the target cell surface molecule or -structure is a target cell-specific surface molecule or -structure.

An embodiment is the combination comprising or consisting of the endosomal and/or lysosomal escape enhancing conjugate and the binding moiety according to the invention, wherein the endosomal and/or lysosomal escape enhancing conjugate and the binding moiety are, independently from one another, able to bind to the same epitope, preferably able to specifically bind to the same epitope.

An embodiment is the combination comprising or consisting of the endosomal and/or lysosomal escape enhancing conjugate and the binding moiety according to the invention, wherein the endosomal and/or lysosomal escape enhancing conjugate is able to bind to a first epitope, preferably able to specifically bind to a first epitope, which is different from a second epitope to which the binding moiety is able to bind, preferably able to specifically bind.

An embodiment is the combination comprising or consisting of the endosomal and/or lysosomal escape enhancing conjugate and the binding moiety according to the invention, wherein the first and the second epitope are present on two different target cell surface molecules or -structures, preferably on two different target cell-specific surface molecules or -structures, wherein the two different target cell surface molecules or -structures, or the two different target cell-specific surface molecules or -structures, are co-expressed on at least one target cell.

An embodiment is the combination comprising or consisting of the endosomal and/or lysosomal escape enhancing conjugate and the binding moiety according to the invention, wherein the target cell surface molecule(s) or -structure(s), preferably target cell-specific surface molecule(s) or -structure(s), is/are selected from HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CD38, FGFR3, CD123, DLL3, CEACAM5, HER3, CD74, PTK7, Notch3, FGF2, C4.4A, FLT3, CD71.

An embodiment is the combination comprising or consisting of the endosomal and/or lysosomal escape enhancing conjugate and the binding moiety according to the invention, wherein the endosomal and/or lysosomal escape enhancing conjugate comprises cetuximab or trastuzumab or anti-CD71 monoclonal antibody for binding to cell-surface receptor CD71 (transferrin receptor) as a ligand, and/or wherein the binding moiety comprises cetuximab or trastuzumab or anti-CD71 monoclonal antibody.

An embodiment is the combination comprising or consisting of the endosomal and/or lysosomal escape enhancing conjugate and the binding moiety according to the invention, wherein the endosomal and/or lysosomal escape enhancing conjugate and the binding moiety comprises respectively any combination of immunoglobulins selected from:
i. cetuximab as a ligand and trastuzumab;
ii. cetuximab as a ligand and cetuximab;
iii. trastuzumab as a ligand and trastuzumab;
iv. trastuzumab as a ligand and cetuximab;
v. anti-CD71 monoclonal antibody for binding to cell-surface receptor CD71 (transferrin receptor) as a ligand and trastuzumab;
vi. anti-CD71 mAb as a ligand and cetuximab;
vii. anti-CD71 mAb as a ligand and anti-CD71 mAb;
viii. cetuximab as a ligand and anti-CD71 mAb; or
ix. trastuzumab as a ligand and anti-CD71 mAb.

An embodiment is the combination comprising or consisting of the endosomal and/or lysosomal escape enhancing conjugate and the binding moiety according to the invention wherein the binding moiety comprises at least one bound glycoside, which glycoside is/are a bisdesmosidic triterpene.

An embodiment is the combination according to the invention, wherein the glycoside is a bisdesmosidic triterpene saponin.

An embodiment is the combination comprising or consisting of the endosomal and/or lysosomal escape enhancing conjugate and the binding moiety according to the invention, wherein the glycoside is a bisdesmosidic triterpene saponin belonging to the type of a 12,13-dehydrooleanane with an aldehyde function in position 23.

An embodiment is the combination comprising or consisting of the endosomal and/or lysosomal escape enhancing conjugate and the binding moiety according to the invention, wherein the saponin is a saponin that is isolatable from Gypsophila species or Saponaria species.

An embodiment is the combination comprising or consisting of the endosomal and/or lysosomal escape enhancing conjugate and the binding moiety according to the invention, wherein the at least one glycoside is any one or more of saponins SA1641, SO1861 and GE1741, or a combination thereof, more preferably the at least one glycoside is/are SO1861, or a diastereomer of such saponin(s).

An embodiment is the combination comprising or consisting of the endosomal and/or lysosomal escape enhancing conjugate and the binding moiety according to the invention, wherein the endosomal and/or lysosomal escape enhancing conjugate is devoid of any payload such as a payload selected from any one or more of the effector moieties as outlined for the endosomal and/or lysosomal escape enhancing conjugate of the invention.

An embodiment is the combination comprising or consisting of the endosomal and/or lysosomal escape enhancing conjugate and the binding moiety according to the invention, wherein the endosomal and/or lysosomal escape enhancing conjugate comprises more than one saponin moiety, preferably 2, 4, 8, 16, 32, 64 saponin moieties, preferably terminal saponin moieties.

An embodiment is the combination comprising or consisting of the endosomal and/or lysosomal escape enhancing conjugate and the binding moiety according to the invention, wherein the at least one glycoside bound to the binding moiety is bound via a cleavable bond, wherein preferably said cleavable bond is subject to cleavage under acidic-, reductive-, enzymatic- or light-induced conditions, such as acidic conditions at a pH of lower than 6.5 such as between 4.0 and 6.0, and wherein the cleavable bond preferably is a disulfide bond or a peptide bond, or an amide bond.

An embodiment is the combination comprising or consisting of the endosomal and/or lysosomal escape enhancing conjugate and the binding moiety according to the invention, wherein the cleavable bond is a covalent bond, preferably an imine bond, a hydrazone bond, an oxime bond, a 1,3-dioxolane bond or an ester bond.

An embodiment is the combination comprising or consisting of the endosomal and/or lysosomal escape enhancing conjugate and the binding moiety according to the invention, wherein the endosomal and/or lysosomal escape enhancing conjugate comprises a defined number of glycosides or a defined range of glycosides, and/or, wherein the binding moiety comprises a defined number of glycosides or a defined range.

An embodiment is the combination comprising or consisting of the endosomal and/or lysosomal escape enhancing conjugate and the binding moiety according to the invention, wherein the aforementioned defined range(s) is/are between 1-64 glycoside(s), preferably between 1-32, more preferably between 1-16, more preferably between 1-8, more preferably between 2-6, more preferably between 2-5, more preferably between 3-5, more preferably between 3-4 glycosides.

An embodiment is the combination comprising or consisting of the endosomal and/or lysosomal escape enhancing conjugate and the binding moiety according to the invention, wherein said effector molecule that is bound to the binding moiety is a pharmaceutically active substance, such as a toxin such as a proteinaceous toxin, a small molecule drug, a macromolecular drug, a polypeptide or a polynucleotide such as an RNA, a BNA, a DNA, an LNA.

An embodiment is the combination comprising or consisting of the endosomal and/or lysosomal escape enhancing conjugate and the binding moiety according to the invention, wherein said target cell is an aberrant cell, a diseased cell or a disease-related cell, preferably a tumor cell or a tumor-associated cell (e.g. tumor vascular cell), or an immune cell (e.g. a T regulatory cell), or an autoimmune cell.

An embodiment is the combination comprising or consisting of the endosomal and/or lysosomal escape enhancing conjugate and the binding moiety according to the invention, wherein said at least one effector molecule bound to the binding moiety is bound to said binding moiety via a cleavable bond, wherein preferably said cleavable bond is subject to cleavage under acidic-, reductive-, enzymatic- or light-induced conditions, such as acidic conditions at a pH of lower than 6.5 such as between 4.0 and 6.0, and/or wherein the cleavable bond is a disulfide bond or a peptide bond or an amide bond.

An embodiment is the combination comprising or consisting of the endosomal and/or lysosomal escape enhancing conjugate and the binding moiety according to the invention, wherein said glycoside that is bound to the binding moiety, and the one or more glycosides comprised by the endosomal and/or lysosomal escape enhancing conjugate is/are capable of augmenting endosomal and/or lysosomal escape and/or intracellular trafficking of said effector molecule that is bound to the binding moiety.

An embodiment is the combination comprising or consisting of the endosomal and/or lysosomal escape enhancing conjugate and the binding moiety according to the invention, comprising at least two endosomal and/or lysosomal escape enhancing conjugates according to the invention, preferably two such conjugates or three such conjugates, and optionally comprising at least one binding moiety according to the invention, or comprising at least one endosomal and/or lysosomal escape enhancing conjugate according to the invention and at least two binding moieties according to the invention.

An embodiment is the combination comprising or consisting of the endosomal and/or lysosomal escape enhancing conjugate and the binding moiety comprising at least one endosomal and/or lysosomal escape enhancing conjugate according to the invention, such as one such conjugate or two such conjugates, and one or more of at least one saponin as outlined for the endosomal and/or lysosomal escape enhancing conjugate of the invention, at least one effector moiety as outlined for the endosomal and/or lysosomal escape enhancing conjugate of the invention, and at least one ligand as outlined for the endosomal and/or lysosomal escape enhancing conjugate of the invention.

As already mentioned before, a combination according to the invention enables more efficient targeting of a toxin to a target cell and is thus especially useful as a medicament, or to be incorporated in a pharmaceutical composition. Potential side-effects will be decreased due to lowering of the dosage of the effector molecule without lowering the efficacy.

An embodiment is the pharmaceutical composition according to the invention, further comprising at least one further active pharmaceutically ingredient, such as a further immunoglobulin.

In one embodiment, therefore, a combination according to the invention for use as a medicament is provided. In a preferred embodiment, a combination for use according to the invention in a method of treating cancer is provided. Preferably, said first binding molecule and said second binding molecule are to be administered concomitant or sequentially, preferably concomitant.

In another embodiment, a pharmaceutical composition is provided, comprising a combination according to the invention and a pharmaceutically acceptable excipient. In a preferred embodiment, a pharmaceutical composition according to the invention is provided, wherein the composition is for use in a method of treating cancer.

Further provided is a method of treating cancer, the method comprising administering a combination according to invention or a pharmaceutical composition according to the invention to a patient in need thereof.

An embodiment is the combination comprising or consisting of the endosomal and/or lysosomal escape enhancing conjugate and the binding moiety according to the invention or the composition of the invention for use as a medicament.

An aspect of the invention relates to a pharmaceutical composition comprising a combination comprising or consisting of the endosomal and/or lysosomal escape enhancing conjugate and the binding moiety according to the invention or the composition of the invention, and a pharmaceutically acceptable excipient.

An embodiment is the pharmaceutical composition comprising or consisting of the endosomal and/or lysosomal escape enhancing conjugate and the binding moiety according to the invention, further comprising at least one further active pharmaceutically ingredient, such as a further immunoglobulin or an ADC or a small-molecule drug.

An aspect of the invention relates to a combination or composition for use according to the invention, or pharmaceutical composition according to the invention, for use in a method of treating cancer or for use in a method of treating an autoimmune disease.

An embodiment is the combination for use according to the invention, wherein said endosomal and/or lysosomal escape enhancing conjugate and said binding moiety are to be administered concomitant or sequentially, preferably concomitant, or the composition for use according to the invention wherein said endosomal and/or lysosomal escape enhancing conjugate(s) and said saponin(s), effector moiety/moieties, ligand(s) are to be administered concomitant or sequentially, preferably concomitant.

An aspect of the invention relates to a method of treating cancer, the method comprising administering an effective dose of a combination comprising or consisting of the endosomal and/or lysosomal escape enhancing conjugate and the binding moiety according to the invention to a patient in need thereof.

An aspect of the invention relates to a method of treating cancer, the method comprising administering an effective dose of a pharmaceutical composition according to the invention, to a patient in need thereof.

An aspect of the invention relates to a kit comprising a first container containing an endosomal and/or lysosomal escape enhancing conjugate according to the invention and a second container containing a binding moiety according to the invention, the kit further comprising instructions for using the binding molecules. A further aspect relates to a kit comprising a first container containing a first binding molecule comprising a saponin according to the invention and a second container containing a second binding molecule comprising a toxin according to the invention, the kit further comprising instructions for using the binding molecules. An aspect of the invention relates to a kit comprising a first container containing an endosomal and/or lysosomal escape enhancing conjugate according to the invention and a second container containing a binding moiety according to the invention, the kit further comprising instructions for using the content of both containers.

The endosomal and/or lysosomal escape enhancing conjugate, the binding moiety, the first binding molecule and the second binding molecule preferably comprise an immunoglobulin such as an IgG as the ligand moiety comprised by said conjugate, moiety, binding molecules, according to the invention. Preferred are antibodies or binding domains or fragments thereof such as trastuzumab and cetuximab, although antibodies targeting the same (HER2, EGFR) or different cell-surface molecules specifically present on aberrant cells are equally suitable for directing the effector moiety comprised by the binding moiety and comprised by the second binding molecule to the target aberrant cell.

It is part of the invention that the combination of the invention or the endosomal and/or lysosomal escape enhancing conjugate of the invention is further combined with a second complex of a binding molecule or a binding moiety and a saponin, or is further combined with a pharmaceutical compound, an antibody, etc., therewith providing a composition comprising three or more enhancers, pharmaceutically active ingredients, etc., e.g. a conjugate of the invention combined with a binding moiety complexed with an effector molecule, further combined with a pharmaceutical, which is either or not linked to a saponin, and which is either or not coupled to a ligand such as a targeting immunoglobulin, a domain or a fragment thereof. Furthermore, an embodiment is the combination of the invention, wherein the second binding molecule or the binding moiety is provided with two or more effector moieties such as a toxin or immunotoxin, wherein the two or more effector moieties are the same or different.

As indicated before, the drawbacks summarized in the Background section validates the need for novel approaches to synchronize the presence of effector molecule and endosomal escape enhancer in the acidic compartments of the endocytic pathway that solves at least one of the above mentioned drawbacks. Previously, the inventors developed the enhanced delivery of protein-based targeted drugs by particular saponins, which was the subject of European patent EP1623715. The present invention is a significant non-obvious improvement of what has been described in EP1623715 and solves at least one of the before mentioned drawbacks of the approaches known today.

An aspect of the invention relates to an endosomal and/or lysosomal escape enhancing conjugate according to the invention for cytosolic delivery of an E moiety wherein the E moiety is at least one effector moiety, said conjugate comprising a W moiety selected from a polymeric and an oligomeric structure and comprising at least one glycoside coupled to said polymeric or oligomeric structure.

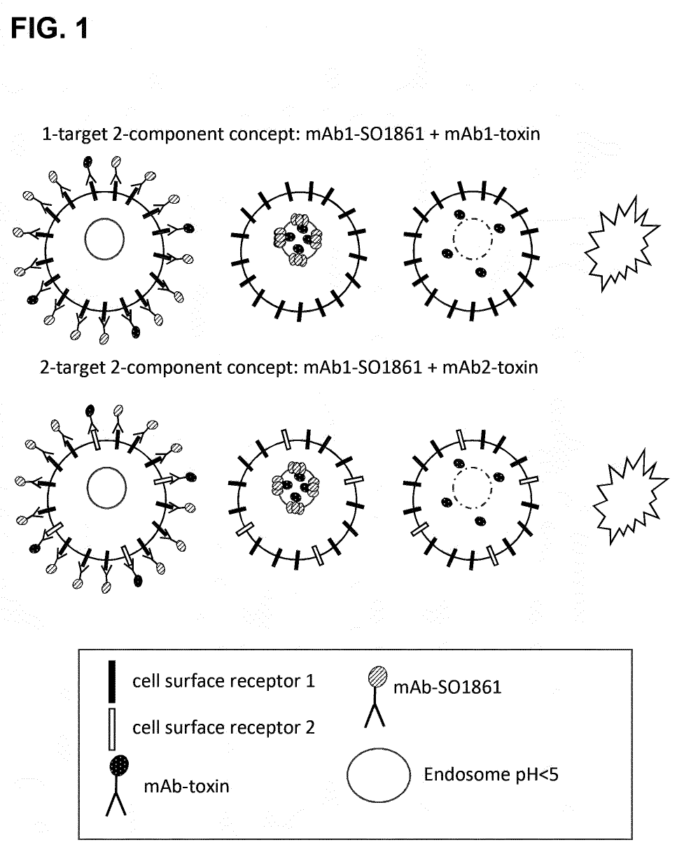

In an embodiment, the invention provides a scaffold for cytosolic delivery of an effector molecule, the scaffold comprising a polymeric or oligomeric structure and at least one glycoside coupled to said polymeric or oligomeric structure. The at least one glycoside molecule is preferably coupled to the polymeric or oligomeric structure through (1) a covalent bond, (2) electrostatic interactions including ionic and hydrogen bonds, (3) van der Waals forces including dipole-dipole interactions, (4) π-electron effects, or (5) hydrophobic effects, more preferably through a covalent bond (see FIG. 1). The scaffold is preferably able to augment endosomal escape of an effector molecule, more preferably escape of the effector molecule into the cytosol of a cell, i.e. in the aqueous component of the cytoplasm of a cell, within which various organelles and particles are suspended. After entering the cytosol, said effector molecule might move to other cell units such as the nucleus.

Before the present invention it was not possible to guide multiple glycoside molecules to a (target) cell. In particular, it was not possible to specifically guide an effector molecule and a particular number or range of glycoside molecules per effector molecule at the same time to the endocytic pathway of a cell. A solution provided for by the invention polymerizes the glycoside molecules and enables re-monomerization after endocytosis. “Polymerizes” in this context means the reversible and/or irreversible multiple conjugation of glycoside molecules to a polymeric or oligomeric structure to form a scaffold or the reversible and/or irreversible multiple conjugation of (modified) glycoside molecules thereby forming a polymeric or oligomeric structure to form a scaffold. “Re-monomerization” in this context means the cleavage of the glycoside molecules from the scaffold after endocytosis and regaining the native chemical state of the unbound glycoside molecules. Due to the complex chemistry of the glycoside molecule the ‘polymerization’ of glycoside molecules and their “re-monomerization” after endocytosis was a challenging task. In particular, the chemical reactions used normally occur in water-free organic solvents, but glycoside molecules and biocompatible polymers are water-soluble molecules. The chemical properties of the unmodified glycoside molecule further prohibited polymerization by itself and, one other possible solution, to bind multiple glycoside molecules (directly) to the effector molecule was estimated not to be very promising, as an effector molecule (drug, toxin, polypeptide or polynucleotide) does typically not provide sufficient binding sites and because the coupling product would become quite heterogeneous. Further, there was a considerable risk that the effector molecule loses its function after coupling. The present invention solves at least one of these drawbacks.

To explain the invention in more detail, the process of cellular uptake of substances and the used terminology in this invention is described first. The uptake of extracellular substances into a cell by vesicle budding is called endocytosis. Said vesicle budding can be characterized by (1) receptor-dependent ligand uptake mediated by the cytosolic protein clathrin, (2) lipid-raft uptake mediated by the cholesterol-binding protein caveolin, (3) unspecific fluid uptake (pinocytosis), or (4) unspecific particle uptake (phagocytosis). All types of endocytosis run into the following cellular processes of vesicle transport and substance sorting called the endocytic pathways. The endocytic pathways are complex and not fully understood. In an earlier understanding, organelles are formed de novo and mature into the next organelle along the endocytic pathway. Nowadays, it is rather hypothesized that the endocytic pathways involve stable compartments that are connected by vesicular traffic. A compartment is a complex, multifunctional membrane organelle that is specialized for a particular set of essential functions for the cell. Vesicles are considered to be transient organelles, simpler in composition, and are defined as membrane-enclosed containers that form de novo by budding from a preexisting compartment. In contrast to compartments, vesicles can undergo maturation, which is a physiologically irreversible series of biochemical changes. Early endosomes and late endosomes represent stable compartments in the endocytic pathway while primary endocytic vesicles, phagosomes, multivesicular bodies (also called endosome carrier vesicles), secretory granules, and even lysosomes represent vesicles. The endocytic vesicle, which arises at the plasma membrane, most prominently from clathrin-coated pits, first fuses with the early endosome, which is a major sorting compartment of approximately pH 6.5. A large part of the cargo and membranes internalized are recycled back to the plasma membrane through recycling vesicles (recycling pathway). Components that should be degraded are transported to the acidic late endosome (pH lower than 6) via multivesicular bodies. Lysosomes are vesicles that can store mature lysosomal enzymes and deliver them to a late endosomal compartment when needed. The resulting organelle is called the hybrid organelle or endolysosome. Lysosomes bud off the hybrid organelle in a process referred to as lysosome reformation. Late endosomes, lysosomes, and hybrid organelles are extremely dynamic organelles, and distinction between them is often difficult. Degradation of an endocytosed molecule occurs inside an endolysosome or lysosome. Endosomal escape is the active or passive release of a substance from the inner lumen of any kind of compartment or vesicle from the endocytic pathway, preferably from clathrin-mediated endocytosis, or recycling pathway into the cytosol. Endosomal escape thus includes but is not limited to release from endosomes, endolysosomes or lysosomes, including their intermediate and hybrid organelles. Unless specifically indicated otherwise and in particular when relating to the endosomal escape mechanism of the glycoside molecule, whenever the word “endosome” or “endosomal escape” is used herein, it also includes the endolysosome and lysosome, and escape from the endolysosome and lysosome, respectively. After entering the cytosol, said substance might move to other cell units such as the nucleus. In formal terms, a glycoside is any molecule in which a sugar group is bound through its anomeric carbon to another group via a glycosidic bond. Glycoside molecules in the context of the invention are such molecules that are further able to enhance the effect of an effector molecule, in particular by facilitating the endosomal escape of the effector molecule. The glycoside molecules interact with the membranes of compartments and vesicles of the endocytic and recycling pathway and make them leaky for said effector molecules resulting in augmented endosomal escape. With the term “the scaffold is able to augment endosomal escape of the effector molecule” is meant that the at least one glycoside molecule, which is coupled to the polymeric or oligomeric structure, is able to enhance endosomal escape of an effector molecule when both molecules are within an endosome, preferably after the at least one glycoside is released from the polymeric or oligomeric structure, e.g., by cleavage of a cleavable bond between the at least one glycoside and the polymeric or oligomeric structure. Even though a bond between the at least one glycoside and the scaffold may be a “stable bond”, that does not mean that such bond cannot be cleaved in the endosomes by, e.g., enzymes. For instance, the glycoside, together with a linker or a part of the polymeric structure may be cleaved off the remaining polymeric structure. It could, for instance be that a protease cuts a proteinaceous polymeric structure, e.g., albumin, thereby releasing the at least one glycoside. It is, however, preferred that the glycoside molecule is released in an active form, preferably in the original form that it had before it was (prepared to be) coupled to the scaffold. With regard to the present invention the term “stable” with respect to bonds between saponins, polymeric or oligomeric structures, ligands, and/or effectors is meant that the bond is not readily broken or at least not designed to be readily broken by, e.g., pH differences, salt concentrations, or UV-light. With regard to the present invention the term “cleavable” with respect to bonds between saponins, polymeric or oligomeric structures, ligands and/or effectors is meant that the bond is designed to be readily broken by, e.g., pH differences, salt concentrations, and the like. The skilled person is well aware of such cleavable bonds and how to prepare them.

An effector molecule in the context of this invention is any substance that affects the metabolism of a cell by interaction with an intracellular effector molecule target, wherein this effector molecule target is any molecule or structure inside cells excluding the lumen of compartments and vesicles of the endocytic and recycling pathway but including the membranes of these compartments and vesicles. Said structures inside cells thus include the nucleus, mitochondria, chloroplasts, endoplasmic reticulum, Golgi apparatus, other transport vesicles, the inner part of the plasma membrane and the cytosol. Cytosolic delivery of an effector molecule in the context of the invention preferably means that the effector molecule is able to escape the endosome, which as defined previously also includes escaping the endolysosome and the lysosome, and is preferably able to reach the effector molecule target as described herein. The invention is a new type of molecule, referred to as scaffold that serves to bring both an effector molecule and at least one glycoside molecule in an endosome at the same time in a pre-defined ratio. Within the context of the present invention, the polymeric or oligomeric structure of the scaffold is a structurally ordered formation such as a polymer, oligomer, dendrimer, dendronized polymer, or dendronized oligomer or it is an assembled polymeric structure such as a hydrogel, microgel, nanogel, stabilized polymeric micelle or liposome, but excludes structures that are composed of non-covalent assemblies of monomers such as cholesterol/phospholipid mixtures. The terms “polymer, oligomer, dendrimer, dendronized polymer, or dendronized oligomer” have their ordinary meaning. In particular a polymer is a substance which has a molecular structure built up chiefly or completely from a large number of equal or similar units bonded together and an oligomer is a polymer whose molecules consist of relatively few repeating units. There is no consensus about one specific cut-off for “many” and “a few” as used in the above definition of polymer and oligomer, respectively. However, as the scaffold may comprise a polymeric or an oligomeric structure, or both, the full range of numbers of similar units bonded together applies to such structure. i.e. from 2 monomeric units to 100 monomeric units, 1000 monomeric units, and more. A structure comprising 5 or less, for instance maybe called an oligomeric structure, whereas a structure comprising 50 monomeric units maybe called a polymeric structure. A structure of 10 monomeric units maybe called either oligomeric or polymeric. A scaffold as defined herein, further may comprise at least one glycoside molecule. A scaffold is bound to at least a single S moiety, the S moiety being a glycoside such as a saponin, such as SO1861. A scaffold preferably includes a polymeric or oligomeric structure such as poly- or oligo(amines), e.g., polyethylenimine and poly(amidoamine), and biocompatible structures such as polyethylene glycol, poly- or oligo(esters), such as poly(lactids), poly(lactams), polylactide-co-glycolide copolymers, and poly(dextrin), poly- or oligosaccharides, such as cyclodextrin or polydextrose, and poly- or oligoamino acids or DNA oligo- or polymers. An assembled polymeric structure as defined herein comprises at least one scaffold or at least one functionalized scaffold and, optionally, other individual polymeric or oligomeric structures. Other individual polymeric or oligomeric structures of said assembly may be (a) scaffolds (thus comprising at least one glycoside molecule), (b) functionalized scaffolds (thus comprising at least one glycoside molecule, and a ligand and/or an effector molecule, (c) polymeric or oligomeric structures comprising at least one ligand and/or at least one effector, or (d) polymeric or oligomeric structures without a glycoside molecule, without a ligand, and without an effector molecule. A functionalized assembled polymeric structure is an assembled polymeric structure that contains (a) at least one functionalized scaffold or (b) at least one scaffold and at least one polymeric structure comprising at least one ligand and/or at least one effector. Polymeric or oligomeric structures within an assembled polymeric structure that do not comprise any of the above mentioned molecules (i.e. no glycosides, ligands or effectors) are in particular added as structural components of the assembled structures, which help to build up or to stabilize the assembled structure (“glue-like”). The inventors have found that in particular scaffolds that, after coupling to the glycoside molecules, comprise polymeric structures with many primary, secondary and/or tertiary amine groups, do not particularly enhance endosomal escape. Particularly non-preferred in this respect are polymeric and oligomeric structures comprising secondary amine groups. As said before, the acidic environment seems to be a prerequisite for the synergistic action between glycoside and effector and it is believed that such amine groups disturb the acidic environment in the late endosomes. Therefore, a scaffold that is able to disturb the acidic environment, e.g., because of the presence of amine groups, is preferably not encompassed by a scaffold according to the invention. However, primary amine groups can, of course, be blocked or shielded, e.g. by thiolation or PEGylation. After appropriate blocking or shielding of the primary amine groups, which method is known by the skilled person, such scaffold is encompassed by the claims.

In a particularly preferred embodiment, the invention provides a scaffold that does not substantially inhibit endosomal escape of the effector molecule and, preferably, comprises less than 10, more preferably less than 5, more preferably less than 2, most preferably no primary, secondary or tertiary amine group. For sake of clarity it is emphasized that a primary amine group that is blocked by, e.g., thiolation or PEGylation, is no longer called an amine group.

Whether or not a scaffold is able to disturb the acidic environment and inhibit the endosomal escape function of the at least one glycoside can be easily determined with an assay as described in Example 19. The inhibition is described as “fold amount increases of glycoside necessary to induced 50% cell killing”. It is preferred that the scaffold does not lead to an increase that is at least the increase in glycoside molecules necessary to obtain 50% cell killing observed when using Chloroquine as a positive control. Alternatively, and preferably, the scaffold does not lead to an at least 4-fold increase of glycoside molecules to induce 50% cell killing, more preferably does not lead to an at least 2-fold increase. The fold increase is to be measured in assay, essentially as described in Example 19, wherein Chloroquine, as a positive control, induces a 2-fold increase in glycoside amount to observe 50% cell killing.

With the term “improving or enhancing an effect of an effector molecule” is meant that the glycoside molecule increases the functional efficacy of that effector molecule (e.g. the therapeutic index of a toxin or a drug; the metabolic efficacy of a modifier in biotechnological processes; the transfection efficacy of genes in cell culture research experiments), preferably by enabling or improving its target engagement. Acceleration, prolongation, or enhancement of antigen-specific immune responses are preferably not included. Therapeutic efficacy includes but is not limited to a stronger therapeutic effect with lower dosing and/or less side effects. “Improving an effect of an effector molecule” can also mean that an effector molecule, which could not be used because of lack of effect (and was e.g. not known as being an effector molecule), becomes effective when used in combination with the present invention. Any other effect, which is beneficial or desired and can be attributed to the combination of effector molecule and a scaffold, as provided by the invention is considered to be “an improved effect”. In a preferred embodiment, the scaffold enhances an effect of the effector molecule which effect is intended and/or desired. In case of a proteinaceous scaffold, the proteinaceous polymeric structure as such may have, for instance, an effect on colloid osmotic pressure in the blood stream. If such effect is not the intended or desired effect of the ultimate functionalized scaffold, the proteinaceous structure of the scaffold is not the effector molecule as defined in the invention. Or, for instance in case of a DNA- or RNA-based scaffold, parts of that DNA or RNA may have an (unintended) function, e.g., by interfering with expression. If such interference is not the intended or desired effect of the ultimate functionalized scaffold, the DNA- or RNA polymeric structure of the scaffold is not the effector molecule as defined in the invention.

A number of preferred features can be formulated for endosomal escape enhancers, i.e. a glycoside according to the invention: (1) they are preferably not toxic and do not invoke an immune response, (2) they preferably do not mediate the cytosolic uptake of the effector molecule into off-target cells, (3) their presence at the site of action is preferably synchronized with the presence of the effector molecule, (4) they are preferably biodegradable or excretable, and (5) they preferably do not substantially interfere with biological processes of the organism unrelated to the biological activity of the effector molecule with which the endosomal escape enhancer is combined with, e.g. interact with hormones. Examples of glycoside molecules that fulfill the before mentioned criteria, at least to some extent, are bisdesmosidic triterpenes, preferably bisdesmosidic triterpene saponins.

In a preferred embodiment, therefore, the glycoside molecules are bisdesmosidic triterpenes, preferably bisdesmosidic triterpene saponins. The saponins preferably belong to the type of a 12,13-dehydrooleanane with an aldehyde function in position 23. Even more preferred, the glycoside molecules are saponins that can be isolated from Gypsophila or Saponaria species. However, these saponins may be isolated from said or other species, but, may of course also be expressed in other organisms, preferably genetically modified plants or plant cells, more preferably in a large scale plant cell fermentation production process. Alternatively, such glycosides may be synthesized chemically according to very complex multistep syntheses as, e.g., summarized by Yang et al. [2]. In a most preferred embodiment, a scaffold according to the invention is provided, wherein the glycoside molecules are SA1641 [1], SO1861 [3], GE1741 [4] or any of their diastereomers.

The scaffold is fundamentally independent of the type of the effector molecule. Thus, the scaffold is the basis product for a new platform technology. Since the at least one glycoside mediates intracellular delivery, the scaffold technology according to the invention is the first system known that mediates controlled intracellular effector molecule delivery by glycosides. As mentioned before, synchronization is the missing link between a very successful delivery strategy for mice and its application in humans. Moreover, direct binding of glycoside molecules to the effector molecule and/or ligand, e.g. an antibody, might result in heterogeneous products and impede the functionality of effector molecule and ligand. It can also happen that there are not sufficient binding sites at the effector molecule and ligand. The scaffold already provides an optimized and functionally active unit that can be linked to the effector molecule or ligand at a single and defined position.

Thus, it is preferred to synchronize the presence of both, the at least one glycoside, preferably a saponin, and the effector molecule, preferably a toxin, in compartments or vesicles of the endocytic pathway of the target cell. However, it has been very difficult to synchronize the presence of the molecules in the late endosomes, in order to obtain the synergistic effects in vivo. In one aspect, the invention preferably solves at least one of the following problems with respect to combining the effector molecule and the glycoside molecules in one compound: (1) the number of required glycoside molecules per effector molecule is a defined number or range (e.g., preferably at least 2, more preferably at least 3, more preferably at least 5, more preferably at least 6, more preferably at least 10, more preferably at least 15, more preferably at least 20, more preferably at least 25, more preferably at least 27, most preferably at least 30 or more) so that a simple chemical linkage is not expedient; (2) the only reasonable chemical group within, e.g., the saponins that can be used for (covalent), in particular single and cleavable, retainable coupling is required for the endosomal escape activity; (3) the effector molecule may not possess a suitable counter group for coupling; and (4) glycosides may lose their necessary potential to interact with cholesterol when not freely diffusible. All these restrictions are most likely the reason why glycosides have not been used in combination with pharmaceutically active substances in clinical investigations although the striking enhancer effect of, e.g., saponins is known for more than 10 years. A scaffold according to the present invention solves these difficulties, at least in part.

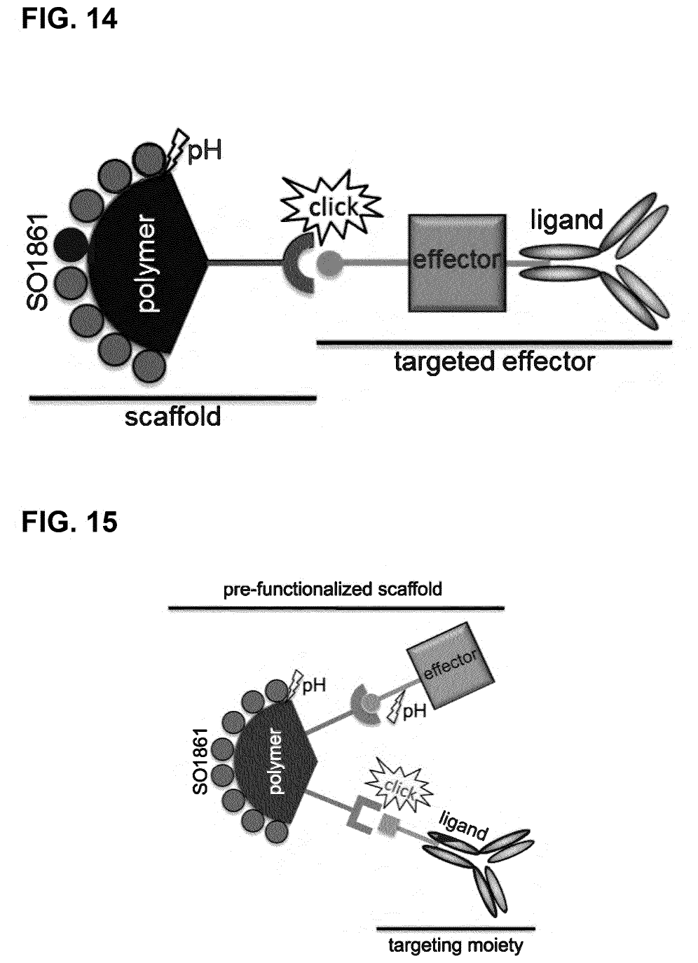

In its basic form, the scaffold comprises a polymeric and/or oligomeric structure that bears at least one glycoside molecules, e.g., particular saponins, such as SO1861 (FIG. 14, left side of the picture). In a preferred embodiment, a scaffold is provided, wherein the glycoside molecules are bound to the polymeric or oligomeric structure via a cleavable bond, wherein preferably said cleavable bond is subject to cleavage under acidic, reductive, enzymatic or light-induced conditions, more preferably under acidic conditions. Preferably the cleavable bond is an imine, hydrazone, oxime, 1,3-dioxolane or ester.

In a more preferred embodiment, the glycoside molecule is a saponin and the linkage between saponin and polymeric or oligomeric structure within the scaffold preferably occurs via an acid-labile bond that is stable at pH 7.4 and, preferably releases the saponin below pH 6.5, more preferably between pH 6.5 and 5.0. This is, e.g., realized via an imine formed by an amino group of the polymeric or oligomeric structure and the aldehyde group of the saponin. Other chemical bonds that fulfill the pH-condition can also be used for aldehyde coupling, e.g. particular hydrazones or acetals, requiring hydrazides and hydroxyl groups as the functional group of the polymeric or oligomeric structure, respectively. If the bond is a cleavable bond, a saponin is preferably attached to the polymeric or oligomeric structure of the scaffold via an aldehyde function or via one of the carboxyl groups in saponin, more preferably through the aldehyde function, preferably an aldehyde function in position 23. Alternatively, a saponin is preferably attached to the polymeric or oligomeric structure of the scaffold via a linker that connects the polymeric or oligomeric structure of the scaffold either via the aldehyde function or via the carboxylic acid function of the glycoside molecule.

In another preferred embodiment, a scaffold is provided, wherein the at least one glycoside molecule is bound to the polymeric or oligomeric structure via a stable bond. In a more preferred embodiment, the at least one glycoside molecule is a saponin and the stable bond between saponin and polymeric or oligomeric structure of the scaffold preferably occurs via an amide coupling or amine formation. This is, e.g., realized via carbodiimide mediated amide bond formation by an amino group of the polymeric or oligomeric structure and the activated glucuronic acid group of the saponin. Chemical bonds that fulfill the stable bond definition can also be used for aldehyde coupling, e.g. particular amines derived after reductive amination, requiring primary amino groups as the functional group of the polymeric or oligomeric structure. If the bond is a stable bond, the saponin is preferably attached to scaffold via one of the carboxyl groups of the saponin.

In one preferred embodiment, a scaffold according to the invention is provided, wherein the scaffold further comprises a click chemistry group for coupling to the effector molecule and/or to a ligand. A click chemistry group is a functional chemical group suitable for click chemistry, which is defined as a reaction that is modular, wide in scope, gives very high yields, generates only inoffensive byproducts, offers high selectivity, and high tolerance over different functional groups, and is stereospecific. The required process characteristics include simple reaction conditions, readily available starting materials and reagents, the use of no solvent or a solvent that is benign (such as water) or easily removed, and simple product isolation [5]. The click chemistry group is preferably a tetrazine, azide, alkene, or alkyne, or reactive derivates of them such as methyl-tetrazine or maleimide (alkene), more preferably an alkyne, or a cyclic derivative of these groups, such as cyclooctyne (e.g. aza-dibenzocyclooctyne, difluorocyclooctyne, bicyclo[6.1.0]non-4-yne, dibenzocyclooctyne).

A scaffold according to the invention thus comprises at least one glycoside molecule. With “at least one” in this context is meant that the scaffold must comprise one glycoside molecule but may also comprise a couple (e.g. two, three or four) of glycoside molecules or a multitude (e.g. 10, 20 or 100) of glycoside molecules. Depending on the application, a scaffold may be designed such that it comprises a defined number of glycoside molecules. Preferably, a scaffold according to the invention comprises a defined number or range of glycoside molecules, rather than a random number. This is especially advantageous for drug development in relation to marketing authorization. A defined number in this respect means that a scaffold preferably comprises a previously defined number of glycoside molecules. This is, e.g., achieved by designing a polymeric structure with a certain number of possible moieties for the glycoside(s) to attach. Under ideal circumstances, all of these moieties are coupled to a glycoside molecule and the scaffold than comprises the prior defined number of glycoside molecules. It is envisaged to offer a standard set of scaffolds, comprising, e.g., two, four, eight, sixteen, thirty-two, sixty-four, etc., glycoside molecules so that the optimal number can be easily tested by the user according to his needs. In one embodiment the glycoside is present in a defined range as, e.g., under non-ideal circumstances, not all moieties present in a polymeric structure bind a glycoside molecule. Such ranges may for instance be 2-4 glycoside molecules per scaffold, 3-6 glycoside molecules per scaffold, 4-8 glycoside molecules per scaffold, 6-8 glycoside molecules per scaffold, 6-12 glycoside molecules per scaffold and so on. In such case, a scaffold according to the invention thus comprises 2, 3 or 4 glycoside molecules if the range is defined as 2-4.

In a preferred embodiment, a scaffold according to the invention is provided, wherein the number of monomers of the polymeric or oligomeric structure is an exactly defined number or range. Preferably, the polymeric or oligomeric structure comprises structures such as poly(amines), e.g., polyethylenimine and poly(amidoamine), or structures such as polyethylene glycol, poly(esters), such as poly(lactides), poly(lactams), polylactide-co-glycolide copolymers, poly(dextrin), or structures such as natural and/or artificial polyamino acids, DNA polymers, stabilized RNA polymers or PNA (peptide nucleic acid) polymers, either appearing as linear, branched or cyclic polymer, oligomer, dendrimer, dendron, dendronized polymer, dendronized oligomer or assemblies of these structures, either sheer or mixed. Preferably, the polymeric or oligomeric structures are biocompatible, wherein biocompatible means that the polymeric or oligomeric structure does not show substantial acute or chronic toxicity in organisms and can be either excreted as it is or fully degraded to excretable and/or physiological compounds by the body's metabolism. Assemblies can be built up by covalent cross-linking or non-covalent bonds and/or attraction. They can therefore also form nanogels, microgels, or hydrogels, or they can be attached to carriers such as inorganic nanoparticles, colloids, liposomes, micelles or particle-like structures comprising cholesterol and/or phospholipids. Said polymeric or oligomeric structures preferably bear an exactly defined number or range of coupling moieties for the coupling of glycoside molecules. Preferably at least 50%, more preferably at least 75%, more preferably at least 85%, more preferably at least 90%, more preferably at least 95%, more preferably at least 98%, more preferably at least 99%, most preferably 100% of the exactly defined number or range of coupling moieties in the polymeric or oligomeric structure is occupied by a glycoside molecule in a scaffold according to the invention.

Preferably, a dendron is a branched, clearly defined tree-like polymer with a single chemically addressable group at the origin of the tree, called the focal point. A dendrimer is a connection of two or more dendrons at their focal point. A dendronized polymer is a connection of the focal point of one or more dendrons to a polymer. In a preferred embodiment, a scaffold according to the invention is provided, wherein the polymeric or oligomeric structure comprises a linear, branched or cyclic polymer, oligomer, dendrimer, dendron, dendronized polymer, dendronized oligomer or assemblies of these structures, either sheer or mixed, wherein assemblies can be built up by covalent cross-linking or non-covalent attraction and can form nanogels, microgels, or hydrogels, and wherein, preferably, the polymer is a derivative of a poly(amine), e.g., polyethylenimine and poly(amidoamine), and structures such as polyethylene glycol, poly(esters), such as poly(lactids), poly(lactams), polylactide-co-glycolide copolymers, and poly(dextrin), and structures such as natural and/or artificial polyamino acids or DNA polymers, stabilized RNA polymers or PNA (peptide nucleic acid) polymers. Preferably, the polymeric or oligomeric structures are biocompatible. In one preferred embodiment, a scaffold according to the invention is provided, wherein said effector molecule is a pharmaceutically active substance, such as a toxin, a drug, a polypeptide and/or a polynucleotide. In a more preferred embodiment, the effector molecule is a toxin, a micro RNA, or a polynucleotide encoding a protein.

A pharmaceutically active substance in this invention is an effector molecule that is used to achieve a beneficial outcome in an organism, preferably a vertebrate, more preferably a human being. Benefit includes diagnosis, prognosis, treatment, cure and/or prevention of diseases and/or symptoms. The pharmaceutically active substance may also lead to undesired harmful side effects. In this case, pros and cons must be weighed to decide whether the pharmaceutically active substance is suitable in the particular case. If the effect of the pharmaceutically active substance inside a cell is predominantly beneficial for the whole organism, the cell is called a target cell. If the effect inside a cell is predominantly harmful for the whole organism, the cell is called an off-target cell. In artificial systems such as cell cultures and bioreactors, target cells and off-target cells depend on the purpose and are defined by the user.

An effector molecule that is a polypeptide may be, e.g., a polypeptide that recover a lost function, such as for instance enzyme replacement, gene regulating functions, or a toxin. Examples of polypeptides as effector molecules are, e.g., Cas9; toxins (e.g. saporin, dianthin, gelonin, (de)bouganin, agrostin, ricin (toxin A chain); pokeweed antiviral protein, apoptin, diphtheria toxin, pseudomonas exotoxin) metabolic enzymes (e.g. argininosuccinate lyase, argininosuccinate synthetase), enzymes of the coagulation cascade, repairing enzymes; enzymes for cell signaling; cell cycle regulation factors; gene regulating factors (transcription factors such as NF-κB or gene repressors such as methionine repressor).

An effector molecule that is a polynucleotide may, e.g., be a polynucleotide that comprises coding information, such as a gene or an open reading frame encoding a protein. It may also comprise regulatory information, e.g. promotor or regulatory element binding regions, or sequences coding for micro RNAs. Such polynucleotide may comprise natural and artificial nucleic acids. Artificial nucleic acids include peptide nucleic acid (PNA), BNA, Morpholino and locked nucleic acid (LNA), as well as glycol nucleic acid (GNA) and threose nucleic acid (TNA). Each of these is distinguished from naturally occurring DNA or RNA by changes to the backbone of the molecule. Examples of nucleotides as effector molecules are, e.g., DNA: single stranded DNA (e.g. DNA for adenine phosphoribosyltransferase); linear double stranded DNA (e.g. clotting factor IX gene); circular double stranded DNA (e.g. plasmids); RNA: mRNA (e.g. TAL effector molecule nucleases), tRNA, rRNA, siRNA, miRNA, antisense RNA.

A toxin in this invention is a pharmaceutically active substance that is able to kill a cell. Preferably, a targeted toxin is a toxin that is only or at least predominantly toxic for target cells but not for off-target cells. An effector molecule useful in the present invention preferably relies on late endosomal escape for exerting its effect. Some effectors, such as, e.g., a pseudomonas exotoxin, are rerouted to other organelles prior to the “late endosomal stage” and, thus, would normally not benefit from a scaffold according to the present invention. However, such toxin may be adapted for use with the present invention, e.g., by deleting the signal peptide responsible rerouting. In particular toxins that are highly toxic and would require only one molecule to escape the endosomes to kill a cell maybe modified to be less potent. It is preferred to use a toxin that kills a cell if at least 2, more preferably at least 5, more preferably at least 10, more preferably at least 20, more preferably at least 50, most preferably at least 100 toxin molecules escape the endosome. It is further preferred that the functionalized scaffold comprises a ratio of at least 2:1, more preferably at least 5:1, more preferably at least 10:1, more preferably at least 20:1, most preferably at least 50:1 glycoside molecules for each effector molecule. In particular in a functionalized scaffold comprising an assembled polymeric structure, wherein the glycoside molecules and the effector molecules are attached to different polymeric structures within said assembly, it is preferred to have a ratio of at least 10:1, more preferably at least 20:1, more preferably at least 50:1, more preferably at least 100:1, most preferably at least 200:1 glycoside molecules with respect to effector molecule in such assembly. Further, in order to reduce off-target toxicity, cell membrane non-permeable small molecule toxins are preferred effector molecules over cell membrane permeable toxins.

The invention further provides a functionalized scaffold comprising at least one scaffold according to the invention, coupled to either a) at least one effector molecule, b) at least one ligand, c) at least one effector molecule and in addition at least one ligand (FIG. 15), d) at least one effector molecule that itself bears at least one ligand (FIG. 14), ore) at least one ligand that itself bears at least one effector molecule. Such coupling in a)-e) maybe achieved through a cleavable or stable bond. Preferably, coupling in a)-e) independently occurs via click chemistry bonds. Preferably, the functionalized scaffold is able to enhance endosomal escape of the effector. In one preferred embodiment, a functionalized scaffold according to the invention is provided, wherein said at least one effector molecule is a pharmaceutically active substance, such as a toxin, a drug, a polypeptide and/or a polynucleotide. In a more preferred embodiment, the effector molecule is a toxin or a polynucleotide coding for a protein.

The term “ligand” as used in this invention has its ordinary meaning and preferably means a molecule or structure that is able to bind another molecule or structure on the cell surface of a target cell, wherein said molecule or structure on the cell surface can be endocytosed and is preferably absent or less prominent on off-target cells. Preferably, said molecule or structure on the cell surface is constitutively endocytosed. More preferably a ligand in this invention induces endocytosis of said molecule or structure on the cell surface of target cells after binding to said molecule or structure. This is for instance the case for Epidermal Growth Factor Receptor (EGFR), present on the surface of a variety of cancer cells. Examples of molecules or structures on the cell surface of target cells that are constitutively endocytosed, are for instance Claudin-1 or major histocompatibility complex class II glycoproteins. A ligand can, e.g., be an antibody, a growth factor or a cytokine. Combining a toxin with a ligand is one possibility to create a targeted toxin. A toxin that is only toxic in a target cell because it interferes with processes that occur in target cells only can also be seen as a targeted toxin (as in off-target cells it cannot exert its toxic action, e.g. apoptin). Preferably, a targeted toxin is a toxin that is combined with a ligand in order to be active in target cells and not in off-target cells (as it is only bound to and endocytosed by target cells). In a functionalized scaffold comprising a ligand and an effector molecule, the ligand guides the effector molecule and scaffold to the target cells. After internalization, the at least one glycoside, preferably a saponin, mediates the endosomal escape of the effector molecule.

In the basic version of the scaffold, i.e., a non-functionalized scaffold, the thus produced scaffold can be supplied to, e.g., a drug manufacturer, who will then be responsible for the coupling of the effector molecule alone or effector molecule and ligand to the scaffold. The drug manufacturer can, if required, add cleavable units to release the effector molecule from the scaffold and/or ligand, e.g. by inserting disulfide bridges between effector molecule and ligand and/or effector molecule and click position. The invention also provides a (pre-)functionalized version of the scaffold, wherein this functionalized scaffold already bears an effector molecule, e.g. a tumor cell-killing toxin (FIG. 15). The endosomal effector molecule release is preferably already included. The functionalized scaffold can be supplied to the pharmaceutical industry, e.g. for further development of existing and future therapeutic antibodies and to any supplier or owner of antibodies to functionalize the targeting antibody. Functionalized scaffolds can also be used by biotechnology companies or for research.

Before the present invention, the required enhancer for effective delivery of intracellularly active effector molecules, e.g., a saponin molecule, is administered separately (and this type of application is therefore referred to as two-component system hereafter) and is systemically distributed throughout the whole body. Therefore, it is not targeted to a specific cell, and meets more or less by chance the targeted effector molecule inside the endosomes of the target cells, i.e. at the site of cytosolic uptake. The only possibility to influence the efficacy of the two-component system until now is to find out the pharmacokinetics of effector molecule and endosomal escape enhancer and to adapt the time points of injection accordingly. This must be done for each new effector molecule and each disease, i.e. target cell, separately and will, even when optimized, only result in less than 1% of the endosomal escape enhancing molecules being at the target site, i.e. inside the endocytic pathway of the target cells.

The new functionalized scaffold provides a number of advantages compared to the two-component system:
1. Use of the functionalized scaffold results in a one-component system, i.e. the effector molecule and endosomal escape enhancer, i.e., the at least one glycoside, are delivered at the same time in a pre-defined ratio to the endosomes.
2. The at least one glycoside molecule is now also targeted by joint use of the targeting ligand of the effector molecule; thus the glycosides are no longer distributed throughout the whole body and taken up randomly by cells, which is expected to reduce possible side effects and will broaden the therapeutic window.
3. The number of glycoside molecules per effector molecule can now be exactly defined and therefore be reduced to the required minimum; side effects by surplus glycoside molecules can be avoided. A defined number of glycoside molecules per effector molecule also facilitates marketing authorization for a specific medicament.
4. The present invention allows to offer a preformed effector molecule-loaded scaffold (functionalized scaffold) to be used with any available ligand, which makes the invention optimal for platform development.
5. If the scaffold or functionalized scaffold is attached to a carrier, it is also possible that the carrier bears the ligand and/or the effector molecule. In such case, the carrier is considered a linker.

One other application of the present invention is, e.g., gene therapy. The efficient intracellular delivery of biological macromolecules, such as, e.g., polynucleotides, is currently still a major hurdle. In contrast to conventional unspecific DNA transfection systems, the present invention is not limited to DNA and is specific for target cells. Known viral systems are efficient and specific for target cells, however, they are only suitable for DNA. Moreover, they bear the risk of immune and inflammatory responses, possess a potential oncogenic activity and require complex and expensive procedures for preparation in each individual case. The novelty of the here presented technology is based on its fundamentality, flexibility and ease of use.

In a preferred embodiment, a functionalized scaffold according to the invention is provided, wherein said at least one ligand is capable of specifically binding to a target cell specific surface molecule or structure, wherein preferably the functionalized scaffold is, after binding, endocytosed together with the surface molecule. Preferably, said target cell is a diseased or disease-related cell, preferably a tumor cell, a tumor-associated cell (e.g. tumor vascular cell), an immune cell (e.g. a T regulatory cell), or a cell with a monogenic defect. With the term “target cell specific surface molecule” is meant that the molecule is preferably expressed in the target cell and to a lesser extent in a non-target cell, either qualitatively or quantitatively. Examples of such are the EGFR receptor that is upregulated on tumor cells but also expressed (in a lower level) on, e.g., fibroblast in the skin and HER2, which is overexpressed in breast cancer cells. However, many target cell specific surface molecules are known in the art and the skilled person is very well capable of choosing a target cell specific surface molecule for a specific purpose, i.e., to discriminate a target cell from a non-target cell for a specific disease or application. As used herein, “monogenic defect” has its usual meaning which is a modification in a single gene occurring in substantially all cells of the body. The mutation may be present on one or both chromosomes (one chromosome inherited from each parent). Though relatively rare, monogenic defects affect millions of people worldwide. Scientists currently estimate that over 10,000 of human diseases are known to be monogenic disease. Non-limiting examples of monogenic diseases know to date are: sickle cell disease, cystic fibrosis, polycystic kidney disease, and Tay-Sachs disease.

In a preferred embodiment, a functionalized scaffold according to the invention is provided, wherein said at least one ligand is an antibody or a derivate or fragment thereof (e.g. VHH or scFv), a cytokine, a growth factor, or an antibody-like molecule such as an aptamer or a designed ankyrin repeat protein (DARPin). DARPins are genetically engineered antibody mimetic proteins typically exhibiting highly specific and high-affinity target protein binding. They are derived from natural ankyrin proteins, which are responsible for diverse cellular functions. They constitute a new class of potent, specific and versatile small-protein (typically 14 to 18 kDa) therapies, and are used as investigational tools in various research, diagnostic and therapeutic applications. Other non-limiting examples of antibodies or derivatives thereof known to date are: (i) a Fab′ or Fab fragment, a monovalent fragment consisting of a variable light domain, a variable heavy domain, a constant light domain and a constant heavy domain 1, or a monovalent antibody as described in WO2007059782; (ii) F(ab′)2 fragments, bivalent fragments comprising two Fab fragments linked by a disulfide bridge at the hinge region; (iii) an Fd fragment consisting essentially of the variable heavy domain and the constant heavy 1 domain; and (iv) a Fv fragment consisting essentially of the variable light and variable heavy domains of a single arm of an antibody. Furthermore, although the two domains of the Fv fragment, variable light and variable heavy, are coded for by separate genes, they may be joined, using recombinant methods, by a synthetic linker that enables them to be made as a single protein chain in which the variable light and variable heavy regions pair to form monovalent molecules (known as single chain antibodies or single chain Fv (scFv)). Preferably, the effector molecule, which effect is enhanced by the glycoside molecules (e.g. saponins), detaches from the scaffold and/or ligand when endocytosed. This can be achieved by a cleavable bond that breaks, e.g. under acidic, reductive, enzymatic or light-induced conditions. In a preferred embodiment, therefore, a functionalized scaffold according to the invention is provided, wherein said at least one effector molecule is bound to said scaffold and/or to said at least one ligand via a cleavable bond, wherein preferably said cleavable bond is subject to cleavage under acidic, reductive, enzymatic or light-induced conditions. Preferably the cleavable bond is an imine, hydrazone, oxime, 1,3-dioxolane, disulfide or ester, more preferably a disulfide or hydrazone bond.

In another preferred embodiment, a functionalized scaffold according to the invention is provided, wherein said at least one effector molecule is bound to said scaffold and/or to said at least one ligand via a stable bond, e.g. through an amide coupling or amine formation. This is, e.g., realized via carbodiimide mediated amide bond formation by an amino group of the polymeric or oligomeric structure and an activated carboxylic acid group on the effector molecule or ligand.

In a preferred embodiment, the invention provides a scaffold or functionalized scaffold according to the invention, further comprising a carrier, such as a nanoparticle, liposome, micelle, colloid, or a particle-like structure comprising cholesterol and/or phospholipids.

An aspect of the invention relates to an endosomal and/or lysosomal escape enhancing conjugate according to the invention for cytosolic delivery of an E moiety wherein the E moiety is at least one effector moiety, said conjugate comprising a W moiety selected from a polymeric and an oligomeric structure and comprising at least one glycoside coupled to said polymeric or oligomeric structure.

An embodiment is the endosomal and/or lysosomal escape enhancing conjugate, said conjugate comprising said W moiety, according to the invention, wherein the at least one glycoside is a bisdesmosidic triterpene.

An embodiment is the endosomal and/or lysosomal escape enhancing conjugate, said conjugate comprising said W moiety, according to the invention, wherein the at least one glycoside is a bisdesmosidic triterpene saponin.

An embodiment is the endosomal and/or lysosomal escape enhancing conjugate, said conjugate comprising said W moiety, according to the invention wherein the at least one glycoside is a bisdesmosidic triterpene saponin belonging to the type of a 12,13-dehydrooleanane with an aldehyde function in position 23.

An embodiment is the endosomal and/or lysosomal escape enhancing conjugate, said conjugate comprising said W moiety, according to the invention, wherein the at least one glycoside is a saponin that can be isolated from Gypsophila or Saponaria species.

An embodiment is the endosomal and/or lysosomal escape enhancing conjugate, said conjugate comprising said W moiety, according to the invention, wherein the at least one glycoside is SA1641, SO1861, and/or GE1741, or any of their diastereomers, or any combination thereof, preferably the glycoside is SO1861.

An embodiment is the endosomal and/or lysosomal escape enhancing conjugate, said conjugate comprising said W moiety, according to the invention, wherein the at least one glycoside is bound to the polymeric or oligomeric structure via a non-cleavable bond or a cleavable bond, preferably a cleavable bond, wherein preferably said cleavable bond is subject to cleavage under acidic, reductive, enzymatic or light-induced conditions, wherein the acidic conditions are preferably at a pH of between 4.0 and 6.5, such as pH 4.5-5.5.

An embodiment is the endosomal and/or lysosomal escape enhancing conjugate, said conjugate comprising said W moiety, according to the invention, wherein the cleavable bond is a covalent bond, preferably an imine, hydrazone, oxime, 1,3-dioxolane or ester, disulfide bond, amide bond, peptide bond.

An embodiment is the endosomal and/or lysosomal escape enhancing conjugate, said conjugate comprising said W moiety, according to the invention, wherein said conjugate further comprises a click chemistry group for coupling to at least one E moiety selected from at least one payload as outlined for the endosomal and/or lysosomal escape enhancing conjugate of the invention and/or at least one ligand moiety as outlined for the endosomal and/or lysosomal escape enhancing conjugate of the invention.

An embodiment is the endosomal and/or lysosomal escape enhancing conjugate, said conjugate comprising said W moiety, according to the invention, wherein the click chemistry group is a tetrazine, azide, alkene or alkyne, or a cyclic derivative of these groups.

An embodiment is the endosomal and/or lysosomal escape enhancing conjugate, said conjugate comprising said W moiety, according to the invention, wherein the at least one glycoside is a defined number of glycoside molecules or a defined range of glycoside molecules.

An embodiment is the endosomal and/or lysosomal escape enhancing conjugate, said conjugate comprising said W moiety, according to the invention, wherein the polymeric or oligomeric structure comprises a linear, branched or cyclic polymer, oligomer, dendrimer, dendron, dendronized polymer, dendronized oligomer or assemblies of these structures, either sheer or mixed, wherein assemblies are built up by covalent cross-linking or by non-covalent bonds and/or attraction and/or can form hydrogels or nanogels.

An embodiment is the endosomal and/or lysosomal escape enhancing conjugate, said conjugate comprising said W moiety, according to the invention, wherein said effector molecule is a pharmaceutically active substance, such as a toxin such as dianthin, saporin, a drug, a polypeptide and/or a polynucleotide such as an RNA, BNA, DNA, LNA.

An embodiment is the endosomal and/or lysosomal escape enhancing conjugate, said conjugate comprising said W moiety, according to the invention, further comprising a carrier.

An aspect of the invention relates to a functionalized endosomal and/or lysosomal escape enhancing conjugate comprising at least one endosomal and/or lysosomal escape enhancing conjugate, said conjugate comprising said W moiety, according to the invention, comprising or coupled to at least one E moiety wherein the E moiety is either a) at least one effector molecule, b) at least one ligand, c) at least one effector molecule and in addition at least one ligand, d) at least one effector molecule that itself bears at least one ligand, or e) at least one ligand that itself bears at least one effector molecule.

An embodiment is the functionalized endosomal and/or lysosomal escape enhancing conjugate, comprising said W moiety, according to the invention, wherein coupling in a)-e) independently occurs via click chemistry bonds.

An embodiment is the functionalized endosomal and/or lysosomal escape enhancing conjugate according to the invention, wherein said at least one effector molecule is a pharmaceutically active substance, such as a toxin such as saporin or dianthin, a drug, a polypeptide such as Cre-recombinase, or a polynucleotide such as an RNA, a DNA, an LNA, a BNA such as HSP27-silencing ASO (BNA).

An embodiment is the functionalized endosomal and/or lysosomal escape enhancing conjugate according to the invention comprising at least one ligand, wherein said at least one ligand is capable of specifically binding to a target cell-specific surface molecule or -structure, wherein preferably the functionalized endosomal and/or lysosomal escape enhancing conjugate is endocytosed together with the surface molecule or surface structure.

An embodiment is the functionalized endosomal and/or lysosomal escape enhancing conjugate according to the invention, wherein said target cell is an aberrant cell, a diseased or disease-related cell, preferably a tumor cell, a tumor-associated cell (e.g. tumor vascular cell), an immune cell (e.g. a T regulatory cell), or a cell with a monogenic defect.

An embodiment is the functionalized endosomal and/or lysosomal escape enhancing conjugate according to the invention, wherein said at least one ligand is an antibody or a binding domain or a derivate or binding fragment thereof or a Vh domain, a cytokine, a growth factor such as EGF, or an antibody-like molecule such as an aptamer or a designed ankyrin repeat protein.

An embodiment is the functionalized endosomal and/or lysosomal escape enhancing conjugate according to the invention, wherein said at least one effector molecule is bound to said at least one endosomal and/or lysosomal escape enhancing conjugate and/or to said at least one ligand via a cleavable bond, wherein preferably said cleavable bond is subject to cleavage under acidic-, reductive-, enzymatic- or light-induced conditions, wherein preferably the acidic conditions are at a pH of 6.5 or less such as 4.0-6.0 or 4.5-5.5.

As said before, the at least one glycoside molecule that is comprised within a scaffold according to the invention increases the efficacy of at least current and new effector molecules as defined in this invention. Potential side-effects will be decreased due to lowering of dosing of the effector molecule without lowering the efficacy. Therefore, the invention provides a scaffold according to the invention or a functionalized scaffold according to the invention for use in medicine or for use as a medicament. Also provided is the use of a scaffold according to the invention or a functionalized scaffold according to invention for manufacturing a medicament. Especially cancer medicines, and in particular the classical chemotherapy medicaments, are notorious for their side effects. Because of targeting and synchronization in time and place of both the pharmaceutically active substance and the glycoside molecule, a scaffold or functionalized scaffold according to the invention is especially valuable for use as a medicament, in particular for use in a method of treating cancer. The invention thus provides a scaffold according to the invention or a functionalized scaffold according to the invention for use in a method of treating cancer. The invention also provides a scaffold according to the invention or a functionalized scaffold according to the invention for use in a method of treating acquired or hereditary disorders, in particular monogenic deficiency disorders.

An aspect of the invention relates to an endosomal and/or lysosomal escape enhancing conjugate, said conjugate comprising said W moiety, according to the invention or a functionalized endosomal and/or lysosomal escape enhancing conjugate encompassing an endosomal and/or lysosomal escape enhancing conjugate, said conjugate comprising said W moiety, according to the invention for use as a medicament.

An aspect of the invention relates to an endosomal and/or lysosomal escape enhancing conjugate, said conjugate comprising said W moiety, according to the invention or a functionalized endosomal and/or lysosomal escape enhancing conjugate encompassing an endosomal and/or lysosomal escape enhancing conjugate, said conjugate comprising said W moiety, according to the invention, for use in a method of treating cancer.

An embodiment is the endosomal and/or lysosomal escape enhancing conjugate, said conjugate comprising said W moiety, according to the invention, or a functionalized endosomal and/or lysosomal escape enhancing conjugate encompassing an endosomal and/or lysosomal escape enhancing conjugate, said conjugate comprising said W moiety, according to the invention, for use according to the invention, the use comprising administering to a cancer patient in need thereof an effective amount of the endosomal and/or lysosomal escape enhancing conjugate, said conjugate comprising said W moiety, according to the invention or administering to a cancer patient in need thereof an effective amount of the functionalized endosomal and/or lysosomal escape enhancing conjugate encompassing an endosomal and/or lysosomal escape enhancing conjugate, said conjugate comprising said W moiety, according to the invention.

An aspect of the invention relates to a method for producing an endosomal and/or lysosomal escape enhancing conjugate, said conjugate comprising said W moiety, for enhancing the endosomal escape of an effector molecule as outlined for the endosomal and/or lysosomal escape enhancing conjugate of the invention, the method comprising:
providing a W moiety as outlined for the endosomal and/or lysosomal escape enhancing conjugate of the invention the W moiety selected from a polymeric structure or an oligomeric structure; and
coupling at least one glycoside as outlined for the endosomal and/or lysosomal escape enhancing conjugate of the invention to said W moiety.

An aspect of the invention relates to a method for producing a functionalized endosomal and/or lysosomal escape enhancing conjugate encompassing an endosomal and/or lysosomal escape enhancing conjugate, said conjugate comprising said W moiety, for enhancing the endosomal escape of an effector molecule, the method comprising:
providing an endosomal and/or lysosomal escape enhancing conjugate, said conjugate comprising said W moiety, comprising at least one glycoside bound to a polymeric or oligomeric structure, preferably an endosomal and/or lysosomal escape enhancing conjugate according to the invention; and
coupling at least one E moiety as outlined for the endosomal and/or lysosomal escape enhancing conjugate of the invention, the E moiety being either a) at least one effector molecule, b) at least one ligand, c) at least one effector molecule and in addition at least one ligand, d) at least one effector molecule that itself bears at least one ligand, or e) at least one ligand that itself bears at least one effector molecule to said endosomal and/or lysosomal escape enhancing conjugate.

An embodiment is the endosomal and/or lysosomal escape enhancing conjugate, said conjugate comprising said W moiety, according to the invention, a functionalized endosomal and/or lysosomal escape enhancing conjugate encompassing an endosomal and/or lysosomal escape enhancing conjugate, said conjugate comprising said W moiety, according to the invention, an endosomal and/or lysosomal escape enhancing conjugate, said conjugate comprising said W moiety, or functionalized endosomal and/or lysosomal escape enhancing conjugate encompassing an endosomal and/or lysosomal escape enhancing conjugate, said conjugate comprising said W moiety, for use according to the invention, or method for producing an endosomal and/or lysosomal escape enhancing conjugate, said conjugate comprising said W moiety, or for producing a functionalized endosomal and/or lysosomal escape enhancing conjugate encompassing an endosomal and/or lysosomal escape enhancing conjugate, said conjugate comprising said W moiety, according to the invention, wherein the endosomal and/or lysosomal escape enhancing conjugate is able to augment endosomal escape of the effector.

An embodiment is the pharmaceutical composition comprising an endosomal and/or lysosomal escape enhancing conjugate, said conjugate comprising said W moiety, according to the invention, or a functionalized endosomal and/or lysosomal escape enhancing conjugate encompassing an endosomal and/or lysosomal escape enhancing conjugate, said conjugate comprising said W moiety, according to the invention, and a pharmaceutical acceptable carrier.

A further application in medicine is the substitution of intracellular enzymes in target cells that produce these enzymes in insufficient amount or insufficient functionality. The resulting disease might be hereditary or acquired. In most cases, only symptomatic treatment is possible and for a number of rare diseases, insufficient treatment options lead to a shortened life span of concerned patients. An example for such a disease is phenylketonuria, which is an inborn error of metabolism that results in decreased metabolism of the amino acid phenylalanine. The disease is characterized by mutations in the gene for the hepatic enzyme phenylalanine hydroxylase. Phenylketonuria is not curable to date. The incidence is approximately 1:10,000 with the highest known incidence in Turkey with 1:2,600. A functionalized scaffold with phenylalanine hydroxylase or with a polynucleotide that encodes phenylalanine hydroxylase can be used to target liver cells by use of a suitable ligand and to substitute the defect enzyme in hepatocytes. This is one example of use of the scaffold according to the invention for substitution or gene therapy. In a preferred embodiment, a scaffold according to the invention or a functionalized scaffold according to the invention for use in a method of gene therapy or substitution therapy is provided.

The invention can also be used for biotechnological processes. A possible application is the biomolecular engineering of intracellular switches in eukaryotes [10]. Transcriptional switches target gene expression at the level of mRNA polymerization, translational switches target the process of turning the mRNA signal into protein, and post-translational switches control how proteins interact with one another to attenuate or relay signals. When optimized, these cellular switches can turn a cellular function “on” and “off” based on cues designated by the developer. These cues include small molecules, hormones and drugs. To apply the switch, the cue must enter the target cell. Therefore, in current applications, only small, diffusible molecules can be used that are neither specific for target cells nor do they have high specificity for the selected switch. A functionalized scaffold with a more complex and thus more specific, non-diffusible effector molecule can be used to target a particular switch and the use of a suitable ligand can restrict the effect to target cells. In one embodiment, the invention provides the use of a scaffold or functionalized scaffold according to the invention for enhancing an effect of an effector molecule, preferably in vitro. Preferably, the use is for enhancing an effect of transcriptional switches in vitro.

Another application is the use of the invention in basic research. For functional analyses of cellular processes, it is often required to bring a protein into cells, a method called protein transfection. For instance, to investigate the molecular mechanisms of the chicken virus protein apoptin that leads to apoptosis in eukaryotic cells, it is required to bring the purified protein into the target cell. Existing protein transfection kits are, however, characterized by low efficacy, missing specificity for target cells and high toxicity and can therefore not be used for a number of applications, in particular when metabolic pathways are part of the investigation. A functionalized scaffold with apoptin and use of a suitable ligand can be used to conduct such investigations. In one embodiment, the invention provides a use of a scaffold or functionalized scaffold according to the invention for polypeptide transfection, preferably in vitro. Also provided is a use of a scaffold or functionalized scaffold according to the invention for polynucleotide transfection, preferably in vitro.

The present invention also provides a method of treating cancer, the method comprising administering a medicament comprising a scaffold according to the invention or, preferably, a functionalized scaffold according to the invention to a patient in need thereof.

The scaffold or functionalized scaffold stands for a platform technology that may
provide highly efficient cytosolic delivery of macromolecules
facilitate cellular research and biotechnical applications
have a therapeutic potential in multiple diseases
be used to induce cellular destruction (e.g. of cancer cells)
reduce unwanted side effects by lowering therapeutic levels required for diseased cells
reduce the risk of an immune response to the effector molecule (as less effector molecule is needed, but maybe also because the route of antigen presentation through the endosomes onto MHC molecules is disrupted)
open the possibility for highly efficient manipulation of genes
resurrect failed drug candidates by increasing their efficacy
be made of biocompatible and degradable and/or excretable materials
rely on a mild and non-hazardous effector molecule release triggered by endosomal pH.

Flexibility is ensured by the possibility to use any type of a ligand (e.g. antibodies or aptamers) and effector molecule. A sophisticated implementation of click chemistry may be used to provide a user-friendly interface to apply this technology to own ligands and effector molecules. The novel platform technology offers a variety of possibilities, such as production of the clickable scaffold as a stand-alone product, which allows the user to simply couple any of his effector molecules and/or ligands at his discretion (FIG. 14), or production of a functionalized scaffold, wherein the basic scaffold is already coupled to an effector molecule, which allows the user to couple his ligand to guide the effector molecule to the desired target cells (FIG. 15). A possible functionalized scaffold is a scaffold linked to a ribosome-inactivating protein, e.g. dianthin. This toxic enzyme with a high potential for targeted cell killing can be used to click any future antibodies or antibodies already existing on the market that are designed to specifically recognize tumor cells, such as trastuzumab, cetuximab, rituximab, gemtuzumab or obinutuzumab (next generation ADC technology). As a nucleic acid effector molecule, micro-RNA (miRNA, a polynucleotide) or miRNA inhibitors can for instance be used to create functionalized scaffolds for efficient and low dose cytosolic delivery. MiRNAs or miRNA inhibitors have high potential as novel therapeutics, capable of changing gene programs within the cell, and thereby changing cellular function.

The invention further provides a method for producing a scaffold, preferably a scaffold according to the invention, the scaffold comprising at least one glycoside molecule capable of improving an effect of an effector molecule, bound to a polymeric or oligomeric structure, the method comprising: providing the polymeric or oligomeric structure; and coupling the at least one glycoside molecule to said polymeric or oligomeric structure. Preferably, the at least one glycoside molecule augments endosomal escape of said effector molecule. In particular, the thus obtained scaffold augments endosomal escape of said effector molecule. Preferably, the at least one glycoside molecule is a bisdesmosidic triterpene, more preferably a bisdesmosidic triterpene saponin, more preferably belonging to the type of a 12,13-dehydrooleanane with an aldehyde function in position 23, more preferably, a saponin that can be isolated from Gypsophila or Saponaria species, most preferably SA1641 and/or SO1861, or any of their diastereomers. Preferably, the at least one glycoside molecule is coupled to the polymeric or oligomeric structure, via a cleavable bond, wherein preferably said cleavable bond is subject to cleavage under acidic, reductive, enzymatic or light-induced conditions, more preferably, wherein the cleavable bond is an imine, hydrazone, oxime, 1,3-dioxolane, disulfide or ester, more preferably a disulfide or hydrazone bond. If the bond is a cleavable bond, the saponin is preferably attached to the scaffold via the aldehyde function in position 23 or via one of the carboxyl groups in saponin, more preferably through the aldehyde function.

In another preferred embodiment, a scaffold is provided, wherein the at least one glycoside molecule is bound to the polymeric or oligomeric structure via a stable bond. In a more preferred embodiment, the at least one glycoside molecule is a saponin and the stable bond between saponin and scaffold preferably occurs via an amide coupling or amine formation. This is, e.g., realized via carbodiimide mediated amide bond formation by an amino group of the polymeric or oligomeric structure and the activated glucuronic acid group of the saponin. Chemical bonds that fulfill the stable condition can also be used for aldehyde coupling, e.g. particular amines derived after reductive amination, requiring primary amine groups as the functional group of the polymeric or oligomeric structure. If the bond is a stable bond, the saponin is preferably attached to the scaffold via one of the carboxyl groups of the saponin.

Preferably, the scaffold further comprises a click chemistry group for coupling to the effector molecule and/or to a ligand. Preferably, the click chemistry group is a tetrazine, azide, alkene, or alkyne, or a cyclic derivative of these groups, such as cyclooctyne (e.g. aza-dibenzocyclooctyne, difluorocyclooctyne, bicyclo[6.1.0]non-4-yne, or dibenzocyclooctyne).

In one preferred embodiment, a method according to the invention for producing a scaffold is provided, wherein the number of glycoside molecules is a defined number or a defined range. Preferably, the polymeric or oligomeric structure comprises a linear, branched or cyclic polymer, oligomer, dendrimer, dendron, dendronized polymer, dendronized oligomer or assemblies of these structures, either sheer or mixed, wherein assemblies can be built up by covalent cross-linking or non-covalent attraction and can form hydrogels or nanogels, and wherein, preferably, the polymer is a derivate of a polyethylenimine, polyethylene glycol, polyamino acid or DNA polymer or wherein the oligomer or polymer is a derivate of a dextran, lactic acid, nucleic acid or peptide nucleic acid. In a preferred embodiment, the effector molecule is a pharmaceutically active substance, such as a toxin, a drug, a polypeptide and/or a polynucleotide.

Also provided is a method for producing a functionalized scaffold the method comprising: providing a scaffold comprising multiple glycoside molecules and a polymeric or oligomeric structure, preferably a scaffold according to the invention or obtainable by a method according to the invention for producing a scaffold; and coupling either a) at least one effector molecule, b) at least one ligand, c) at least one effector molecule and in addition at least one ligand, d) at least one effector that itself bears at least one ligand, or e) at least one ligand that itself bears at least one effector to said scaffold. Preferably, coupling in a)-e) independently occurs via click chemistry bonds. In one embodiment, which is encompassed under option c) above, the effector molecule and the ligand are both bound to a linker that in itself is bound to the scaffold. The skilled person is perfectly able to design such trifunctional linkers, based on the present disclosure and the common general knowledge. Such trifunctional linker can exhibit, for instance, a maleimido group that can be used for conjugation to targeting ligands that exhibit thiol groups to perform a thiol-ene reaction. In addition, the trifunctional linker could exhibit a dibenzocyclooctyne (DBCO) group to perform the so-called strain-promoted alkyne-azide cycloaddition (SPAAC, click chemistry) with an azido bearing saponin. Finally, the trifunctional linker could obtain a third functional group such as a trans-cyclooctene (TCO) group to perform the so-called inverse electron demand Diels-Alder (IEDDA) reaction with a tetrazine (Tz) bearing effector molecule. In a preferred embodiment, said at least one effector molecule is a pharmaceutically active substance, such as a toxin, drug, polypeptide, or polynucleotide. In a more preferred embodiment, the at least one effector molecule is a toxin or a polynucleotide. Preferably, said at least one ligand is capable of specifically binding to a target cell specific surface molecule or structure that is able to undergo endocytosis, preferably an antibody or fragment thereof, a cytokine, a growth factor, an aptamer or a designed ankyrin repeat protein. Preferably, said target cell is a diseased or disease-related cell, preferably a tumor cell, a tumor-associated cell (e.g. tumor vascular cell), an immune cell (e.g. a T regulatory cell), or a cell with a monogenic defect. In a preferred embodiment, a method according to the invention is provided for producing a functionalized scaffold, wherein said at least one effector molecule is coupled to a scaffold and/or to said at least one ligand via a cleavable bond, wherein preferably said cleavable bond is subject to cleavage under acidic, reductive, enzymatic or light-induced conditions. In a preferred embodiment, a method according to the invention for producing a scaffold or functionalized scaffold is provided, the method further comprising coupling said scaffold or functionalized scaffold to a carrier, wherein said carrier preferably is a kind of nanoparticle, liposome, micelle, colloid or a particle-like structure comprising cholesterol and/or phospholipids.

The invention also provides a pharmaceutical composition comprising a scaffold according to the invention or a functionalized scaffold according to the invention, and a pharmaceutical acceptable carrier. Such pharmaceutical composition is for use in the treatment of a patient, in particular for use in the treatment of cancer or acquired or hereditary disorders, in particular monogenic deficiency disorders.

An aspect of the invention relates to an endosomal and/or lysosomal escape enhancing conjugate according to the invention, wherein n is 1, said conjugate comprising at least two E moieties, and wherein the at least one S moiety is/are (a) terminal moiety/moieties and is/are any of
a bisdesmosidic triterpene saponin belonging to the type of a 12,13-dehydrooleanane with an aldehyde function, in position 23,
a saponin isolatable from species Gypsophila,
a saponin isolatable from species Saponaria,
a saponin selected from:
   SA1641 or a diastereomer thereof,
   SO1861 or a diastereomer thereof, and
   GE1741 or a diastereomer thereof;
   and preferably the S moiety is SO1861;
      wherein
      the at least one L moiety is at least one W moiety,

wherein the at least one W moiety is any one or more of:
a reactive group ‘*’ on the at least one S moiety, preferably selected from
   an aldehyde group,
   a carboxylic acid group,
   an alkenyl group,
   an hydroxyl group,
for linking the at least one S moiety to at least a first moiety L via the reactive group ‘*’,
a linker, such as a chemical linker or a linear stretch of amino-acid residues complexed through peptide bonds, the linker comprising a reactive group for direct linking of the at least one S moiety to a single E moiety through preferably a single bond, preferably the linker is N-ε-maleimidocaproic acid hydrazide for conjugating a sulfhydryl, such as in a cysteine, to a carbonyl such as in an aldehyde or in a ketone, or preferably the linker is succinimidyl 3-(2-pyridyldithio)propionate;
a scaffold, consisting of, or comprising
   an oligomeric structure, or
   a polymeric structure,
      wherein the oligomeric structure and the polymeric structure comprises, or is selected from, any of:
         a polymer;
         an oligomer;
         a dendrimer;
         a dendron;
         a dendronized polymer;
         a dendronized oligomer;
         an assembly of any of a polymer, an oligomer, a dendrimer, a dendron, a dendronized polymer, a dendronized oligomer,
         wherein the polymer, oligomer, dendrimer, dendron, dendronized polymer, dendronized oligomer, are any of
         linear;
         branched; or
         cyclic,

preferably the oligomeric structure is a dendron,

wherein the scaffold comprises a single reactive group ‘*’ for coupling a single S moiety, or
wherein the scaffold comprises more than one reactive group ‘*’, each group ‘*’ for coupling a single S moiety,
wherein the scaffold comprises a single binding site for binding a single E moiety, or
wherein the scaffold comprises multiple binding sites for binding multiple E moieties,
said binding sites for one or more E moieties on the scaffold moiety W being reactive groups ‘*’ on the scaffold moiety W for provision of a bond with at least one E moiety, such as a covalent bond, a non-covalent bond, an electrostatic interaction, a hydrogen bond, a salt bridge, a van der Waals interaction, a hydrophobic interaction, preferably a covalent bond,
wherein the at least one S moiety is linked, coupled or bound to the reactive group ‘*’ on the W moiety through a bond, such as a covalent bond, a non-covalent bond, an electrostatic interaction, a hydrogen bond, a salt bridge, a van der Waals interaction, a hydrophobic interaction, preferably a covalent bond,

wherein said (covalent) bond between the S moiety and the W moiety is optionally a cleavable bond, wherein said cleavable bond is preferably subject to cleavage under any one or more of:
acidic conditions, preferably at a pH of lower than 6.5 such as pH 4.0-6.5 and preferably at a pH of ≤5.5;
reductive conditions;
enzymatic conditions; and
light-induced conditions,
wherein the cleavable bond is optionally selected from:

an imine bond;

a hydrazone bond;

a 1,3-dioxolane bond; and

an ester bond, and/or
wherein the cleavable bond is a disulfide bond or a peptide bond or an amide bond,
   or
   wherein the at least one L moiety is an O moiety,

wherein the O moiety is a trifunctional linker comprising three reactive groups ‘*’ for linking one S moiety and two E moieties, or for linking two S moieties and one E moiety, or wherein the O moiety is a linker with at least three functionalities comprising at least three reactive groups ‘*’ for linking at least one S moiety and at least two E moieties, or for linking at least two S moieties and at least one E moiety
or wherein the at least one L moiety is one or more W moieties and/or one or more O moieties, wherein more than two W moieties and O moieties together are coupled together in a linear fashion or are coupled together in a branched manner relative to coupled E moieties,

wherein the at least two E moieties are at least one of at least one effector moiety and at least one of at least one ligand,

wherein the effector moiety or the effector moieties is/are selected from any one or more of:
a molecule with pharmaceutical activity, such as a drug molecule, including, but not being limited to a macromolecule or a small molecule;
a toxin, such as a macromolecular cell-killing agent, a protein toxin, an immunotoxin, saporin, dianthin, ribosomal inactivating protein, a small molecule cell-killing agent, a small molecule toxin;
a nucleotide, preferably an oligonucleotide, an RNA, a DNA, an LNA, a BNA, (bridged nucleic acid), an aptamer, a nucleic acid, a plasmid, a vector, a gene, an ASO (allele-specific oligonucleotide), an antisense oligonucleotide (ASO), an miRNA (microRNA), an siRNA (small interfering RNA);
an enzyme;
a second protein; and
a second peptide,
   wherein the ligand(s) is/are selected from any one or more of:
a binding partner for a target cell surface molecule, the target cell surface molecule preferably selected from any of HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CD38, FGFR3, CD123, DLL3, CEACAM5, HER3, CD74, PTK7, Notch3, FGF2, C4.4A, FLT3, CD71, such as binding partner EGF for cell surface receptor EGFR or transferrin for transferrin receptor; and
an immunoglobulin or a binding domain or binding fragment thereof, for binding to a cell surface molecule such as cell-surface receptor HER2 and cell-surface receptor EGFR, such as immunoglobulin trastuzumab for binding to HER2 and immunoglobulin cetuximab for binding to EGFR and anti-CD71 mAb for binding to CD71,
   wherein the effector moiety/moieties and the ligand(s) are directly coupled to any of the scaffold, the at least one S moiety, the trifunctional linker O, the linker such as a chemical linker, a first proteinaceous molecule such as a first peptide, a first polypeptide, and a first protein,

and/or wherein a first effector moiety or a first ligand is directly coupled to any of the scaffold, the at least one S moiety, the trifunctional linker O, the linker, preferably a chemical linker, the first proteinaceous molecule such as the first peptide, the first polypeptide, the first protein, and wherein optionally a second, a third and further effector moiety/moieties and/or optionally a second, a third and further ligand(s) is/are coupled to said first, second or third effector moiety or is/are coupled to said first, second, or third ligand, either directly, or through a linker, in linear fashion in any order of two or more effector moieties and/or two or more ligands, and/or in branched fashion,
wherein optionally one or more S moiety/moieties is/are coupled to said first, second, third and further effector moiety/moieties and/or to said first, second, third and further ligand(s), preferably S moiety/moieties is/are coupled directly to an effector moiety or to a ligand, or is/are coupled to an effector moiety or to a ligand via an L moiety such as a linker, a trifunctional linker, and/or a scaffold, wherein the scaffold is preferably a dendron or a dendrimer and wherein the S moiety is preferably linked to the scaffold via a linker or a trifunctional linker, wherein the bond between an S moiety and an L moiety is preferably a cleavable bond, wherein said cleavable bond is preferably subject to cleavage under any one or more of:
acidic conditions, preferably at a pH of lower than 6.5 such as pH 4.0-6.5 and preferably at a pH ≤5.5;
reductive conditions;
enzymatic conditions; and
light-induced conditions,
wherein the cleavable bond is optionally selected from:
   an imine bond;
   a hydrazone bond;
   a 1,3-dioxolane bond; and
   an ester bond,
   and/or wherein the cleavable bond is a disulfide bond or a peptide bond or an amide bond.
      An embodiment is the endosomal and/or lysosomal escape enhancing conjugate according to the previous aspect of the invention, wherein the S moiety is a saponin selected from:
SA1641 or a diastereomer thereof,
SO1861 or a diastereomer thereof, and
GE1741 or a diastereomer thereof;
   and preferably the S moiety is the saponin SO1861,

wherein the L moiety is selected from a linker, a trifunctional linker, a scaffold, wherein the scaffold is preferably a dendron or a dendrimer, wherein the bond between an S moiety and an L moiety is a non-cleavable bond or a cleavable bond and preferably a cleavable bond, wherein said cleavable bond is preferably subject to cleavage under any one or more of:
acidic conditions, preferably at a pH of lower than 6.5 such as pH 4.0-6.0, preferably at a pH ≤5.5;
reductive conditions;
enzymatic conditions; and
light-induced conditions,
wherein the cleavable bond is optionally selected from:
   an imine bond;
   a hydrazone bond;
   a 1,3-dioxolane bond; and
   an ester bond,
   and/or wherein the cleavable bond is a disulfide bond or a peptide bond or an amide bond.
      wherein the at least two E moieties are at least a ligand moiety selected from EGF, an immunoglobulin or a binding domain or binding fragment thereof, for binding to a cell surface molecule such as cell-surface receptor HER2 and cell-surface receptor EGFR, such as immunoglobulin trastuzumab for binding to HER2 and immunoglobulin cetuximab for binding to EGFR and monoclonal antibody anti-CD71 mAb for binding to transferrin receptor, and any one or more effector moiety/moieties selected from
a molecule with pharmaceutical activity, such as a drug molecule, including, but not being limited to a macromolecule or a small molecule;
a toxin, such as a macromolecular cell-killing agent, a protein toxin, an immunotoxin, saporin, dianthin, ribosomal inactivating protein, a small molecule cell-killing agent, a small molecule toxin;
a nucleotide, preferably an oligonucleotide, an RNA, a DNA, an LNA, a BNA (bridged nucleic acid), an aptamer, a nucleic acid, a plasmid, a vector, a gene, an ASO (allele-specific oligonucleotide), an antisense oligonucleotide (ASO), an miRNA (microRNA), an siRNA (small interfering RNA);
an enzyme,
   wherein the effector moiety/moieties is/are preferably (a) terminal moiety/moieties, and wherein the ligand moiety is preferably bound to at least one S moiety and to at least one effector moiety.

An embodiment is the endosomal and/or lysosomal escape enhancing conjugate according to the previous aspect or according to the previous embodiment, wherein the S moiety is the saponin SO1861, the L moiety is a linker and/or a trifunctional linker, and/or a dendron or a dendrimer, wherein the bond between an S moiety and an L moiety is a non-cleavable bond or a cleavable bond and preferably a cleavable bond, wherein said cleavable bond is preferably subject to cleavage under any one or more of:
acidic conditions, preferably at a pH of lower than 6.5 such as pH 4.0-6.0, preferably at a pH ≤5.5;
reductive conditions;
enzymatic conditions; and
light-induced conditions,
wherein the cleavable bond is optionally selected from:
   an imine bond;
   a hydrazone bond;
   a 1,3-dioxolane bond; and
   an ester bond,
   and/or wherein the cleavable bond is a disulfide bond or a peptide bond or an amide bond.
      wherein the at least two E moieties are at least a ligand moiety selected from EGF and an immunoglobulin selected from trastuzumab and cetuximab and anti-CD71 mAb, and any one or more effector moiety/moieties selected from saporin, dianthin, ribosomal inactivating protein, an RNA, a DNA, an LNA, a BNA (bridged nucleic acid), such as a BNA for silencing HSP27,
      wherein the effector moiety/moieties is/are (a) terminal moiety/moieties, and wherein the ligand moiety is bound to any one of 1, 2, 4, 8, 16, 32, 64 S moieties, preferably 1, 4, 8, 16, 32 moieties, and bound to at least one effector moiety, preferably a single effector moiety such as a dianthin moiety, a saporin moiety, an enzyme, a BNA moiety.

An embodiment is the endosomal and/or lysosomal escape enhancing conjugate according to the previous embodiment, wherein the at least one S moiety is/are 1, 4, 16 SO1861 moieties terminally linked in the conjugate, the L moiety is a dendron and/or a linker and/or a trifunctional linker for linking S moieties to the dendron and/or for linking a ligand and/or effector moieties to the dendron, wherein the ligand is EGF, trastuzumab or cetuximab or anti-CD71 monoclonal antibody, wherein the effector moiety is selected from dianthin, saporin, HSP27-silencing BNA, Cre-recombinase.

An embodiment is the endosomal and/or lysosomal escape enhancing conjugate according to the invention, essentially having the molecular format of molecular structure (II):

(saponin-linker-)ₐ immunoglobulin-effector moiety (STRUCTURE (II)),

wherein a=1-4,

or essentially having the molecular format of molecular structure (III):

(saponin-dendron(-saponin)ₓ)_{b}-immunoglobulin-effector moiety (STRUCTURE (III)),

wherein x=between 1 and 100, preferably 1-63, 1-31, 1-15, 1-7, or 3; b=1-4,

or essentially having the molecular format of molecular structure (IV):

(saponin-trifunctional linker(-effector moiety))_{c}-immunoglobulin (STRUCTURE (IV)),

wherein c=1-4. Preferably, x is 3. Preferably, b is 1, 2 or 4, although in some embodiments, b is 3.

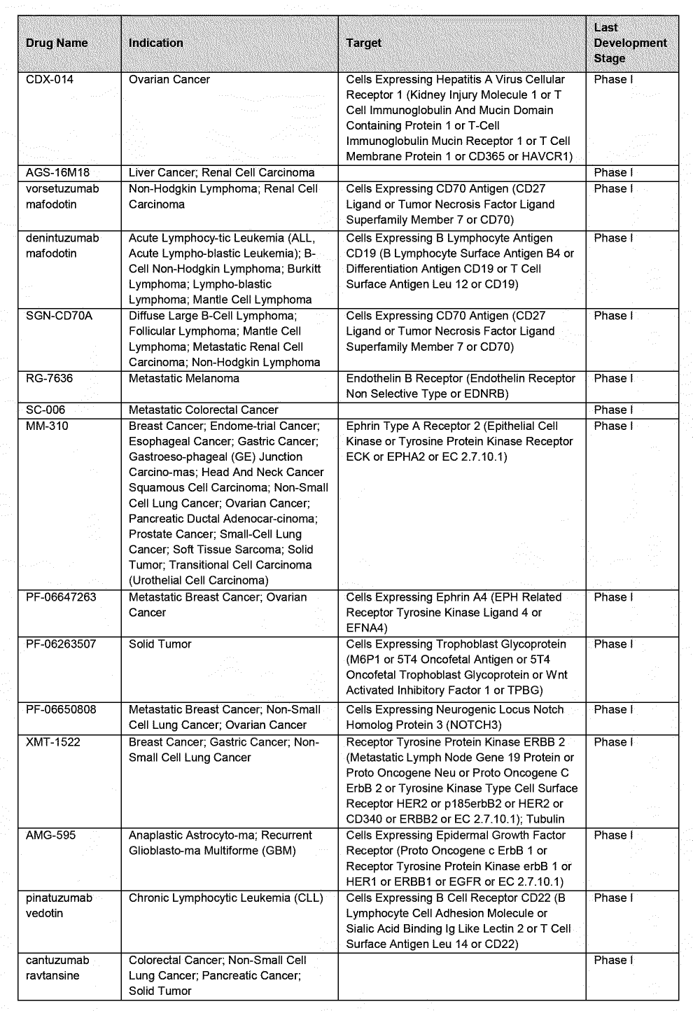

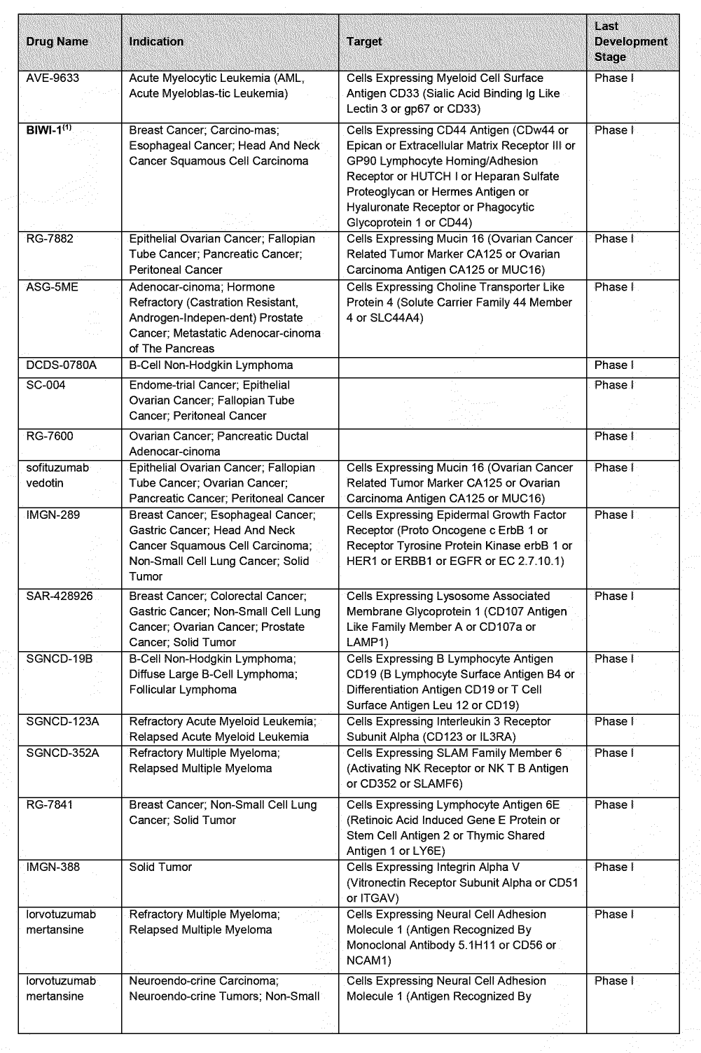

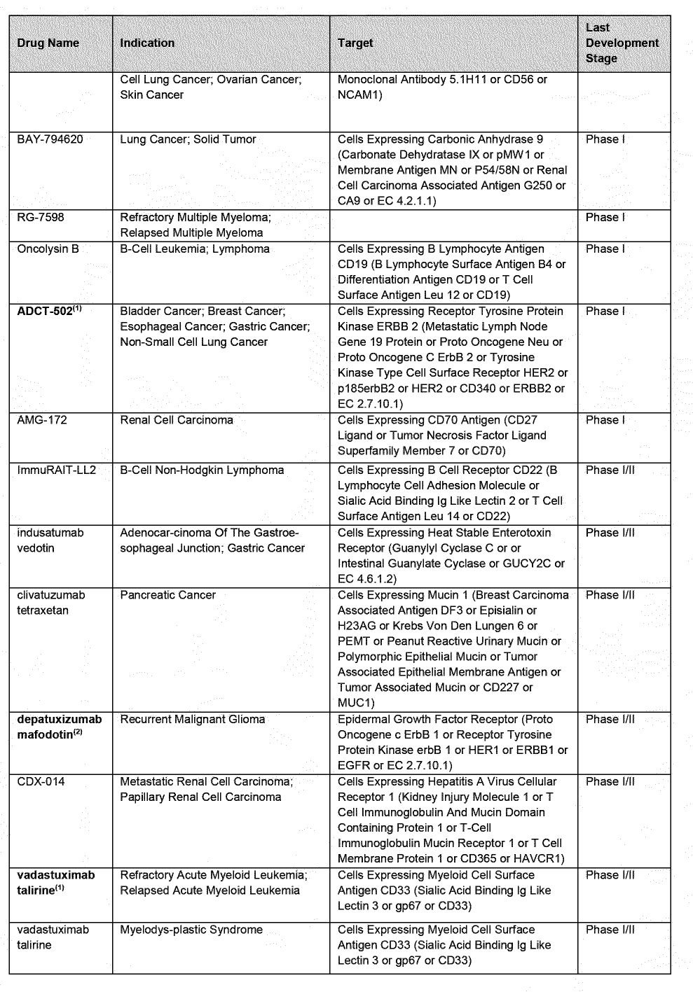

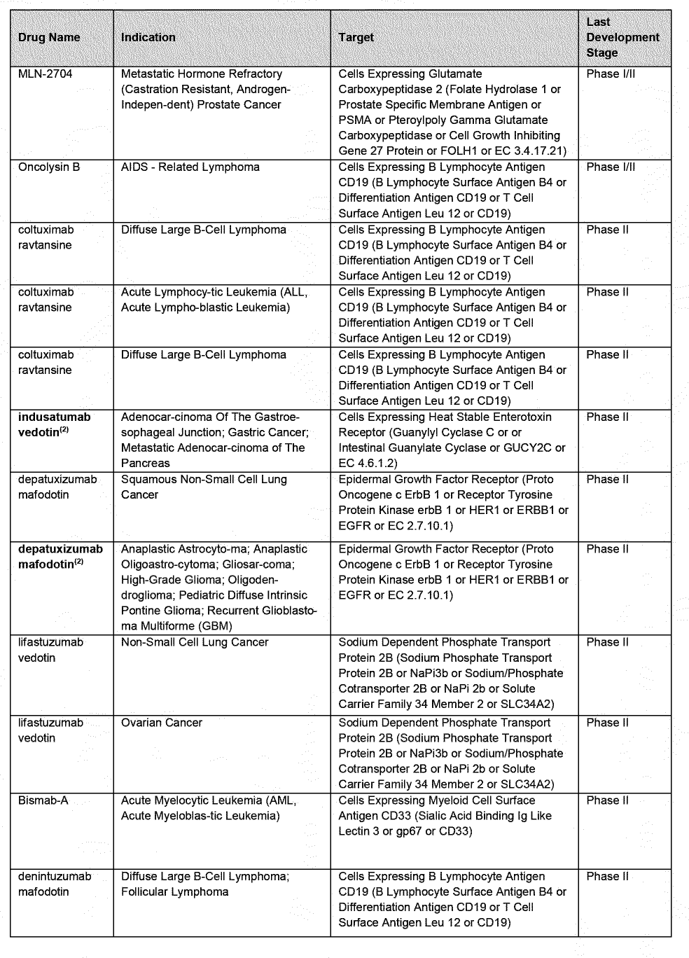

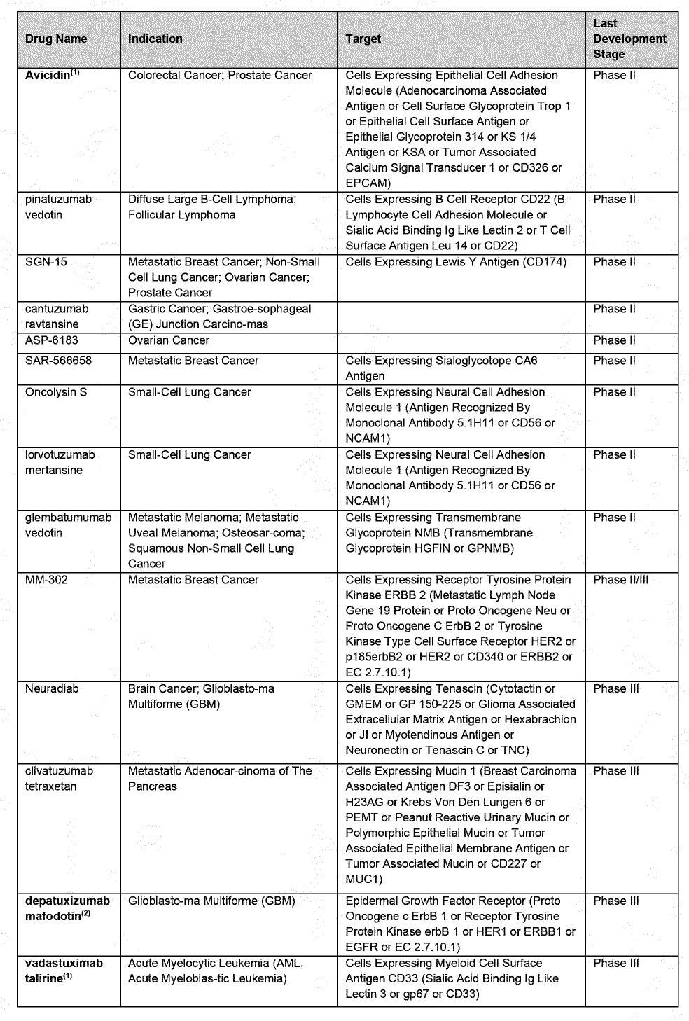

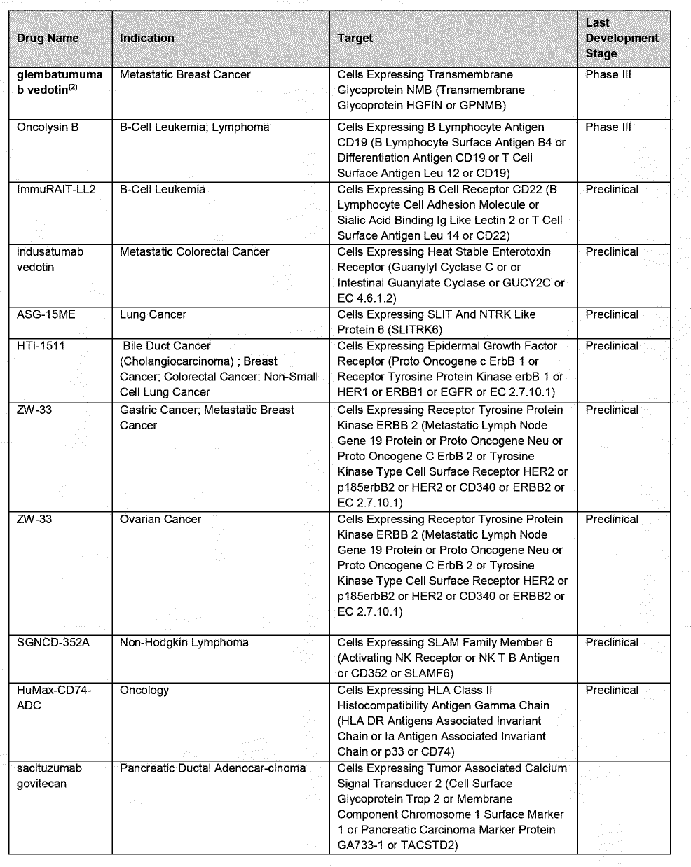

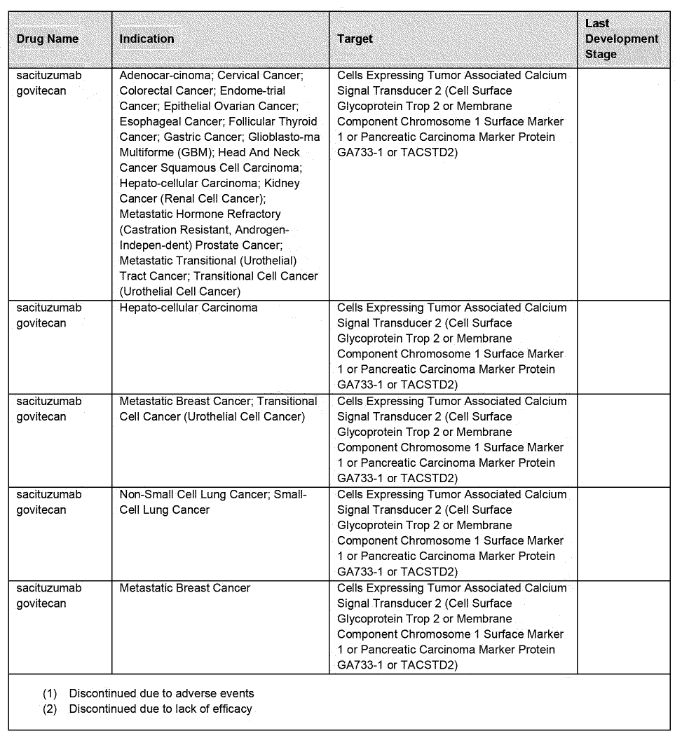

An embodiment is the endosomal and/or lysosomal escape enhancing conjugate according to the invention, consisting of an immunoglobulin complexed with one or more saponin moieties, preferably 2-32 SO1861 moieties such as 4-16 moieties, and with a payload selected from a toxin, an oligonucleotide, an enzyme, a drug compound, wherein the immunoglobulin is an antibody such as a pharmaceutically acceptable antibody such as cetuximab, trastuzumab or an antibody listed in FIG. 57, anti-CD71 monoclonal antibody.

An embodiment is the endosomal and/or lysosomal escape enhancing conjugate according to the invention, wherein the conjugate is devoid of any effector moiety and wherein the conjugate comprises a ligand such as EGF or an immunoglobulin, the ligand complexed with one or more saponin moieties, preferably 2-32 SO1861 moieties such as 4-16 moieties, wherein the immunoglobulin is an antibody such as a pharmaceutically acceptable antibody such as cetuximab, trastuzumab or an antibody listed in FIG. 57, anti-CD71 monoclonal antibody.

### Summary of a Number of Embodiments of the Invention

mAb: trastuzumab (HER2) or cetuximab (EGFR)

### Ligand: EGF

Protein toxin: Ribosome inactivating protein, saporin or dianthin
endosomal and/or lysosomal escape enhancing conjugates of saponin with a ligand: mAb-SO1861 endosomal and/or lysosomal escape enhancing conjugates

contain a cleavable hydrazone linker

| | |
|---|---|
| Trastuzumab-SO1861 | DAR 4.0 |
| Cetuximab -SO1861 | DAR 3.7; |

the endosomal and/or lysosomal escape enhancing conjugates of saponin with a ligand combined with:
mAb/ligand-protein toxin conjugates

contain a non-cleavable chemical linker
or are recombinant fusion proteins

| | |
|---|---|
| Trastuzumab-Saporin | DAR 3.0 |
| Cetuximab-Saporin | DAR 2.6 |
| Trastuzumab-Dianthin | DAR 1.0 |
| EGF-Dianthin (fusion protein) | DAR 1.0 |
| IgG-Saporin | DAR 2.2 |
| Anti-CD71 monoclonal | DAR 2.1 |
| antibody ligated to saporin | |

Several of the many benefits of the application of the conjugates of the invention, the compositions of the invention and the combinations of the invention and the kits of the invention are, amongst others and without wishing to exclude further benefits:
Conjugating saponins to antibodies may provide certain benefits. Without wishing to be bound by any theory, as supported by presented exemplifying experimental data, advantages of using conjugated (targeted) saponin versus free saponin include (but are not limited to):
Safety: free saponin does not distinguish between target cells and healthy cells. As a result, endosomal escape activity of free saponin is not only exerted in target cells, such as aberrant cells, but also in healthy cells. This implies the risk that certain endosomal molecules that would otherwise remain inside the healthy cell endosome would now enter the cytosol of such healthy cell, to potentially cause damage/side effects;
Delivery to target cells: conjugated saponin is actively delivered to target cells; non-target expressing cells are expected to receive a substantially lower percentage of saponins; a higher proportion of administered saponin is available for target cells;
Delivery to the right compartment of the target cell: mAb-conjugation causes saponin to be internalized into the endosomal compartment of target cells; free saponin molecules could enter any cell without arriving in endosomes, which would imply that such free saponin molecules would not be functional in facilitating endosomal escape;
Efficacy, which results from
   Increased safety. Tolerability in terms of the absolute dose of saponin may be higher since mAb-conjugation prevents the saponin molecule from randomly entering cells—as a result, mAb-saponin can be dosed higher, and the efficacy is higher compared to exposing cells to free un-targeted saponin when endosomal escape enhancing activity is concerned;
   Increased circulation half-life. Half-life of the mAb-saponin conjugate is much longer than for free saponin: consequently a higher proportion of total administered saponin will end up in target cells;
   Synchronization: delivery of the effector and saponin to target cells occurs in a better synchronized fashion when compared to administering un-targeted free saponin and an effector moiety; mAb-effector and free saponin have different pharmacokinetics and consequently, the accumulation of free saponin versus mAb-effector inside target cell endosomes is unbalanced. Conjugation of the saponin to a cell targeting ligand increases the “endosomal escape yield” per dosed saponin molecule.
      Summarizing, the endosomal and/or lysosomal escape enhancing conjugates, the binding molecules, the scaffolds according to the invention can for example alternatively be described as follows:
      Molecular assemblies of the invention (Endosomal and/or lysosomal escape enhancing conjugate of the invention) have the general structure of molecular structure (V):

B(*)n-(*F)m structure (V),

where n≥m; and
where B is G and * of G, wherein the * is any of —CHO, —COOH, —OH, alkenyl group; and
where each *F is

### 1. any of W, Z; or

2. *A(*)i-(*Y)k with i≥1, k≥1, i≥k, where at least one Y is W; or
3. *A(*)i-(*Z)k with i≥1, k≥1, i≥k, where at least one Y is W; or
4. *L(*)i-(*Y)k with i≥0, k≥0, i≥k, where at least one Y is W; or
5. *L(*)i-(*Z)k with i≥1, k≥1, i≥k, where at least one Y is W; or
6. *L(*)i-(*G)k with i≥0, k≥0, i≥k; or
7. *E(*)i-(*Y)k with i≥1, k≥1, i≥k, where at least one Y is W; or
8. *E(*)i-(*Z)k with i≥1, k≥1, i≥k, where at least one Y is W; and
wherein
B is a bioactive saponin;
F is a functional moiety;
* is a reactive group;
A is an adapter, which is for example any of a bifunctional linker, a trifunctional linker or a multifunctional linker, wherein such a linker or such linkers optionally include spacers and/or cleavable bonds, and/or wherein said linker or linkers optionally are a concatenation of adapters;
S is a (polymeric) scaffold;
L is a ligand;
E is an effector;
G is a glycoside;
W is *L(*)p-(*E)q or *E(*)p-(*L)q with p≥1, q≥0, p≥q;

### X is any of A, S, L, E, G;

### Y is any of W, X; and

Z is *S(*)p-(*Y)q with p≥1, q≥1, p≥q,
wherein the scaffold is here any of the scaffolds as outlined in the aspects, embodiments of the invention and as claimed, the L moiety is here any of the ligands as outlined in the aspects of the invention, embodiments of the invention and as claimed, the E moiety is here any of the effector moieties as outlined in the aspects of the invention, embodiments of the invention and as claimed, and wherein the G moiety is a glycoside as outlined in the aspects of the invention, embodiments of the invention and as claimed. The scaffold is a scaffold consisting of, or comprising
an oligomeric structure, or
a polymeric structure,
   wherein the oligomeric structure and the polymeric structure comprises, or is selected from, any of:
      a polymer;
      an oligomer;
      a dendrimer;
      a dendron;
      a dendronized polymer;
      a dendronized oligomer;
      an assembly of any of a polymer, an oligomer, a dendrimer, a dendron, a dendronized polymer, a dendronized oligomer,
         wherein the polymer, oligomer, dendrimer, dendron, dendronized polymer, dendronized oligomer, are any of
         linear;
         branched; or
         cyclic.
            For example, the scaffold is any one of a polymeric structure or oligomeric structure such as poly- or oligo(amines), e.g., polyethylenimine and poly(amidoamine), and biocompatible structures such as polyethylene glycol, poly- or oligo(esters), such as poly(lactids), poly(lactams), polylactide-co-glycolide copolymers, and poly(dextrin), poly- or oligosaccharides, such as cyclodextrin or polydextrose, and poly- or oligoamino acids or DNA oligo- or polymers.
            The invention is further illustrated by the following examples, which should not be interpreted as limiting the present invention in any way.

### EXAMPLES

### Materials:

Trastuzumab and cetuximab were from Roche and Eli Lilly and Co., respectively. SO1861 was isolated and purified by Analyticon Discovery GmbH from raw plant extract obtained from Saponaria officinalis L.

### Methods

### SO1861-EMCH Synthesis

SO1861 was from Saponaria officinalis L (Analyticon Discovery GmbH), and was coupled to EMCH according to conventional steps known in the art.

### Conjugation of SO1861 to Antibodies

Custom production of trastuzumab-SO1861 and cetuximab-SO1861 was performed by FleetBioprocessing (UK). SO1861-EMCH was conjugated to cysteines of the antibody. SPT-EMCH was applied.

### Conjugation of Saporin to Trastuzumab, Cetuximab and Anti-CD71 Monoclonal Antibody

Custom trastuzumab-saporin and cetuximab-saporin conjugates were produced and purchased from Advanced Targeting Systems (San Diego, Calif.). IgG-saporin and saporin was purchased from Advanced Targeting Systems

### FACS Analyses

FACS analysis was performed on a BD FACSCanto II, data analysis with FlowJo V10 software, FACS antibodies were: 1) Isotype: APC Mouse IgG1, K Isotype Ctrl (FC) (400122, Biolegend). EGFR: APC anti-human EGFR (352906, Biolegend) HER2: APC anti-human CD340 (erbB2/HER-2) (324408, Biolegend).

### Dianthin Production

Dianthin was expressed in a bacterium culture and the protein was purified following conventional cell culturing and protein purification steps known in the art.

### Conjugation of Antibody to Dianthin

Conjugation of antibody and dianthin was according to common procedures known in the art.

### Cell Culture

Cells were cultured in DMEM (PAN-Biotech GmbH) supplemented with 10% fetal bovine serum (FBS) (PAN-Biotech GmbH) at 37° C. and 5% CO₂.

### Cell Viability Assay

Cells were seeded in a 96 well plate at 5.000-10.000 c/w in 100 μL/well and incubated overnight at 37° C. The next day 10× concentrated treatment-mix samples were prepared in PBS, which contain antibody-conjugated SO1861 (i.e. a ‘first binding molecule’ of the invention or an ‘endosomal and/or lysosomal escape enhancing conjugate’ of the invention) and targeted-toxin (i.e. a ‘second binding molecule’) both at 10× final concentration. The media was removed from the cell culture plate and replaced by 180 μL culture media, followed by the addition of 20 μL treatment-mix/well. For control, 10× treatment-mix samples were prepared that contained the corresponding concentrations of only antibody-conjugated SO1861, only antibody, only SO1861, only targeted-toxin, or PBS without compound as vehicle control. In the case that endosomal acidification inhibitors (chloroquine (Sigma Aldrich) or bafilomycin A1 (Enzo Life Sciences)) were used, the cell culture media in step 1 of treatment was replaced by 180 μL media containing 1 μM chloroquine or 0.2 μM bafilomycin A1. The plate was incubated for 1 hour at 37° C., before the 10× treatment-mix samples were added. The remaining incubation and treatment steps were performed according to standard procedures known in the field.

After treatment the cells were incubated for 72 hr at 37° C. before the cell viability was determined by a MTS-assay, performed according to the manufacturer's instruction (CellTiter 96® AQueous One Solution Cell Proliferation Assay, Promega). Briefly, the MTS solution was diluted 20× in DMEM without phenol red (PAN-Biotech GmbH) supplemented with 10% FBS (PAN-Biotech GmbH). The cells were washed once with 200 μL PBS per well, after which 100 μL diluted MTS solution was added per well. The plate was incubated for approximately 20-30 minutes at 37° C. Subsequently, the optical density at 492 nm was measured on a Thermo Scientific Multiskan FC plate reader (Thermo Scientific). For quantification the background signal of ‘medium only’ wells was subtracted from all other wells, before the ratio of untreated/treated cells was calculated, by dividing the background corrected signal of untreated wells over the background corrected signal of the treated wells.

### Results

### Example 1. 1 Target 2-Component System

1 target 2-components system is the combination treatment of mAb1-protein toxin and mAb1-SO1861, whereas the 2 target 2-component system is the combination of mAb1-protein toxin and mAb2-SO1861 or mAb2-protein toxin+mAb1-SO1861 (FIG. 1)

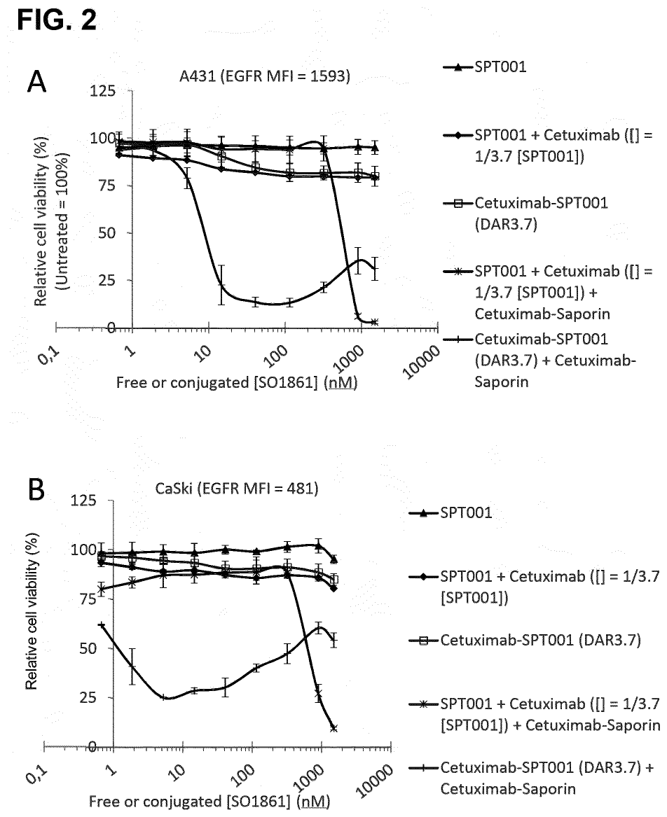

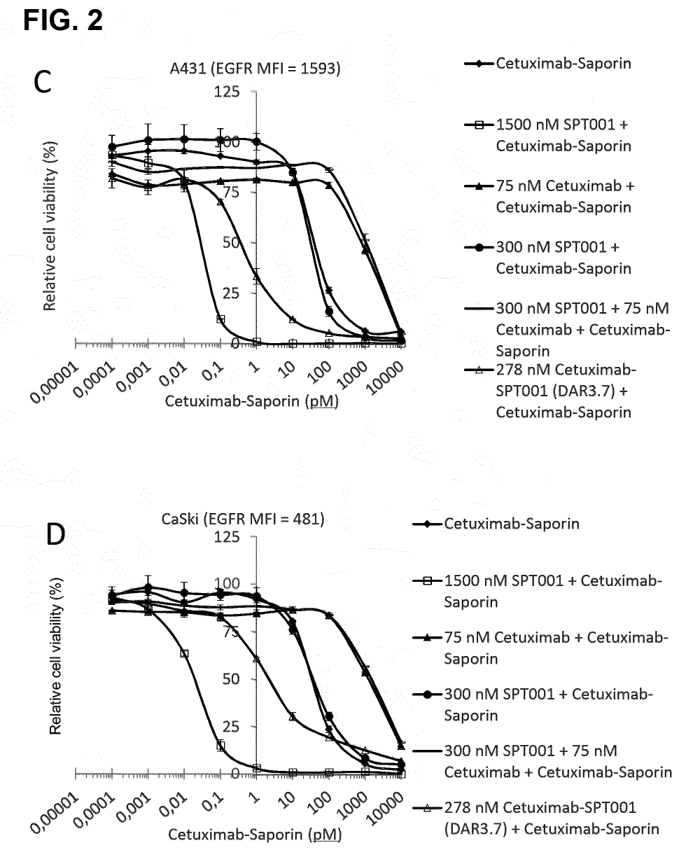

Cetuximab-SO1861 (monoclonal antibody recognizing and binding EGFR, conjugated to the saponin molecule, SO1861; an endosomal and/or lysosomal escape enhancing conjugate according to the invention) was titrated (calculated on concentration SO1861) on a fixed concentration of 10 μM cetuximab-saporin (monoclonal antibody recognizing and binding EGFR, conjugated to the protein toxin, saporin) and cell killing on high EGFR expressing cells was determined. High EGFR expressing cells (A431 or CaSki) showed efficient cell killing when 10 μM cetuximab-saporin was combined with high concentrations of non-targeted unconjugated SO1861 (A431: [SO1861] IC50=600 nM and CaSki: [SO1861] IC50=700 nM; FIG. 2A, 2B; Table 1). However, when cetuximab-saporin was combined with cetuximab-SO1861 strong cell killing was induced already at low concentrations of SO1861 (A431: [SO1861] IC50=5 nM and CaSki [SO1861] IC50=8 nM; FIG. 2A, 2B; Table 1). This shows that targeted conjugated SO1861 is more effective in inducing endosomal and/or lysosomal escape compared to non-targeted unconjugated SO1861. Next, cetuximab-saporin was titrated on a fixed concentration of 300 nM cetuximab-SO1861 and targeted protein toxin mediated cell killing on EGFR expressing cells was determined. High EGFR expressing cells (A431 or CaSki) show cell killing only at high cetuximab-saporin concentrations in combination with non-targeted unconjugated 300 nM SO1861 (A431: [toxin] IC50=40 μM; CaSki: [toxin] IC50=40 μM; FIG. 2C, 2D; Table 2) whereas 300 nM cetuximab-SO1861 in combination with low cetuximab-saporin concentrations already induced efficient cell killing (A431: [toxin] IC50=0.4 μM; CaSki: [toxin] IC50=2 μM; FIGS. 2C and 2D; Table 2). Highest cell killing efficiency is achieved when high concentrations of non-targeted unconjugated SO1861 (1500 nM) is combined with low concentrations of cetuximab-saporin (A431: [toxin] IC50=0.03 μM; CaSki: [toxin] IC50=0.02 μM; FIG. 2C, 2D; Table 2). All this shows that when conjugated to cetuximab, relatively low concentrations of SO1861 efficiently can kill high EGFR expressing cells in combination with relatively low concentrations of cetuximab-saporin. High concentrations (1500 nM) of non-targeted unconjugated SO1861 in combination with low concentrations of cetuximab-saporin is still most effective since receptor competition does not play a role for SO1861 to enter the cell, since in the 1-target 2-component system both conjugates compete for the same EGFR receptor. The receptor competition principle is also clearly shown in the cetuxmab-toxin titration treatments without (A431: [toxin] IC50=40 μM; CaSki: [toxin] IC50=40 μM) or with 75 nM cetuximab (A431: [toxin] IC50=1000 μM; CaSki: from IC50=1000 μM; FIG. 2C, 2D).

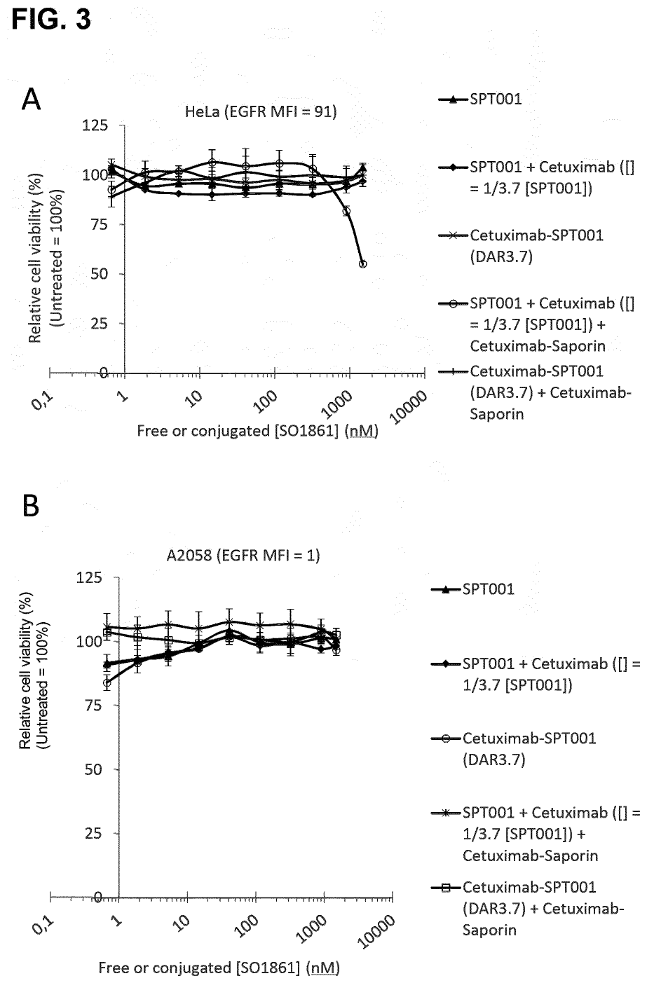

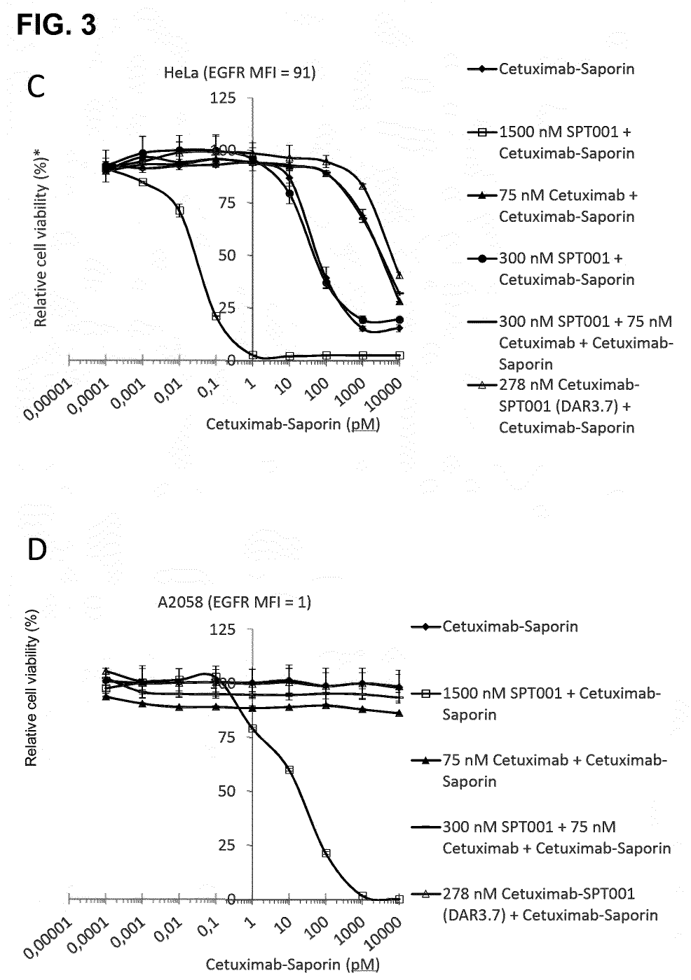

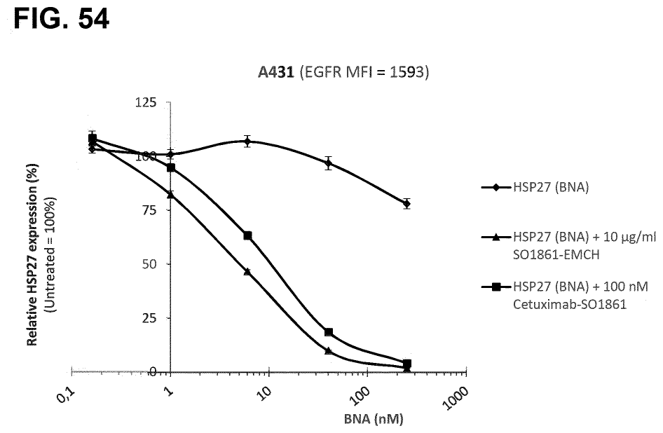

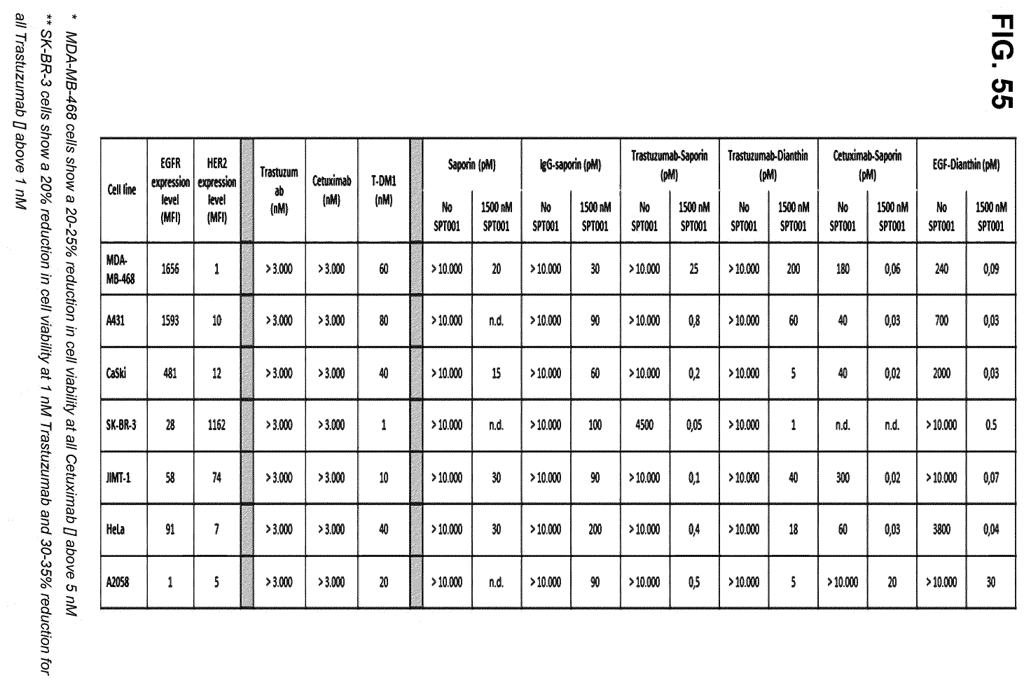

Next, cetuximab-SO1861 was titrated (calculated on concentration SO1861) on a fixed concentration of 10 μM cetuximab-saporin and cell killing on low/no EGFR expressing cells was determined. Low EGFR expressing cells (HeLa) showed only cell killing when 10 μM cetuximab-saporin was combined with high concentrations of non-targeted unconjugated SO1861, whereas A2058 cells that do not express EGFR (A2058) were not sensitive at all (HeLa: [SO1861] IC50=1000 nM; A2058: [SO1861] IC50 >1000 nM; FIG. 3A, 3B; Table 1). The combination of 10 μM cetuximab-saporin with increasing concentrations of cetuximab-SO1861 did not induce any significant cell killing in both cell lines (HeLa: [SO1861] IC50=1000 nM; A2058: [SO1861] IC50 >1000 nM; FIG. 3A, 3B; Table 1). This shows that in the absence of sufficient receptor expression, effective intracellular SO1861 concentrations are not reached (threshold) to induce endosomal protein toxin escape and toxin-mediated cell killing. Next, cetuximab-saporin was titrated on a fixed concentration of 300 nM cetuximab-SO1861 and targeted protein toxin mediated cell killing on low/no EGFR expressing cells was determined. Low EGFR expressing cells (HeLa) show cell killing only at very high cetuximab-saporin concentrations in combination with 278 nM cetuximab-SO1861 or 300 nM unconjugated SO1861, whereas A2058 cells (EGFR) are not sensitive at any of the tested concentrations (HeLa: [toxin] IC50=60 μM; A2058: [toxin] IC50 >10.000 μM; FIG. 3C, 3D; Table 2). High concentrations of unconjugated SO1861 (1500 nM) in combination with low concentrations of cetuximab-saporin in low EGFR expressing cells (HeLa) show efficient cell killing, whereas in A2058 cells only at very high cetuximab-saporin concentrations in combination with 1500 nM SO1861 non-targeted, a-specific cell killing is induced (Hela: [toxin] IC50=0.03 μM; A2058: [toxin] IC50=20 μM; FIG. 3C, 3D; Table in FIG. 54). All this shows that cells with low or no EGFR receptor expression are not susceptible for the combination of cetuximab-SO1861+cetuximab-saporin, due to a lack of sufficient EGFR receptor that facilitates the entry of sufficient SO1861 and toxin within the cell.

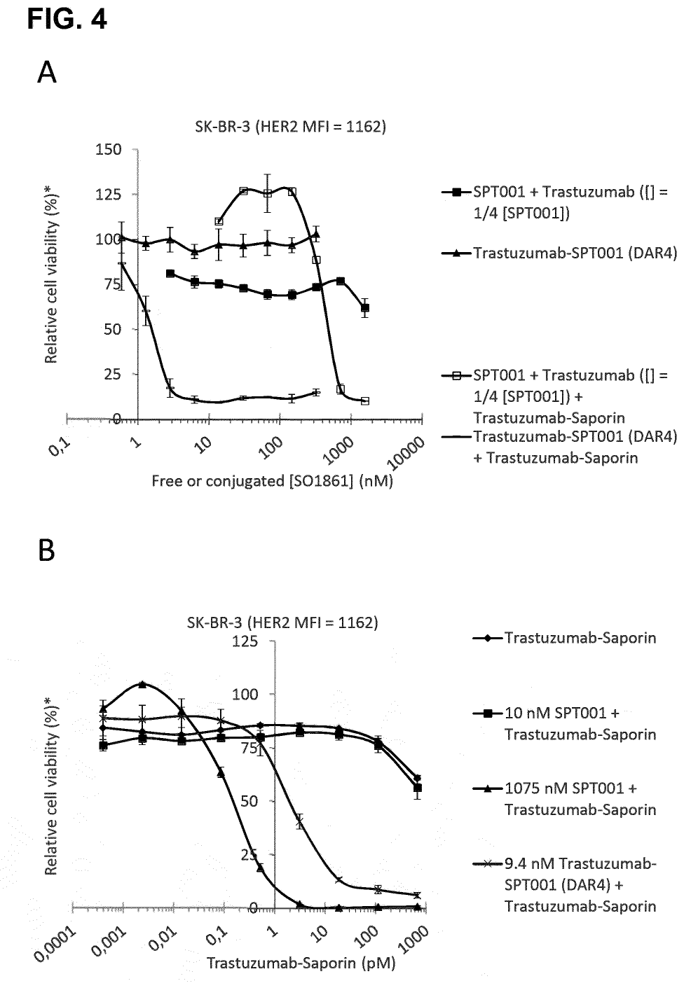

Trastuzumab-SO1861 (monoclonal antibody recognizing and binding HER2, conjugated to the saponin molecule, SO1861; an endosomal and/or lysosomal escape enhancing conjugate according to the invention) was titrated (calculated on concentration SO1861) on a fixed concentration of 50 μM trastuzumab-saporin (monoclonal antibody recognizing and binding HER2, conjugated to the protein toxin, saporin) and cell killing on high HER2 expressing cells was determined. High HER2 expressing cells (SK-BR-3) showed efficient cell killing when 50 μM trastuzumab-saporin was combined with high concentrations of non-targeted unconjugated SO1861 (SKBR3; FIG. 4A, 4B; Table 1). However, when trastuzumab-saporin was combined with trastuzumab-SO1861 strong cell killing was induced already at low concentrations of SO1861 (SK-BR-3; FIG. 4A, 4B; Table 1). This shows that targeted conjugated SO1861 is more effective in inducing endosomal and/or lysosomal escape compared to non-targeted unconjugated SO1861. Next, trastuzumab-saporin was titrated on a fixed concentration of 50 nM trastuzumab-SO1861 and targeted protein toxin mediated cell killing on HER2 expressing cells was determined. High HER2 expressing cells (SKBR3 or BT474) show cell killing only at high trastuzumab-saporin concentrations in combination with non-targeted unconjugated 10 nM SO1861 (Table 2) whereas 10 nM trastuzumab-SO1861 in combination with low trastuzumab-saporin concentrations already induced efficient cell killing (Table 2). Highest cell killing efficiency is achieved when high concentrations of non-targeted unconjugated SO1861 (1500 nM) is combined with low concentrations of trastuzumab-saporin (Table 2). All this shows that when conjugated to trastuzumab, low concentrations of SO1861 efficiently can kill high HER2 expressing cells in combination with relatively low concentrations of trastuzumab-saporin. High concentrations (1500 nM) of non-targeted unconjugated SO1861 in combination with low concentrations of trastuzumab-saporin is still most effective, since receptor competition does not play a role for SO1861 to enter the cell, since in the 1-target 2-component system both conjugates compete for the same EGFR receptor. The receptor competition principle is also clearly shown in the trastuzumab-toxin titration treatments without or with 2.5 nM trastuzumab (SKBR3: [toxin] IC50=1000 nM).

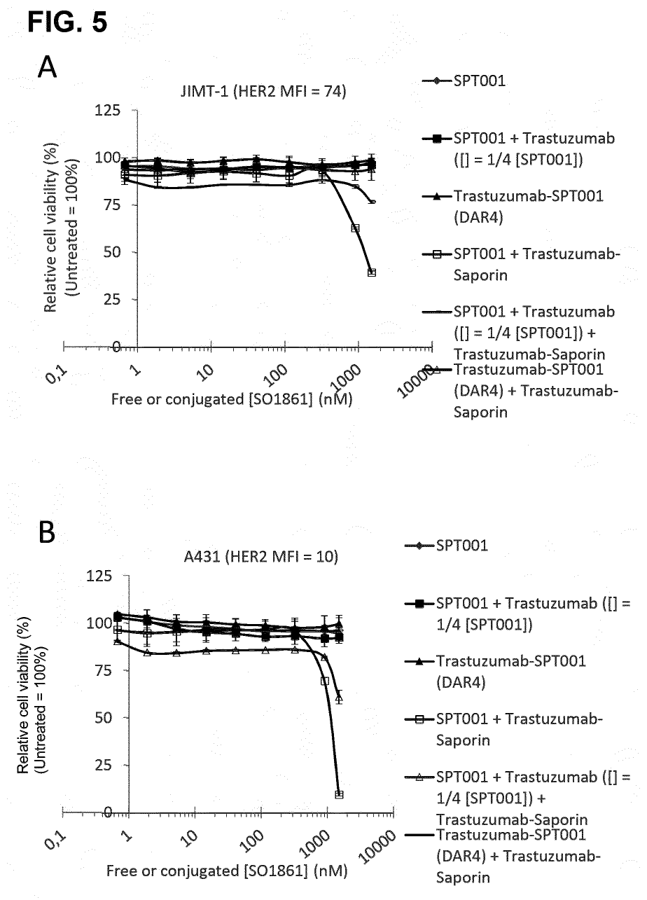

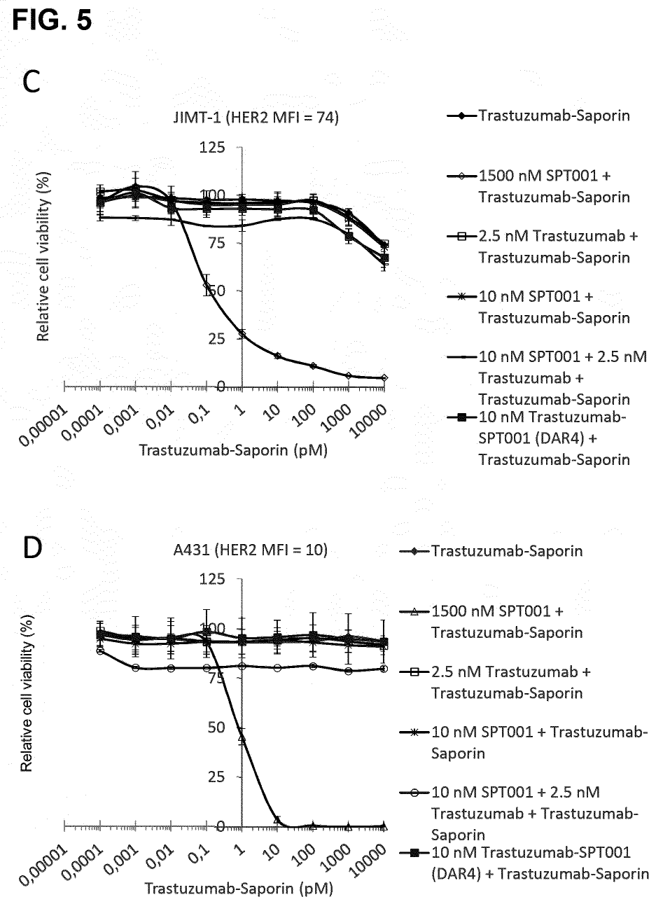

Next, trastuzumab-SO1861 was titrated (calculated on concentration SO1861) on a fixed concentration of 50 μM trastuzumab-saporin and cell killing on low/no EGFR expressing cells was determined. Low EGFR expressing cells (JIMT-1; A431) showed only cell killing when 50 μM trastuzumab-saporin was combined with high concentrations of non-targeted unconjugated SO1861 (JIMT-1: [SO1861] IC50 >1000 nM; A431: [SO1861] IC50 >1000 nM; FIG. 5A, 5B; Table 1). The combination of 50 μM trastuzumab-saporin with increasing concentrations of trastuzumab-SO1861 did not induce any significant cell killing in both cell lines (JIMT-1: [SO1861] IC50 >1000 nM; A431: [SO1861] IC50 >1000 nM; FIG. 5A, 5B; Table 1). This shows that in the absence of sufficient receptor expression, effective intracellular SO1861 concentrations are not reached (threshold) to induce endosomal protein toxin escape and toxin-mediated cell killing. Next, trastuzumab-saporin was titrated on a fixed concentration of 10 nM trastuzumab-SO1861 and targeted protein toxin mediated cell killing on low/no HER2 expressing cells was determined. Low HER2 expressing cells (JIMT-1; A431) show no significant cell killing at high trastuzumab-saporin concentrations in combination with 10 nM trastuzumab-SO1861 (JIMT-1: [toxin] IC50 >10.000 μM; A431: [toxin] IC50 >10.000 μM; FIG. 5C, 5D; Table 2). High concentrations of unconjugated SO1861 (1500 nM) in combination with low concentrations of trastuzumab-saporin in low HER2 expressing cells (show efficient cell killing (JIMT-1: [toxin] IC50=0.1 μM; A431: [toxin] IC50=0.8 μM; FIG. 5C, 5D; Table in FIG. 54). All this shows that cells with low HER2 receptor expression are not susceptible for the combination of trastuzumab-SO1861+trastuzumab-saporin, due to a lack of sufficient HER2 receptor that facilitates the entry of sufficient SO1861 and toxin within the cell.

### Example 2. 2 Target 2-Component System

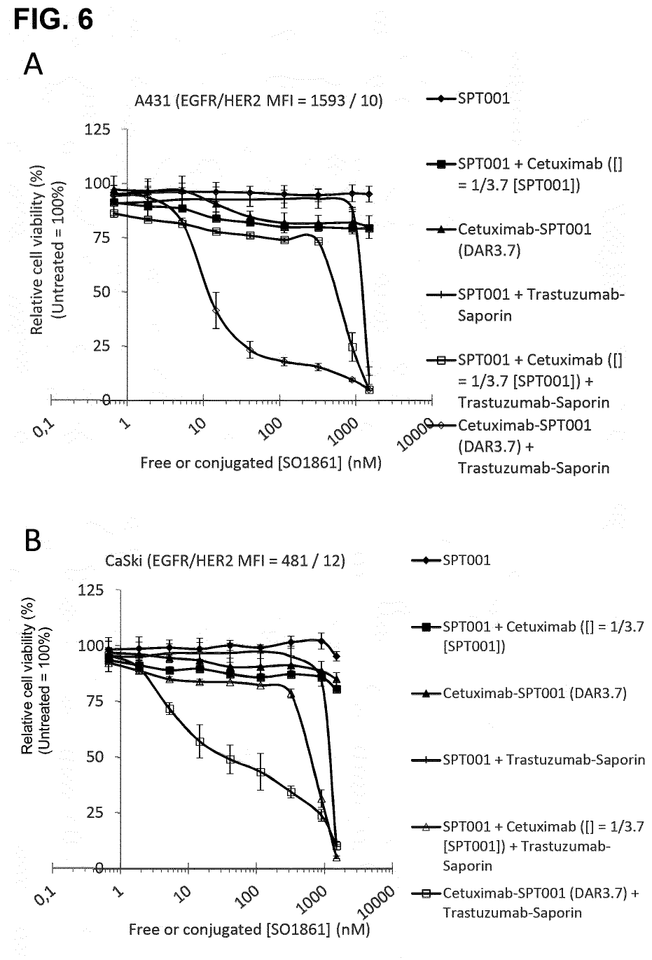

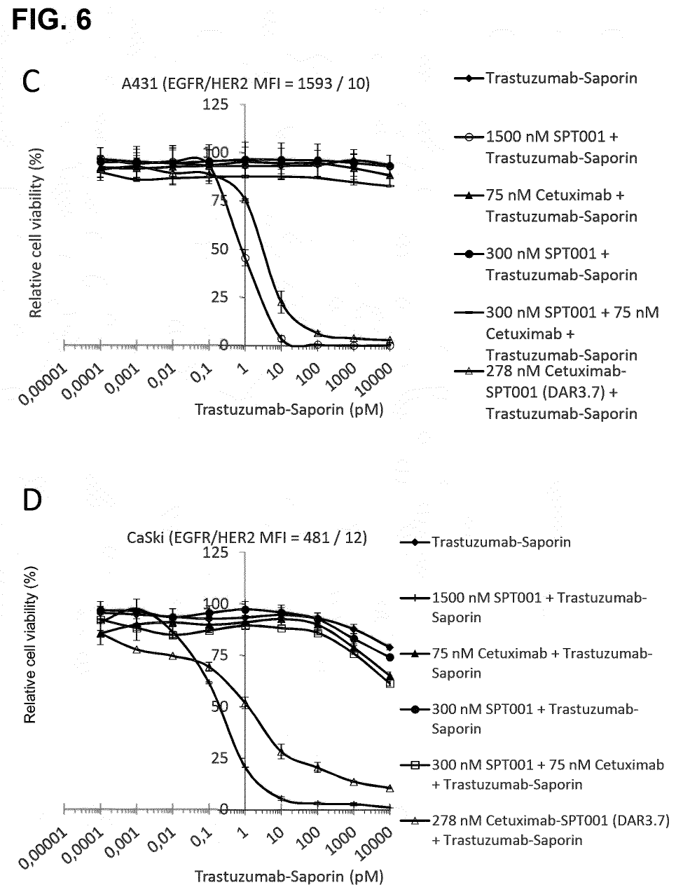

Cetuximab-SO1861 was titrated (calculated on concentration SO1861) on a fixed concentration of 50 μM trastuzumab-saporin and cell killing on high EGFR/low HER2 expressing cells was determined. A431 and CaSki cells showed efficient cell killing when 50 μM trastuzumab-saporin was combined with high concentrations of non-targeted unconjugated SO1861 (A431 and CaSki: [SO1861] IC50=1000 nM; FIG. 6A, 6B; Table 1). However, when trastuzumab-saporin was combined with cetuximab-SO1861 strong cell killing was induced already at low concentrations of SO1861 (A431: [SO1861] IC50=12 nM and CaSki [SO1861] IC50=40 nM; FIG. 6A, 6B; Table 1). This shows that targeted conjugated SO1861 is more effective in inducing endosomal and/or lysosomal escape compared to non-targeted unconjugated SO1861. Next, trastuzumab-saporin was titrated on a fixed concentration of 300 nM cetuximab-SO1861 and targeted protein toxin mediated cell killing was determined on high EGFR/low HER2 expressing cells (A431 and CaSki) No effective cell killing was observed with high trastuzumab-saporin concentrations in combination with non-targeted unconjugated 300 nM SO1861 (A431 and CaSki: [toxin] IC50 >10.000 μM; FIG. 6C, 6D; Table 2) whereas 300 nM cetuximab-SO1861 in combination with low trastuzumab-saporin concentrations already induced efficient cell killing (A431: [toxin] IC50=3 μM; CaSki: [toxin] IC50=1 μM; FIGS. 6C and 6D; table 2). In A431 cells, comparable cell killing efficiency is achieved when high concentrations (1500 nM) of non-targeted unconjugated SO1861 is combined with low concentrations of trastuzumab-saporin (A431: [toxin] IC50=1 μM; see FIG. 6C; Table 2). In CaSki cells the response was slightly stronger compared to the combination of cetuximab-SO1861 and Trastuzumab-saporin, due to the fact that the EGFR expression in these cells is significantly lower compared to A431 and thus targeted delivery of SO1861 to CaSki cells is less sufficient (CaSki: [toxin] IC50=0.2 μM see FIG. 6D; Table 2). All this shows that when conjugated to cetuximab, low concentrations of SO1861 efficiently can kill high EGFR expressing cells in combination with relatively low concentrations of trastuzumab-saporin. High concentrations (1500 nM) of non-targeted unconjugated SO1861 in combination with low concentrations of trastuzumab-saporin has comparable activity, since receptor competition does not play a role for SO1861 to enter the cell, since in the 2-target 2-component system both conjugates are delivered via different receptors, SO1861 via EGFR and toxin via HER2 receptor.

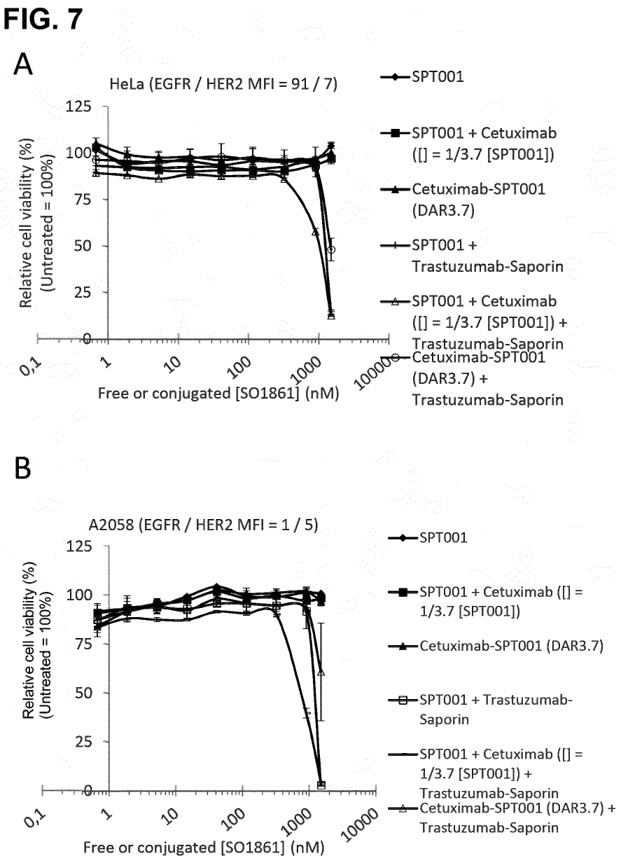

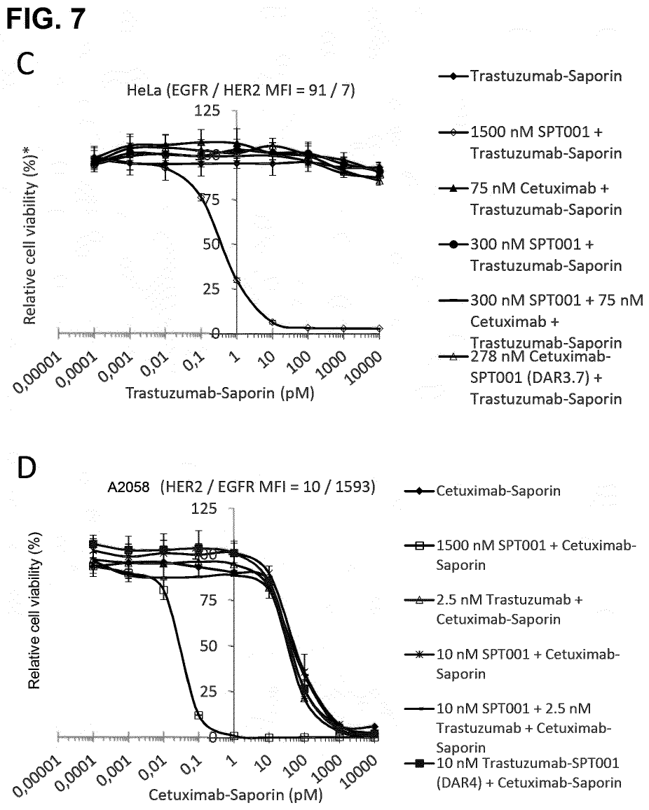

Next, cetuximab-SO1861 was titrated (calculated on concentration SO1861) on a fixed concentration of 50 μM trastuzumab-saporin and cell killing on low/no EGFR/HER2 expressing cells was determined. Low EGFR/HER2 expressing cells showed only cell killing when 50 μM trastuzumab-saporin was combined with high concentrations of non-targeted unconjugated SO1861 (HeLa: [SO1861] IC50 >1000 nM; A2058: [SO1861] IC50 >1000 nM; FIG. 7A, 7B; Table 1). The combination of 50 μM trastuzumab-saporin with increasing concentrations of cetuximab-SO1861 only showed significant cell killing at high concentrations of cetuximab-SO1861 in both cell lines (HeLa: [SO1861] IC50 >1000 nM; A2058: [SO1861] IC50 >1000 nM; FIG. 7A, 7B; Table 1). This shows that in the absence of sufficient receptor expression, effective intracellular SO1861 concentrations are not reached (threshold) to induce endosomal protein toxin escape and toxin-mediated cell killing. Next, trastuzumab-saporin was titrated on a fixed concentration of cetuximab-SO1861 and targeted protein toxin mediated cell killing on low/no EGFR/HER2 expressing cells was determined. Low/no EGFR/HER2 expressing cells (HeLa and A2058) show no significant cell killing at high trastuzumab-saporin concentrations in combination with 278 nM cetuximab-SO1861 (HeLa: [toxin] IC50 >10.000 μM; A2058: [toxin] IC50 >10.000 μM; FIG. 7C, 7D; Table 2). High concentrations of unconjugated SO1861 (1500 nM) in combination with low concentrations of trastuzumab-saporin show efficient cell killing (HeLa: [toxin] IC50=0.4 μM; A2058: [toxin] IC50=0.5 μM; FIG. 7C, 7D; Table in FIG. 54). All this shows that cells with low/no EGFR/low HER2 expression are not susceptible for the combination of cetuximab-SO1861+trastuzumab-saporin, due to a lack of sufficient EGFR receptor that facilitates the entry of sufficient SO1861 to ensure efficient cytoplasmic delivery of the toxin within the cell.

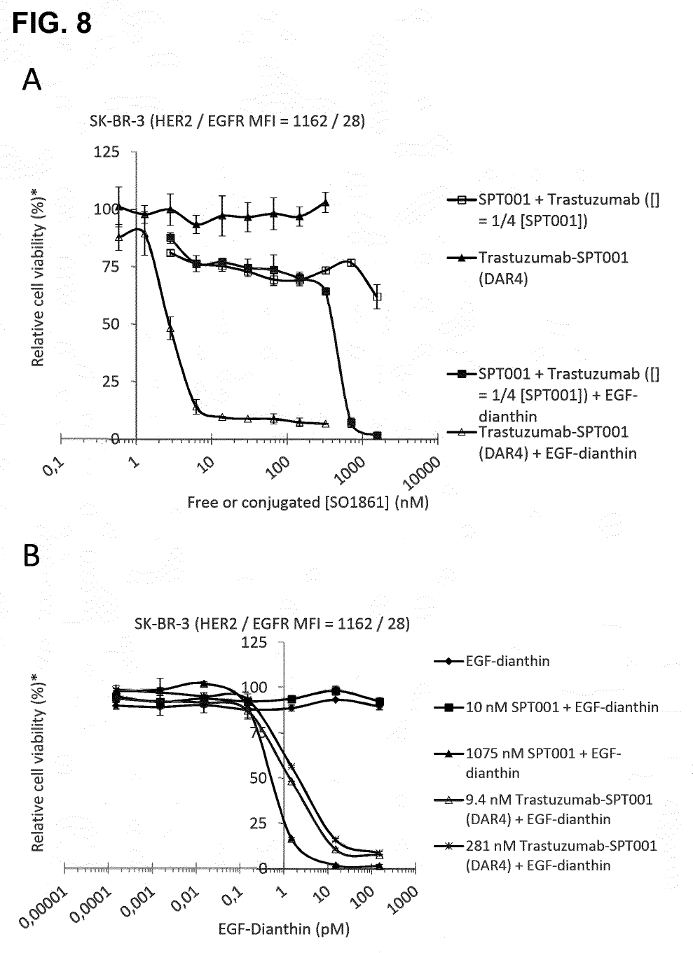

Next, Trastuzumab-SO1861 was titrated (calculated on concentration SO1861) on a fixed concentration of 1.5 μM EGF-dianthon and cell killing on high HER2/low EGFR expressing cells was determined. The results of testing this 2-target 2-component (HER2 high expression and EGFR low expression) system are displayed in FIG. 8. HER2 targeted cell killing in SK-BR-3 (HER2⁺⁺⁺/EGFR^{+/-}) cells by a therapeutic combination according to the invention. FIG. 8A) Trastuzumab-SO1861 titration in combination with a fixed concentration of 1.5 μM EGFdianthin shows that a 400-fold reduced concentration of conjugated SO1861 is required, versus unconjugated SO1861, to induce cell killing by EGFdianthin. FIG. 8B), EGFdianthin titration in combination with 9.4 nM trastuzumab-SO1861 can kill cells in contrast to 10 nM unconjugated SO1861+EGFdianthin. 1075 nM unconjugated SO1861+EGFdianthin has comparable cell killing efficiency compared to the therapeutic combination, 9.4 nM trastuzumab-SO1861+EGFdianthin, since both conjugates do not compete for the same receptor. Only simultaneous targeted delivery of both conjugates leads to efficient cell-killing, in contrast to monotherapy with either conjugate alone. SK-BR-3 and BT474 cells showed efficient cell killing when 1.5 μM EGF-dianthin was combined with high concentrations of non-targeted unconjugated SO1861 (SK-BR-3: [SO1861] IC50=800 nM; FIG. 8A; Table 1). However, when EGF-dianthin was combined with trastuzumab-SO1861 strong cell killing was induced already at low concentrations of conjugated SO1861 (SK-BR-3: [SO1861] IC50=2 nM; FIG. 8A; Table 1). This shows that targeted conjugated SO1861 is more effective in inducing endosomal and/or lysosomal escape compared to non-targeted unconjugated SO1861. Next, EGF-dianthin was titrated on a fixed concentration of trastuzumab-SO1861 and targeted protein toxin mediated cell killing was determined on SKBR3 and BT474 cells. No effective cell killing was observed with high EGF-dianthin concentrations in combination with non-targeted unconjugated 10 nM SO1861 (SKBR3 and BT474: [toxin] IC50 >10.000 μM; FIG. 8B; Table 2) whereas 9.4 nM trastuzumab-SO1861 in combination with low EGF-dianthin concentrations already induced efficient cell killing (SK-BR-3: [toxin] IC50=3 μM; BT474: [toxin] IC50=1 μM; FIG. 8B; Table 2). Comparable cell killing efficiency is achieved when high concentrations (1075 nM) of non-targeted unconjugated SO1861 is combined with low concentrations of EGF-dianthin; FIG. 8B; Table 2). All this shows that when conjugated to trastuzumab, low concentrations of SO1861 efficiently can kill high HER2 expressing cells in combination with relatively low concentrations of EGF-dianthin. High concentrations (1500 nM) of non-targeted unconjugated SO1861 in combination with low concentrations of EGF-dianthin has comparable activity, since receptor competition does not play a role for SO1861 to enter the cell, since in the 2-target 2-component system both conjugates are delivered via different receptors, SO1861 via HER2 and toxin via EGFR receptor. Similarly, the combination of trastuzumab-SO1861 and cetuximab-saporin was tested (results are summarized in Table 2).

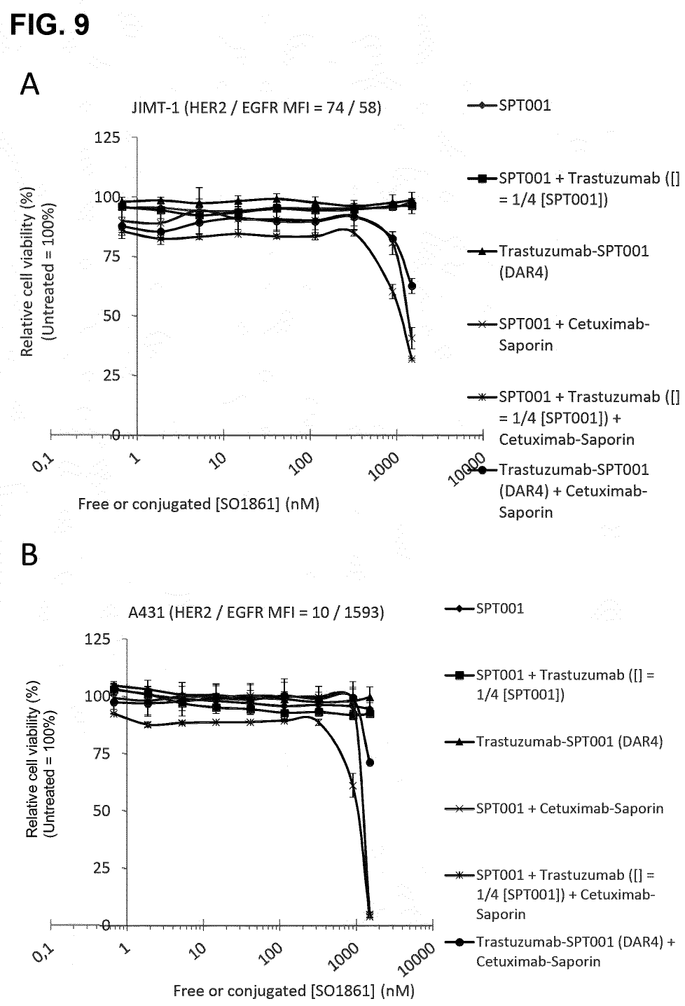

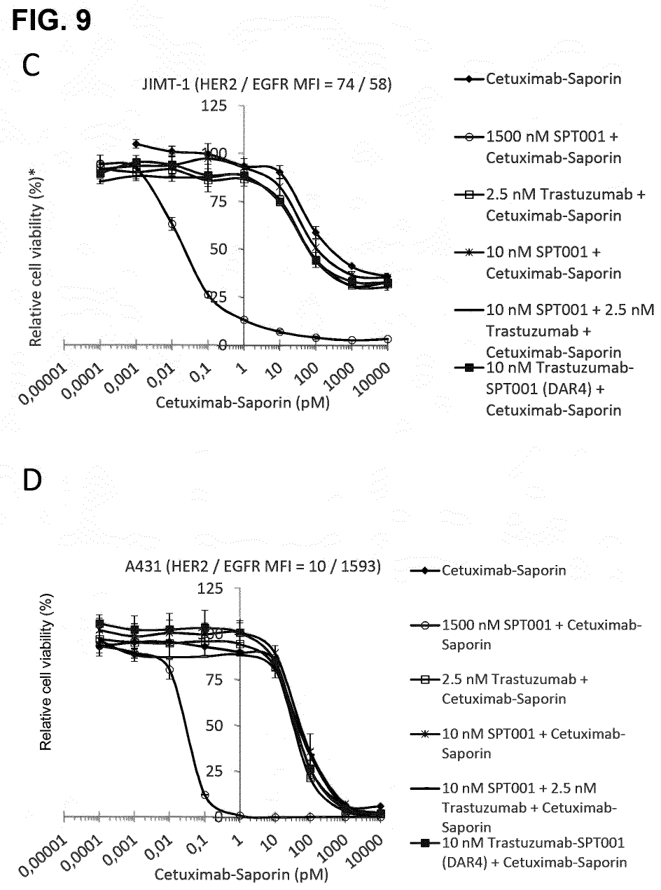

Next, Trastuzumab-SO1861 was titrated (calculated on concentration SO1861) on a fixed concentration of 5 μM cetuximab-saporin and cell killing on low HER2, low/high EGFR expressing cells was determined showed cell killing when 5 μM cetuximab-saporin was combined with high concentrations of non-targeted unconjugated SO1861 (JIMT-1: [SO1861] IC50 >1000 nM; A431: [SO1861] IC50 >1000 nM; FIG. 9A, 9B; Table 1). The combination of 5 μM cetuximab-saporin with increasing concentrations of trastuzumab-SO1861 showed cell killing only at high concentrations of cetuximab-SO1861 in both cell lines (JIMT-1: [SO1861] IC50 >1000 nM; A431: [SO1861] IC50 >1000 nM; FIG. 9A, 9B; Table 1). This shows that in the absence of sufficient receptor expression, effective intracellular SO1861 concentrations are not reached (threshold) to induce endosomal protein toxin escape and toxin-mediated cell killing. Next, cetuximab-saporin was titrated on a fixed concentration of trastuzumab-SO1861 and targeted protein toxin mediated cell killing on JIMT-1 and A431 was determined. Cell killing was observed only at high cetuximab-saporin concentrations in combination with 10 nM trastuzumab-SO1861 (JIMT-1: [toxin] IC50 >90 μM; A431: [toxin] IC50 >20 μM; FIG. 9C, 9D; Table 2). High concentrations of unconjugated SO1861 (1500 nM) in combination with low concentrations of cetuximab-saporin show efficient cell killing (JIMT-1: [toxin] IC50=0.02 μM; A431: [toxin] IC50=0.03 μM; FIG. 9C, 9D; Table in FIG. 54). All this shows that cells with low HER2, low/high EGFR expression are not susceptible for the combination of trastuzumab-SO1861+trastuzumab-saporin, due to a lack of sufficient HER2 receptor that facilitates the entry of sufficient SO1861 to ensure efficient cytoplasmic delivery of the toxin within the cell. Even a very high EGFR expression in A431 cells did not result in efficient cell killing by cetuximab-saporin, since the threshold of SO1861 was not reached due to a lack of HER2 receptors that could facilitate the uptake of SO1861 via trastuzumab-SO1861.

### Example 3

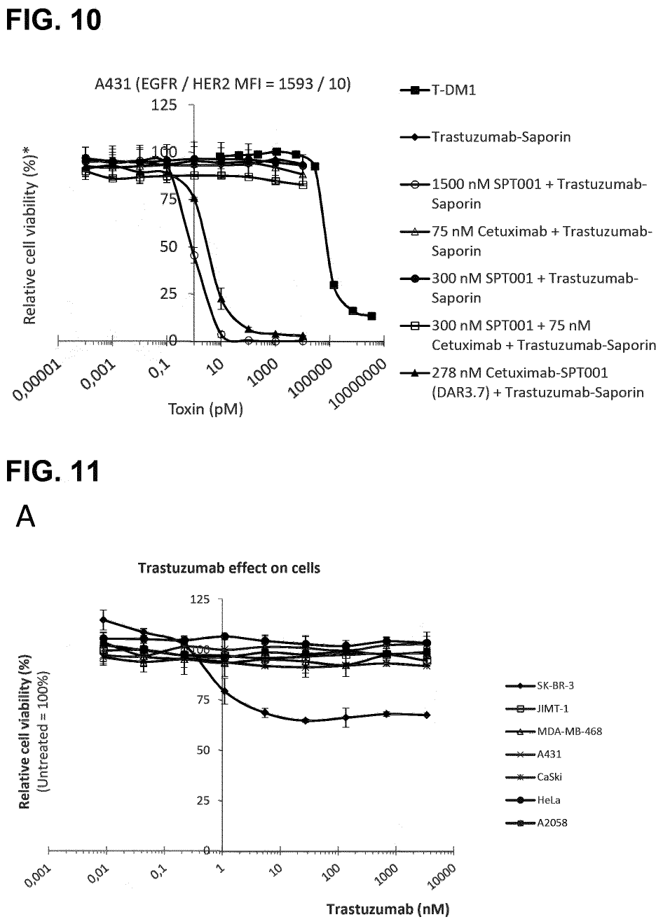

The 2 targeted 2 component system results in cell killing of very low target expressing cells. In A431 cells T-DM1 kills cells at nanomolar concentrations, whereas the targeted 2 component system can efficiently kill cells at picomolar concentrations (7000 fold decrease in toxin concentration) (FIG. 10)

### Example 4. Mechanism of Action

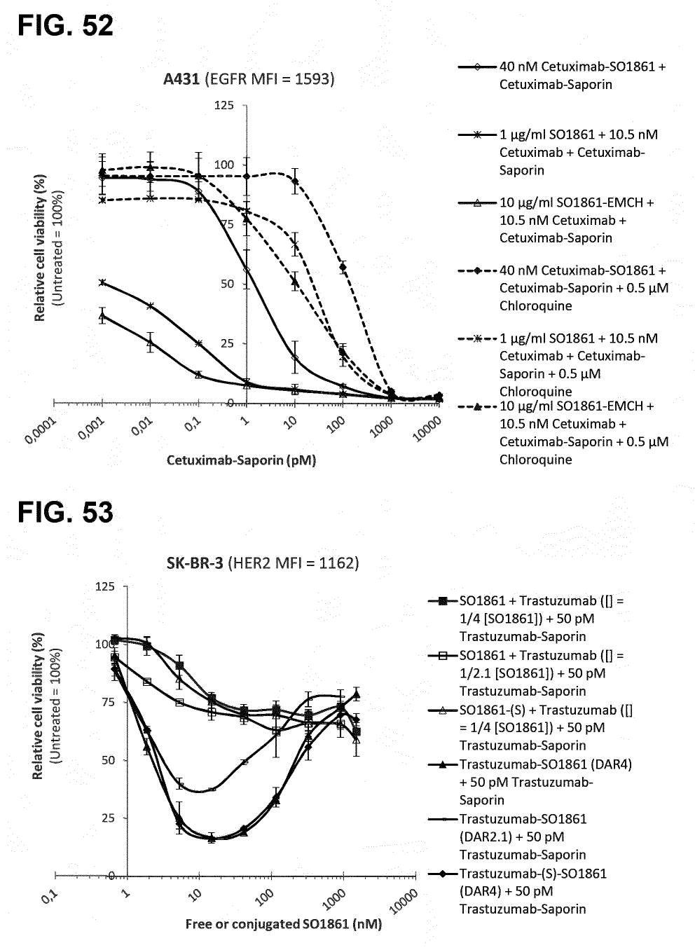

When endosomal acidification is blocked the targeted 2 component system is not active, due to the fact that SPT001 (a plant-derived saponin) is only active at low endosomal pH. (FIG. 52)

### Example 5

Endosomal acidification inhibitors block the targeted 2-component system activity showing that SO1861 function is reduced when acidification of endosomes is blocked.

### Example 6

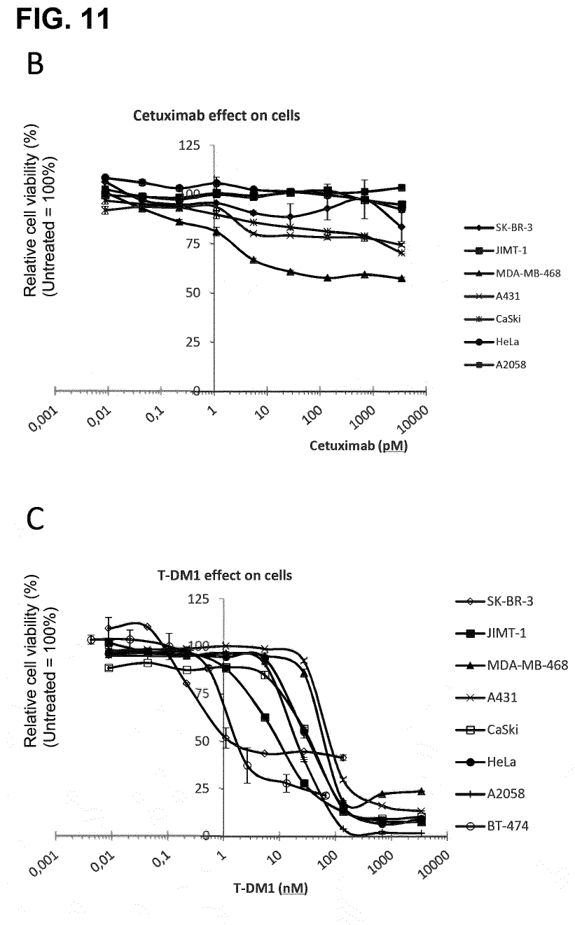

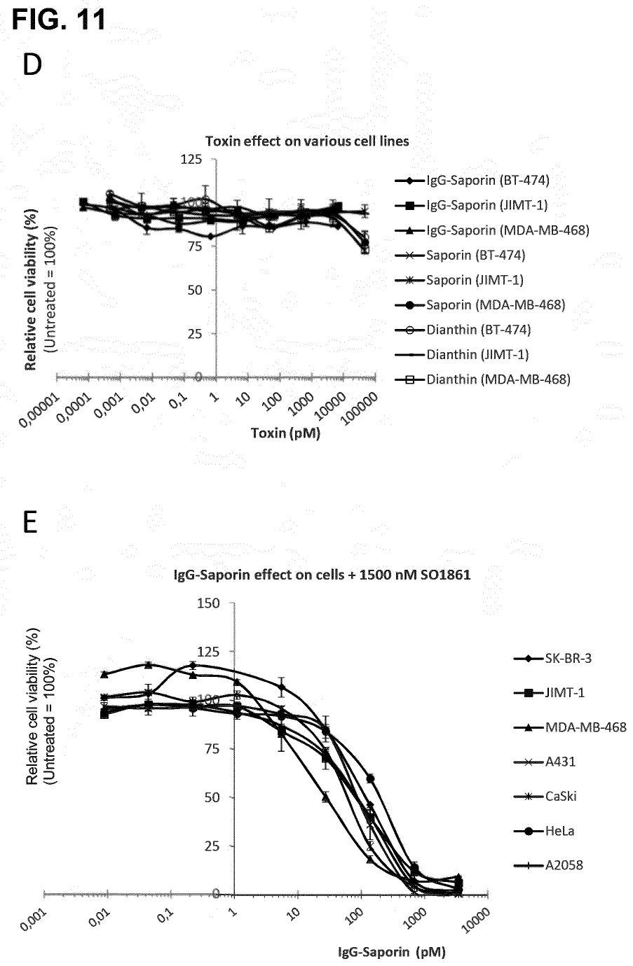

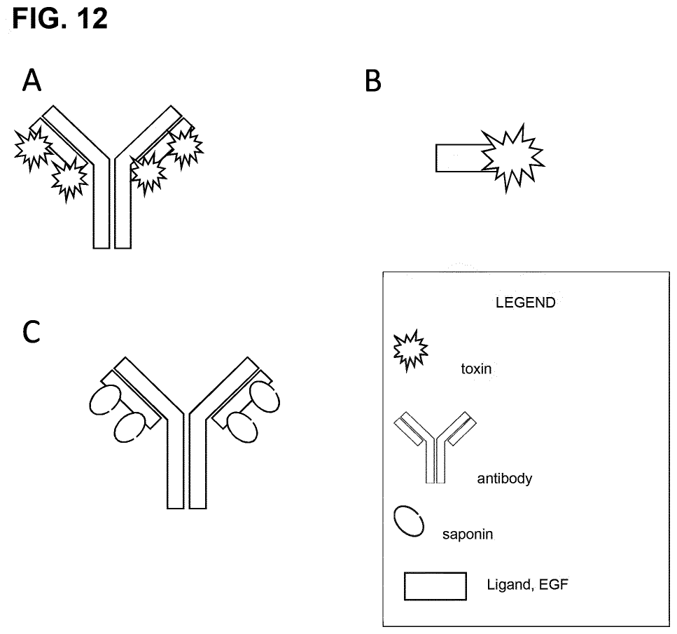

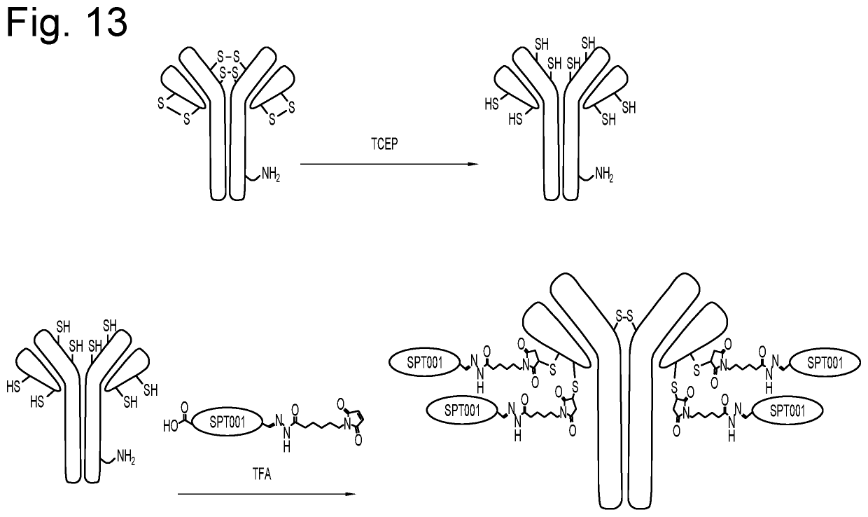

FIG. 11A-E displays the relative cell viability when trastuzumab (FIG. 11A), cetuximab (FIG. 11B) or T-DM1 (FIG. 11C), free toxins saporin (FIG. 11D) and dianthin (FIG. 11D), saporin coupled to a non-cell binding IgG (FIG. 11D), and saporin coupled to a non-cell binding IgG combined with free saponin SO1861 (FIG. 11E) are contacted with the indicated cell lines SK-BR-3, JIMT-1, MDA-MB-468, A431, CaSki, HeLa, A2058, BT-474.

Trastuzumab and cetuximab do not or hardly influence cell viability when exposed to most of the cell lines, with some effect on cell viability when trastuzumab is exposed to SK-BR-3 cells at relatively high dose, and with some effect on cell viability when cetuximab is exposed to MDA-MB-468 cells at relatively high dose.

TDM-1, or ado-trastuzumab emtansine, is a targeted therapy approved by the U.S. Food and Drug Administration to treat: HER2-positive metastatic breast cancer that has previously been treated with Herceptin (chemical name: trastuzumab) and taxane chemotherapy; early-stage HER2-positive breast cancer after surgery if residual disease was found after neoadjuvant (before surgery) treatment with Herceptin and taxane chemotherapy. The TDM-1 is a combination of Herceptin and the chemotherapy medicine emtansine. FIG. 11C shows that the TDM-1 results in decreased cell viability for all cell lines tested at nM concentrations. Noteworthy, TDM-1 was tested at nM concentrations in order to be able to detect effects on the cells, whereas the conjugates, molecules, assemblies, complexes, combinations and compositions of the invention show cell-viability decreasing activity and effects already when exposed to cells at pM concentrations of e.g. an mAb-toxin of the invention.

The free toxins saporin and dianthin and the toxin saporin coupled to a control IgG with no affinity for any of the cell surface molecules on the cell lines tested, do not or hardly have any influence on cell viability over a wide range of concentrations toxin tested, up to 10000 μM. When the toxin saporin is coupled to a non-cell binding IgG, combining the conjugate with the free saponin SO1861 results in an IgG-saporin dose dependent decrease of the relative cell viability (FIG. 11E).

TABLE 1
Data summary of IC50 value of untargeted SO1861targeted 2-component
system mAb-SO1861 titration with fixed [mAb-toxin].

| | | | | | | | 1-target | |
|---|---|---|---|---|---|---|---|---|
| | | | | | | | 2-component | |
| | | | | | | | Both SO1861 | |
| | | | Untargeted SO1861 | | | | and toxin to EGFR | |
| | | | SO1861 + | SO1861 + | | | 10 pM | 10 pM |
| | EGFR | HER2 | 50 pM | 50 pM | SO1861 + | SO1861 + | Cetuximab- | Dianthin: |
| | expres- | expres- | Trastuzu- | Trastuzu- | 10 pM | 10 pM | Saporin + | EGF + |
| | sion | sion | mab- | mab- | Cetuximab- | EGF - | Cetuximab- | Cetuximab- |
| Cell | level | level | saporin | dianthin | saporin | dianthin | SO1861 | SO1861 |
| line | (MFI) | (MFI) | (IC50, nM) | (IC50, nM) | (IC50, nM) | (IC50, nM) | (IC50, nM) | (IC50, nM) |
| MDA- | 1656 | 1 | >1.000 | >1.000 | >1.000 | >1.000 | 3 | 6 |
| MB-468 | | | | | | | | |
| A431 | 1593 | 10 | >1.000 | >1.000 | 600 | >1.000 | 5 | 8 |
| CaSki | 481 | 12 | >1.000 | >1.000 | 700 | >1.000 | 5-10 | 10 |
| SK-BR-3 | 28 | 1162 | 700 | n.d. | 800 | 650 | >1.000 | >1.000 |
| JIMT-1 | 58 | 74 | >1.000 | >1.000 | >1.000 | >1.000 | >1.000 | >1.000 |
| HeLa | 91 | 5 | >1.000 | >1.000 | >1.000 | >1.000 | >1.000 | >1.000 |
| A2058 | 1 | 5 | >1.000 | >1.000 | >1.000 | >1.000 | >1.000 | >1.000 |

| | 1-target | | 2-target | | |
|---|---|---|---|---|---|
| | 2-component | | 2-component | | |
| | Both SO1861 | | SO1861 to | | |
| | and toxin | | EGFR; | | |
| | to HER2 | | toxin to | SO1861 to HER2; | |
| | 50 pM | 50 pM | HER2 | toxin to EGFR | |
| | Trastuzu- | Trastuzu- | 50 pM | 5 pM | 1.5 pM |
| | mab- | mab- | Trastuzu- | Cetuximab- | Dianthin: |
| | Saporin + | Dianthin + | mab- | Saporin + | EGF + |
| | Trastuzu- | Trastuzu- | Saporin + | Trastuzu- | Trastuzu- |
| | mab- | mab- | Cetuximab- | mab- | mab- |
| Cell | SO1861 | SO1861 | SO1861 | SO1861 | SO1861 |
| line | (IC50, nM) | (IC50, nM) | (IC50, nM) | (IC50, nM) | (IC50, nM) |
| MDA- | >1.000 | >1.000 | 18 | >1.000 | >1.000 |
| MB-468 | | | | | |
| A431 | >1.000 | >1.000 | 12 | >1.000 | >1.000 |
| CaSki | >1.000 | >1.000 | 40 | >1.000 | >1.000 |
| SK-BR-3 | 2* | 3* | >1.000 | n.d. | 3 |
| JIMT-1 | >1.000 | >1.000 | >1.000 | >1.000 | >1.000 |
| HeLa | >1.000 | >1.000 | >1.000 | >1.000 | >1.000 |
| A2058 | >1.000 | >1.000 | >1.000 | >1.000 | >1.000 |

FIG. 55 displays a Table with a Data summary of IC50 values for mAb, toxin, ligand toxin or mAB-toxin monotherapy with or without SO1861. In FIG. 55, * refers to: MDA-MB-468 cells show a 20-25% reduction in cell viability at all Cetuximab [ ] above 5 nM; and ** refers to: SK-BR-3 cells show a 20% reduction in cell viability at 1 nM Trastuzumab and 30-35% reduction for all Trastuzumab [ ] above 1 nM.

TABLE 2
Data summary of IC50 values for the targeted 2-component system, mAb-toxin titration with fixed [mAb-SO1861], IC50 for
each value is calculated as a percentage relative to the component(s) with constant concentration for that treatment (100%)

| | | | 1 -target 2-component | | | | 2-target 2-component | | | |
|---|---|---|---|---|---|---|---|---|---|---|
| | | | | | Both SO1861 | | | | | |
| | | | Both SO1861 | | and toxin to | | SO1861 to EGFR; | | SO1861 to HER2; | |
| | | | and toxin to | | HER2 | | toxin to HER2 | | toxin to EGFR | |
| | | | EGFR | | 300 nM | 300 nM | 278 nM | 1388 nM | 300 nM | 300 nM |
| | | | 278 nM | 278 nM | Trastuzu- | Trastuzu- | Cetuximab- | Cetuximab- | Trastuzu- | Trastuzu- |
| | EGFR | HER2 | Cetuximab- | Cetuximab- | mab- | mab- | SO1861 + | SO1861 + | mab- | mab- |
| | expres- | expres- | SO1861 + | SO1861 + | SO1861 + | SO1861 + | Trastuzu- | Trastuzu- | SO1861 + | SO1861 + |
| | sion | sion | Cetuximab- | EGF- | Trastuzumab- | Trastuzumab- | mab- | mab- | Cetuximab- | EGF- |
| Cell | level | level | Saporin | Dianthin | Saporin | Dianthin | Saporin | Dianthin | Saporin | Dianthin |
| line | (MFI) | (MFI) | (IC50, pM) | (IC50, pM) | (IC50, pM) | (IC50, pM) | (IC50, pM) | (IC50, pM) | (IC50, pM) | (IC50, pM) |
| MDA- | 1656 | 1 | 0.5 | 1 | >10.000 | >10.000 | 55 | 500 | 100 | 750 |
| MB- | | | | | | | | | | |
| 488 | | | | | | | | | | |
| A431 | 1593 | 10 | 0.4 | 0.4 | >10.000 | >10.000 | 3 | 30 | 20 | 2.300 |
| CaSki | 481 | 12 | 2 | 1 | >10.000 | >10.000 | 1 | 25 | 14 | 1.600 |
| SK-BR-3 | 28 | 1162 | n/d. | n.d. | 20 | n.d. | n.d. | n.d. | n.d. | 2 |
| JIMT-1 | 58 | 74 | >10.000 | >10.000 | >10.000 | >10.000 | 3.000 | >10.000 | 90 | 4.000 |
| HeLa | 91 | 7 | 6.000 | >10.000 | >10.000 | >10.000 | >10.000 | 10.000 | 50 | 4.000 |
| A2058 | 1 | 5 | >10.000 | >10.000 | >1.0000 | >10.000 | >10.000 | >10.000 | >10000 | >10.000 |

### Example 7

In FIG. 57 a Table displaying antibody-drug conjugates is provided for which it was known in Q3-2019 that the clinical development was interrupted for one reason or the other. These ADCs are typically provided with bound saponin moiety/moieties, typically selected from SA1641, SO1861, GE1741 and combinations thereof, and linking of the saponin SO1861 to these ADCs is preferred, according to the invention. Especially those ADCs that have been in clinical development, and for which clinical development was halted due to reasons relating to lack of efficacy and/or occurrence of (unacceptable) side effects for the patients to whom the ADC was administered, are typical ligands provided with an effector moiety selected from a toxin and an oligonucleotide, which are incorporated in an endosomal/lysosomal escape enhancing conjugate according to the invention, i.e. to which one or more saponin moieties are linked, wherein the saponin moieties enable improved endosomal and/or lysosomal escape once a (tumor) cell is contacted with the ADC from FIG. 57, now endowed with the saponin(s). Thus, an ADC selected from FIG. 57 is coupled to one or more saponins such that an endosomal/lysosomal escape enhancing conjugate of the invention is provided, which thus provides enhanced intracellular trafficking properties to the payload such as a toxin, oligonucleotide, an enzyme, BNA, etc. (i.e. an effector moiety ‘E’), The conjugate thus provides possibly enhanced intracellular trafficking properties for intracellular drug molecule(s), according to the invention. This example is displaying one of the many possibilities for providing conjugates which contribute to improved intracellular trafficking once an effector moiety is delivered at the targeted cell and into the interior of the cell, e.g. in the endosomal /lysosomal compartment such as the late endosomes and/or lysosomes. In FIG. 57, the overview of antibody-drug conjugates of which development was discontinued, inactive, withdrawn or the filing was rejected due to various reasons, provides a series of examples of combinations of ligands and payloads (toxins, oligonucleotides) which once incorporated in a conjugate of the invention, have improved endosomal escape enhancing capabilities when the payload is concerned.

Alternatively or additively, the ADCs of FIG. 57 are combined with an endosomal/lysosomal escape enhancing conjugate of the invention, the conjugate containing one or more saponin moieties according to the invention and an effector moiety, the effector moiety selected from any of a cell-surface targeting ligand or an immunoglobulin, a binding domain or binding fragment thereof. In this embodiment, the conjugate of the invention does not encompass an effector moiety which is a payload. The invention thus also relates to a method for treating a patient in need thereof with an effective dose of an ADC such as an ADC listed in FIG. 57, in combination with an effective dose of an endosomal /lysosomal escape enhancing conjugate of the invention comprising saponin(s) and an aberrant cell-targeting ligand such as EGF, an antibody or any of the cell-targeting ligands listed in FIG. 57. Typically, the saponin is SO1861. Typically the ligand in the conjugate of the invention is EGF, trastuzumab, cetuximab, anti-CD71 monoclonal antibody (CD71 mAb).

Preferred ADCs for incorporation in the manufacturing of an endosomal/lysosomal escape enhancing conjugate of the invention and for incorporation in a combination therapy for treating e.g. cancer in a human subject in need of treatment, the ADC treatment combined with treatment of the cancer of the patient with an endosomal/lysosomal escape enhancing conjugate of the invention, are those ADCs for which clinical development is disrupted due to side effects and/or lack of efficacy. Examples are: BIWI-1, ADCT-502, Avicidin, vadastuximab talirine, for which adverse events were at the basis for discontinuing the development, and depatuxizumab mafodotin, indusatumab vedotin, glembatumumab vedotin, for which clinical development was discontinued relating to lack of efficacy.

According to the invention, combining these ADCs with an efficacious and sufficient amount of a saponin-comprising conjugate specific for the same cell as to which the ADC has to bind for exerting pharmaceutical activity, improves therapeutic efficacy and may reduce side effects, typically by applying a reduced dose of the ADC, either when part of a conjugate of the invention, or when combined with a conjugate of the invention. Since the conjugate of the invention improves intracellular trafficking of molecules that have therapeutic effect inside cells in which these molecules are delivered, the dose of an ADC that was administered to patients and that resulted in adverse events, can be lowered while presence of the targeted saponin improves intracellular trafficking of the payload of the ADC to the side inside the cell where therapeutic activity is desired, e.g. endosomal escape enhancing effects of the saponin aids in establishing an increased intracellular dose of the payload at the location inside the cell where the payload can exert its therapeutic effect, according to the invention. As said, the saponin is either part of the ADC, providing a conjugate of the invention, or the ADC is combined with a saponin targeting the same aberrant cell as the ADC, therewith delivering saponin to the endosomal/lysosomal machinery and contributing to the build up of payload concentration inside the cell to be killed, altered, etc.

### Example 8—Saponin with Modified Chemical Groups Remains Active

Linkage of saponin to a ligand does not interfere substantially with the ability of the saponin to enhance endosomal escape (linked saponin or saponin freed from the conjugate inside the endosome). The saponin is either linked to the ligand directly, or via a (cleavable) linker, via a scaffold, a carrier protein, etc., etc. Results of experiments are summarized in Table 3, here below.

Chemically modified saponin SO1861 did show reactivity in a cell-based bioassay, with relative cell viability as the read out. HeLa cells were incubated for 72 h with the following constructs and cell viability before and after the 72 h-incubation was assessed. In the experiments, cells were exposed to 1.5 μM dianthin-EGF conjugate. A negative control were cells incubated with buffer vehicle and 10 microgram/ml saponin, without dianthin-EGF. Cell viability was set to 100% for the control in which both saponin and EGF-dianthin were omitted. Positive controls were 10 microgram/ml of non-modified saponin SO1861+dianthin-EGF. Cell viability after 72 h was essentially 0%. For the chemically modified saponin variants, 10 microgram/ml saponin was tested in combination with 1.5 μM dianthin-EGF. SO1861 provided with an EMCH moiety at the aldehyde group induced reduced cell viability at 10 microgram/ml.

These data demonstrate that the saponin can be modified at the free aldehyde group or at the free carbonyl group without losing the endosomal escape enhancing activity.

### Example 9—QS-21 Saponins with Endosomal/Lysosomal Escape Enhancing Activity

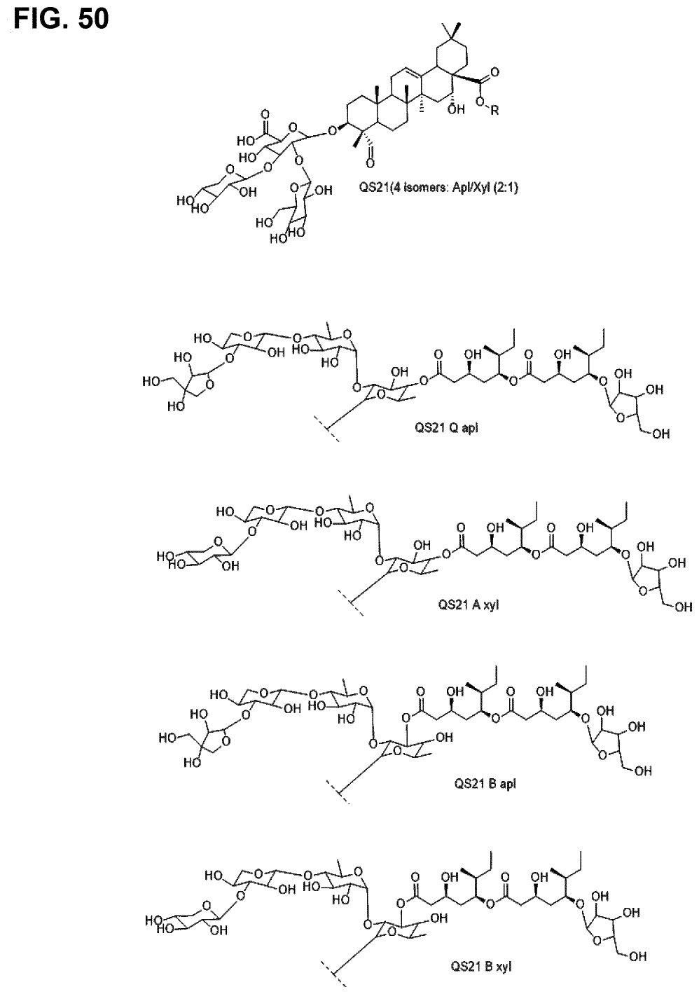

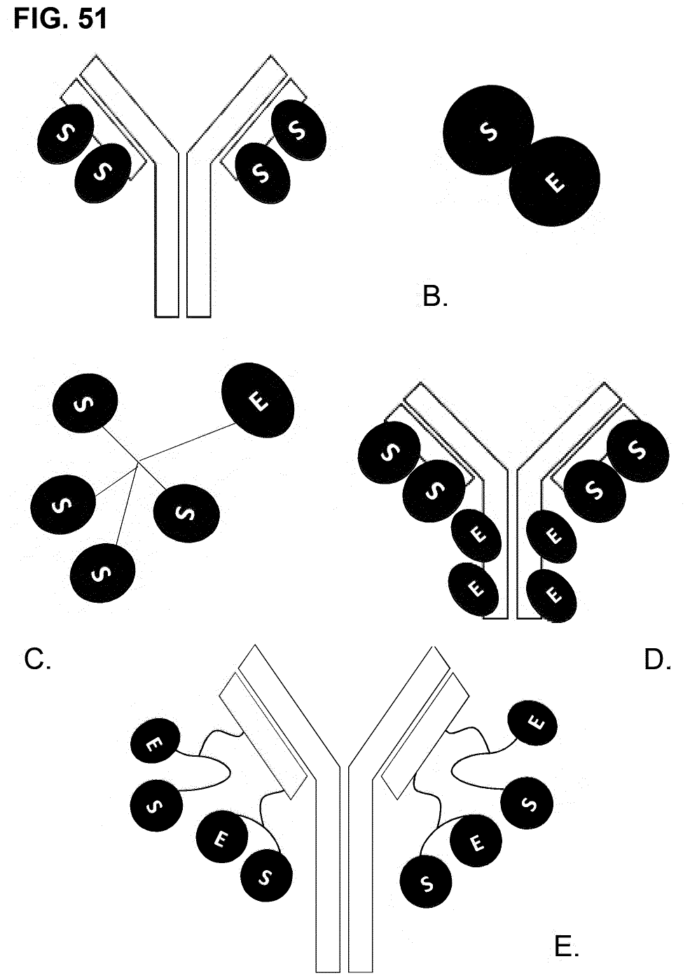

FIG. 50 displays the common molecular structure of a series of QS-21 saponins: structure of 4 QS-21 isomers. Such saponins may be applied in the endosomal/lysosomal escape enhancing conjugate, composition, combination of the invention, based on endosomal/lysosomal escape enhancing properties of an individual saponin present in QS-21, or based on a combination of two or more of the saponins comprised by QS-21, or QS-21.

The inventors demonstrated that QS-21 at 2.5 microgram/ml dose was capable of enhancing endosomal escape, as tested with mammalian cells in a cell-based bioassay. The effector moiety exposed to the cells was EGF-dianthin.

TABLE 3
Cell killing activity (+ or −) of unconjugated SO1861 and SO1861 conjugated
via the aldehyde or the carboxylic acid group to a linker (EMCH = labile,
hydrazone; HATU = stable, amide; N3/Azide = labile, hydrazone) when
coadministrated with a targeted toxin (EGFdianthin) and results in enhanced
cell killing compared to untreated control of EGFR expressing cells (e.g. A431, HeLa)

| | | | SO1861- | SO1861- | SO1861- |
|---|---|---|---|---|---|
| | Buffer only | SO1861 | EMCH | N3/Azide | HATU |
| Conjugation site | NA | NA | Aldehyde | Aldehyde | Carboxylic |
| on SO1861 | | | | | acid |
| | | | | | (glucoronic |
| | | | | | acid) |
| 10 μg/ml SO1861 | − | − | − | − | − |
| 10 μg/ml SO1861 + | − | + | + | + | + |
| 1.5 pM | | | | | |
| EGFdianthin | | | | | |

### Example 10—Endosomal Escape Enhancing Capacity of Saponins is pH Dependent

The saponin SO1861 was tested for its endosomal escape enhancing activity in a cell-based bioassay using A431 cells. Results of the experiments are summarized in FIG. 52. Activity of the saponin was tested under influence of endosomal pH. As a control, 1 microgram/ml free saponin or 10 microgram/ml SO1861 conjugated with an EMCH moiety were combined with a combination of free monoclonal antibody cetuximab and the ADC cetuximab-saporin. At a dose of 1 μM of the ADC, cell viability was reduced with about 90% and an IC50 of about 0.07 μM. In a separate experiment, adding chloroquine to the cell culture medium at a dose of 0.5 micromolar, in addition to the aforementioned combination of 1 microgram/ml free saponin or 10 microgram/ml SO1861 conjugated with an EMCH moiety combined with a combination of free monoclonal antibody cetuximab and the ADC cetuximab-saporin, resulted in an increase of the IC50 to about 30 μM for the ADC dose and at a dose of about 700 μM ADC cell viability decreased to about 10%.

In a similar experiment, 40 nM saponin SO1861 was now linked to cetuximab and combined with a concentration series of the ADC cetuximab-saporin. The influence of 0.5 micromolar chloroquine was assessed. It was observed that under influence of the chloroquine the efficacy of the SO1861-cetuximab in enhancing endosomal escape of the ADC was decreased with about a factor 200. That is to say, the IC50 of the ADC when cell viability is concerned, dropped from about 1 μM ADC when chloroquine was omitted in the cell culture medium, whereas the IC50 increased to about 200 μM ADC in the presence of 0.5 micromolar chloroquine in the cell culture medium of the A431 cells.

In FIG. 52: Cells with high EGFR expression (A431) can efficiently be killed with SO1861 or SO1861-EMCH+cetuximab-saporin or with the therapeutic combination according to the invention, however coadministration of the endosomal acidification inhibitor chloroquine strongly reduces the activity of SO1861, SO1861-EMCH as well as the therapeutic combination according to the invention.

These results together demonstrate the pH dependence of the endosomal escape enhancing efficacy of the saponin. Both the free saponin and the EMCH modified saponin on the aldehyde group displayed endosomal escape enhancing activity, which was lowered under influence of chloroquine. Similarly, saponin bound to targeting antibody showed reduced activity in the presence of chloroquine, and without chloroquine in the cell culture medium, the saporin-antibody conjugate was effective in enhancing the endosomal escape of the ADC, as measured by assessing decrease of cell viability. Thus, increasing endosomal pH by adding chloroquine to the cultured cells inhibits the endosomal escape enhancing activity of saponin, saponin provided with a modified aldehyde group and saponin conjugated to a ligand, here a targeting antibody for the EGFR.

These experiments show the endosomal escape enhancing effect with regard to the ADC, under influence of free saponin or conjugated saponin, and under influence of endosomal/lysosomal pH. Increasing endosomal pH lowers the endosomal escape of the ADC. For the saponin it is known that the endosomal escape enhancing activity is apparent at pH lower than 6.5 such as pH 4.5-5.5.

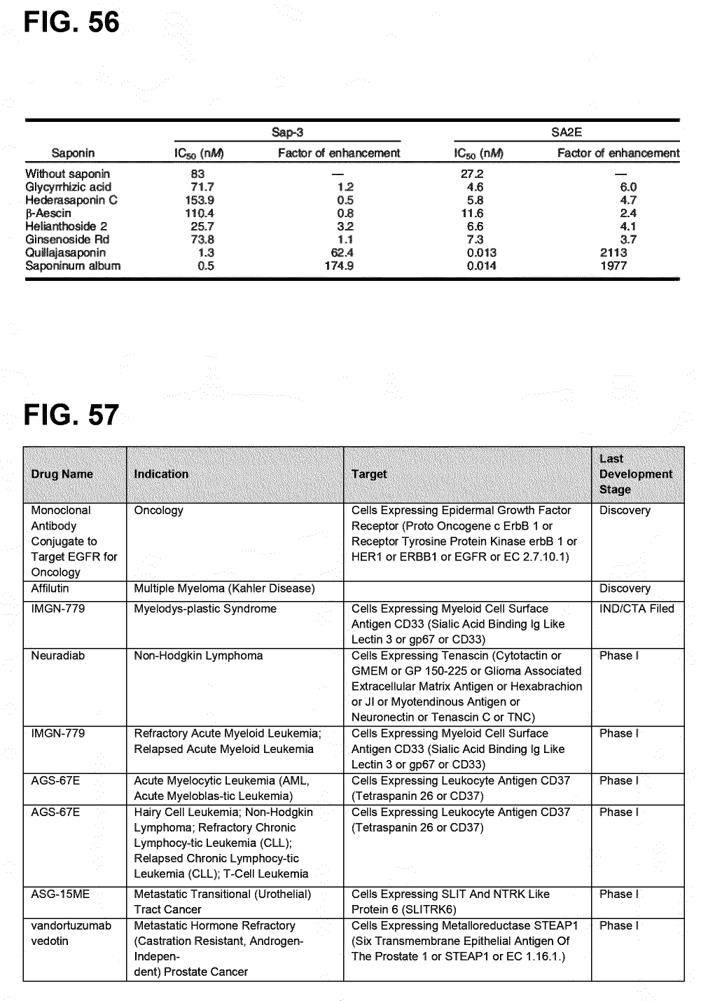

These results are in line with previous reported observations by one of the inventors, published in BACHRAN, 2006 (CHRISTOPHER BACHRAN, MARK SUTHERLAND, IRING HEISLER, PHILIPP HEBESTREIT, MATTHIAS F. MELZIG, AND HENDRIK FUCHS, The Saponin-Mediated Enhanced Uptake of Targeted Saporin-Based Drugs Is Strongly Dependent on the Saponin Structure, Exp Biol Med 231:412-420, 2006), showing that saponins Quillajasaponin and Saponium album enhance cell toxicity of toxins towards NIH-3T3 cells with a factor 20-30 up to over 500, whereas different saponins such as glycyrrhizic acid and helianthoside do hardly have any positive effect, i.e. enhancing effect, on toxicity of the saporins towards the incubated cells. See the Table in FIG. 56, adapted from Bachran, 2006, here below in that regard. Noteworthy, saponins GE1741 (gypsophilia) and SO1861 (salonaria) are extracted from the saponinum album.

In combination, the current example shows that the saponins that are capable of modulating, i.e. enhancing the toxic activity of a toxin towards cells, are a specific class of saponins which exhibit their endosomal escape enhancing activity in an endosomal pH dependent manner, i.e. activity is displayed at normal low endosomal pH whereas activity decreases at increased pH under influence of contacting cells with chloroquine. Furthermore, saponins with endosomal/lysosomal escape enhancing activity remain active also after modulating the free aldehyde group of the saponin or after conjugating the saponin with an antibody.

FIG. 56 displays a Table showing IC50 values and Saponin-Mediated Factors of enhancement for the toxin saporin-3 (Sap-3) and a chimeric saporin adapter-EGF SA2E on NIH-3T3 cells in the absence and presence of different Saponins.

In summary, according to the invention, saponins with endosomal/lysosomal escape enhancing activity are capable of enhancing escape of molecules from acidic vesicles or acidic compartments such as early endosomes, late endosomes, endolysosomes, lysosomes or recycling endosomes. Such saponins displaying this activity are part of the invention and incorporated in conjugates, assemblies, compositions and combinations according to the invention. Furthermore, such saponins with endosomal/lysosomal escape enhancing activity are provided with a modified or activated chemical unit, or a linker, etc., such as the aldehyde group on SO1861 or the carboxyl group on SO1861 and GE1741 provided with a linker, an EMCH moiety, an AMPD moiety, etc., for the purpose of linking the saponin with a scaffold or antibody or binding domain thereof, or a peptide, an enzyme or a payload or a drug molecule or a targeting molecule or a toxin, etc., etc., according to the invention. The common denominator amongst such conjugates, complexes, molecules of the invention is their capacity to enhance the intracellular activity of an effector moiety or effector molecule which is either conjugated, linked, complexed with the saponin such as SO1861, or which is co-administered to cells that are exposed to the saponin, for example when bound to a cell targeting ligand such as EGF or an antibody with affinity for a tumor cell specific cell surface molecule. The effector moiety of molecule is for example a toxin, a BNA, an enzyme.

### Example 11—Saponin Complexed with Trastuzumab has Endosomal Escape Enhancing Activity when the Saponin is Linked to the Antibody Via a Labile Linker or a Stable Linker

The inventors established that the saponin SO1861 keeps its endosomal escape enhancing activity when the effect of saporin on cell viability of SK-BR-3 is assessed, when the saponin is linked to an antibody using a stable linker or a labile linker, which is cleavable in the endosome. Results are summarized in FIG. 53. Free unconjugated saponin combined with 50 μM trastuzumab-saporin conjugate (ADC) was added to an SK-BR-3 cell culture and cell viability was assessed after incubation. The concentration of the free unconjugated saponin was titrated and cell viability under influence of saponin concentration was assessed. Even at a dose of about 100 nM, 300 nM and 1000 nM SO1861, in the presence of 50 μM trastuzumab-saporin, cell viability decreased only with about 30-40%. In contrast, when trastuzumab-saponin is combined with 50 μM trastuzumab-saporin, cell viability decreases to a larger extent, up to about minus 80%.

FIG. 53: HER2 targeted cell killing in SKBR3 (HER2⁺⁺⁺) cells by a therapeutic combination according to the invention. Trastuzumab-SO1861 (labile bond between saponin and the antibody, DAR4) or Trastuzumab-(S)-SO1861 (stable bond between SO1861 and the antibody; DAR4) titration in combination with a fixed concentration of 50 μM Trastuzumab-Saporin shows efficient cell killing, showing that stable (conjugated to the carboxylic acid group of SO1861) or labile (conjugated to the aldehyde group of SO1861) conjugation of SO1861 to an antibody result in similar potency in SKBR-3 cells. Trastuzumab-SO1861 (labile bond between the saponin and the antibody, DAR 2.1) in combination with a fixed concentration of 50 μM Trastuzumab-Saporin also show cell killing effects, indicating that the amount of conjugated SO1861 molecules per antibody influences the extent of cell killing by the fixed dose of ADC.

### Example 12—Gene Silencing by a BNA (HSP27) is Enhanced by High-Dose Free Saponin and Low-Dose Saponin Conjugated with a Targeting Ligand, i.e. Cetuximab

FIG. 54 displays the results of an experiment in which HSP27 silencing BNA was added to A431 cells which express HSP27. The influence of co-administering free saponin SO1861 modified on its aldehyde group with an EMCH moiety or SO1861 coupled to cell-surface receptor EGFR targeting antibody cetuximab, with the BNA (HSP27) on HSP27 gene silencing in the A431 cells was assessed. Cetuximab-SO1861+HSP27 BNA oligo induces enhanced gene silencing of HSP27 target gene. Ten pg/ml (4829 nM) SO1861-EMCH+HSP27 BNA oligo or 100 nM cetuximab-SO1861+HSP27 BNA oligo induces strong (100-fold) enhancement of HSP27 gene silencing (resp. IC50=4 nM; IC50=10 nM) in high EGFR expressing cells (A431), and when compared to exposing the cells with the BNA in the absence of free saponin or saponin conjugated with the targeting ligand, i.e. cetuximab.

These experiments show that the endosomal escape enhancing saponin SO1861 is capable of modulating, i.e. stimulating the gene silencing efficacy of the BNA (HSP27) in HSP27 expressing cells. Moreover, providing a saponin which comprises a cell targeting ligand, here an EGFR binding antibody, results in a gene silencing effect at 100 mM cetuximab-SO1861 which is comparable to the silencing effect achieved when BNA (HSP27) is exposed to the A431 cells in the context of free modified SO1861, i.e. SO1861-EMCH at a dose of 10 microgram/ml. Expression of the cell surface marker EGFR was established in a FACS set-up.

### Example 13—Linker Systems Applicable for the Saponin Complexes of the Invention

Many linkers and linker chemistries are known in the art, for linking and coupling molecules together. As outlined in the embodiments and examples here above indeed a series of possible linker chemistries have been applied with the saponins according to the invention. In addition, conventional linker chemistries based on NHS-EDC coupling are applicable for providing conjugates, combinations, compositions of the invention, such as saponins with endosomal escape enhancing activity linked to an NHS moiety, and subsequently coupled to an antibody provided with an EDC moiety, or vice versa (saponin-EDC for coupling to NHS-IgG, for example). Similarly and also beneficially applicable is using the established coupling technique implying biotin and Streptavidin pairs. That is to say, either saponin or a linker, scaffold, antibody, payload, etc., can be provided with a biotin moiety, and the binding partner can be provided with a Streptavidin moiety, i.e. the linker, scaffold, antibody, payload, etc., if the saponin has a biotin molecule bound to it, or vice versa.

In addition, further linkers applicable for linking a glycoside including the saponins of the invention, are known in the art. International patent application WO1993005789A1 for example discloses the following linkers suitable for linking saponins to a further molecule, while retaining saponin activity. Suitable linkers are:

-NH—(CH₂)_{q}—NH—,

wherein q is 2-10;

-O—(CH₂)ᵣ-NH—,

wherein r is 2-10;

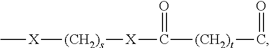

wherein X=NH, S or O, s=2-5, t=2-12;

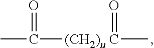

wherein u=2-12;

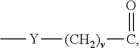

wherein Y is NH or S, v—1-3; or

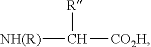

wherein R″ is hydrogen, C₁₋₄alkyl, or C₁₋₄ alkyl substituted by phenyl, hydroxyphenyl, indolyl, mercapto, C₁₋₄ alkylthio, hydroxy, carboxy, amino, guanidino, imidazolyl or carbamyl; or wherein R and R″ together form a pyrrolidinyl or piperidinyl ring.

### Example 14

Conjugates of the invention. FIG. 51 displays five typical molecular assemblies or conjugates, complexes of the invention. These conjugates are manufactured and purified, for testing in cell-based bioassays, in vivo animal models, etc.

FIG. 51A is a cartoon representing the endosomal/lysosomal escape enhancing conjugate according to the invention, comprising at least one saponin moiety ‘S’ complexed with a targeting ligand such as an IgG, wherein the saponin is linked directly to the antibody, or via a (cleavable) linker. The saponins are typically linked to the —SH groups of the cysteines in the ligand. Typically, the at least one saponin is selected from SA1641, SO1861, GE1741 and combinations thereof, and the saponin SO1861 is preferred. Typical cell-surface molecule targeting ligands selected for incorporation in the conjugate of the invention are immunoglobulins specific for (tumor) cell-surface receptors such as trastuzumab, cetuximab, anti-CD71 monoclonal antibody, or EGF for binding to EGFR. Also the antibodies listed in FIG. 57 are preferred for manufacturing a conjugate of the invention according to FIG. 51A.

Examples of endosomal/lysosomal escape enhancing conjugates of FIG. 51A that are manufactured and tested for activity by the current inventors are at least cetuximab-SO1861, wherein the saponin is linked via a cleavable hydrazine linkage to the antibody; trastuzumab-SO1861, wherein the saponin is linked via a cleavable hydrazine linkage to the antibody; CD71 mAb-SO1861, wherein the saponin is linked via a cleavable hydrazine linkage to the antibody; trastuzumab-saporin, wherein the payload is linked to the antibody via a disulphide bond, in a combination with trastuzumab-SO1861; cetuximab-saporin, wherein the payload is linked to the antibody via a disulphide bond, in a combination with cetuximab-SO1861; CD71 mAb-saporin, wherein the payload is linked to the antibody via a disulphide bond, in a combination with trastuzumab-SO1861; trastuzumab-saporin, wherein the payload is linked to the antibody via a disulphide bond, in a combination with cetuximab-SO1861; cetuximab-saporin, wherein the payload is linked to the antibody via a disulphide bond, in a combination with trastuzumab-SO1861; CD71 mAb-saporin, wherein the payload is linked to the antibody via a disulphide bond, in a combination with cetuximab-SO1861, wherein the saponin is linked via a cleavable hydrazine linkage to the antibody.

FIG. 51B displays a different embodiment of the invention, showing the endosomal/lysosomal escape enhancing conjugate according to the invention, comprising at least one saponin ‘S’ moiety complexed with an effector moiety ‘E’, wherein the effector moiety is a payload such as an immune-toxin or proteinaceous toxin, peptide toxin, small molecule toxin. Typically the payload is selected from dianthin, saporin, ribosomal inactivating protein, or is an RNA, BNA, oligonucleotide, or an enzyme. The saponin and the payload are coupled directly or via a linker such as a cleavable linker, cleavable under acidic conditions, such as at a pH of 4.5-5.5. Typically, the at least one saponin is selected from SA1641, SO1861, GE1741 and combinations thereof, and the saponin SO1861 is preferred.

Examples of endosomal/lysosomal escape enhancing conjugates of FIG. 51B that are manufactured and tested for activity by the current inventors are at least BNA-SO1861 wherein the oligonucleotide or ASO (BNA) silences HSP27 and wherein the saponin is coupled via a cleavable hydrazine linkage to the payload.

FIG. 51C displays the cartoon of an endosomal/lysosomal escape enhancing conjugate according to the invention, comprising at least one saponin ‘S’ moiety complexed with an effector moiety ‘E’ via a multifold linker such as a dendron comprising four binding sites for saponin moieties and a single effector moiety. Typically, the at least one saponin is selected from SA1641, SO1861, GE1741 and combinations thereof, and the saponin SO1861 is preferred. The effector moiety is selected from any of a toxin, drug molecule, oligonucleotide such as a BNA, RNA, an enzyme.

Examples of endosomal/lysosomal escape enhancing conjugates of FIG. 51C that are manufactured and tested for activity by the current inventors are at least a dendron as a scaffold, bound to four terminal saponins and an LNA moiety or a BNA moiety, wherein the saponin is SO1861. The BNA moiety silences HSP27 (ASO (BNA)).

FIG. 51D is a cartoon representing the endosomal/lysosomal escape enhancing conjugate according to the invention, comprising at least one saponin moiety ‘S’ complexed with a targeting ligand such as an IgG, wherein the saponin is linked directly to the antibody, or via a (cleavable) linker, the antibody further complexed with at least one effector moiety ‘E’ via (cleavable) bond(s). The saponins are typically linked to the —SH groups of the cysteines in the ligand, here an antibody. The effector moiety/moieties is/are typically linked to the —SH groups of the cysteines in the ligand, here an antibody. Typically, the at least one saponin is selected from SA1641, SO1861, GE1741 and combinations thereof, and the saponin SO1861 is preferred. Typical cell-surface molecule targeting ligands selected for incorporation in the conjugate of the invention are immunoglobulins specific for (tumor) cell-surface receptors such as trastuzumab, cetuximab, anti-CD71 monoclonal antibody, or EGF for binding to EGFR. In FIG. 51D the cell-targeting ligand is an antibody specific for a cell-surface receptor. Typical targeted cell-surface molecules are HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CD38, FGFR3, CD123, DLL3, CEACAM5, HER3, CD74, PTK7, Notch3, FGF2, C4.4A, FLT3, CD71. Also the antibodies listed in FIG. 57 are preferred for manufacturing a conjugate of the invention according to FIG. 51D. Typically the effector moiety/moieties is/are selected from dianthin, saporin, ribosomal inactivating protein, or is/are an RNA, BNA, oligonucleotide, or an enzyme. The saponin and the payload are coupled directly to the antibody or are linked to the antibody via a linker such as a cleavable linker, cleavable under acidic conditions, such as at a pH of 4.5-5.5.

Examples of endosomal/lysosomal escape enhancing conjugates of FIG. 51D that are manufactured and tested for activity by the current inventors are at least cetuximab, anti-CD71 monoclonal antibody, and trastuzumab coupled to terminal SO1861 and coupled to a payload such as HSP27 silencing ASO (BNA), dianthin, the enzyme Cre-recombinase.

FIG. 51E is a cartoon representing the endosomal/lysosomal escape enhancing conjugate according to the invention, comprising at least one saponin moiety ‘S’ complexed with a targeting ligand such as an IgG via a scaffold moiety such as a dendron, wherein the saponin is linked directly to the dendron, or via a (cleavable) linker. The dendron moiety/moieties is/are typically linked to the —SH groups of the cysteines in the ligand (the antibody). Typically, the saponins are selected from SA1641, SO1861, GE1741 and combinations thereof, and the saponin SO1861 is preferred. Typical cell-surface molecule targeting ligands selected for incorporation in the conjugate of the invention are immunoglobulins specific for (tumor) cell-surface receptors such as trastuzumab, anti-CD71 monoclonal antibody, cetuximab. Also the antibodies listed in FIG. 57 are preferred for manufacturing a conjugate of the invention according to FIG. 51E. The conjugates comprise the antibody which is further complexed with at least one effector moiety ‘E’ wherein the effector moiety/moieties is/are linked to the same scaffold such as a dendron to which the at least saponin moiety is coupled, the effector moiety coupled to the dendron via (cleavable) bond(s) such as via a linker. Typically the antibody binds to any of cell-surface molecules HER2, EGFR, CD20, CD22, Folate receptor 1, CD146, CD56, CD19, CD138, CD27L, PSMA, CanAg, integrin-alphaV, CA6, CD33, mesothelin, Cripto, CD3, CD30, CD33, CD239, CD70, CD123, CD352, DLL3, CD25, ephrinA4, MUC1, Trop2, CD38, FGFR3, CD123, DLL3, CEACAM5, HER3, CD74, PTK7, Notch3, FGF2, C4.4A, FLT3, CD71.

Examples of endosomal/lysosomal escape enhancing conjugates of FIG. 51E that are manufactured and tested for activity by the current inventors are at least trastuzumab provided with at least a dendron, the at least one dendron bound to terminal saponin moiety/moieties and terminal payload moiety/moieties. The saponin is typically SO1861, the payload is typically BNA capable of silencing HSP27 (ASO (BNA)). The SO1861 is coupled to the dendron via a cleavable hydrazine linkage.

A further example is the endosomal/lysosomal escape enhancing conjugate of the invention consisting of an effector moiety and more than one saponin moieties linked to the effector moiety via a scaffold. The effector moiety is a ligand selected from an immunoglobulin such as trastuzumab, anti-CD71 monoclonal antibody, cetuximab, any of the antibodies listed in FIG. 57. The saponin is SO1861. The ligand has more than one dendron bound to it, each dendron moiety having more than one saponin moiety bound to it. For example, the antibody has one, two, three or four dendrons bound to it, e.g. via disulphide linkages, and for example each dendron has two, three or four saponins terminally bound to it, such that the conjugate of the invention for example encompasses an aberrant cell targeting antibody comprising two, three or four dendrons bound to it, with each dendron carrying four terminal saponins. This adds up to eight, twelve or sixteen saponin moieties attached to a single ligand moiety, i.e. a cell-surface receptor specific antibody targeting aberrant cells such as tumor cells.

### Example 15

Conjugates of the invention comprising a cell-surface molecule targeting ligand such as EGF or an antibody such as cetuximab, trastuzumab, anti-CD71 mAb, any antibody listed in FIG. 57, are suitable for delivery of endosomal and/or lysosomal escape enhancing saponin inside the targeted cell, thereby stimulating intracellular trafficking of a payload such as a toxin, that was coupled to the conjugate or that was separately exposed to target cells and also internalized by said cells. Said endosomal and/or lysosomal escape enhancing activity is controllable and can be modulated and adjusted when the extent of said activity is concerned. For example, the conjugate of the invention can be decorated with a selectable number of saponin moieties such as any number between 1 and for example 100, such as 2, and multiples of 4. An increasing number of saponin moieties comprised by a conjugate of the invention results in an increase of the stimulatory effect of the conjugate when delivery of an effector moiety at the desired intracellular side of action is concerned. Alternatively or additively, the affinity and/or the avidity of the ligand comprised by the conjugate of the invention is tuneable and controllable, when the binding interaction of the conjugate with a targeted cell is concerned. For the manufacturing of a conjugate for example an immunoglobulin can be selected which as a relatively moderate affinity for a certain selected cell surface receptor, or an immunoglobulin can be selected which as a relatively high affinity for the same selected cell surface receptor or for a different selected cell surface receptor, such that the speed and amount of internalized conjugate over time is tuned to the purpose. For the manufacturing of a conjugate for example an immunoglobulin can be selected comprising for example a single Vh domain with binding affinity for a certain selected cell surface receptor, or an comprising for example two or more Vh domains each with binding affinity for the same selected cell surface receptor or for a different selected cell surface receptor, such that the speed and amount of internalized conjugate over time is tuned to the purpose. For the manufacturing of a conjugate for example an immunoglobulin can be selected which has binding affinity fora certain selected cell surface receptor which is moderately expressed on the surface of the target cell, or for the same selected cell surface receptor or for a different selected cell surface receptor, which is highly expressed on the cell surface, for example by exposing the cells with an agent that stimulates the expression of the receptor therewith increasing the cell surface density of the targeted receptor, such that the speed and amount of internalized conjugate over time is tuned to the purpose.

### Example 16

### Materials and Methods

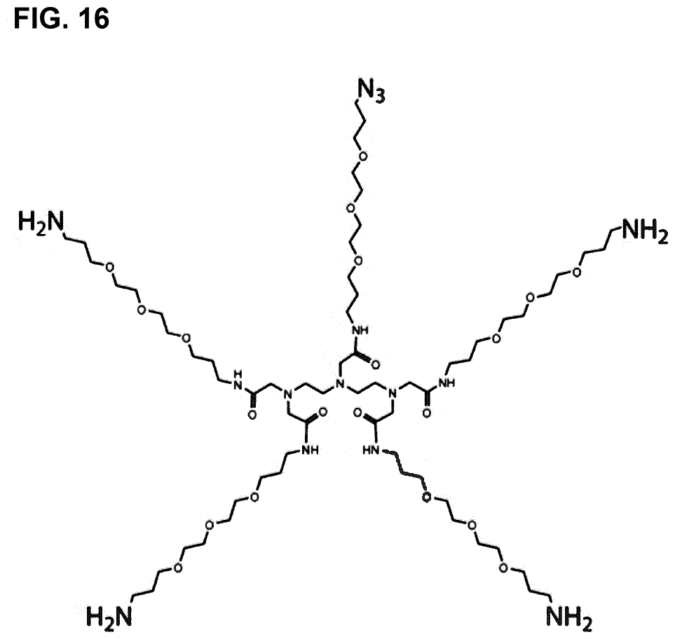

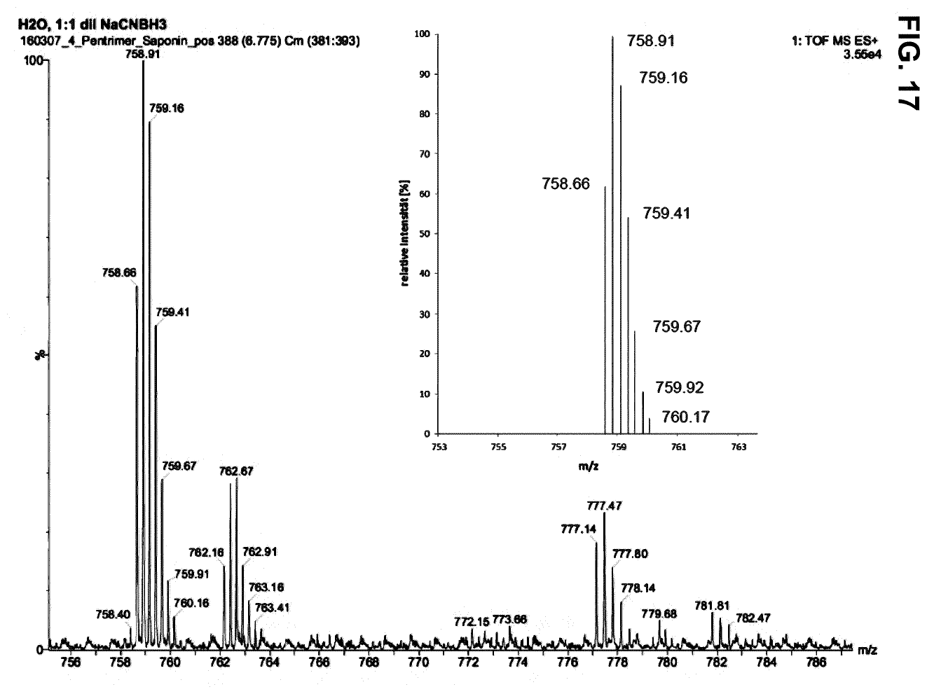

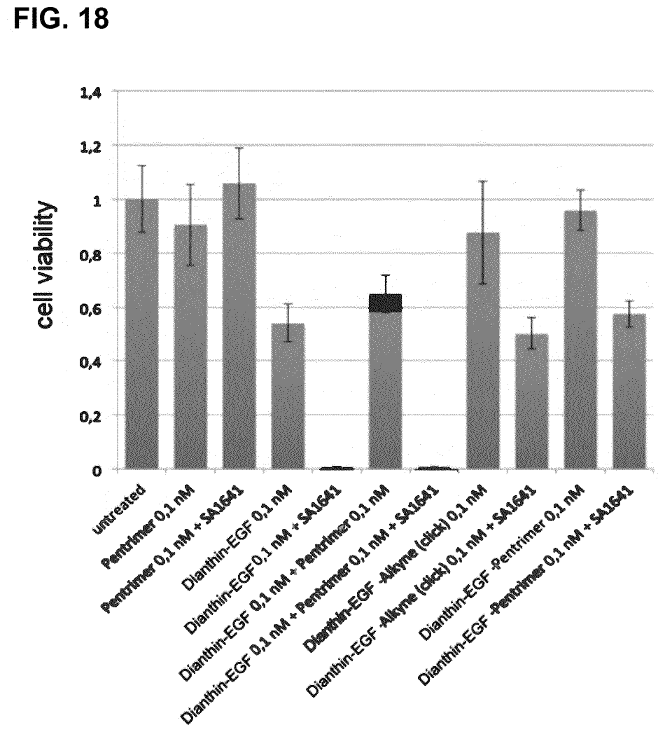

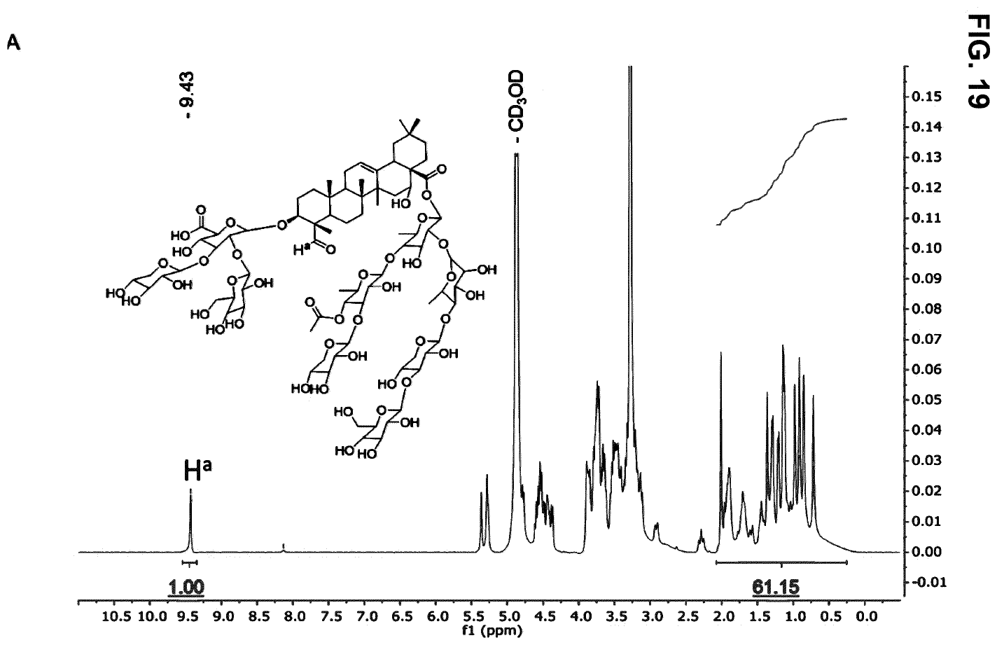

The inventors investigated a model scaffold consisting of four molecular arms for saponin binding via a Schiff base (imine) and one arm for click chemistry. The polymeric structure (FIG. 16) is a pentavalent polyethylene glycol-based dendrimer of the first generation (i.e. number of repeated branching cycles) that was purchased from Iris Biotech GmbH (Marktredwitz, Germany). The saponin (in this example SA1641) was purified from a saponin composite raw extract from Gypsophila species called Saponinum album obtained from Merck (Darmstadt, Germany). The powdered raw extract (2.5 g) was hydrolyzed in water (100 mL) with sodium hydroxide (0.2 g). The solution was stirred for 20 h at 40° C. and then supplemented with glacial acetic acid until pH 5.0 was reached. To remove tannins, the solution was shaken in a separatory funnel with 30 mL butanol. The aqueous phase was recaptured and butanol extraction repeated two times. The butanol phases were supplemented with anhydrous sodium sulfate, filtered and pooled. Butanol was evaporated and the remaining saponin powder resolved in 20% methanol to a final concentration of 30 mg/mL. After short sonication, different saponins were separated by high performance liquid chromatography (HPLC). Tubes (excluding column) were rinsed with warm water (40° C.) at a flow of 1.5 ml/min and then including Eurospher RP-C18-column (5 μm, 250×8 mm) with isopropanol (100%). Saponins were applied to the column and eluted with a methanol gradient (20% methanol to 70% methanol within 30 min at 1.5 mL/min in water supplemented with 0.01% trifluoroacetic acid followed by 70% methanol for further 60 min) [31]. Aliquots of the fractions were analyzed for their SA1641 content by electrospray ionization mass spectrometry (ESI-MS). Fractions containing pure SA1641 were pooled and methanol evaporated. The aqueous solution was frozen as a thin film in a rotating round-bottom flask by use of dry ice. After storage for 16 h at −80° C., the sample was lyophilized. To produce the scaffold as defined in the invention, the polymeric structure (0.2 mM) and SA1641 (3.2 mM) were solved in water (approx. pH 8) and equal volumes mixed and shaken for 24 h at 26° C. Then sodium cyanoborohydride (NaCNBH₃; 0.1 M) was added in 4-fold molar excess referred to SA1641 and the sample incubated for further 24 h. The structure was then verified by ultra performance liquid chromatography (UPLC)/ESI-MS. The samples were applied to a RP-C4-column and eluted with a methanol gradient (25% methanol to 80% methanol within 15 min in water supplemented with 0.01% trifluoroacetic acid followed by 80% methanol for further 10 min). The fractions were analyzed by use of LockSpray™ that is an ion source designed specifically for exact mass measurement with electrospray ionization using LC-time-of-flight (LC-TOF) mass spectrometers from Waters Corporation.

### Results

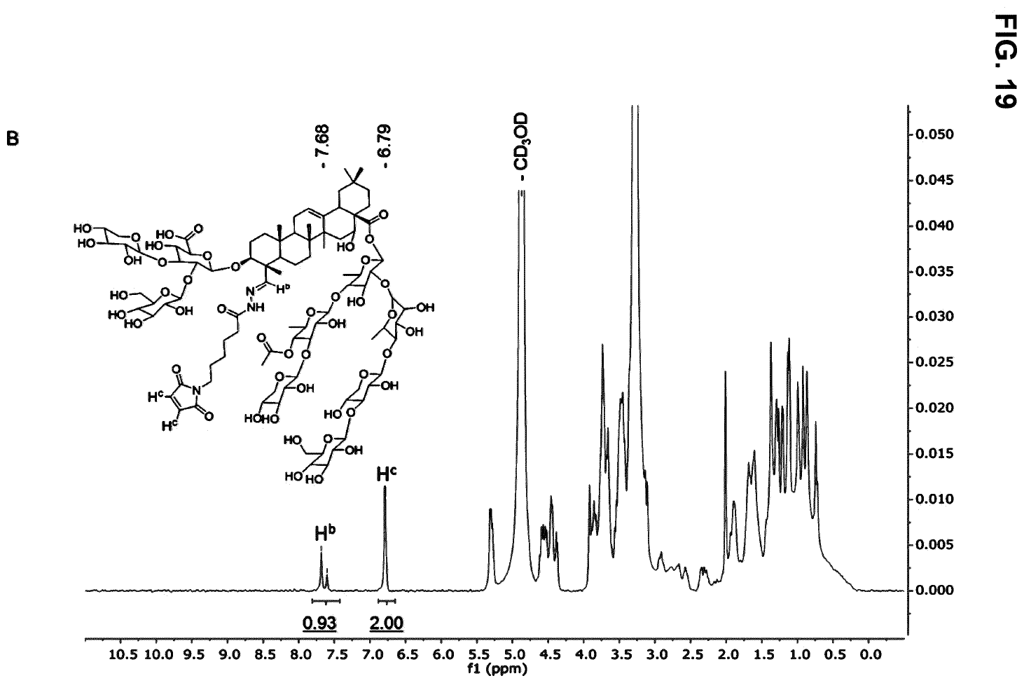

The inset of FIG. 17 shows the theoretically expected mass spectrum obtained from a calculation with the isotope pattern calculator enviPat Web 2.0. The pattern considers the charge of the molecule and the natural occurrence of isotopes, which is the reason that more than one peek is expected for a single substance. The experimental data (FIG. 4) obtained by UPLC/ESI-MS show almost exactly the same peaks at m/z 758-760 with same intensity as predicted, thus proving successful SA1641 coupling to the polymeric structure.

### Example 17

### Materials and Methods

As an example for a pharmaceutical active substance, the inventors used the targeted toxin dianthin-Epidermal Growth Factor (dianthin-EGF). The plasmid His-dianthin-EGF-pET11d [1] (100 ng) was added to 20 μL Escherichia coli Rosetta™ 2 (DE3) pLysS Competent Cells (Novagen, San Diego, Calif., USA). Cells were transformed by a heat-shock (30 min on ice, 90 s at 42° C. and 1 min on ice). Thereafter, 300 μL lysogeny broth (LB) was added and the suspension incubated for 1 h at 37° C. while shaking at 200 rpm. A preheated lysogeny broth agar plate with 50 pg/mL ampicillin was inoculated with 100 μl bacteria suspension and the plate incubated overnight at 37° C. Lysogeny broth (3 mL) with 50 μg/mL ampicillin was inoculated with a colony from the plate and the bacteria were incubated for 8 h at 37° C. and 200 rpm. The suspension (50 μL) was added to 500 mL of lysogeny broth with 50 μg/mL ampicillin and incubated overnight at 37° C. and 200 rpm. Subsequently, the volume was scaled-up to 2.0 L and bacteria grew under the same conditions until an optical density at wavelength 600 nm of 0.9 was reached. Thereafter, protein expression was induced by the addition of isopropyl β-D-1-thiogalactopyranoside (IPTG) at a final concentration of 1 mM. Protein expression lasted for 3 h at 37° C. and 200 rpm. Finally, the bacterial suspension was centrifuged at 5,000×g and 4° C. for 5 min, resuspended in 20 mL PBS (137 mM NaCl, 2.7 mM KCl, 8.1 mM Na₂HPO₄, 1.47 mM KH₂PO₄) and stored at −20° C. until use. For purification, bacterial suspensions were thawed and lysed by sonication. Lysates were centrifuged (15,800×g, 4° C., 30 min) and imidazole added to a final concentration of 20 mM. The supernatant was incubated with 2 mL of Ni-nitrilotriacetic acid agarose under continuous shaking for 30 min at 4° C. in the presence of 20 mM imidazole. Subsequently, the material was poured into a 20-mL-column and washed three times with 10 mL wash buffer (50 mM NaH₂PO₄, 300 mM NaCl, 20 mM imidazole) and dianthin-EGF eluted by 10-mL-portions of increasing concentrations of imidazole (31, 65, 125 and 250 mM) in wash buffer. Eluate fractions (2 mL) were dialyzed overnight at 4° C. against 2.0 L PBS. Desalted dianthin-EGF was concentrated by an Amicon® Ultra-15 (10 kDa) and the protein concentration quantified.

To introduce a suitable click chemistry group into dianthin-EGF, alkyne-PEG₅-N-hydroxysuccinimidyl ester in 8-fold molar excess referred to dianthin-EGF was dissolved in dimethyl sulfoxide and added to 9 volumes of dianthin-EGF (1 mg in 0.2 M NaH₂PO₄/Na₂HPO₄, pH 8). After incubation at room temperature for 4 h, non-bound alkyne was separated by use of a PD10 column (GE-Healthcare, Freiburg, Germany). Click chemistry with the polymeric structure was conducted by copper(I)-catalyzed alkyne-azide cycloaddition. Alkyne-dianthin-EGF (0.02 mM), dendrimer (0.05 mM), CuSO₄ (0.1 mM), tris(3-hydroxypropyltriazolylmethyl)amine (0.5 mM) and sodium ascorbate (5 mM) were incubated under gentle agitation for 1 h at room temperature in 0.1 M NaH₂PO₄/Na₂HPO₄, pH 8. Low molecular mass substances were then separated using a PD10 column.

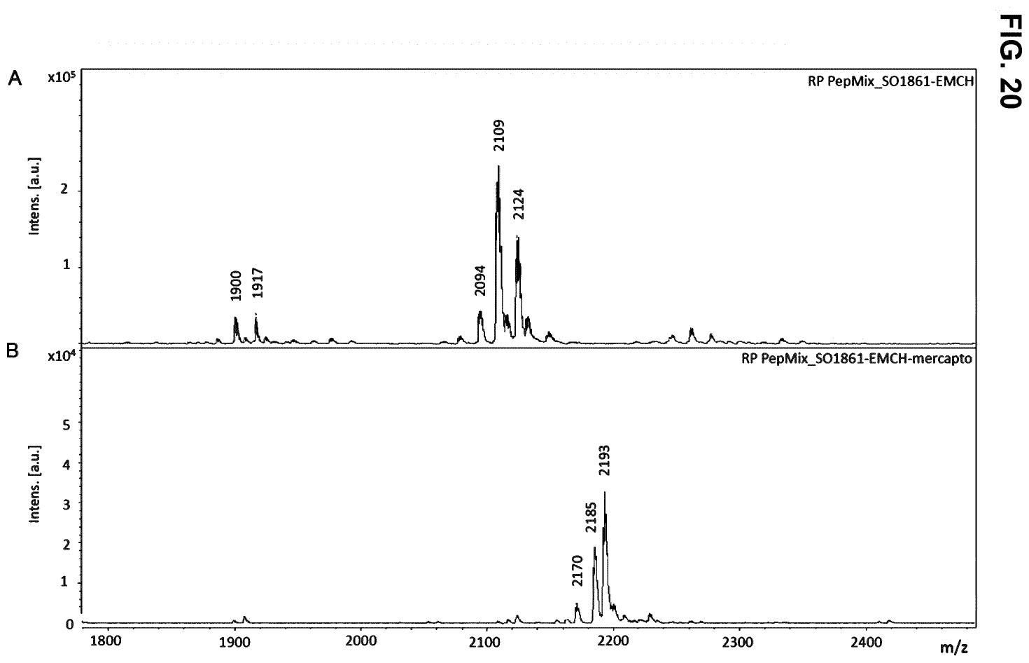

To test the efficacy of the invention, the inventors conducted a viability assay with HER14 cells. These cells are fibroblasts stably transfected with the human epidermal growth factor receptor and therefore target cells for the targeted toxin dianthin-EGF. HER14 cells (2,000 cells/100 μL/well) were seeded into wells of 96-well-cell culture plates and incubated for 24 h in DMEM medium supplemented with 10% fetal calf serum and 1% penicillin/streptomycin at 37° C., 5% CO₂ and 98% humidity. The different test substances (see results and FIG. 18) were then added in triplicates in a volume of 25 μL and supplemented with further 25 μL of medium. After an incubation of 72 h, 30 μL 3-(4,5-dimethylthiazol-2-yl)-2,5-diphenyltetrazolium bromide (0.5 mg/mL in water) was added per well and incubated for 2 h. Thereafter, the medium was carefully removed and replaced by an aqueous solution containing 10% (v/v) isopropanol, 5% (w/v) sodium dodecyl sulfate and 400 mM HCl, and incubated for 5 min. Solubilized formazan was photometrically quantitated at 570 nM in a microplate reader (Spectra MAX 340 PC, Molecular Devices, Sunnyvale, Calif., USA). Untreated cells were normalized to 1 and all samples referred to the untreated control. Significance was determined by unpaired two-sample t-tests.

### Results

The polymeric structure, in the example a pentameric dendrimer (pentrimer), does not have any cytotoxic effect on the target cells, neither in absence nor in presence of SA1641 (FIG. 18, column 2 and 3). In the absence of the scaffold, the targeted toxin (dianthin-EGF) shows half maximal toxicity at a concentration of 0.1 nM (column 4). In the presence of SA1641 the same concentration results in death of all cells indicating the general ability of SA1641 to act as an enhancer of the endosomal escape (column 5). The presence of the polymeric structure does not affect the toxicity of dianthin-EGF neither in the presence nor in the absence of SA1641 (columns 6 and 7), indicating that the scaffold does not affect the toxicity of dianthin-EGF. To couple the model polymeric structure via click chemistry to the example pharmaceutically active substance of dianthin-EGF, the substance had to be coupled with an alkyne group before. In consequence of this modification, dianthin-EGF lost some activity (compare columns 8 and 9 with 6 and 7, respectively), however, the undirected alkyne modification does not affect the idea of the invention and is also not required in future applications. We had to introduce the alkyne in an undirected way for test purposes only with the risk to impede the pharmaceutically active center of the toxin. A manufacturer of a pharmaceutically active substance can introduce the click position during synthesis directly into the substance at a position of his choice where the activity of the substance remains unaffected. There was no additional loss of activity when clicking the alkyne-modified pharmaceutically active substance to the polymeric structure indicating that the polymeric structure itself was not toxic (column 10 and 11).

### Example 18

### Materials and Methods

### Materials

The following chemicals were used as purchased: methanol (MeOH, LiChrosolv, Merck), N-ε-maleimidocaproic acid hydrazide (EMCH, 95%, TCI Chemicals), trifluoroacetic acid (TFA, 99.8%, Carl Roth), 2-mercaptoethanol (98%, Sigma-Aldrich), poly(amidoamine) (PAMAM dendrimer, ethylenediamine core, generation 5.0 solution, Sigma-Aldrich), cyanine 3 carboxylic acid (Cy3-COOH, 95%, Lumiprobe), 1-[Bis(dimethylamino)methylene]-1H-1,2,3-triazolo[4,5-b]pyridinium 3-oxid hexafluorophosphate, N-[(Dimethylamino)-1H-1,2,3-triazolo-[4,5-b]pyridin-1-ylmethylene]-N-methylmethanaminium hexafluorophosphate N-oxide (HATU, 97%, Sigma-Aldrich), bovine serum albumin fraction V (BSA, Carl Roth), dimethylsulfoxide (DMSO, 99%, Carl Roth), 2-Iminothiolane hydrochloride (98%, Sigma-Aldrich), rhodamine b (RhodB, 95%, Merck), Dulbecco's phosphate buffered saline (PBS, Gibco), hydrochloric acid (HCl, 37%, Merck), NHS-PEG₁₃-DBCO (Click Chemistry Tools), Alexa Fluor™ 488 5-TFP (Thermo-Fischer), azido-PEG3-SS-NHS (Conju-Probe), sodium cyanoborohydride (NaCNBH₃, 95%, Sigma-Aldrich), ammonium persulfate (APS, 98%, Sigma-Aldrich), N,N,N′,N′-tetramethylethylenediamine (TMEDA, 99%, Sigma-Aldrich), customized peptide SESDDAMFCDAMDESDSK (95%, PeptideSynthetics), azido-dPEG₁₂-NHS (95%, Quanta Biodesign), PFd-G4-Azide-NH-BOC Dendron (G4-dendron, 95%, Polymer Factory), Cyanin5-DBCO (Cy5-DBCO, 95%, Lumiprobe), Chloroform (CHCl₃, 99.5%, Sigma), Amicon Ultra 0.5 mL centrifugal filters (3 kDa MWCO, Sigma), mPEG-SCM (mPEG₂ₖ-NHS, 95.6%, Creative PEG Works), Amicon Ultra 15 mL centrifugal filters (10 kDa MWCO, Sigma).

### Methods

### MALDI-TOF-MS

MALDI-TOF spectra were recorded on a MALDI-Mass Spectrometer (Bruker Ultrafex III). Typically, the sample dissolved in MilliQ water in nanomolar to micromolar range was spotted on the target (MTP 384 target plate polished steel T F, Bruker Daltons) using either super-DHB (99%, Fluka) or sinapinic acid (SA, 99%, Sigma-Aldrich) as the matrix dissolved in acetonitrile (MADLI-TOF-MS tested, Sigma)/0.1% TFA (7:3 v/v) via the dried-droplet-method. PepMix (Peptide Calibration Standard, Bruker Daltons) or ProteMass (Protein Calibration Standard, Sigma-Aldrich) served as calibration standards. RP mode refers to reflector positive mode. RN mode refers to reflector negative mode. LP mode refers to linear positive mode.

### H-NMR

¹H NMR analysis was performed using a Bruker 400 MHz NMR spectrometer. The sample preparation, in which 2 mg of sample had been dissolved in 0.8 mL of methanol-D₄ (99%, Deutero), was performed 24 h prior to the measurement.

### UV-Vis

UV-Vis measurements were performed on a NanoDrop ND-1000 spectrophotometer in the spectral range of 200-750 nm.

### Size Exclusion Chromatography

Size exclusion chromatography (SEC) was performed with Sephadex G 25 Superfine from GE Healthcare and on prepacked PD10 columns (GE Healthcare, Sephadex G 25 M). The material was activated by swelling in the respective eluent prior to performing chromatography.

### Dialysis

Regenerated cellulose membranes: MWCO=1 and 2 kDa (Spectra/Por), and MWCO=12-14 kDa (Carl Roth) were used to perform dialysis. Typically, dialysis was carried out for 24 h with 1 L of solvent that was exchanged after first 6 h of the process.

### Lyophilization

Freeze-drying was performed on an Alpha 1-2 LD plus (Martin Christ Gefriertrocknungsanlagen GmbH). Typically, samples were frozen with liquid nitrogen and placed into the freeze-dryer at high vacuum.

### SO1861-EMCH Synthesis

SO1861 from Saponaria officinalis L (59 mg, 31.7 μmol) and EMCH (301 mg, 888 μmol) were placed in a round flask with stirrer and dissolved in 13 mL methanol. TFA (400 μL, cat.) was added to the solution and the reaction mixture was stirred for 3 h at 800 rpm and room temperature on a RCT B magnetic stirrer (IKA Labortechnik). After stirring for 3 h, the mix was diluted either with MilliQ water or PBS and dialyzed extensively for 24 h against either with MilliQ water or PBS using regenerated cellulose membrane tubes (Spectra/Por 7) with a MWCO of 1 kDa. After dialysis, the solution was lyophilized to obtain a white powder. Yield 62.4 mg (95%). Dried aliquots were further used for characterization via ¹H NMR and MALDI-TOF-MS.

¹H NMR (400 MHz, methanol-Da) (FIG. 19 A, SO1861): δ=0.50-5.50 (m, saponin triterpenoid and sugar backbone protons), 9.43 (1H, s, aldehyde proton of saponin, H^{a}).

¹H NMR (400 MHz, methanol-Da) (FIG. 19 B. SO1861-EMCH, PBS workup): δ=0.50-5.50 (m, saponin triterpenoid and sugar backbone protons), 6.79 (2H, s, maleimide protons, H^{c}), 7.62-7.68 (1H, m, hydrazone proton, H^{b}).

MALDI-TOF-MS (RP mode) (FIG. 20 A): m/z 2124 Da ([M+K]⁺, saponin-EMCH), m/z 2109 Da ([M+K]⁺, SO1861-EMCH), m/z 2094 Da ([M+Na]⁺, SO1861-EMCH)

MALDI-TOF-MS (RN mode) (FIG. 25 C): m/z 2275 Da ([M−H]⁻, saponin-EMCH conjugate), 2244 Da ([M−H]⁻, saponin-EMCH conjugate), 2222 Da ([M−H]⁻, saponin-EMCH conjugate), 2178 Da ([M−H]⁻, saponin-EMCH conjugate), 2144 Da ([M−H]⁻, saponin-EMCH conjugate), 2122 Da ([M−H]⁻, saponin-EMCH conjugate), 2092 Da ([M−H]⁻, saponin-EMCH conjugate), 2070 Da ([M−H]⁻, SO1861-EMCH), 2038 Da ([M−H]⁻, SO1832-EMCH), 1936 Da ([M−H]⁻, SO1730-EMCH), 1861 Da ([M−H]⁻, SO1861).

### SO1861-EMCH-Mercaptoethanol

To SO1861-EMCH (0.1 mg, 48 nmol) 200 μL mercaptoethanol (18 mg, 230 μmol) was added and the solution was shaken for 1 h at 800 rpm and room temperature on a ThermoMixer C (Eppendorf). After shaking for 1 h, the solution was diluted with methanol and dialyzed extensively for 4 h against methanol using regenerated cellulose membrane tubes (Spectra/Por 7) with a MWCO of 1 kDa. After dialysis, an aliquot was taken out and analyzed via MALDI-TOF-MS.

MALDI-TOF-MS (RP mode): m/z 2193 Da ([M+K]⁺, SO1861-EMCH-mercaptoethanol), m/z 2185 Da ([M+K]⁺, SO1861-EMCH-mercaptoethanol), m/z 2170 Da ([M+Na]⁺, SO1861-EMCH-mercaptoethanol). See FIG. 20B.

### BSA-SO1861 Synthesis

2-iminothiolane (231 pg, 1.1 μmol) dissolved in 47 μL PBS was added to a BSA-RhodB solution (10 mg, 0.15 μmol) in 200 μL PBS and the mix was shaken for 40 min at 800 rpm and room temperature on a ThermoMixer C (Eppendorf). After shaking for 40 min, the reaction mix was immediately run through a Sephadex G25 superfine size exclusion column (16 mL column volume) and SO1861-EMCH (1 mg, 0.5 μmol) dissolved in 100 μL PBS was added to the collected BSA-SH fraction. The reaction mixture was shaken for 12 h at 800 rpm and room temperature on a ThermoMixer C (Eppendorf). After shaking for 12 h the BSA-SO1861 concentrated using centrifugal filtration at 4,000 rpm (15° C.) via Amicon Ultra 15 filters with a MWCO of 3 kDa. The conjugate was stored as solution in the fridge and aliquots were taken for analysis. Yield: not determined.

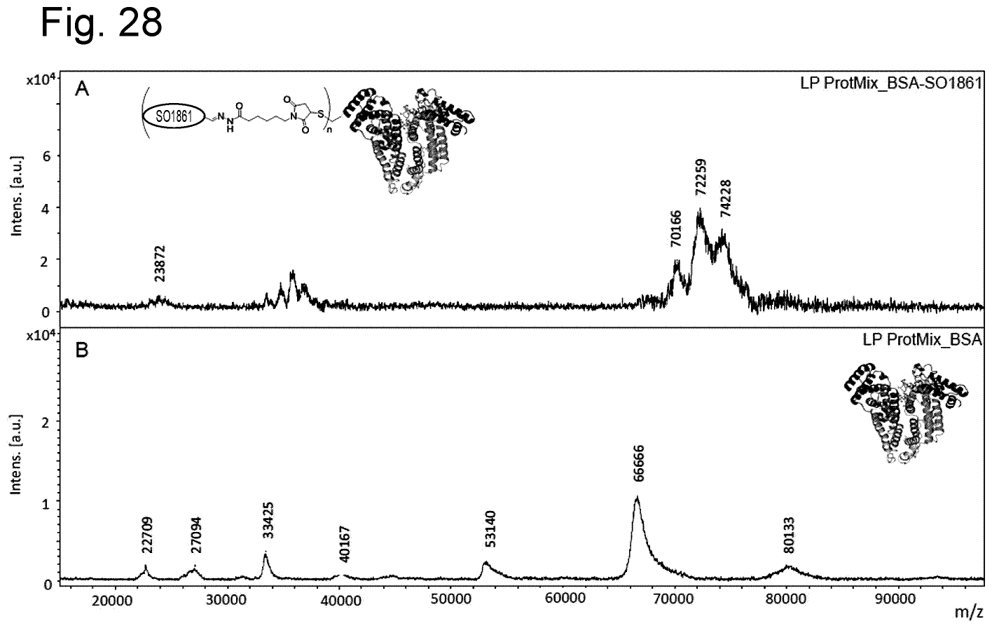

MALDI-TOF-MS (FIG. 28 A) (LP mode): m/z 74.2 kDa ([M+H]⁺, BSA-SO1861 with 4 SO1861 attached), 72.2 kDa ([M+H]⁺, BSA-SO1861 with 3 SO1861 attached), 70.2 kDa ([M+H]⁺, BSA-SO1861 with 2 SO1861 attached), 37.0 kDa ([M+H]²⁺, BSA-SO1861 with 4 SO1861 attached), 35.9 kDa ([M+H]²⁺, BSA-SO1861 with 3 SO1861 attached), 34.7 kDa ([M+H]²⁺, BSA-SO1861 with 2 SO1861 attached).

### Cy3-PAMAM

720 μL PAMAM dissolved in methanol (30 mg, 1.04 μmol) was placed into a 250 mL round flask and methanol was removed via a rotary evaporator (20 mbar, 60° C.). Remaining PAMAM was dissolved in 9 mL DMSO. HATU (7.6 mg, 20 μmol) dissolved in 0.5 mL DMSO was added to a Cy3-COOH (0.6 mg, 1.2 μmol) solution in DMSO and the mix was shaken for 1 h at 800 rpm at room temperature on a ThermoMixer C (Eppendorf). After shaking for 1 h, the HATU-Cy3 solution was added to the stirring PAMAM solution and the reaction mix was stirred for 12 h at room temperature. After stirring for 12 h, the reaction mix was diluted with MilliQ water and dialyzed extensively for 24 h against MilliQ water using regenerated cellulose membrane tubes (Spectra/Por 6) with a MWCO of 2 kDa. After dialysis, the volume of the conjugate solution was reduced via a rotary evaporator (20 mbar, 60° C.) and the concentrated conjugate solution was run through a Sephadex G25 superfine size exclusion column (16 mL column volume). The first fraction was collected and lyophilized to obtain the viscous pink PAMAM-Cy3 conjugate. PAMAM-Cy3 conjugate formation was confirmed by chromatography on thin layer chromatography (methanol/water, v/v 1:1), and the appearance of a faster band on a Sephadex G 25 superfine column. Yield 21.3 mg (63%). The dye per PAMAM molar ratio determined by UV-Vis spectrophotometry was 0.43.

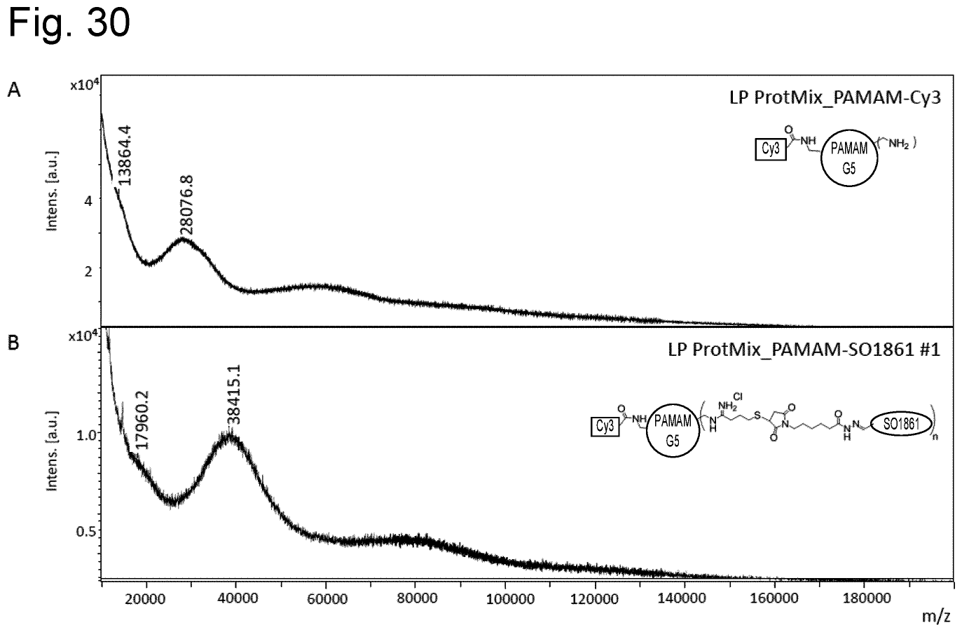

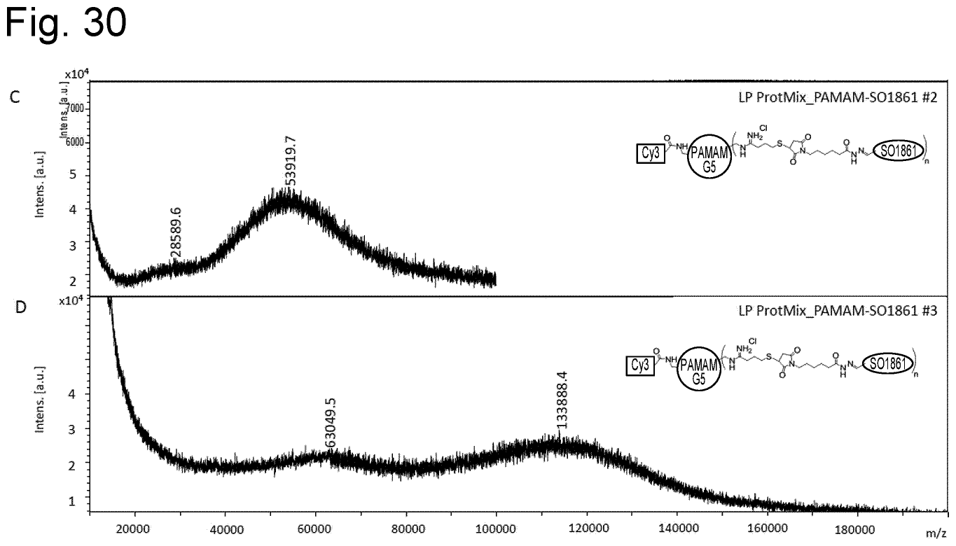

MALDI-TOF-MS (FIG. 30 A) (LP mode): m/z 28.0 kDa ([M+H]⁺, Cy3-PAMAM).

### Cy3-PAMAM-SO1861 Synthesis

Procedure is described exemplary for Cy3-PAMAM-(SO1861)₅. 2-iminothiolane (1 mg, 6.7 μmol) dissolved in 250 μL MilliQ water was added to a PAMAM-Cy3 solution (0.5 mg, 17 nmol) in 125 μL MilliQ water and the mix was shaken for 40 min at 800 rpm and room temperature on a ThermoMixer C (Eppendorf). After shaking for 40 min, the reaction mix was immediately run through a Sephadex G25 superfine size exclusion column (16 mL column volume) and SO1861-EMCH (176 pg, 85 nmol) dissolved in 40 μL MilliQ water was added to the collected Cy3-PAMAM-SH fraction. The reaction mixture was shaken for 12 h at 800 rpm and room temperature on a ThermoMixer C (Eppendorf). After shaking for 12 h, the reaction mix was diluted with MilliQ water and dialyzed extensively for 24 h against MilliQ water using regenerated cellulose membrane tubes (ZelluTrans, Carl Roth) with a MWCO of 12-14 kDa. After dialysis, the Cy3-PAMAM-SO1861 solution was concentrated using centrifugal filtration at 4000 rpm (15° C.) via Amicon Ultra 15 filters with a MWCO of 3 kDa. The conjugate was stored as solution in the fridge and aliquots were taken for analysis. Yield: 0.5 mg (75%).

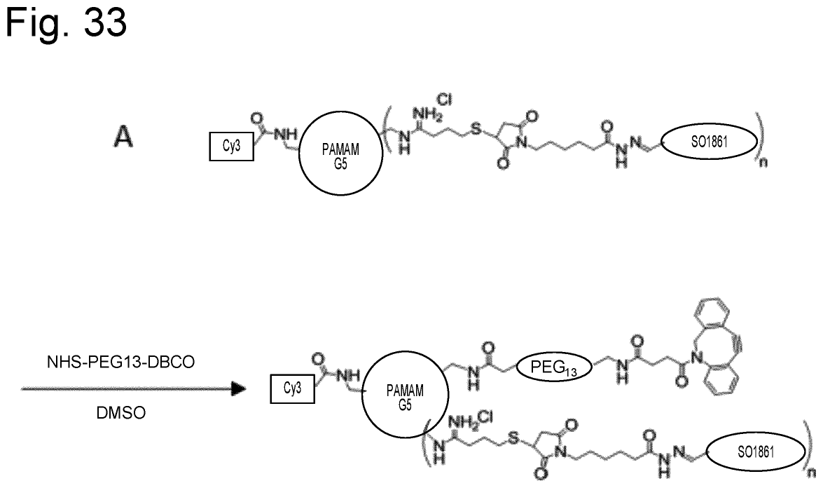

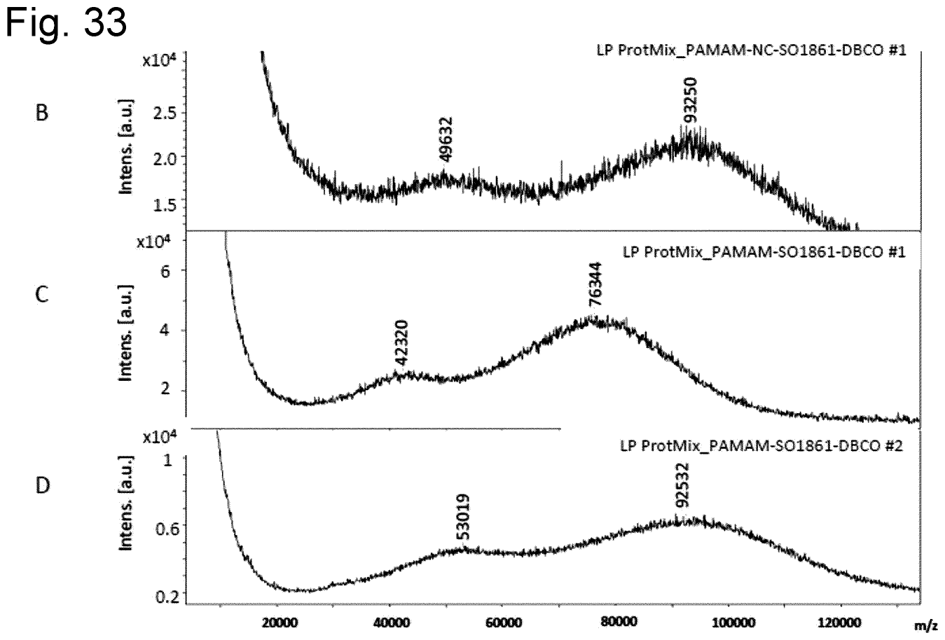

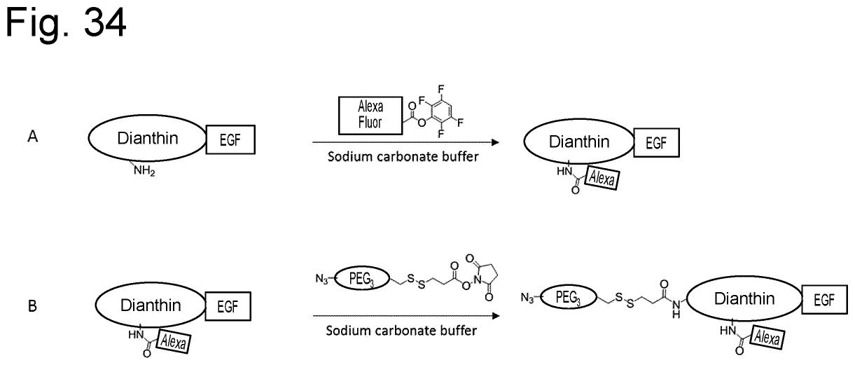

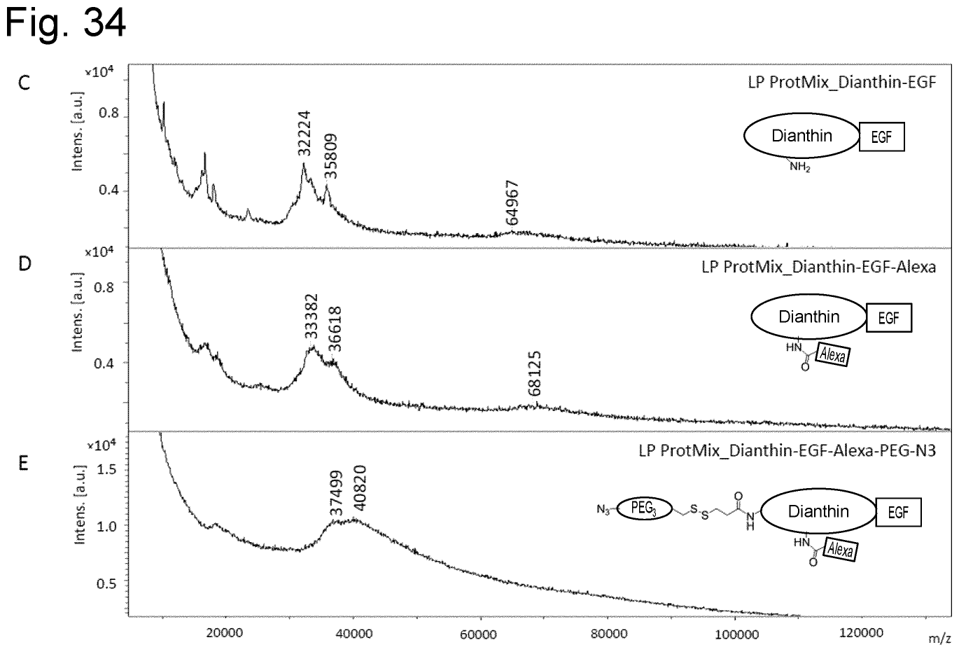

MALDI-TOF-MS spectra are illustrated in FIGS. 33 B-D, and FIG. 34. MALDI-TOF-MS of Cy3-PAMAM-(SO1861)₆ (FIG. 33 B) (LP mode): m/z 38.4 kDa ([M+H]⁺, Cy3-PAMAM-SO1861), 17.9 kDa ([M+H]²⁺, Cy3-PAMAM-SO1861).

The synthesis of Cy3-PAMAM-(SO1861)₅, Cy3-PAMAM-(SO1861)₁₃, Cy3-PAMAM-(SO1861)₅₁, and Cy3-PAMAM-(SO1861)₂₇, has been performed via the above described methodology but differ in the feed equivalents of the starting materials 2-iminothiolane and SO1861-EMCH. The respective feed equivalents of the starting materials and the respective mass of the conjugates are highlighted in Table 4.

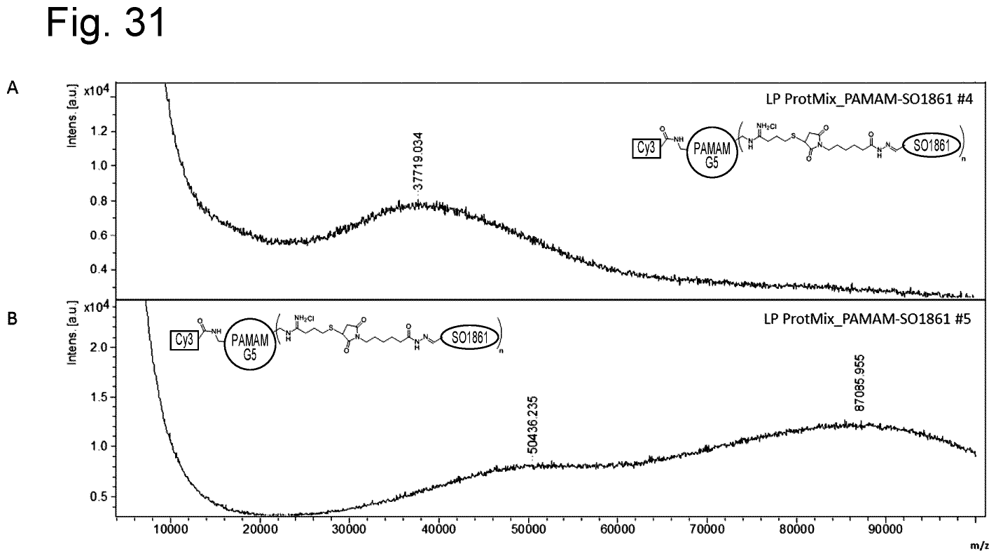

TABLE 4
Reaction parameter for Cy3-PAMAM-SO1861 synthesis.

| | | | SO1861 | |
|---|---|---|---|---|
| 2-Iminothiolane | SO1861-EMCH | Mass of | molecules | |
| feed equivalents | feed equivalents | conjugate via | attached | Resulting |
| to Cy3-PAMAM | to Cy3-PAMAM | MALDI-TOF-MS | per PAMAM | conjugate |
| 384 | 6 | 38.7 kDa | ~5 | Cy3-PAMAM- |
| | | | | (SO1861)₆, |
| | | | | FIG. 30 B |
| 384 | 20 | 53.9 kDa | ~13 | Cy3-PAMAM- |
| | | | | (SO1861)₁₃, |
| | | | | FIG. 30 C |
| 384 | 57 | 133.9 kDa | ~51 | Cy3-PAMAM- |
| | | | | (SO1861)₅₁, |
| | | | | FIG. 30 D |
| 8 | 5 | 37.7 kDa | ~5 | Cy3-PAMAM- |
| | | | | (SO1861)₅, |
| | | | | FIG. 31 A |
| 32 | 30 | 87.0 kDa | ~27 | Cy3-PAMAM- |
| | | | | (SO1861)₂₇, |
| | | | | FIG. 31 B |

### Cy3-PAMAM-NC-SO1861 Synthesis

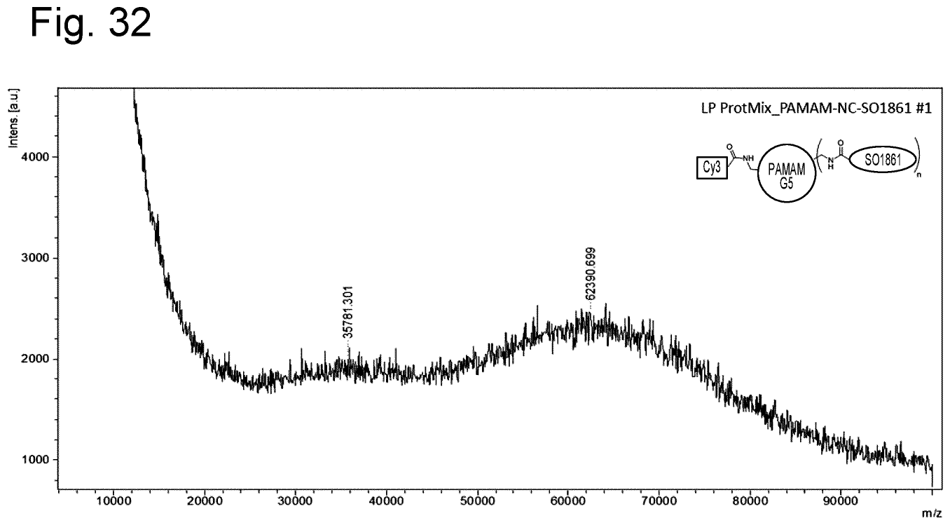

Cy3-PAMAM (0.5 mg, 18 nmol), SO1861 (2.3 mg, 1.24 μmol), and HATU (64.6 mg, 170 μmol) were dissolved separately in 200 μL DMSO. SO1861 and HATU solutions were mixed and shaken for 20 min at 800 rpm and room temperature on a ThermoMixer C (Eppendorf). After shaking for 20 min, Cy3-PAMAM solution was added to the shaking SO1861-HATU solution and the reaction mixture was allowed to shake for 12 h at 800 rpm and room temperature on a ThermoMixer C (Eppendorf). After shaking for 12 h, the reaction mix was diluted with MilliQ water and dialyzed extensively for 24 h against MilliQ water using regenerated cellulose membrane tubes (ZelluTrans, Carl Roth) with a MWCO of 12-14 kDa. After dialysis, the Cy3-PAMAM-NC-SO1861 solution was concentrated using centrifugal filtration at 4,000 rpm (15° C.) via Amicon Ultra 15 filters with a MWCO of 3 kDa. The Cy3-PAMAM-NC-(SO1861)₁₇ conjugate was stored as solution in the fridge and aliquots were taken for analysis. Yield: 0.77 mg (69%). MALDI-TOF-MS (FIG. 32) (LP mode): m/z 62.3 kDa ([M+H]⁺, Cy3-PAMAM-NC-SO1861), 35.7 kDa ([M+H]²⁺, Cy3-PAMAM-NC-SO1861).

### G4-Dendron Dye Labeling and Deprotection

PFd-G4-Azide-NH-BOC (G4-dendron) (9.75 mg, 2.11 μmol) was placed into a 2 mL reaction tube (Eppendorf) and dissolved in 200 μL DMSO. 100 μL of a Cy5-DBCO solution in DMSO (1.72 μmol * mL⁻¹, 170 nmol) was added to the G4-dendron solution and the mix was shaken for 12 hours at room temperature and 800 rpm on a ThermoMixer C (Eppendorf). After shaking for 12 h, the reaction mix was diluted with MilliQ water and dialyzed extensively for 24 h against MilliQ water using regenerated cellulose membrane tubes (Spectra/Por 7) with a MWCO of 1 kDa. After dialysis, the solution was lyophilized to obtain a blue powder. The crude product was used as obtained from lyophilization for the deprotection step.

Partially Cy5 labeled lyophilized G4-dendron was dissolved in 12 mL CHCl₃ in 50 mL round flask with stirrer. 12 mL TFA was added and the reaction mix was stirred for 3 h at 800 rpm and room temperature on a RCT B magnetic stirrer (IKA Labortechnik). After stirring for 3 h, the solvent was removed under reduced pressure (50° C., 30 mbar) on a rotary evaporator (Heidolph WB 2000). After evaporation, the batch was dissolved in MilliQ water and run through a PD10 size exclusion column. G4-dendron conjugate formation was confirmed by chromatography on thin layer chromatography (methanol/water, v/v 1:1), and the appearance of a faster band on a PD10 column. Obtained fraction of size exclusion chromatography was lyophilized to obtain a blue powder.

Yield 5.7 mg (93%). The dye per G4-dendron molar ratio determined by UV-Vis spectrophotometry was 0.012.

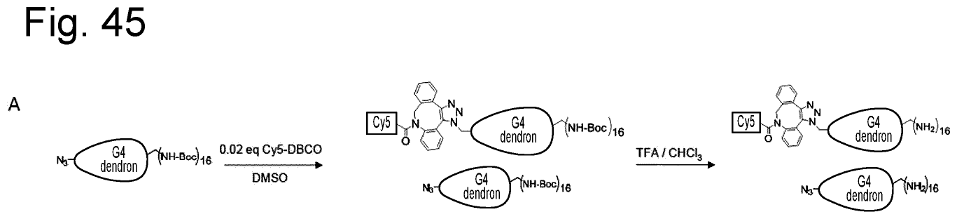

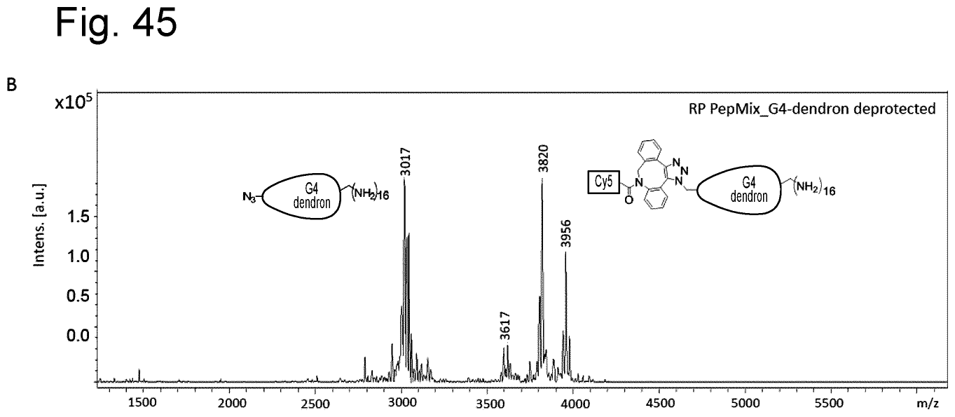

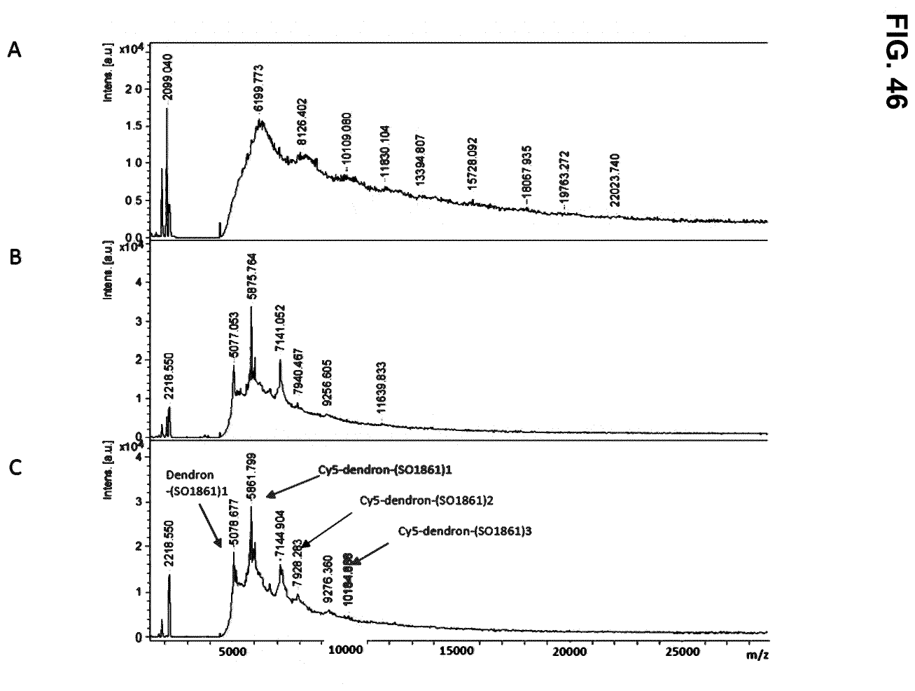

MALDI-TOF-MS (FIG. 45 B) (RP mode): m/z 3956 Da ([M+Na]⁺, Cy5-G4-dendron+PF₆⁻ counterion), 3820 Da ([M+Na]⁺, Cy5-G4-dendron-PF₆⁻ counterion), 3617 Da ([M+H]⁺, G4-dendron impurity), 3017 ([M+H]⁺, G4-dendron).

### G4-Dendron-SO1861 Synthesis

Procedure is described exemplary for the lowest G4-dendron to SO1861-EMCH ratio. 2-iminothiolane (2.65 mg, 19.2 μmol) dissolved in 300 μL MilliQ water was added to a partially Cy5 labeled G4-dendron solution (0.577 mg, 192 nmol) in 252 μL MilliQ water and the mix was shaken for 40 min at 800 rpm and room temperature on a ThermoMixer C (Eppendorf). After shaking for 40 min, the reaction mix was immediately run through a PD10 size exclusion column and SO1861-EMCH (1.19 mg, 575 nmol) dissolved in 100 μL MilliQ water was added to the collected G4-dendron-SH fraction. The reaction mixture was shaken for 12 h at 800 rpm and room temperature on a ThermoMixer C (Eppendorf). After shaking for 12 h, the reaction mix was concentrated via centrifugal filtration using Amicon Ultra centrifugal filters (3 kDa MWCO). The conjugate was stored as solution in the fridge and aliquots were taken for analysis. Yield: 90 nmol (47%).

MALDI-TOF-MS spectra are illustrated in FIG. 46. MALDI-TOF-MS of G4-dendron-SO1861 (FIG. 46 C) (LP mode): m/z 10.19 kDa ([M+H]⁺, Cy5-G4-dendron-[SO1861]₃), 9.27 kDa ([M+H]⁺, G4-dendron-[SO1861]₃), 7.92 kDa ([M+H]⁺, Cy5-G4-dendron-[SO1861]₂), 7.14 kDa ([M+H]⁺, G4-dendron-[SO1861]₂), 5.86 kDa ([M+H]⁺, Cy5-G4-dendron-[SO1861]₁), 5.07 kDa ([M+H]⁺, G4-dendron-[SO1861]₁).

The synthesis of other G4-dendron-(SO1861)ₙ conjugates has been performed via the above described methodology but differs in the feed equivalents of the starting material SO1861-EMCH. The respective feed equivalents of the starting materials and the respective mass of the conjugates are highlighted in Table 5.

TABLE 5
Reaction parameter for G4-dendron-SO1861 synthesis.

| | | | SO1861 | |
|---|---|---|---|---|
| 2-Iminothiolane | SO1861-EMCH | Mass of | molecules | |
| feed equivalents | feed equivalents | conjugates via | attached per | Resulting MS |
| to G4-dendron | to G4-dendron | MALDI-TOF-MS | G4-dendron | spectrum |
| 100 | 3 | 5.07-10.18 kDa | ~1-3 | FIG. 46 C |
| 100 | 10 | 5.07-11.64 kDa | ~1-4 | FIG. 46 B |
| 100 | 22 | 6.20-22.02 kDa | ~1-9 | FIG. 46 A |

### PAMAM Thiolation

Procedure is described exemplary for the highest PAMAM to 2-iminothiolane ratio. To a PAMAM (333 μg, 12.8 nmol) solution dissolved in 30 μL methanol 2-iminothiolane (0.53 mg, 3.84 μmol) dissolved in 128 μL MilliQ water was added. The reaction mixture was shaken for 12 h at 800 rpm and room temperature on a ThermoMixer C (Eppendorf). After shaking for 12 h, the reaction mix was washed 4 times with MilliQ water via centrifugal filtration using Amicon Ultra centrifugal filters (3 kDa MWCO) at 15° C. and 13500 rpm. After washing the sample was lyophilized to obtain a white solid. Yield was not determined.

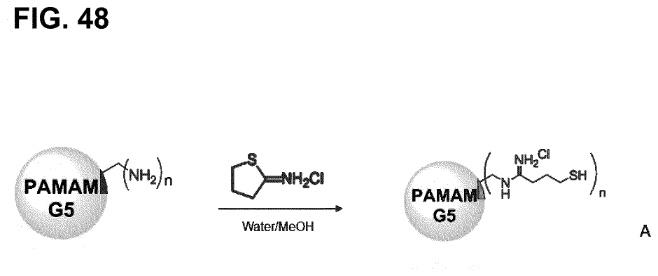

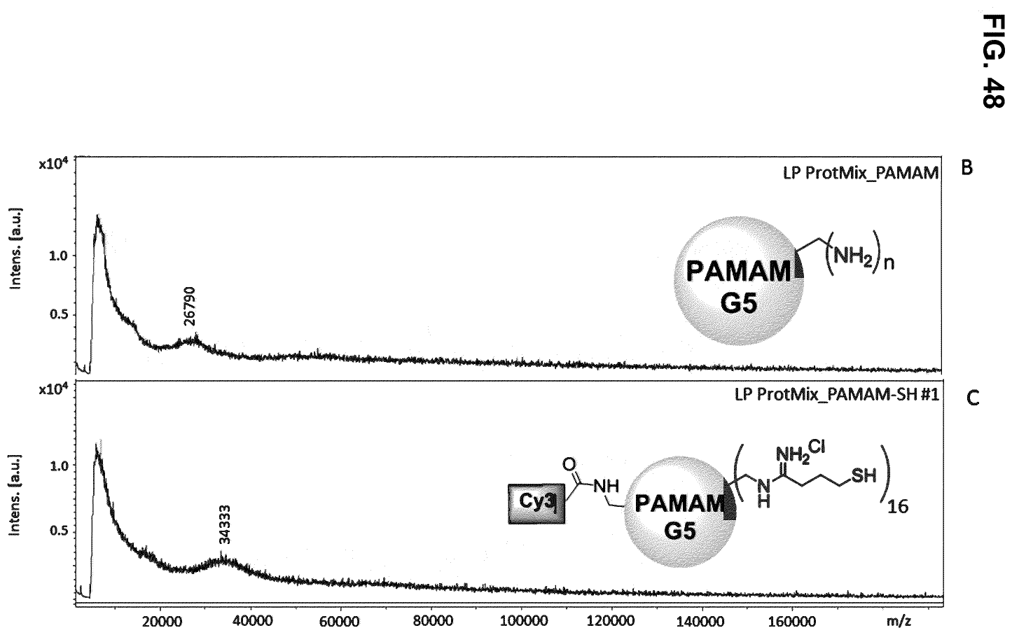

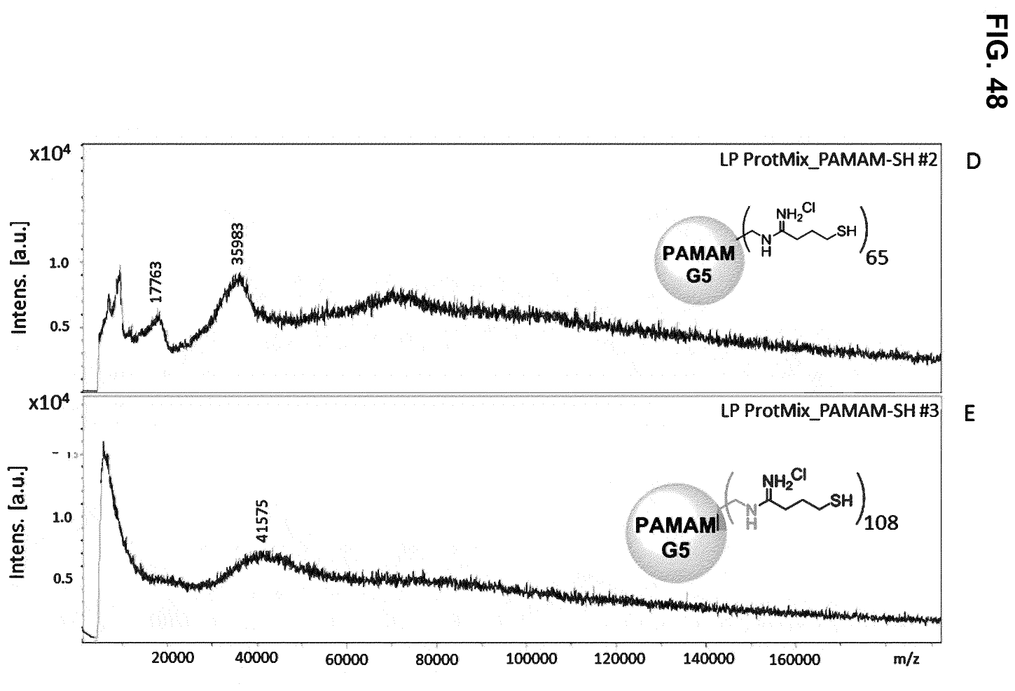

MALDI-TOF-MS spectra are illustrated in FIG. 48. MALDI-TOF-MS of PAMAM-(SH)₁₀₈ (FIG. 48 C) (LP mode): m/z 41.5 kDa ([M+H]⁺, PAMAM-[SH]₁₀₈).

The synthesis of other PAMAM-iminothiolane conjugates has been performed via the above described methodology but differs in the feed equivalents of the starting material 2-iminothiolane. For the lowest 2-iminothiolane feed reaction Cy3-PAMAM has been used.

The respective feed equivalents of the starting materials and the respective mass of the conjugates are highlighted in Table 6.

TABLE 6
Reaction parameter for PAMAM-SH synthesis.

| 2-Iminothiolane | Mass of | | |
|---|---|---|---|
| feed equivalents | conjugates via | Iminothiolane molecules | Resulting MS |
| to PAMAM | MALDI-TOF-MS | attached per PAMAM | spectrum |
| 50 | 34.4 kDa | ~16 | FIG. 48 C |
| 100 | 35.9 kDa | ~65 | FIG. 48 D |
| 300 | 41.5 kDa | ~108 | FIG. 48 E |

### PAMAM PEGylation

Procedure is described exemplary for the lowest PAMAM to mPEG₂ₖ ratio. To a PAMAM (333 pg, 12.8 nmol) solution dissolved in 10 μL DMSO mPEG₂ₖ-NHS (0.268 mg, 128 nmol) dissolved in 13 μL DMSO was added. The reaction mixture was shaken for 12 h at 800 rpm and room temperature on a ThermoMixer C (Eppendorf). After shaking for 12 h, the reaction mix was diluted with MilliQ water and dialyzed extensively for 24 h against MilliQ water using regenerated cellulose membrane tubes (Spectra/Por 6) with a MWCO of 2 kDa. After dialysis, the batch was concentrated via centrifugal filtration using Amicon Ultra 15 mL centrifugal filters (10 kDa MWCO). The concentrated batch was run through a PD10 size exclusion column followed by lyophilization to obtain a white fluffy powder. Yield was not determined.

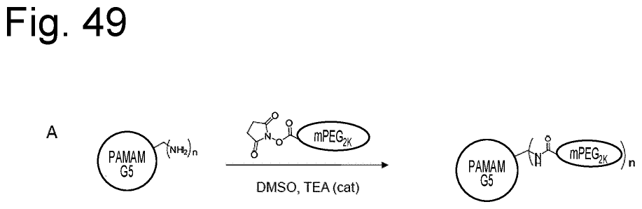

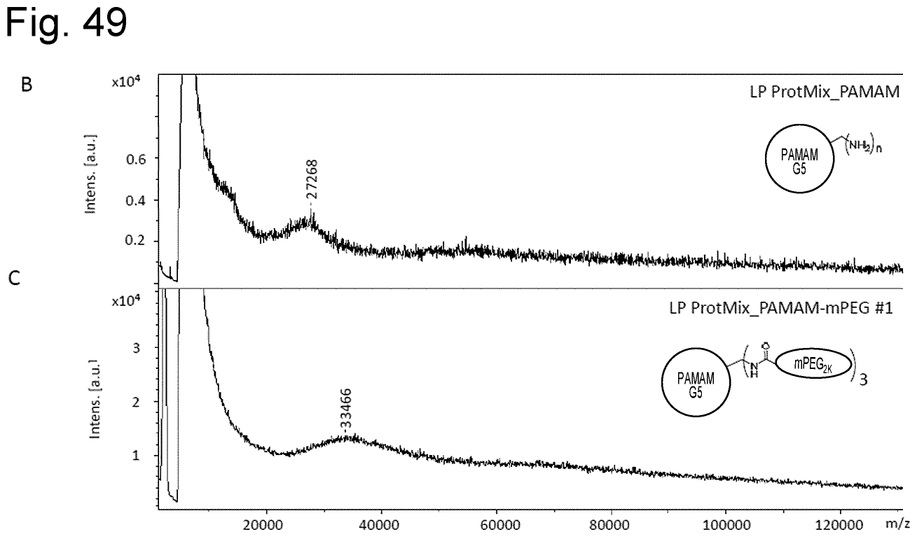

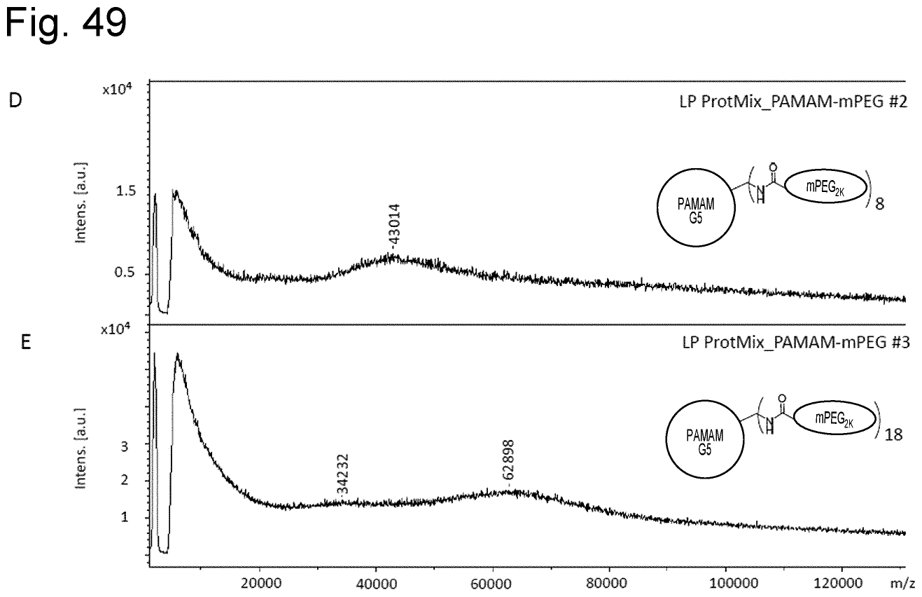

MALDI-TOF-MS spectra are illustrated in FIG. 49. MALDI-TOF-MS of PAMAM-(mPEG₂ₖ)₃ (FIG. 49 C) (LP mode): m/z 33.46 kDa ([M+H]⁺, PAMAM-[mPEG₂ₖ]₃).

The synthesis of other PAMAM-mPEG₂ₖ conjugates has been performed via the above described methodology but differs in the feed equivalents of the starting material mPEG₂ₖ-NHS. The respective feed equivalents of the starting materials and the respective mass of the conjugates are highlighted in Table 7.

TABLE 7
Reaction parameter for PAMAM-mPEG₂ₖ synthesis.

| mPEG₂ₖ-NHS | Mass of | | |
|---|---|---|---|
| feed equivalents | conjugates via | mPEG₂ₖ molecules | Resulting MS |
| to PAMAM | MALDI-TOF-MS | attached per PAMAM | spectrum |
| 10 | 28.5 kDa | ~3 | FIG. 49 C |
| 20 | 43.0 kDa | ~8 | FIG. 49 D |
| 100 | 62.8 kDa | ~18 | FIG. 49 E |

### Cy3-PAMAM-SO1861-DBCO Synthesis

Procedure is described exemplary for Cy3-PAMAM-(SO1861)₂₇-(DBCO)₁₀. Cy3-PAMAM-(SO1861)₂₇ (0.41 mg, 4.71 nmol) was freeze-fried and dissolved in 100 μL DMSO. DBCO-PEG₁₃-NHS ester (0.197 mg, 188 nmol) dissolved in DMSO was added to the Cy3-PAMAM-SO1861 solution and the mixture was shaken at 800 rpm and room temperature on a ThermoMixer C (Eppendorf). After shaking for 3 h, the reaction mix was diluted with MilliQ water and dialyzed extensively for 24 h against MilliQ water using regenerated cellulose membrane tubes (ZelluTrans, Carl Roth) with a MWCO of 12-14 kDa. After dialysis, the Cy3-PAMAM-SO1861-DBCO solution was concentrated using centrifugal filtration at 4,000 rpm (15° C.) via Amicon Ultra 15 filters with a MWCO of 3 kDa. The conjugate was stored as solution in the fridge and aliquots were taken for analysis. Yield: 0.1 mg (22%).

MALDI-TOF-MS (FIG. 33 D) (LP mode): m/z 92.5 kDa ([M+H]⁺, Cy3-PAMAM-SO1861-DBCO), 53.0 kDa ([M+H]²⁺, Cy3-PAMAM-SO1861-DBCO).

The synthesis of Cy3-PAMAM-(SO1861)₅-(DBCO)₃₈, and Cy3-PAMAM-(SO1861)₂₇-(DBCO)₁₀, have been performed via the above described methodology. The respective feed equivalents of the starting material and the respective mass of the conjugates are highlighted in Table 8.

TABLE 8
Reaction parameter for Cy3-PAMAM-SO1861-DBCO synthesis.

| | | | DBCO | |
|---|---|---|---|---|
| Used Cy3- | DBCO-PEG13- | Mass via | molecules | |
| PAMAM- | NHS feed | MALDI-TOF- | attached | Resulting |
| saponin batch | equivalents | MS | per PAMAM | conjugate |
| Cy3-PAMAM- | 40 | 76.3 kDa | ~38 | Cy3-PAMAM- |
| (SO1861)₅ | | | | (SO1861)₅-(DBCO)₃₈, |
| | | | | FIG. 33 C |
| Cy3-PAMAM- | 40 | 92.5 kDa | ~10 | Cy3-PAMAM- |
| (SO1861)₂₇ | | | | (SO1861)₂₇-(DBCO)₁₀, |
| | | | | FIG. 33 D |

### Cy3-PAMAM-NC-SO1861-DBCO Synthesis

Cy3-PAMAM-NC-(SO1861)₁₇ (0.3 mg, 4.8 nmol) was freeze-fried and dissolved in 100 μL DMSO. DBCO-PEG₁₃-NHS ester (0.202 mg, 194 nmol) dissolved in DMSO was added to the Cy3-PAMAM-NC-SO1861 solution and the mixture was shaken at 800 rpm and room temperature on a ThermoMixer C (Eppendorf). After shaking for 3 h, the reaction mix was diluted with MilliQ water and dialyzed extensively for 24 h against MilliQ water using regenerated cellulose membrane tubes (ZelluTrans, Carl Roth) with a MWCO of 12-14 kDa. After dialysis, the Cy3-PAMAM-SO1861-DBCO solution was concentrated using centrifugal filtration at 4,000 rpm (15° C.) via Amicon Ultra 15 filters with a MWCO of 3 kDa. The conjugate was stored as solution in the fridge and aliquots were taken for analysis. Yield: 0.1 mg (22%). Mass spectrometry indicates the conjugation of 30 DBCO moieties per PAMAM molecule.

MALDI-TOF-MS (FIG. 33 B) (LP mode): m/z 93.2 kDa ([M+H]⁺, Cy3-PAMAM-NC-SO1861-DBCO), 49.6 kDa ([M−1-1]²⁺, Cy3-PAMAM-NC-SO1861-DBCO).

### Dianthin-EGF and Dianthin Expression

Plasmid-DNA (His-dianthin-EGF-pET11d or His-dianthin-pET11d) [1] was transformed into chemically competent Escherichia coli NiCo21 (DE3) (New England Biolabs®, Inc.) and grown in 3 mL lysogeny broth supplemented with 50 μg/mL ampicillin at 37° C. for 5 h at 200 rpm. These bacteria were used to inoculate 500 mL lysogeny broth supplemented with 50 μg/mL ampicillin for overnight culture at 37° C. Subsequently, the culture volume was scaled up to 2 L and bacteria were grown until an optical density (A600) of 0.9. Protein expression was induced by the addition of isopropyl β-D-1-thiogalactopyranoside (IPTG) at a final concentration of 1 mM. Cells were further grown for 3 h at 37° C. and 200 rpm. After centrifugation (5 min, 5,000 g, 4° C.) cell pellets were resuspended in 20 mL phosphate buffered saline (Dulbecco's phosphate-buffered saline (PBS) with Ca²⁺ and Mg²⁺, pH 7.4) and stored at −20° C. After thawing, proteins were released by ultrasound device (Branson Sonifier 250, G. Heinemann). The solution was centrifuged (15,800×g, 30 min, 4° C.) and adjusted to 20 mM imidazole concentration. The construct contained an N-terminal His-tag and was purified by nickel nitrilotriacetic acid chromatography (Ni-NTA Agarose, Qiagen, Hilden, Germany). After elution with imidazole (20-250 mM) the eluates were analyzed by sodium dodecyl sulfate polyacrylamide gel electrophoresis (SDS-PAGE) (12%). Fractions containing dianthin-EGF or dianthin were dialyzed against 2 L chitin binding domain buffer (20 mM tris(hydroxymethyl)-aminomethane/HCl, 500 mM NaCl, 1 mM EDTA, 0.1% Tween-20, pH 8.0) at 4° C. Further purification by chitin column affinity chromatography served to remove bacterial proteins with binding activity for Ni-NTA agarose. After elution with chitin binding domain buffer, the fractions were analyzed by SDS-PAGE (12%). Fractions containing dianthin-EGF or dianthin were dialyzed against 5 L PBS at 4° C. Purified proteins were concentrated by Amicon centrifugal filter devices (10 kDa, Millipore, Eschborn, Germany). The protein concentration was determined by a bicinchoninic acid assay (Pierce, Rockford, USA).

### Dianthin-EGF-Alexa488 Synthesis

Dianthin-EGF (240 pg, 6.7 nmol) solution in PBS was placed into an Amicon Ultra 15 filter with a MWCO of 3 kDa and centrifuged at 4,000 rpm and 4° C. for 30 min three times. After each cycle, the Amicon filter was refilled with 0.1 M sodium carbonate buffer at pH 9. After the third centrifugation cycle, the volume was reduced to 0.5 mL via centrifugation. The dianthin-EGF sodium carbonate solution was placed into a 2 mL reaction tube and Alexa Fluor™ 488 5-TFP (50 pg, 56 nmol) dissolved in 10 μL DMSO was added to the protein solution. The mix was shaken at 800 rpm and room temperature on a ThermoMixer C (Eppendorf) for 80 min. After shaking, the mix was run through a Sephadex G25 M size exclusion column (GE Healthcare, PD10 column). The dianthin-EGF-Alexa488 conjugate was stored in solution in 0.1 M sodium carbonate buffer at pH 9 in the fridge and aliquots were taken for analysis. Yield: 210 μg (85%).

MALDI-TOF-MS (FIG. 34 D) (LP mode): m/z 36.8 kDa ([M+H]⁺, dianthin-EGF-Alexa488), m/z 33.6 kDa ([M+H]⁺, dianthin-EGF-Alexa488), 18.8 kDa ([M+H]²⁺, dianthin-EGF-Alexa488), 16.6 kDa ([M+H]²⁺, dianthin-EGF-Alexa488).

### Dianthin-Alexa488 Synthesis

Dianthin (184 pg, 6.2 nmol) solution in PBS was placed into an Amicon Ultra 15 filter with a MWCO of 3 kDa and centrifuged at 4,000 rpm and 4° C. for 30 min three times. After each cycle, the Amicon filter was refilled with 0.1 M sodium carbonate buffer at pH 9. After the third centrifugation cycle, the volume was reduced to 0.5 mL via centrifugation. The dianthin sodium carbonate solution was placed into a 2 mL reaction tube and Alexa Fluor™ 488 5-TFP (16.7 pg, 19 nmol) dissolved in 3.5 μL DMSO was added to the protein solution. The mix was shaken at 800 rpm and room temperature on a ThermoMixer C (Eppendorf) for 80 min. After shaking, the mix was run through a Sephadex G25 M size exclusion column (GE Healthcare, PD 10 column). The dianthin-Alexa488 conjugate was stored in solution in 0.1 M sodium carbonate buffer at pH 9 in the fridge and aliquots were taken for analysis. Yield: not determined

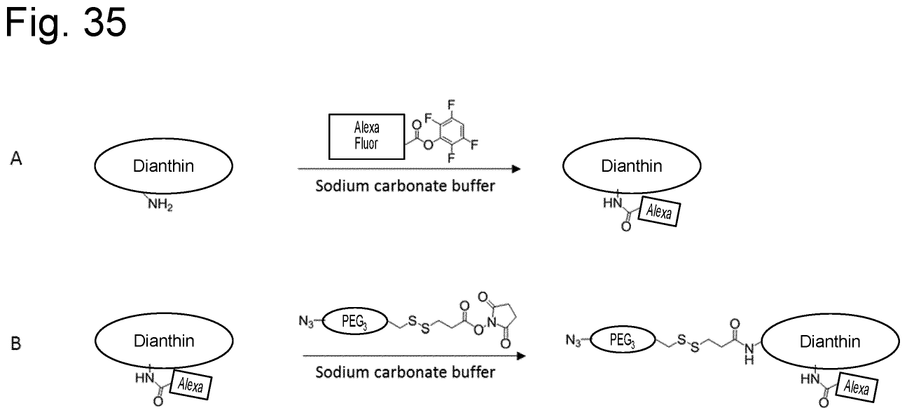

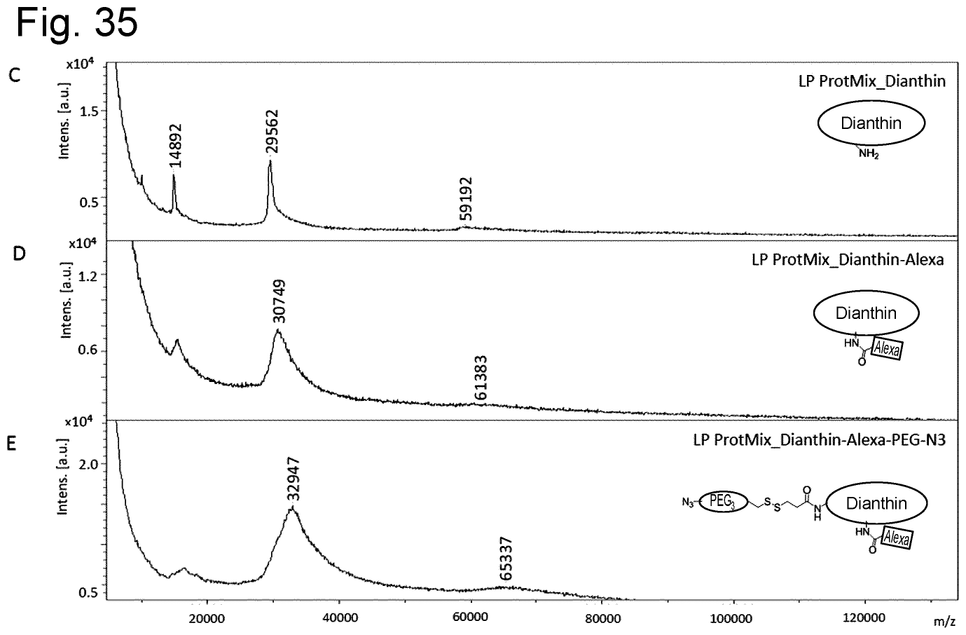

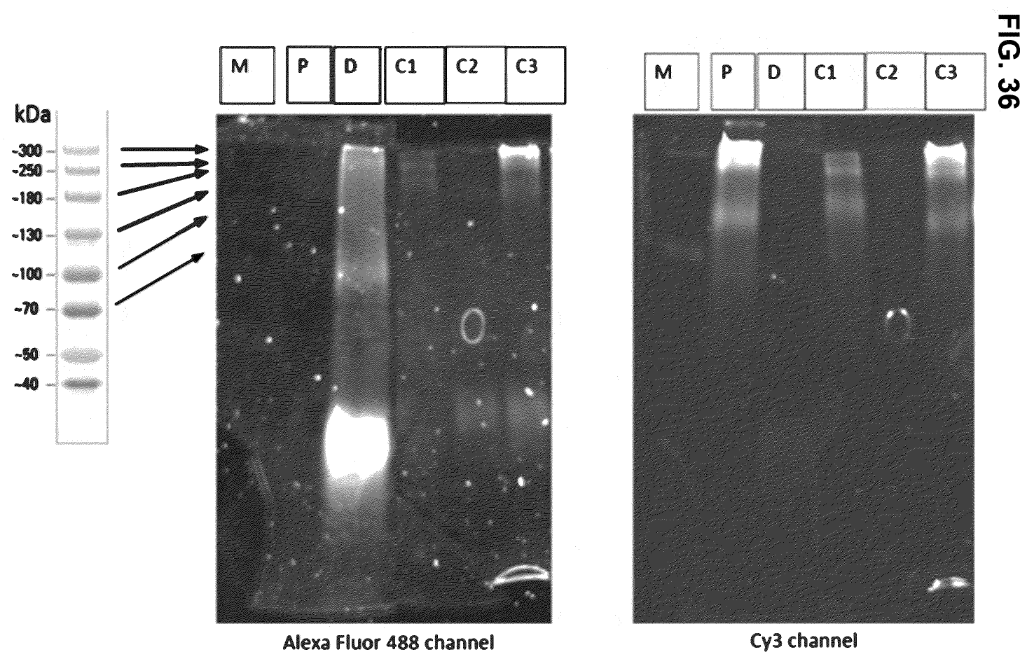

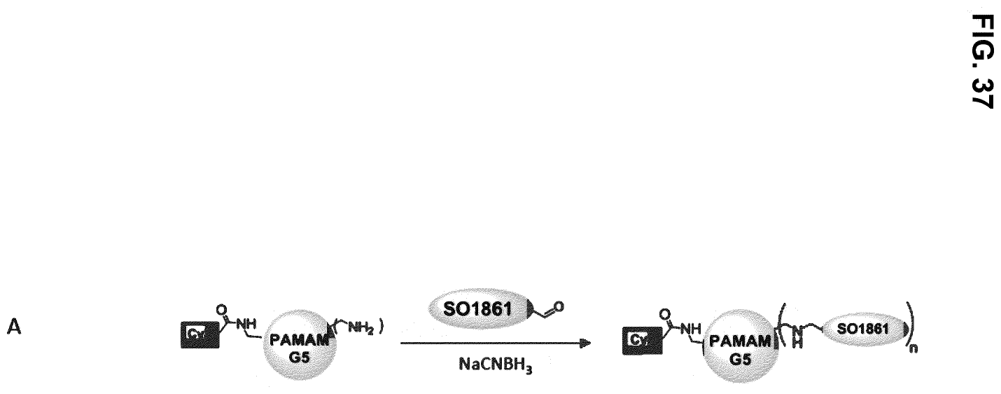

MALDI-TOF-MS (FIG. 35 D) (LP mode): m/z 30.7 kDa ([M+H]⁺, dianthin-Alexa488).

Dianthin-EGF-Alexa488-S-S-PEG-N₃, and Dianthin-EGF-Alexa488-PEG₁₂-N₃ Synthesis

Procedure is described exemplary for dianthin-EGF-Alexa488-S-S-PEG-N₃. Dianthin-EGF-Alexa488 (70 μg, 1.9 nmol) sodium carbonate solution was placed into a 2 mL reaction tube and azido-PEG₃-S-S-NHS (120 μg, 272 nmol) dissolved in 9 μL DMSO was added to the protein solution. The mix was shaken at 800 rpm and 15° C. on a ThermoMixer C (Eppendorf) for 12 h. After shaking, the reaction mix was diluted with PBS and was washed with PBS via centrifugal filtration at 4,000 rpm and 4° C. using Amicon Ultra 15 filter with a MWCO of 3 kDa.

Yield: 54 μg (70%).

MALDI-TOF-MS (FIG. 34 E) (LP mode): m/z 40.8 kDa ([M+H]⁺, dianthin-EGF-Alexa488-S-S-PEG-N₃), m/z 37.5 kDa ([M+H]⁺, dianthin-EGF-Alexa488-S-S-PEG-N₃).

The synthesis of dianthin-EGF-Alexa488-S-S-PEG-N₃, and dianthin-EGF-Alexa488-PEG₁₂-N₃ have been performed via the above described methodology but differed in the used azido-PEG linker. The respective azido-PEG linker, their feed equivalents, and the respective mass of the conjugates are highlighted in Table 9.

TABLE 9
Reaction parameter for dianthin-EGF-Alexa488-PEG-N₃ synthesis

| | | Azido-PEG | Mass of | |
|---|---|---|---|---|
| Used toxin | Azido-PEG linker | linker feed | conjugate via | Resulting |
| batch | used | equivalents | MALDI-TOF-MS | conjugate |
| Dianthin-EGF- | Azido-PEG₃-S-S- | 135 | 40.8 kDa | Dianthin-EGF- |
| Alexa488 | NHS | | | Alexa488-S-S- |
| | | | | PEG₃-N₃ |
| Dianthin-EGF- | Azido-PEG₁₂-NHS | 135 | 43.3 kDa | Dianthin-EGF- |
| Alexa488 | | | | Alexa488-PEG₁₂- |
| | | | | N₃ |

### Dianthin-Alexa488-S-S-PEG-N₃

Dianthin-Alexa488 (24.5 μg, 0.8 nmol) sodium carbonate solution was placed into a 2 mL reaction tube and azido-PEG₃-S-S-NHS (34 μg, 78 nmol) dissolved in 9 μL DMSO was added to the protein solution. The mix was shaken at 800 rpm and 15° C. on a ThermoMixer C (Eppendorf) for 12 h. After shaking, the reaction mix was diluted with PBS and was washed with PBS via centrifugal filtration at 4,000 rpm and 4° C. using Amicon Ultra 15 filter with a MWCO of 3 kDa.

Yield: 10.3 μg (39%).

MALDI-TOF-MS (FIG. 35 E) (LP mode): m/z 32.9 kDa ([M+H]⁺, dianthin-Alexa488-S-S-PEG-N₃).

### Cy3-PAMAM-Saponin-Toxin Conjugate Synthesis

Procedure is described exemplary for Cy3-PAMAM-(SO1861)₂₇-DBCO. Cy3-PAMAM-(SO1861)₂₇-DBCO (17 μg, 0.184 nmol) solution in MilliQ water was mixed with a dianthin-EGF-Alexa488-SS-PEG3-N₃ (3.6 μg, 0.089 nmol) solution in PBS in a 1.5 mL reaction tube and the reaction mix was shaken at 800 rpm and 15° C. on a ThermoMixer C (Eppendorf) for 2 h. After shaking, small aliquots were taken out for analysis via SDS-PAGE and fluorescence imaging on a Molecular Imager® VersaDoc™ MP 4000 imaging system (Bio-Rad) (FIG. 36).

The synthesis of Cy3-PAMAM-(SO1861)₅-S-S-Dianthin-EGF-Alexa488, Cy3-PAMAM-(SO1861)₂₇-S-S-Dianthin-EGF-Alexa488, Cy3-PAMAM-NC-(SO1861)₁₇-S-S-Dianthin-EGF-Alexa488, Cy3-PAMAM-NC-(SO1861)₁₇-S-S-Dianthin-Alexa488, and Cy3-PAMAM-NC-(SO1861)₁₇-Dianthin-EGF-Alexa488, have been performed via the above described methodology but differ in the used PAMAM-saponin-DBCO batch, the used azido-toxin batch, and their feed equivalents. The respective feed equivalents of the starting materials are highlighted in Table 10.

Cy3-PAMAM-NC-SO1861 Synthesis Via Reductive Amination

Cy3-PAMAM (0.19 mg, 13 nmol) and SO1861 (0.73 mg, 0.39 μmol) were dissolved separately in 200 μL 0.1 M acetate buffer at pH 5. SO1861 and Cy3-PAMAM solutions were mixed and shaken for 20 min at 800 rpm and room temperature on a ThermoMixer C (Eppendorf). After shaking for 20 min, NaCNBH₃ (5 mg, 81 μmol) was added to the shaking reaction solution and the reaction mixture was allowed to shake for 12 h at 800 rpm and room temperature on a ThermoMixer C (Eppendorf). After shaking for 12 h, the reaction mix was diluted with MilliQ water and dialyzed extensively for 24 h against MilliQ water using regenerated cellulose membrane tubes (ZelluTrans, Carl Roth) with a MWCO of 12-14 kDa. After dialysis, the Cy3-PAMAM-NC-SO1861 solution was concentrated using centrifugal filtration at 4,000 rpm (15° C.) via Amicon Ultra 15 filters with a MWCO of 3 kDa. The conjugate was stored as solution in the fridge and aliquots were taken for analysis. Yield: not determined

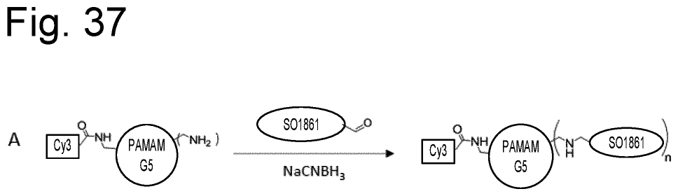

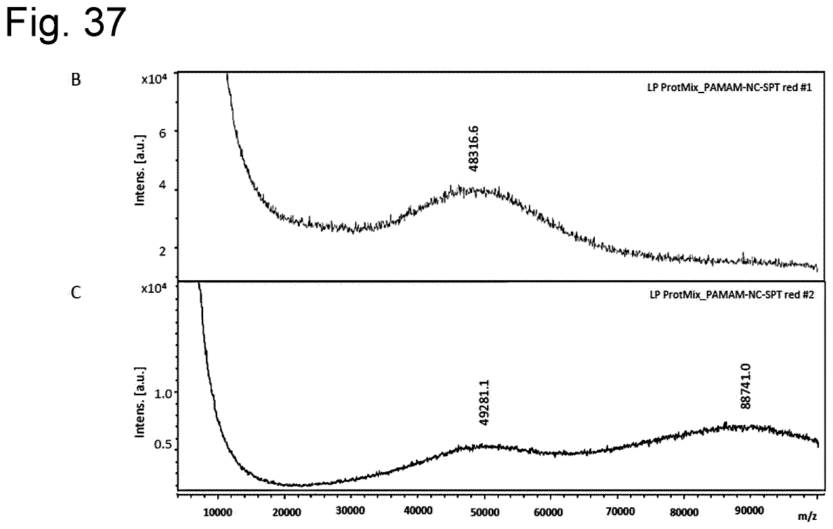

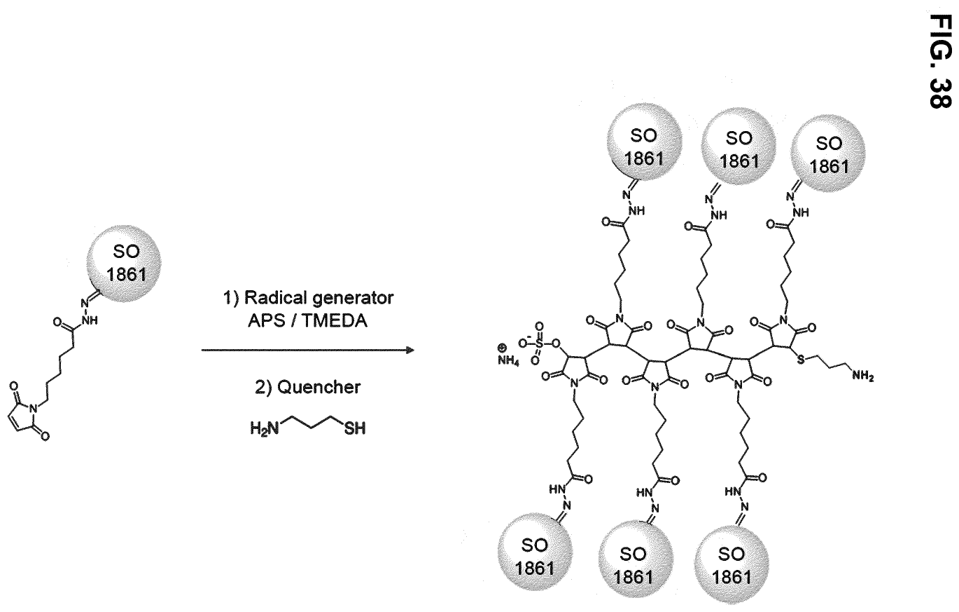

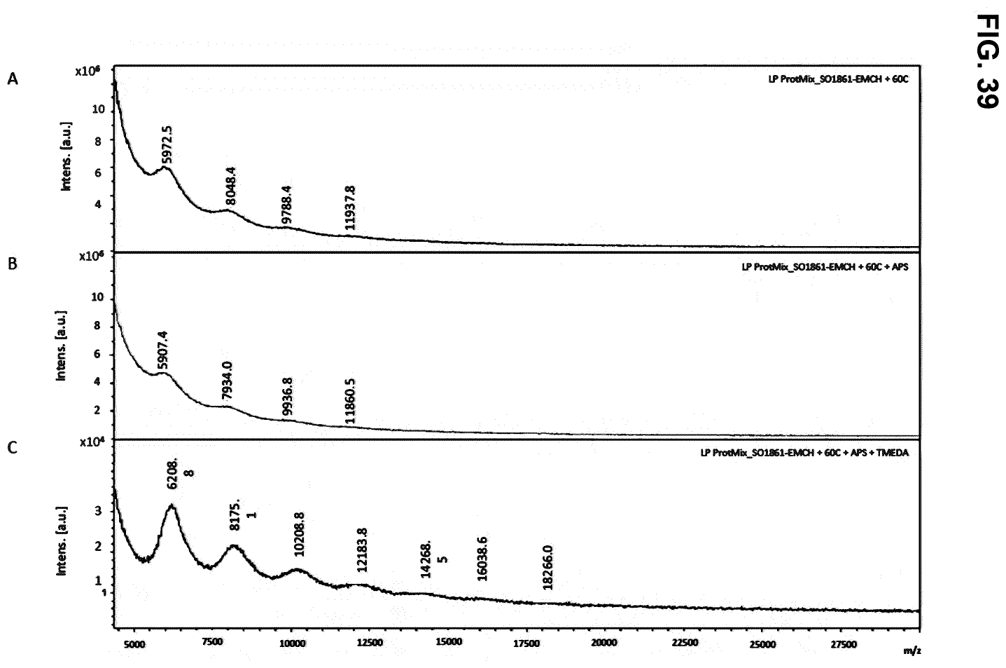

MALDI-TOF-MS (FIG. 37 B, C) (LP mode): m/z 88.7 kDa ([M+H]⁺, Cy3-PAMAM-NC-SO1861), 49.2 kDa ([M−1-1]²⁺, Cy3-PAMAM-NC-SO1861).

The synthesis of Cy3-PAMAM-NC-(SO1861)₃₀, and Cy3-PAMAM-NC-(SO1861)₁₀, have been performed via the above described methodology but differed in the time after which the reducing agent NaCNBH₃ was added to the reaction batch. The respective time of the NaCNBH₃ addition and the respective mass of the conjugates are highlighted in Table 11.

TABLE 10
Reaction parameter for Cy3-PAMAM-saponin-toxin synthesis.

| | PAMAM- | | Azido-toxin | |
|---|---|---|---|---|
| PAMAM-saponin- | saponin-DBCO | Azido-toxin | feed | Resulting |
| DBCO batch used | feed equivalents | batch used | equivalents | conjugate |
| Cy3-PAMAM- | 3 | Dianthin- | 1 | Cy3-PAMAM- |
| (SO1861)₅-(DBCO)₃₈ | | EGF- | | (SO1861)₅-S-S- |
| | | Alexa488-S- | | Dianthin-EGF- |
| | | S-PEG₃-N₃ | | Alexa488 |
| Cy3-PAMAM- | 2.1 | Dianthin- | 1 | Cy3-PAMAM- |
| (SO1861)₂₇- | | EGF- | | (SO1861)₂₇-S-S- |
| (DBCO)₁₀ | | Alexa488-S- | | Dianthin-EGF- |
| | | S-PEG₃-N₃ | | Alexa488 |
| Cy3-PAMAM-NC- | 2.3 | Dianthin- | 1 | Cy3-PAMAM-NC- |
| (SO1861)₁₇- | | EGF- | | (SO1861)₁₇-S-S- |
| (DBCO)₃₀ | | Alexa488-S- | | Dianthin-EGF- |
| | | S-PEG₃-N₃ | | Alexa488 |
| Cy3-PAMAM-NC- | 7.1 | Dianthin- | 1 | Cy3-PAMAM-NC- |
| (SO1861)₁₇- | | Alexa488-S- | | (SO1861)₁₇-S-S- |
| (DBCO)₃₀ | | S-PEG₃-N₃ | | Dianthin-Alexa488 |
| Cy3-PAMAM-NC- | 2.3 | Dianthin- | 1 | Cy3-PAMAM-NC- |
| (SO1861)₁₇- | | EGF- | | (SO1861)₁₇-Dianthin- |
| (DBCO)₃₀ | | Alexa488- | | EGF-Alexa488 |
| | | PEG₁₂-N₃ | | |

TABLE 11
Reaction parameter Cy3-PAMAM-NC-SO1861 synthesis via reductive amination.

| | | | SO1861 | |
|---|---|---|---|---|
| Time of shaking reaction | | Mass via | molecules | |
| mix before NaCNBH₃ | | MALDI-TOF- | attached | Resulting |
| addition | | MS | per PAMAM | conjugate |
| 20 | min | 88.8 kDa | ~30 | Cy3-PAMAM-NC- |
| | | | | (SO1861)₃₀, |
| | | | | FIG. 37 C |
| 12 | h | 48.0 kDa | ~10 | Cy3-PAMAM-NC- |
| | | | | (SO1861)₁₀, |
| | | | | FIG. 37 B |

### Poly(SO1861) Synthesis

SO1861-EMCH (0.13 mg, 63 nmol) was dissolved in 30 μL degassed MilliQ water. APS (0.2 μg, 0.8 nmol) dissolved in 4 μL degassed MilliQ water was added to the SO1861-EMCH solution and the solution was placed into a ThermoMixer C (Eppendorf) at 60° C. Then, TMEDA (cat., 0.5 μL) was added to the mix and the mix was shaken at 800 rpm and 60° C. on a ThermoMixer C (Eppendorf) for 2 h. After 2 h, a small aliquot was taken out for analysis via mass spectrometry.

MALDI-TOF-MS (FIG. 39 C) (LP mode): m/z 18.2 kDa ([M+H]⁺, poly(SO1861)₉), 16.0 kDa ([M+H]⁺, poly(SO1861)₈), 14.2 kDa ([M+H]⁺, poly(SO1861)₇), 12.2 kDa ([M+H]⁺, poly(SO1861)₆), 10.2 kDa ([M+H]⁺, poly(SO1861)₅), 8.2 kDa ([M+H]⁺, poly(SO1861)₄), 6.2 kDa ([M+H]⁺, poly(SO1861)₃).

### SO1861-EMCH Peptide Coupling

Customized peptide with the sequence SESDDAMFCDAMDESDSK (0.6 mg, 0.3 μmol; [SEQ-ID No. 1]) and SO1861-EMCH (0.8 mg, 0.39 μmol) were dissolved separately in 200 μL PBS. SO1861-EMCH and peptide solutions were mixed and shaken for 12 h at 800 rpm and room temperature on a ThermoMixer C (Eppendorf). After shaking small aliquots were taken out for analysis. Yield: not determined

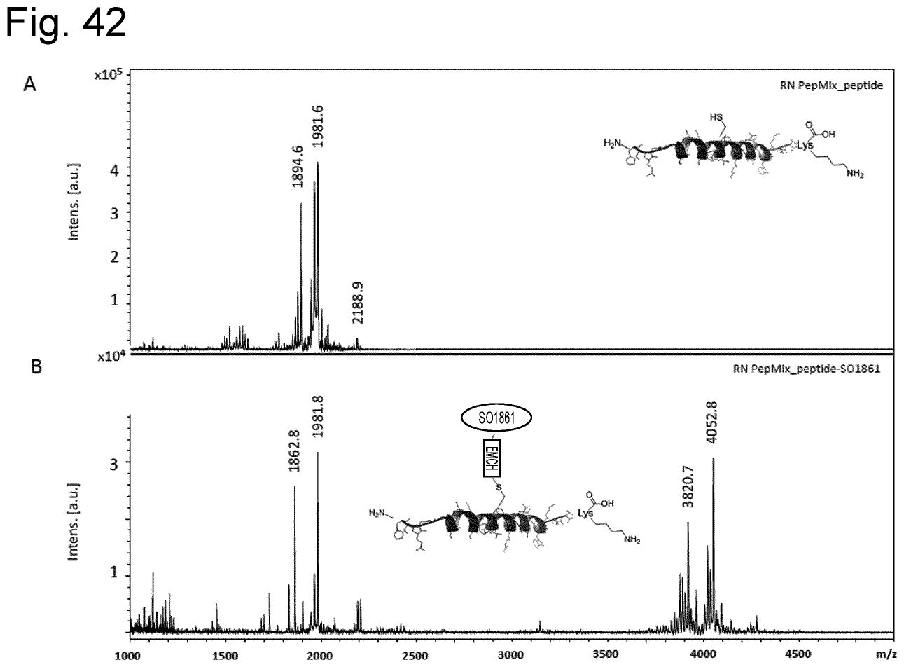

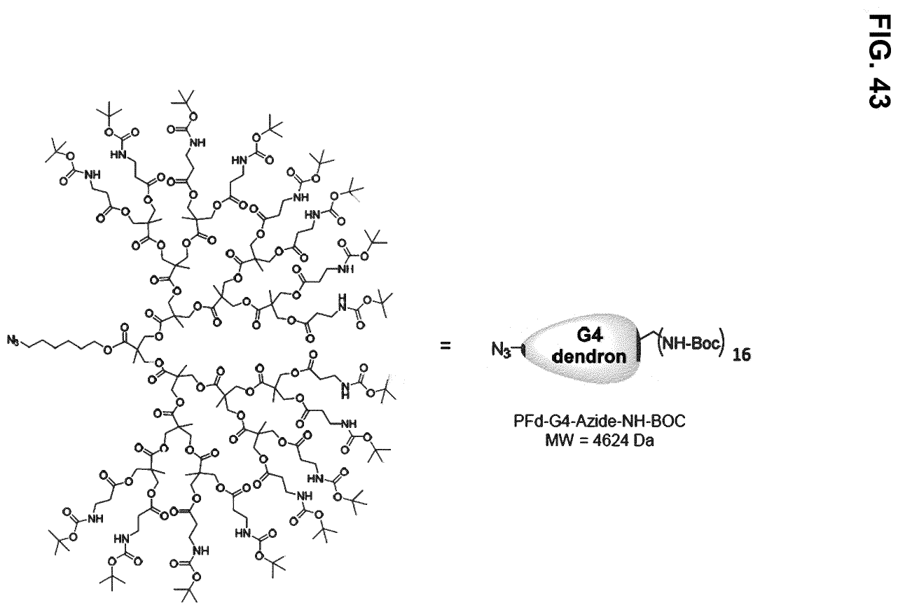

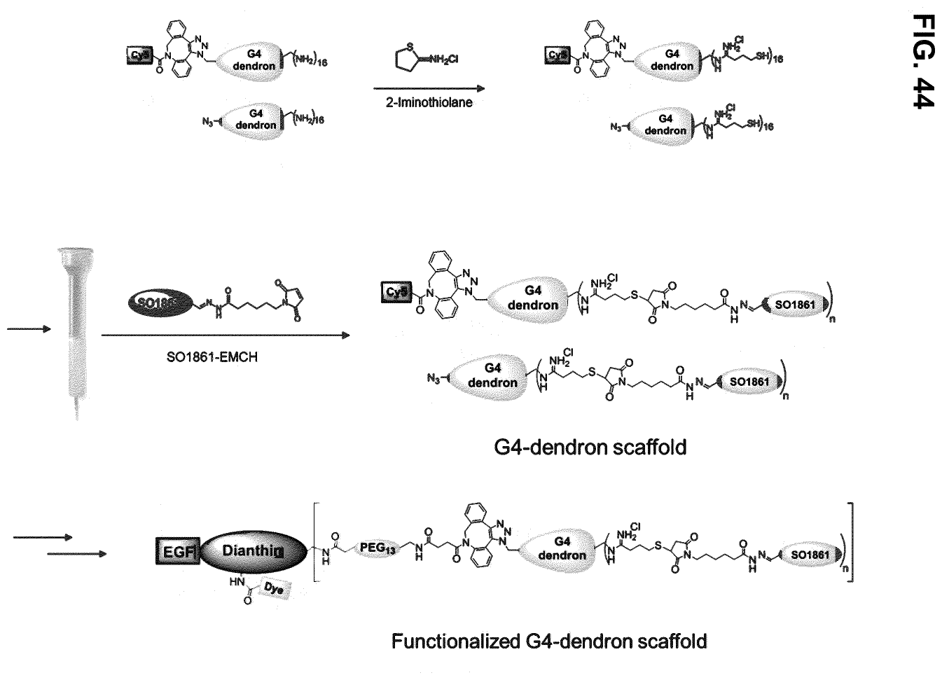

MALDI-TOF-MS (FIG. 42 B) (RN mode): m/z 4.05 kDa ([M+H]⁻, peptide-SO1861), 3.92 kDa ([M+H]⁻, peptide-501730), 1.98 kDa ([M+H]⁻, peptide), 1.86 kDa ([M+H]⁻, SO1861).

### Results

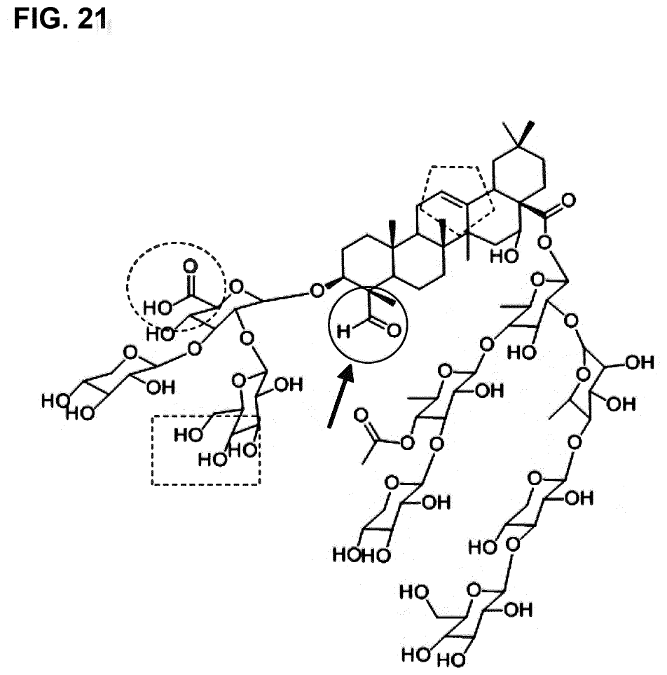

Considering available chemical groups for conjugation reactions to the SO1861 molecule, four chemical groups have been identified. The alcohols and diols of the sugar residues, the aldehyde group on the triterpenoid backbone, the carboxylic acid on one of the sugar residues (glucuronic acid), and the alkene group on the triterpenoid backbone as highlighted in FIG. 21.

In view of the pros and cons of each identified chemical group (Table 12), the aldehyde and alcohol groups are best suitable for reversible conjugation reactions, while the alkene and the carboxylic acid (glucuronic acid) are the groups best suitable for irreversible/stable conjugation reactions. The aldehyde group within the molecule structure of SO1861, however, is the most suitable for reversible conjugation reactions over the alcohols. On the one hand, because there is only one aldehyde present in the structure that allows chemo selective reactions. On the other hand, because the aldehyde can perform reversible conjugation reactions with a variety of chemical groups such as amines, hydrazides, and hydroxylamines forming acid-cleavable moieties like imines, hydrazones, and oximes. This factor enables a freedom of choice over the chemical group for the desired reversible conjugation reaction. Contrary, the alcohols are good candidates for reversible conjugation reaction via the formation of acetals and ketals as well, but lack in chemoselectivity since they are present in a large quantity on the glycosidic structure.

For the formation of an irreversible and stable bond the carboxylic acid is the most suitable since it can form amides and esters with the common tools used in peptide chemistry (e.g. reaction with amines via carbodiimide mediated amide formation).

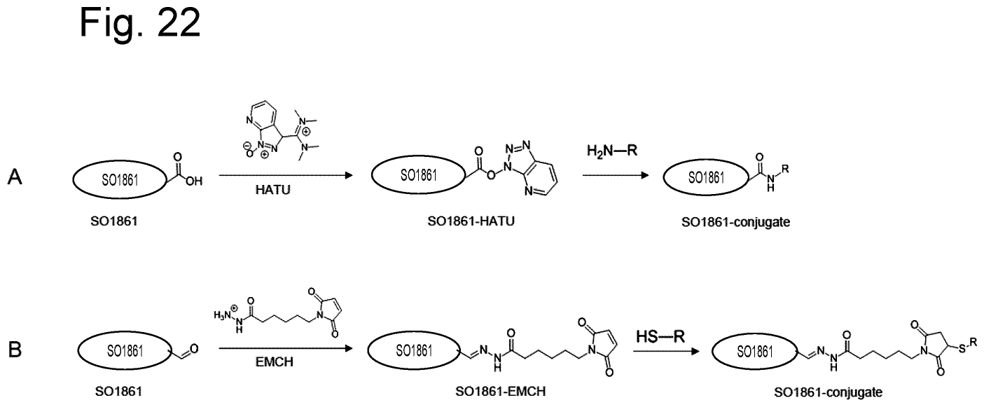

Thus, for the development of an endosomal escape enhancing saponin (such as SO1861) a methodology has been established that allows the generation of a non-cleavable and cleavable ‘ready to conjugate’ saponins (FIG. 22) using the most suitable chemical groups present on SO1861.

For producing non-cleavable ‘ready to conjugate’ saponins the carboxylic group of SO1861 is activated via a reagent used in peptide coupling chemistry to generate an active ester (e.g. 1-[Bis(dimethylamino)methylene]-1H-1,2,3-triazolo[4,5-b]pyridinium 3-oxide hexafluorophosphate, HATU). The resulting active ester of SO1861 is able to react with amines forming stable amide bonded conjugates (FIG. 22 A).

For producing cleavable ‘ready to conjugate’ saponins the aldehyde group of SO1861 is reacted with an EMCH (ε-maleimidocaproic acid hydrazide) linker. The hydrazide group of EMCH forms an acid cleavable hydrazone bond with the aldehyde of SO1861. At the same time the EMCH linker presents a maleimide group that is thiol (sulfhydryl group) reactive and thus can be conjugated to thiols (FIG. 22 B).

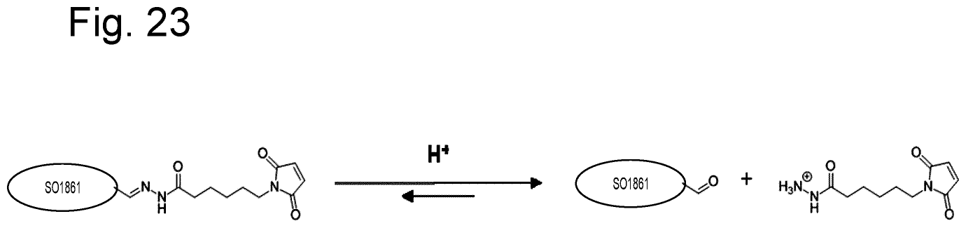

The maleimide group of SO1861-EMCH performs a rapid and specific Michael addition reaction with thiols and thiol bearing polymeric structures when carried out in a pH range of 6.5-7.5 (FIG. 22 B). In addition, the acid sensitive hydrazone linkage between the SO1861 and EMCH can be utilized to perform saponin release from a scaffold in acidic environment (FIG. 23). Thus, the EMCH linker fulfills both the need for a pH cleavable strategy and a conjugation strategy to a polymeric structure.

TABLE 12
Functional groups that are available for saponin conjugation reactions

| Functional Group | Pros | Cons |
|---|---|---|
| Alcohol | Suitable for reversible acetal/ketal | Acetal/ketal formation without |
| (Diols) | formation | chemoselectivity |
| | Suitable for ester formations with | Ester formation without |
| | activated carboxylic acids | chemoselectivity |
| Aldehyde | Suitable for chemoselective | Not suitable for acetal formation in |
| | reversible hydrazone formation with | the presence of unprotected |
| | hydrazides | saponin sugar diols |
| | Suitable for chemoselective | |
| | reversible imine formation with | |
| | amines | |
| | Suitable for chemoselective | |
| | reversible oxime formation with | |
| | hydroxylamines | |
| Alkene | Suitable for chemoselective | Not suitable for reversible |
| | irreversible radical reactions | conjugation reactions |
| | | Not suitable for reactions involving |
| | | a hydrogenation step |
| Carboxylic | Suitable for chemoselective amide/ | Not suitable for reversible |
| acid | ester formation with amines and | conjugation reactions under mild |
| | alcohols after activation | conditions |

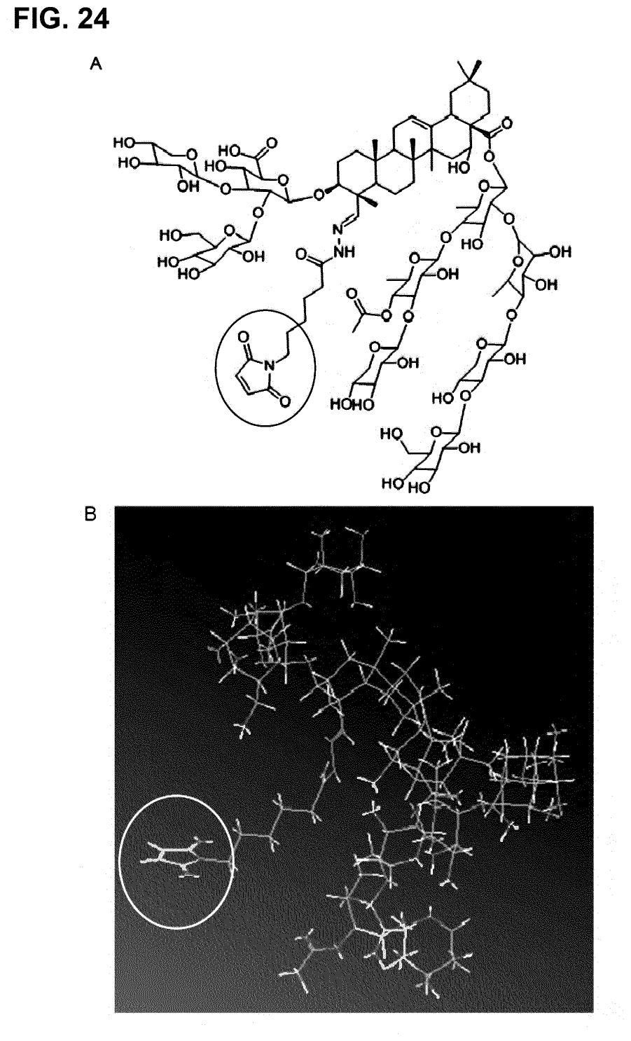

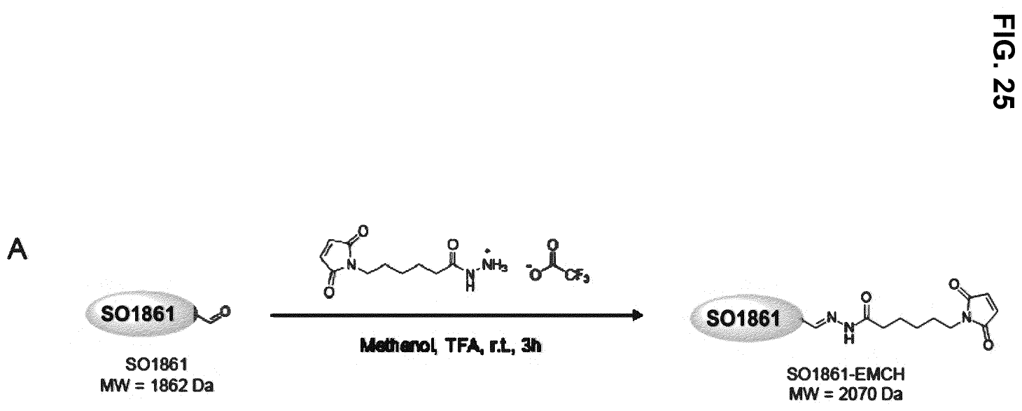

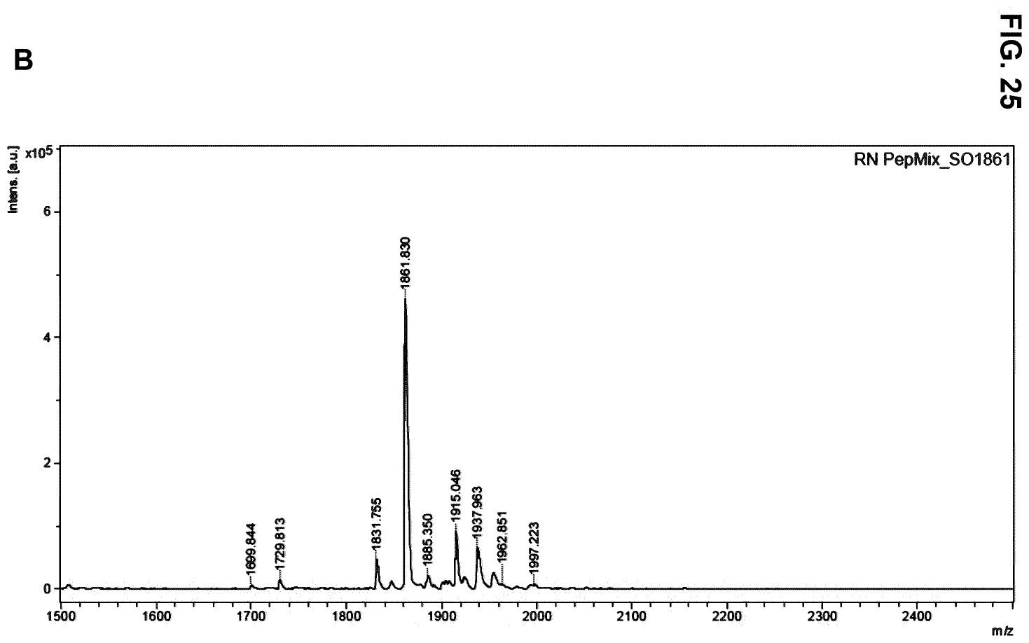

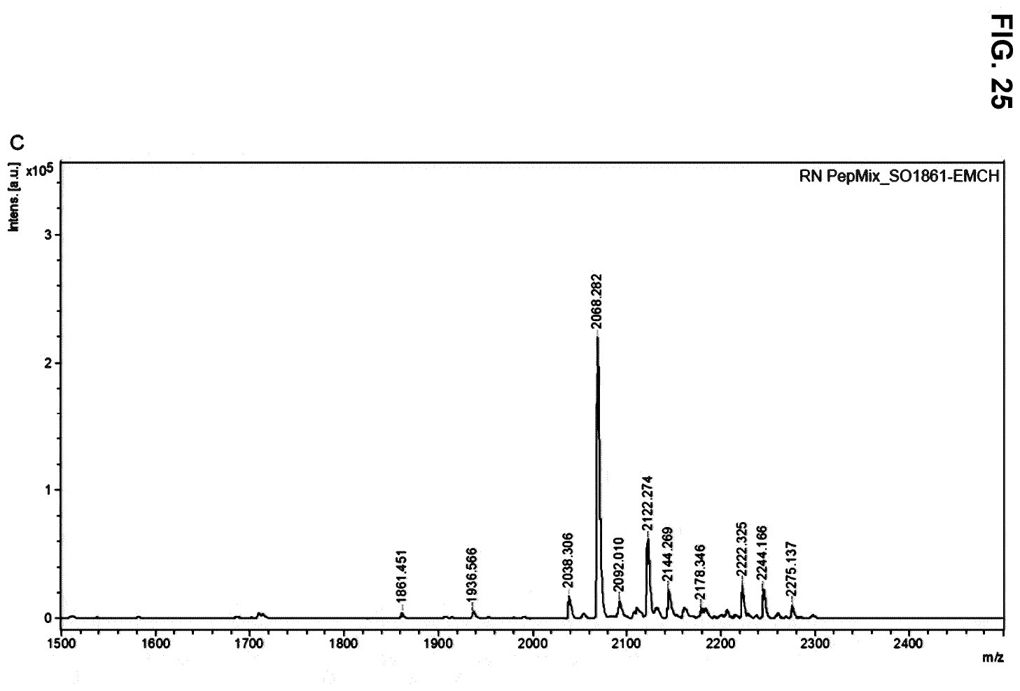

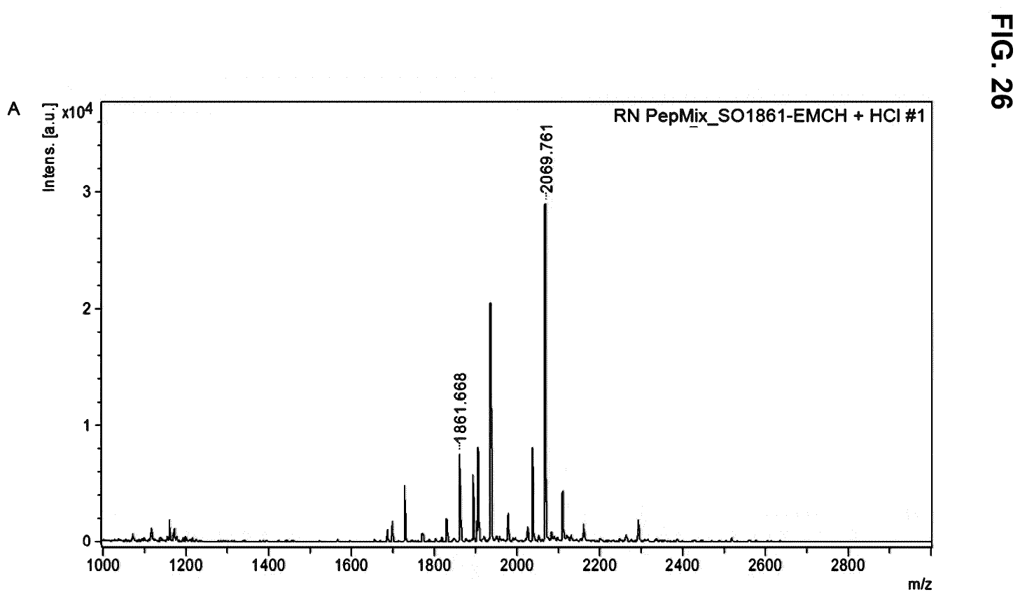

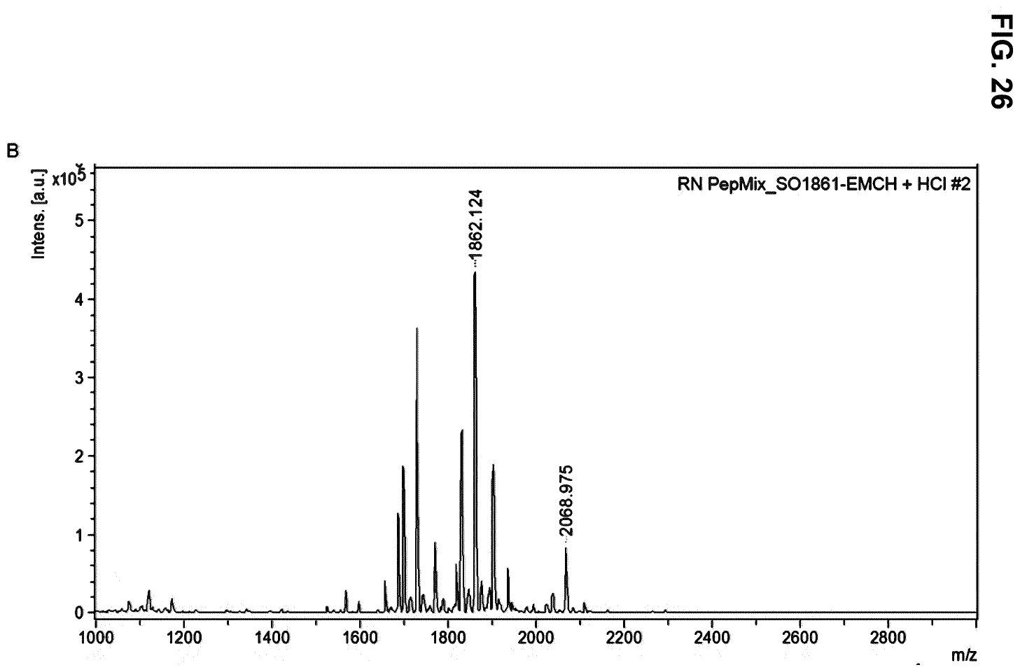

Regarding an ideal EMCH spacer length for conjugation to a polymeric structure, computer simulation (PerkinElmer, ChemBio3D, Ver. 13.0.0.3015) shows that the maleimide group on SO1861-EMCH is located at the periphery of the molecule and thus should be accessible for thiol bearing polymeric structures (FIG. 24).

To synthesize the SO1861-EMCH, a strategy has been developed that allows the conversion of the aldehyde group on the SO1861 to EMCH (FIG. 25 A). The SO1861-EMCH conjugate was isolated and successfully characterized via nuclear magnetic resonance spectroscopy (see materials and methods section, FIG. 19) and matrix-assisted laser desorption/ionization time-of-flight mass spectrometry (MALDI-TOF-MS) as shown on FIGS. 25 B and 25 C, and FIG. 20 A.

For testing the pH dependent hydrolysis of the hydrazone bond, SO1861-EMCH was dissolved in an HCl solution at pH 3 and MALDI-TOF-MS spectra were recorded at two different points in time (FIG. 26). As shown on FIGS. 26 A and 26 B, a clear decreasing tendency of the peak at m/z 2070 Da that corresponds to SO1861-EMCH is visible in FIG. 26 B. Since SO1861 is generated during hydrolysis, an increase of the peak at m/z 1861 Da was recorded that accompanied the decreasing tendency at m/z 2070 Da. These results show that the hydrazone bond is responsive towards hydrolysis and gets cleaved even if it is attached on SO1861.

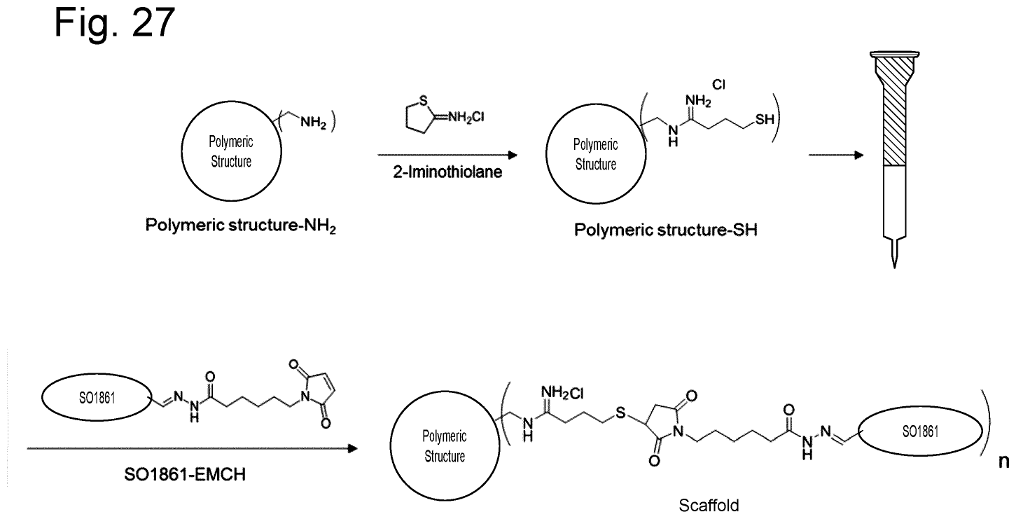

In order to conjugate SO1861-EMCH to a polymeric structure, accessible amines of the polymeric structure are converted into thiols with the aid of the agent 2-iminothiolane. The generated free thiols on the polymeric structure act then as the nucleophile for the thiol-ene Michael-type addition to the maleimide group of SO1861-EMCH (FIG. 27). This developed methodology is suitable for the conjugation of SO1861-EMCH to any available polymeric structure that obtains accessible amine groups and allows furthermore a control over the number of conjugated SO1861 molecules depending on the polymeric structure, respectively.

First proof of concept for conjugation of ‘ready-to conjugate saponins’ to a polymeric structure was obtained by use of the amine of a protein (poly amino acid scaffold example), bovine serum albumin (BSA). After conjugation, mass spectrometry obtained the corresponding peaks of BSA-SO1861 at m/z ˜70, ˜72, and ˜74 kDa (FIG. 28 A). In comparison with the detected mass of BSA with m/z 66 kDa (FIG. 28 B), the obtained masses of BSA-SO1861 correspond to a mixture of BSA-SO1861 conjugates consisting of 2, 3, and 4 SO1861 molecules per BSA.

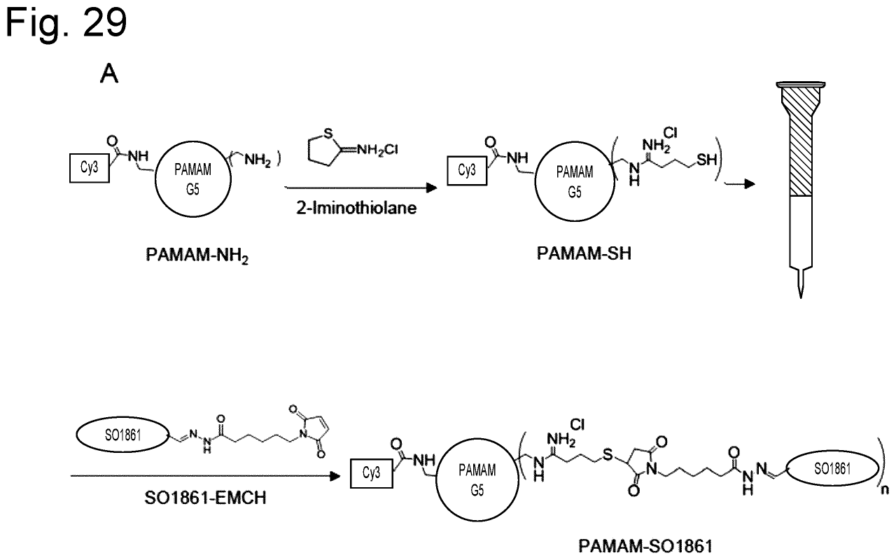

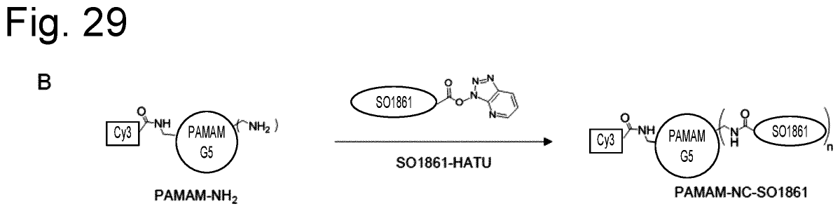

Next proof of concept for conjugation of ‘ready-to conjugate saponins’ to a polymeric structure was obtained by the use of the amine bearing generation 5 (G5) dendrimer poly(amidoamine) (PAMAM with covalently coupled red-fluorescent dye (Cy3)). PAMAM-Cy3 was utilized as the polymeric structure for the conjugation to both SO1861-EMCH and SO1861-HATU and served as a model for conjugation of SO1861 to a polymeric structure (FIG. 29).

All accessible amine groups of Cy3-PAMAM were converted into thiols using a 3 fold excess of 2-iminothiolane per Cy3-PAMAM amines followed by the reaction with SO1861-EMCH. Three different feed equivalents (5, 20 and 57) of SO1861-EMCH were used for the three reaction batches. After reaction, the recorded masses of the Cy3-PAMAM-SO1861 conjugates at MALDI-TOF-MS show an increment of the corresponding masses with increasing the SO1861-EMCH feed (FIG. 30). The three different feeds corresponded to an obtained mass of m/z 38.4 kDa, m/z 53.9 kDa and m/z 133.8 kDa for the Cy3-PAMAM-SO1861 conjugates that correspond to 6, 13 and 51 SO1861 molecules attached per PAMAM dendrimer as shown on FIG. 30 B-D.

In another reaction, only a certain number of PAMAM amines were converted into thiols prior to reaction with SO1861-EMCH. Here, two different feed equivalents of 2-Iminothiolane (8 and 32) and two different feed equivalents of SO1861-EMCH (5 and 30) were used, respectively. After reaction, the respective spectra of the Cy3-PAMAM-SO1861 conjugates at MALDI-TOF-MS show peaks at m/z 37.7 kDa (5 feed equivalents of SO1861-EMCH) and at m/z 87.0 kDa (30 feed equivalents of SO1861-EMCH) as shown on FIG. 31. The obtained masses at m/z 37.7 kDa and m/z 87.0 kDa correspond to Cy3-PAMAM-SO1861 conjugates with 5 and 30 SO1861 molecules attached and demonstrate that with this method almost all feed of SO1861-EMCH were conjugated.

For the generation of a non-pH-cleavable saponin conjugate the carboxylic acid of SO1861 was activated with HATU and then reacted with the amines of Cy3-PAMAM forming a pH stable amide bound between Cy3-PAMAM and SO1861. The resulting mass of the conjugate was detected via MALDI-TOF-MS with a mass of m/z 62.3 kDa that corresponds to Cy3-PAMAM-NC-SO1861 (NC=non-cleavable) conjugate with 17.5 SO1861 molecules attached per PAMAM (FIG. 29 B, FIG. 32).

Next, the saponin conjugated scaffolds were conjugated to linking points for a possible conjugation to targeted therapeutics (e.g. targeted toxins) via the so-called strain-promoted alkyne-azide cycloaddition (SPAAC, click chemistry) to obtain a functionalized scaffold. For this reaction, Cy3-PAMAM-SO1861 (FIG. 33 C, D) and Cy3-PAMAM-NC-SO1861 (FIG. 33 B) were conjugated to a heterobifunctional NHS-PEG₁₃-DBCO linker that generated a strained alkyne on the conjugates' surface (FIG. 33 A). The NHS (N Hydroxysuccinimide) moiety of the linker reacted with remaining amines of the PAMAM-saponin conjugates forming an amide bond between the scaffold and the linker. The resulting DBCO (dibenzocyclooctyne) moiety on the conjugates is able to perform SPAAC with corresponding azides on the targeted therapeutics.

Dianthin-EGF served as a model targeted toxin and dianthin served as a non-targeted toxin. Both toxins were labeled with Alexa Fluor™ 488 using the tetrafluorophenyl ester (TFP) derivative of the dye. The dye labeled proteins were then conjugated to a heterobifunctional NHS-SS-PEG₃-azide linker to obtain the corresponding chemical moiety for the SPAAC to the PAMAM-saponin conjugates. Maldi-TOF-MS measurements showed that one Alexa Fluor™ 488 dye and 9 NHS-SS-PEG3-azide molecules were attached per dianthin-EGF molecule (FIG. 34, FIG. 35). Furthermore, Alexa Fluor™ 488 labeled dianthin-EGF was conjugated to a heterobifunctional NHS-PEG₁₂-azide linker that lacked the disulfide bond and would thus generate a non-toxin-cleavable construct.

The Cy3-PAMAM-NC-SO1861-DBCO and Cy3-PAMAM-SO1861-DBCO conjugates were reacted with Alexa Fluor™ 488 labeled azido-toxins to perform a strain-promoted alkyne-azide cycloaddition. The conjugation between the reacting agents was indicated via gel electrophoresis and the co-localization of the fluorescent signals of Cy3 that is only attached on the PAMAM polymer and Alexa Fluor™ 488 that is only attached on the toxins on a polyacrylamide gel after gel electrophoresis (FIG. 36).

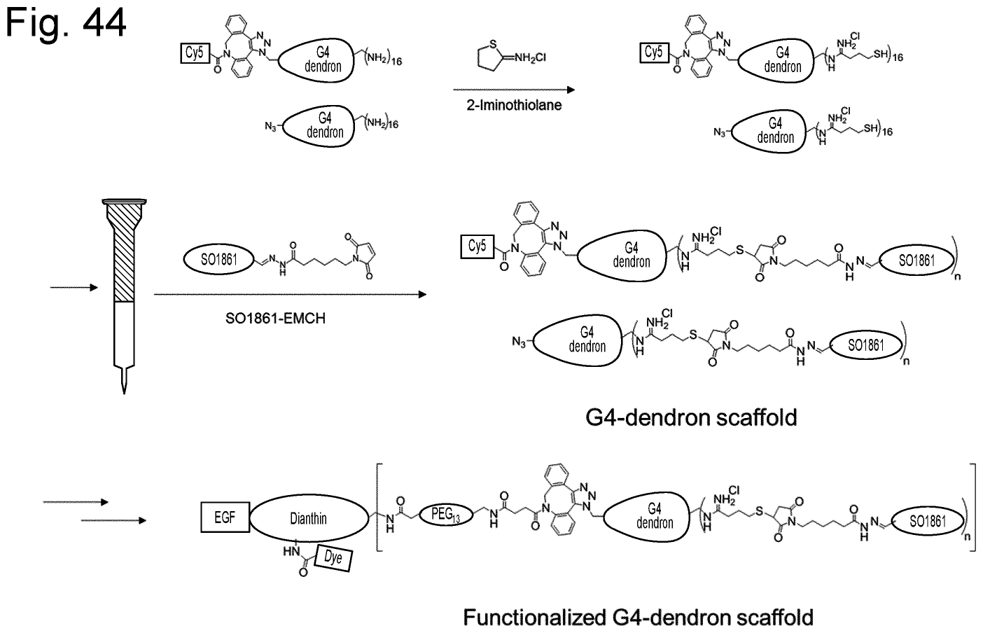

As an alternative polymeric structure to the PAMAM dendrimer, a G4-dendron (PFd-G4-Azide-NH-BOC, Polymer Factory) with 16 functional amino end groups and an azido group at the focal point was utilized for the conjugation to SO1861 (FIG. 43). The advantage of using a dendron over a dendrimer is the focal point that the dendron structure is exhibiting. This focal point allows the direct conjugation to a targeted toxin without the need of its post-modification with orthogonal click functions (FIG. 44). As shown in FIG. 44, the dendron underwent the same methodology as described for the PAMAM dendrimer. Briefly, after partial dye labeling and deprotection (FIG. 45), the amino groups of the dendron were converted into thiols using the thiolating reagent 2-iminothiolane followed by conjugation to SO1861-EMCH. For the conjugation to SO1861-EMCH three different feed equivalents of SO1861-EMCH were used. The dendron-SO1861 conjugates were analyzed via MALDI-TOF-MS. As expected, the conjugate species of 1 and 2 SO1861 molecules per dendron molecule were obtained when low SO1861-EMCH feed equivalents of 3 and 10 were used (FIG. 46 B, C). Higher dendron-SO1861 conjugate species of up to 9 SO1861 molecules per dendron were obtained (FIG. 46 A) when using a feed equivalent of 22 SO1861-EMCH molecules per dendron molecule. In further experiments, the saponin functionalized dendron will be conjugated to targeted toxins over its focal point to yield a functionalized scaffold and will be evaluated biologically.

The previous examples demonstrate that a methodology has been developed that allows the conjugation of a determined amount of SO1861 molecules or other endosomal escape enhancer molecules to a polymeric structure for enhanced cytoplasmic delivery of therapeutic substances such as targeted toxins.

To further test other conjugation methodologies of SO1861 to a polymeric structure, the reductive amination pathway was used. For this, the aldehyde group of SO1861 was directly conjugated to PAMAM amines forming an imine bound. The imine bond formation was followed a reductive amination step through the addition of the reductive agent sodium cyanoborohydride forming a pH-stable amine bond between SO1861 and PAMAM (FIG. 37 A). Similar to the EMCH and HATU approach, this methodology allows a control over the number of conjugated saponins per polymer as shown on FIG. 37 B, C. Here, PAMAM-saponin conjugates were produced which obtained a number of 10 (FIG. 37 B) and 30 (FIG. 37 C) SO1861 molecules per PAMAM.

Another approach for the development of a SO1861 scaffold among the discussed polymer, and protein approach is the poly(SO1861) approach. The idea of this approach is to generate a polymer that consists of SO1861 molecules only, with pH sensitive cleavable bonds that release the SO1861. In addition, the poly(SO1861) should be able to perform conjugation reactions to toxins and biopolymers. The main goal with this approach is to keep it as simple and cost effective as possible. Since a protocol for the generation of acid cleavable SO1861 has been developed already (SO1861-EMCH approach) it would be interesting to see if it is possible to polymerize the SO1861-EMCH through simple addition of a polymerization initiator without further modifying the SO1861 or identifying other conjugation sites on the SO1861 molecule. In the past, several papers have discussed the polymerization of maleimide groups by using radical initiators which attack the double bond of the maleimide group and thus initiate a radical polymerization along the double bonds of the maleimides. Since SO1861-EMCH reveals a maleimide group in its structure this group could potentially be explored for radical polymerization reactions to yield a poly(SO1861) with acid cleavable function. If the polymerization reaction has a reasonable reaction time the generated SO1861 polymers could be quenched with a radical quencher that not only quenches the reaction but also generates a functional group for toxin or biopolymer conjugation. Such a reaction scheme is illustrated in FIG. 38. Here, the system of ammonium persulfate (APS) and tetramethylethylenediamine (TMEDA) is shown in an exemplary way as radical generator and aminopropanethiol serves as a model radical quencher. Using aminopropanethiol as a quencher exemplary, the generated amine group could be specifically further modified to a click-able group or being used to directly conjugate the poly(SO1861) to a toxin.

In free radical polymerization the reaction conditions have a huge influence on the polymer properties and the reaction outcome. For instance, temperature, monomer concentration, and initiator concentration play a major role for forming the polymer and have to be fine-tuned according to the desired polymer properties. As radical polymerizations are usually carried out at temperatures above 50° C., the first reactions have been performed at a temperature of 60° C. It was interesting to see if the SO1861-EMCH polymerization can be initiated spontaneously and if APS and TMEDA would have an influence on the polymerization degree. Thus, three reactions have been carried out, using the same SO1861-EMCH concentration, but differ in their APS/TMEDA composition. In the first reaction only the SO1861-EMCH was heated up to 60° C. for 3 h, while the second reaction contained SO1861-EMCH and APS, and the third reaction contained SO1861-EMCH, APS, and TMEDA. (For these experiments the same amount and concentration of starting materials have been used which are mentioned in the Materials and Methods section “Poly(SO1861) synthesis”). The batches have been analyzed via MALDI-TOF-MS as shown on FIG. 39 A-C. Interestingly it has been shown that SO1861-EMCH started to form oligomers consisting of 2, 3, 4, 5, and 6 SO1861 molecules spontaneously when heated up to 60° C. (FIG. 39 A). The addition of 11⁻³ equivalents APS at the same temperature had no influence on this trend (FIG. 39 B). When using the initiator system of APS/TMEDA, however, SO1861 oligomers of up to 9 SO1861 molecules with a molecular weight of 18.2 kDa could be detected (FIG. 39 C). In addition, the obtained peaks for the oligomers seemed much bigger in comparison with the peaks in FIGS. 39 A and 39 B, indicating a higher consumption of SO1861-EMCH for this reaction.

To further fine-tune the reaction conditions, other initiators such as azo-initiators like 2,2′-azobis[2-methyl-N-(2-hydroxyethyl)propionamide] and azobisisobutyronitrile will be tested, as well as other polymerization techniques such as controlled radical polymerization (atom-transfer radical-polymerization, reversible addition-fragmentation chain transfer, etc.). Moreover, another hydrazide linker as a substitute for EMCH could be considered which obtains a functional group (such as an acryl or acrolyol residue) that is more suitable for radical polymerization than the maleimide group.

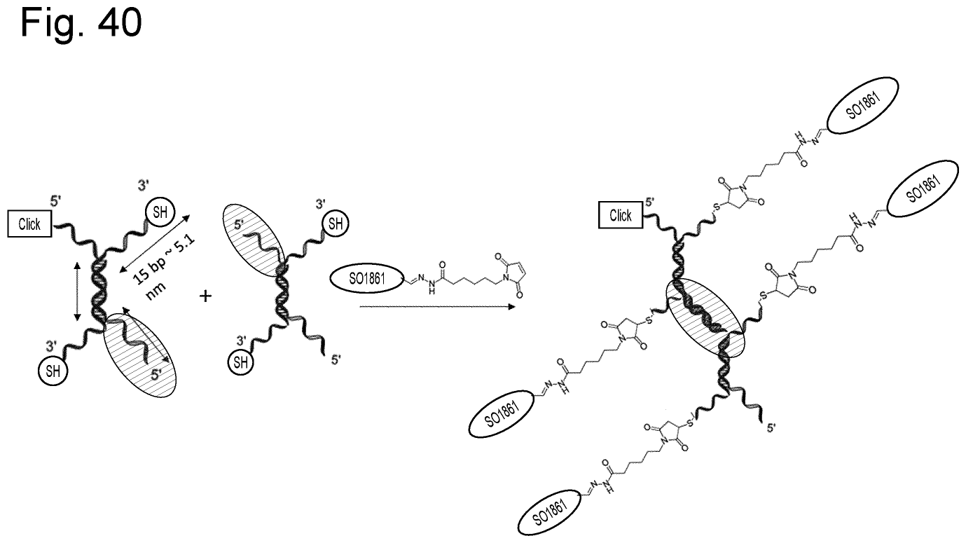

Another approach for the development of a SO1861 scaffold is the DNA approach. The idea of this approach is to utilize the concept of the so-called DNA-origami. DNA-origami as the polymeric or assembled polymeric structure to conjugate saponins to it, can offer several inherent advantages including stability, scalability, and precise control of the final size and shape of the resulting DNA-saponin scaffold. Since these DNA nanocarriers are comprised of natural DNA, they are biocompatible and do not show toxicity to living cells, and can ease the release of cargo from internal cellular compartments. The multivalency of such a structure can further allow fine-tuning targeting capabilities and high capacity for a variety of payloads such as fluorophores and toxins. Thus, in this approach DNA strands are identified that offer chemical functional groups on the 3′ and 5′ endings respectively, and that are able to hybridize only in certain wanted areas of the sequence that allow a control over the final shape of the construct. The chemical groups should be utilized to couple saponins, for instance though a thiol-ene reaction between the already developed SO1861-EMCH and a thiol group on one of the 3′ and 5′ DNA strands. The complementary DNA strand can offer a click function group that can be used for coupling to a targeted toxin. The concept is illustrated in FIG. 40.

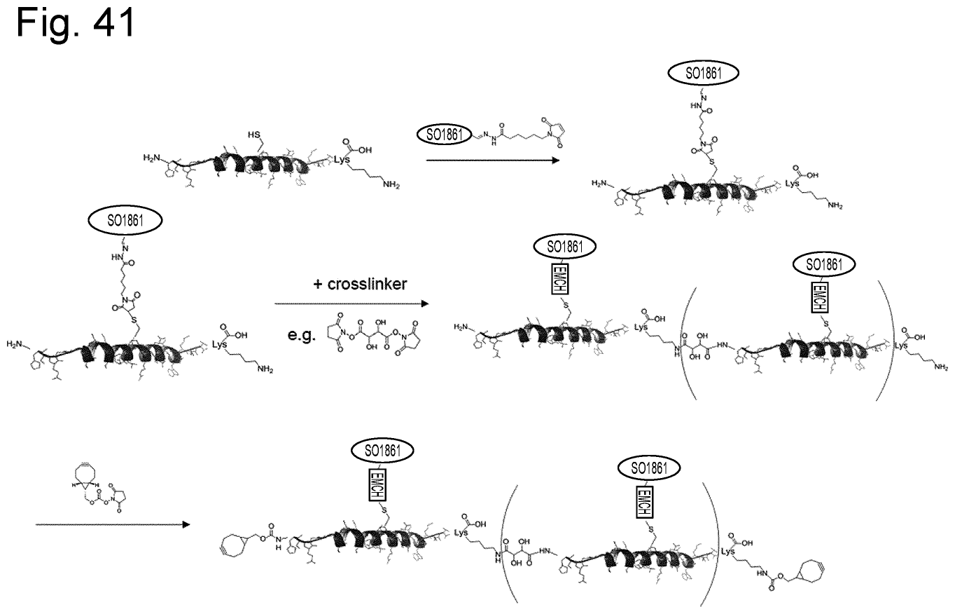

A similar approach is imaginable by using a specific peptide sequence instead of DNA strands that is able to bind and release saponins and that can be polymerized forming a large poly(peptide)-like structure. In this approach, a peptide sequence has been identified and purchased that has a length fitting the calculated size of a SO1861-EMCH molecule, that offers a cysteine residue in the middle of the sequence, and that obtains an amine group at both the N-terminus and C-terminus. The cysteine residue can be utilized to conjugate SO1861-EMCH via a thiol-ene reaction of the maleimide group of SO1861-EMCH and the thiol group of the cysteine residue. The two amine groups can be utilized to polymerize the peptide-SO1861 conjugate with a suitable crosslinker as shown on FIG. 41.

Conjugation studies have shown that the conjugation of SO1861-EMCH to the customized peptide (sequence: SESDDAMFCDAMDESDSK) was successful. The peptide that bears a maleimide reactive cysteine in the middle of the sequence and its conjugation to SO1861-EMCH was analyzed via MALDI-TOF-MS (FIG. 42; [SEQ ID NO. 1]). The MALDI-TOF-MS spectra shows the expected peak for the peptide-SO1861 conjugate at m/z 4053 Da and an additional peak at m/z 3920 Da which is the peptide-SO1861 conjugate of the corresponding saponin-EMCH of SO1730. As SO1861-EMCH has been used in slight excess (1.3 equivalents) and no SO1861-EMCH peak was detected after reaction, it can be assumed that the conjugation was quantitative. For starting first polymerization reactions of the peptide-SO1861, disuccinimidyl tartrate will be utilized as the amine reactive cross-linker.

### Example 19

### Cell Viability Assay

HeLa cells were seeded in DMEM (PAN-Biotech GmbH) supplemented with 10% fetal bovine serum (PAN-Biotech GmbH) and 1% penicillin/streptomycin (PAN-Biotech GmbH), in a 96 well plate at 5,000 c/w in 100 μL/well and incubated overnight at 37° C. and 5% CO₂. The next day 20× concentrated stocks of the PAMAM, PAMAM-conjugates, G4-dendron (prepared in Example 18) or chloroquine (Sigma Aldrich) samples were prepared in DMEM. The media was removed from the cell culture plate and replaced by 160 μL culture media, followed by the addition of 10 μL sample/well (from the 20× concentrated stocks) and a 45 min incubation at 37° C. During this incubation the SO1861 concentration curve was prepared. The SO1861 master stock was heated for 10 min at 50° C., while shaking at 1,250 rpm. Followed by 15 sec sonication and a brief re-heating at 50° C. for 1 min, while shaking at 1,250 rpm. Subsequently a serial dilution of SO1861 was prepared in PBS. The SO1861 concentration curve was prepared as 10× concentrated stock, from which 20 μL was added/well. After a 15 min incubation at 37° C., 10 μL dianthin-EGF (prepared in Example 17) diluted in DMEM to 30 μM) or DMEM containing an equal amount of PBS was added/well, to obtain a final dianthin-EGF concentration of 1.5 μM as well as the indicated SO1861 and the different polymeric structures or chloroquine concentrations in a final volume of 200 μL/well.

After treatment the cells were incubated for 72 hr at 37° C. before the cell viability was determined by a MTS-assay, performed according to the manufacturer's instruction (CellTiter 96® AQueous One Solution Cell Proliferation Assay, Promega). Briefly, the MTS solution was diluted 20× in DMEM without phenol red (PAN-Biotech GmbH) supplemented with 10% FBS. The cells were washed once with 200 μL/PBS well, after which 100 μL diluted MTS solution was added/well. The plate was incubated for approximately 20-30 minutes at 37° C. Subsequently, the OD at 492 nm was measured on a Thermo Scientific Multiskan FC plate reader (Thermo Scientific). For quantification the background signal of ‘medium only’ wells was subtracted from all other wells, before the cell viability percentage of treated/untreated cells was calculated, by dividing the background corrected signal of treated wells over the background corrected signal of the untreated wells (×100).

### FACS Analysis

HeLa cells were seeded in DMEM (PAN-Biotech GmbH) supplemented with 10% fetal calf serum (PAN-Biotech GmbH) and 1% penicillin/streptomycin (PAN-Biotech GmbH), at 500,000 c/plate in 10 cm dishes and incubated for 48 hrs (5% CO₂, 37° C.), until a confluency of 90% was reached. Next, the cells were trypsinized (TrypIE Express, Gibco Thermo Scientific) to single cells. 0.75×10⁶ Cells were transferred to a 15 mL falcon tube and centrifuged (1,400 rpm, 3 min). The supernatant was discarded while leaving the cell pellet submerged. The pellet was dissociated by gentle tapping the falcon tube on a vortex shaker and the cells were washed with 4 mL cold PBS (Mg²⁺ and Ca²⁺ free, 2% FBS). After washing the cells were resuspended in 3 mL cold PBS (Mg²⁺ and Ca²⁺ free, 2% FBS) and divided equally over 3 round bottom FACS tubes (1 mL/tube). The cells were centrifuged again and resuspended in 200 μL cold PBS (Mg²⁺ and Ca²⁺ free, 2% FBS) or 200 μL antibody solution; containing 5 μL antibody in 195 μL cold PBS (Mg²⁺ and Ca²⁺ free, 2% FBS). APC Mouse IgG1, K Isotype Ctrl FC (#400122, Biolegend) was used as isotype control, and APC anti-human EGFR (#352906, Biolegend) was used to stain the EGFR receptor. Samples were incubated for 30 min at 4° C. on a tube roller mixer. Afterwards, the cells were washed 3× with cold PBS (Mg²⁺ and Ca²⁺ free, 2% FBS) and fixated for 20 min at room temperature using a 2% PFA solution in PBS. Cells were washed 2× with cold PBS, and resuspended in 250-350 μL cold PBS for FACS analysis. Samples were analyzed with a BD FACSCanto II flow cytometry system (BD Biosciences) and FlowJo software.

### Results for Example 19

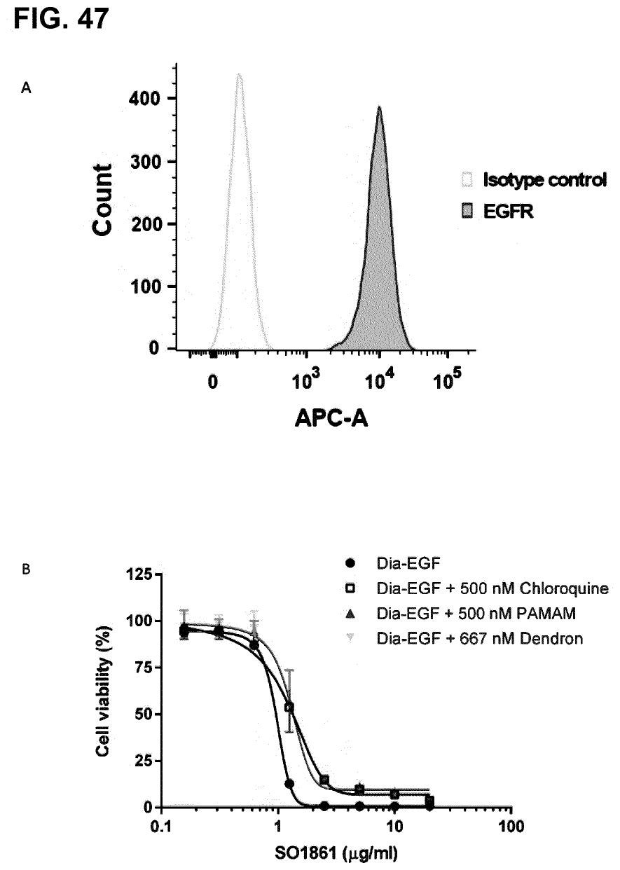

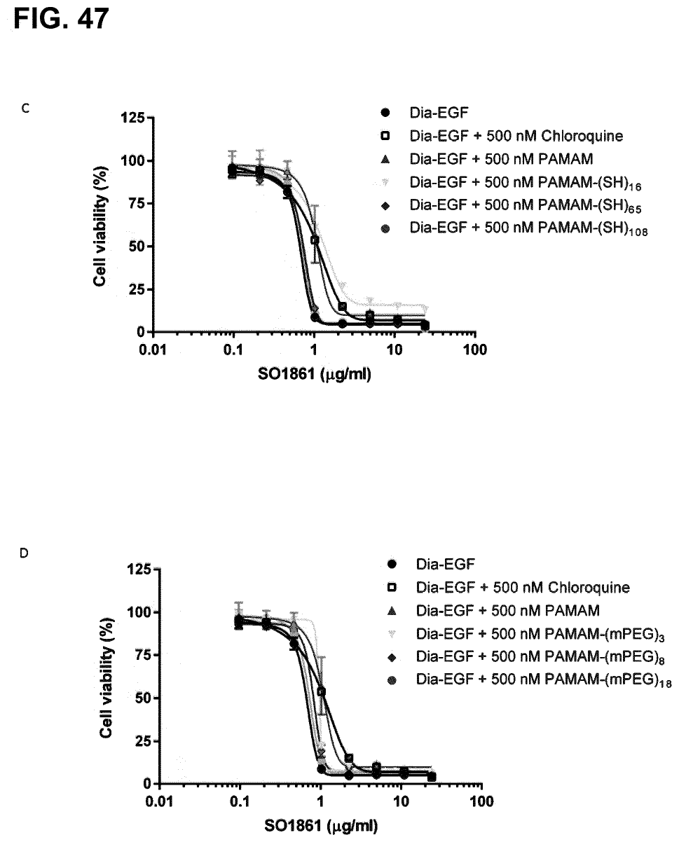

Previously it has been described that the amino groups in amine containing polymeric structures such as PAMAM and PEI (polyethylenimine) are able to block the acidification of the endosomes via the their intrinsic H⁺ buffering capacity [7-9]. Since the endosomal escape enhancing properties of SO1861 are only exposed at low endosomal pH (<pH 5), the scaffold or functionalized scaffold should not contain chemical groups that are able to interfere in acidification of the endosomes and thus block the activity of SO1861. We therefore tested the amine containing polymeric structures of a G5 PAMAM (128 primary amines as well as approximately 126 tertiary amines) and G4-dendron (16 primary amines), in order to determine if these molecules inhibit the endosomal escape enhancing capacity of SO1861. We performed co-administration experiments of PAMAM (native or thiolated) or dendron (native) in combination with dianthin-EGF and various SO1861 concentrations on HeLa cells (all compounds described and prepared in Examples 17 and 18). As control for the inhibition of endosomal acidification chloroquine was used. HeLa cells show sufficient EGFR cell surface levels (FIG. 47 A). We observe that both ‘native’ PAMAM and chloroquine inhibit the SO1861-mediated endosomal escape of the targeted toxin and subsequent cell killing in Hela cells (FIG. 47 B). PAMAM at 500 nM inhibits even to an equal extend as the endosomal acidification inhibitor chloroquine, while 667 nM dendron has no affect at all. To further address if the inhibitory activity of the ‘native’ PAMAM is due to the availability of amino groups in PAMAM, the primary amino groups of PAMAM were partially thiolated through reaction with 2-iminothiolane (FIG. 48), resulting in 16 of 128 (FIG. 48 C), 65/128 (FIG. 48 D), and 108/128 (FIG. 48 E) blocked primary amines. We observe that thiolation of 65 and 108 primary amines overcomes the inhibition of SO1861-mediated endosomal escape, whereas thiolation of up to 16 amines groups still shows the inhibitory effects of SO1861-mediated endosomal escape of the targeted toxin (FIG. 47 C). The primary amino groups of PAMAM were also partially PEGylated through a reaction with mPEG₂ₖ-NHS (FIG. 49), resulting in 3 of 128 (FIG. 49 C), 8/128 (FIG. 49 D), and 18/128 (FIG. 49 E) blocked primary amines. Blocking only 3 primary amines by PEGylation is already sufficient to reverse the inhibition of SO1861-mediated endosomal escape (FIG. 47 D). The shielding effect of PEGylation most likely extends beyond the small number of amines that are converted, as PEGylation is known to introduce a hydration layer that can shield off an entire molecule, if a sufficient level is reached.

These results demonstrate that the presence of a certain number of free amino groups on polymeric structures, such as PAMAM, can block endosomal acidification and thus inhibiting the endosomal escape activity of SO1861 or other glycosides. When the number of amino groups is lower, as shown for the G4-dendron, or if the amino groups have been shielded, such as thiolation or PEGylation, the inhibitory effect is reverted. As a consequence, polymeric structures are suitable that when prepared as scaffolds or functionalized scaffolds, no inhibition of the endosomal escape activity of SO1861 or other glycosides is observed.

### Example 20—Treating a Mammalian Tumor-Bearing Animal with a Conjugate of the Invention in Combination with an ADC Results in Survival and Tumor Regression

Female Balb/c nude mice were injected subcutaneously with a suspension of human A431 tumor cells. Under the skin of the mice, a human epidermal carcinoma developed in the xenograft animal tumor model. After injection of the tumor cells, the xenograft tumor was allowed to develop to a size of approximately 170-180 mm³. The A431 tumor cells have the following characteristics: high EGFR expressors, medium CD71 expressors, low HER2 expressors.

In Table 13, the results of the treatment of control mice and tumor-bearing mice are presented. Tumor-bearing mice were treated with the indicated antibodies directed to either human Her2/neu, human EGFR, or human CD71, which are cell-surface receptors on the xenograft tumor. Cetuximab was conjugated with the endosomal escape enhancing saponin SO1861. The ADCs trastuzumab-saporin and anti-CD71 mAb (IgG)-saporin were tested for their tumor-attacking efficacy in the mice, measured as tumor volume in time after start of the treatment with the ADCs. The dose of the ADCs was sub-optimal in the tumor model. That is to say, from previous experiments, it was established at which sub-optimal dose of the ADCs no tumor-regression or arrest of tumor growth would be observable.

TABLE 13
RESULTS OF TREATING A MAMMALIAN TUMOR-BEARING ANIMAL
WITH A CONJUGATE OF THE INVENTION IN COMBINATION WITH
AN ADC RESULTS IN SURVIVAL AND TUMOR REGRESSION

| | | | tumor size (volume in mm³ or + |
|---|---|---|---|
| | | | for growth, − for regression, and |
| Treatment | Patient/healthy | | ‘stable’ for growth nor |
| group | animal | treatment | regression) |
| 1 | xenograft | vehicle | 2000 mm³ (death/euthanasia) |
| 2 | xenograft | Trastuzumab-saporin | 2000 mm³ (death/euthanasia) |
| 3 | xenograft | Anti-CD71 mAb-saporin | 2000 mm³ (death/euthanasia) |
| 4 | xenograft | Cetuximab-SO1861 | 2000 mm³ (death/euthanasia) |
| 5 | xenograft | Cetuximab | >170 mm³, but <2000 mm³ |
| | | | (death/euthanasia) |
| 6 | xenograft | Trastuzumab-saporin + | Tumor regression from 180 mm³ |
| | | Cetuximab-SO1861 | at the start of treatment back to |
| | | | 80 mm³ (survival) |
| 7 | xenograft | Anti-CD71 mAb-saporin + | Tumor regression from 180 mm³ |
| | | Cetuximab-SO1861 | at the start of treatment back to |
| | | | 40 mm³ (survival) |

These results demonstrate that the combination therapy of an ADC at a dose which is ineffective when treatment of tumor-bearing mice with the ADC alone is considered (tumor growth, death of the mice (euthanasia)) with an endosomal and/or lysosomal escape enhancing conjugate of the invention consisting of a tumor-cell specific receptor targeting antibody complexed with an endosomal and/or lysosomal escape enhancing saponin, i.e. SO1861, the conjugate administered to the mice suffering from cancer, at a non-effective dose when administered alone (tumor growth, death of the mice (euthanasia)), provides an efficient and efficacious treatment regimen, expressed as tumors in regression and prolonged survival of the treated animals (beyond the duration of the experiment). The sub-optimal dose of ADC combined with a conjugate of the invention which has no anti-tumor activity when administered alone, thus provide for an effective treatment option for cancer patients, wherein a relative low dose of the ADC is efficacious. A lower dose of ADC bears the promise of less risk for adverse events, or even no side effects at all. In addition, the stimulatory effect of the conjugate of the invention when the efficacy of the ADC is considered, shows that ADCs which previously have proven to lack efficacy when tumor patient treatment is concerned, may gain renewed attention and value, since ADC efficacy is improved in combination therapy setting, as the current example demonstrated. Reference is made to FIG. 57, summarizing ADCs which were previously investigated in the human clinical setting, but then were retracted from further clinical investigation. Especially the ADCs for which clinical development was terminated due to observed lack of efficacy and/or due to occurrence of unacceptable adverse event are ADCs which may gain renewed value for cancer patients when combined with a conjugate of the invention, such as the cetuximab-saponin tested.

### REFERENCES

1. Weng, A.; Thakur, M.; Beceren-Braun, F.; Bachran, D.; Bachran, C.; Riese, S. B.; Jenett-Siems, K.; Gilabert-Oriol, R.; Melzig, M. F.; Fuchs, H. The toxin component of targeted anti-tumor toxins determines their efficacy increase by saponins. Molecular oncology 2012, 6, 323-332.
2. Yang, Y.; Laval, S.; Yu, B. Chemical synthesis of saponins. Advances in carbohydrate chemistry and biochemistry 2014, 71, 137-226.
3. Gilabert-Oriol, R.; Mergel, K.; Thakur, M.; von Mallinckrodt, B.; Melzig, M. F.; Fuchs, H.; Weng, A. Real-time analysis of membrane permeabilizing effects of oleanane saponins. Bioorganic & medicinal chemistry 2013, 21, 2387-2395.
4. Sama, S.; Jerz, G.; Schmieder, P.; Joseph, J. F.; Melzig, M. F.; Weng, A. Plant derived triterpenes from Gypsophila elegans M. Bieb. enable non-toxic delivery of gene loaded nanoplexes. Journal of Biotechnology 2018, 284, 131-139
5. Kolb, H. C.; Finn, M. G.; Sharpless, K. B. Click Chemistry: Diverse Chemical Function from a Few Good Reactions. Angewandte Chemie 2001, 40, 2004-2021.
6. Bird, R. E.; Hardmann, K. D.; Jacobson, J. W.; Johnson, S.; Kaufman, B. M.; Lee, S. M.; Lee, T.; Pope, S. H.; Riordan, G. S.; Whitlow, M. Single-chain antigen-binding proteins. Science 1988, 242, 423-426.
7. Benjaminsen, R. V.; Mattebjerg, M. A.; Henriksen, J. R.; Moghimi, S. M.; Andresen, T. L. The Possible “Proton Sponge” Effect of Polyethylenimine (PEI) Does Not Include Change in Lysosomal pH. Molecular Therapy 2013, 21, 149-157.
8. Vermeulen, L. M. P.; De Smedt, S. C.; Remaut, K.; Braeckmans, K.; The proton sponge hypothesis: Fable or fact? European Journal of Pharmaceutics and Biopharmaceutics 2018, 129, 184-190.
9. Sonawane, N. D.; Szoka, F. C. Jr.; Verkman A. S. Chloride accumulation and swelling in endosomes enhances DNA transfer by polyamine-DNA polyplexes. The Journal of Biological Chemistry 2003, 278, 44826-44831.
10. Pastuszka, M. K.; Mackay, J. A. Biomolecular engineering of intracellular switches in eukaryotes. Journal of drug delivery science and technology 2010, 20, 163-169.

1.-86. (canceled)

87. A functionalized glycoside moiety having endosomal and/or lysosomal escape enhancing activity and having a molecular structure comprising at least one S moiety and at least one connector moiety L*, with general structure (0):
S-(L*)ₘ structure (0),
wherein the S moiety is a glycoside,
wherein the at least one S moiety is a bisdesmosidic triterpene saponin belonging to the type of a 12,13-dehydrooleanane with an aldehyde function in position 23,
wherein
the at least one L* moiety is at least one W* moiety,
wherein the at least one W* moiety is a scaffold, consisting of, or comprising
poly(amidoamine),
polyethylene glycol, or
a dendron,
wherein if the scaffold comprises primary amine groups, the primary amine groups are blocked or shielded,
wherein the scaffold comprises a single reactive group ‘*’ for coupling a single S moiety, or
wherein the scaffold comprises more than one reactive group ‘*’, each group for coupling a single S moiety,
wherein the at least one S moiety is linked, coupled or bound to the reactive group ‘*’ on the W* moiety through a covalent bond, wherein said covalent bond is a cleavable bond, wherein the scaffold comprises a single binding site for binding a further moiety F, or wherein the scaffold comprises multiple binding sites for binding multiple further moieties F, wherein moiety F is an antibody or EGF,
said binding sites for one or more further moieties F on the scaffold moiety W* being reactive groups on the scaffold moiety W* for provision of a bond with at least one further moiety F,
wherein F moieties are the same or different when the functionalized glycoside moiety encompasses more than one F moiety,
wherein m=1.

88. The functionalized glycoside moiety according to claim 87, wherein if the scaffold comprises primary amine groups, the primary amine groups are blocked or shielded by thiolation or PEGylation.

89. The functionalized glycoside moiety according to claim 87, wherein the single S moiety is a terminal S moiety.

90. The functionalized glycoside moiety according to claim 87, wherein the scaffold comprises or is a polyethylene glycol.

91. The functionalized glycoside moiety of claim 87, wherein the scaffold is a polyethylene glycol.

92. The functionalized glycoside moiety according to claim 87, wherein said cleavable bond is subject to cleavage under any one or more of:
i. acidic conditions;
ii. reductive conditions;
iii. enzymatic conditions; and
iv. light-induced conditions.

92. The functionalized glycoside moiety according to claim 92, wherein the said cleavable bond is subject to cleavage under acidic conditions at a pH of lower than 6.5 such as pH 4.0-6.5, and preferably at a pH ≤5.5.

93. The functionalized glycoside moiety according to claim 87, wherein the cleavable bond is selected from:
v. an imine bond;
vi. a hydrazone bond;
vii. a 1,3-dioxolane bond; and
viii. an ester bond, and/or
ix. wherein the cleavable bond is a disulfide bond or a peptide bond or an amide bond,

94. The functionalized glycoside moiety according to claim 87, wherein the S moiety is saponin SO1861.


SEQUENCE LISTING (1 sequence)

SEQ ID NO: 1 - protein, 18 aa, Artificial Sequence
  Customized peptide
SESDDAMFCDAMDESDSK